(19) United States
(12) Patent Application Publication
(10) Pub. No.: US 2025/0339444 A1
(43) Pub. Date: Nov. 6, 2025

(54) Methods and Compositions for Treating Fatty Liver and Viral Infections

(71) Applicant: The Board of Trustees of the Leland Stanford Junior University, Stanford, CA (US)

(72) Inventors: Jeffrey S. Glenn, Stanford, CA (US); Edward A. Pham, Stanford, CA (US); Menashe Elazar, Stanford, CA (US); Matthew F. Yee, Stanford, CA (US); Mark Smith, Stanford, CA (US); Da Yoon No, Stanford, CA (US)

(21) Appl. No.: 18/862,310

(22) PCT Filed: May 19, 2023
(86) PCT No.: PCT/US2023/022943

§ 371 Date: Nov. 1, 2024

Related U.S. Application Data
(60) Provisional application No. 63/344,467, filed on May 20, 2022.

(51) Int. Cl. A61K 31/55; A61K 31/454; A61K 31/4545; A61K 31/4709; A61K 31/496; A61K 31/5025; A61K 31/519; A61K 31/5377; A61K 31/5386; A61K 31/553; A61K 45/06; A61P 1/16; A61P 31/14; A61P 31/16

(52) U.S. Cl. CPC A61K 31/55; A61K 31/454; A61K 31/4545; A61K 31/4709; A61K 31/496; A61K 31/5025; A61K 31/519; A61K 31/5377; A61K 31/5386; A61K 31/553; A61K 45/06; A61P 1/16; A61P 31/14; A61P 31/16

(57) ABSTRACT

Methods and compositions for treating fatty liver and viral infections are provided. Aspects of the methods includes administering to a subject in need thereof an effective amount of a KxL motif binding agent, e.g., A27, to treat the subject. Also provided are compositions for use in practicing embodiments of the methods.

### INTRODUCTION

Over 100 million individuals in the US have non-alcoholic fatty liver disease (NAFLD), arising due to obesity-associated metabolic dysfunctions. Of these, 10-20% will develop the more aggressive form called non-alcoholic steatohepatitis (NASH) that can ultimately result in liver failure and/or liver cancer. Current therapeutics are inadequate. There remains a big unmet medical need for therapeutics that can prevent and/or reverse NAFLD/NASH.

Viral infections—such as hepatitis B virus (HBV), alphavirus such as Venezuelan equine encephalitis virus (VEEV), coronavirus such as severe respiratory acute respiratory syndrome (SARS)—coronaviruses 1 and 2—continue to be big unmet medical needs.

### CROSS-REFERENCE

This application claims the benefit of U.S. Provisional Patent Application No. 63/344,467, filed May 20, 2022, which application is incorporated herein by reference in its entirety.

### SUMMARY

Methods and compositions for treating fatty liver and viral infections are provided. Aspects of the methods include administering to a subject in need thereof an effective amount of a KxL motif binding agent, e.g., A27, to treat the subject. Also provided are compositions for use in practicing embodiments of the methods.

For example, provided is a method of treatment that includes administering to an individual who has a fatty liver and/or a non-flaviviridae viral infection, a therapeutically effective amount of a compound selected from the group consisting of compounds of Formulae I-VI, or a pharmaceutically acceptable salt thereof:

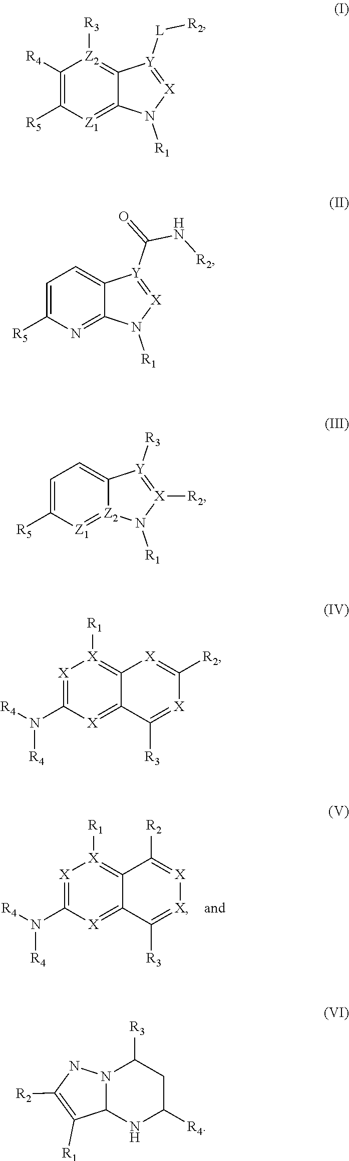

In some embodiments, the individual has a non-flaviviridae viral infection (e.g., an hepadnaviridae virus infection such as HBV, an alphavirus infection such as ONNV, SFV, VEEV, or CHIKV, a coronaviridae infection such as SARS-CoV-2, a paramyxoviridae infection such as RSV, an orthomyxoviridae infection such as influenza, and the like. In some cases, the individual has a hepatitis B virus (HBV), Venezuelan equine encephalitis virus (VEEV), O′nyong nyong virus (ONNV), SARS-CoV-2, Semliki Forest virus (SFV), or chikungunya virus (CHIKV) viral infection. In some cases, the individual has a hepatitis B virus (HBV), Venezuelan equine encephalitis virus (VEEV), SARS-CoV-2, or chikungunya virus (CHIKV) viral infection.

In some embodiments, the compound is co-administered with a second agent, e.g., an antibiotic and/or antiviral agent. In some cases, the compound is administered at a concentration in a range of from at 8 to 100 mg/kg. In some cases, the compound is administered for 3 days or more. In some cases, the compound is A27:

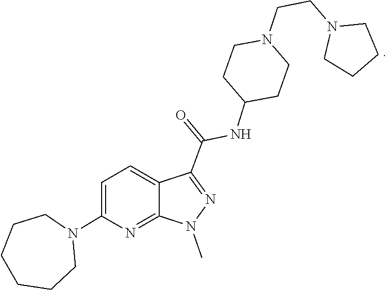 [SMILES: Cn1nc(C(=O)NC2CCN(CCN3CCCC3)CC2)c2ccc(N3CCCCCC3)nc21]

Also provided are novel compounds, e.g., a compound, or pharmaceutically acceptable salt thereof, selected from the group consisting of:

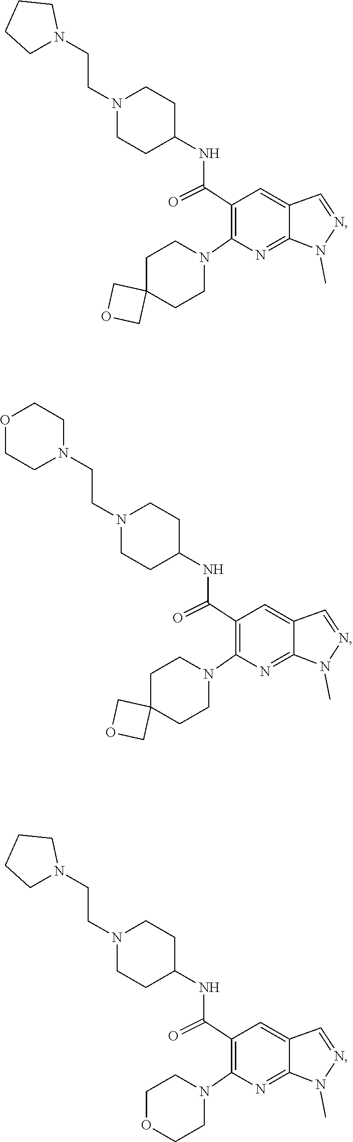

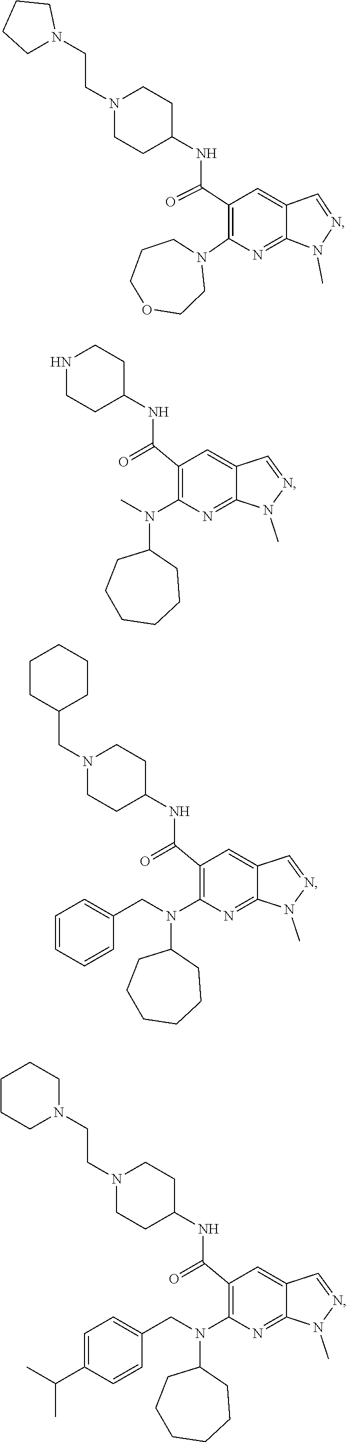

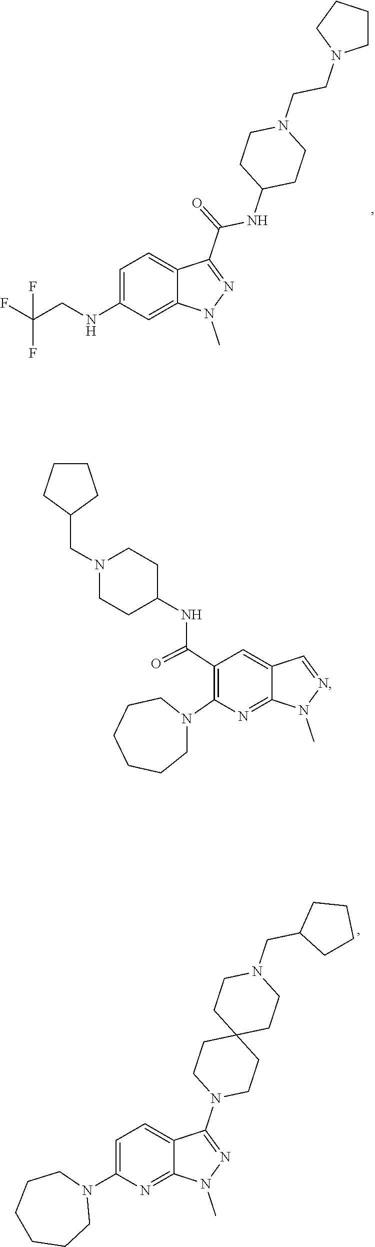 [SMILES: Cn1nc(C(=O)NC2CCN(CCN3CCCC3)CC2)c2ccc(NCC(F)(F)F)cc21 ; Cn1ncc2cc(C(=O)NC3CCN(CC4CCCC4)CC3)c(N3CCCCCC3)nc21 ; Cn1nc(N2CCC3(CCN(CC4CCCC4)CC3)CC2)c2ccc(N3CCCCCC3)nc21]

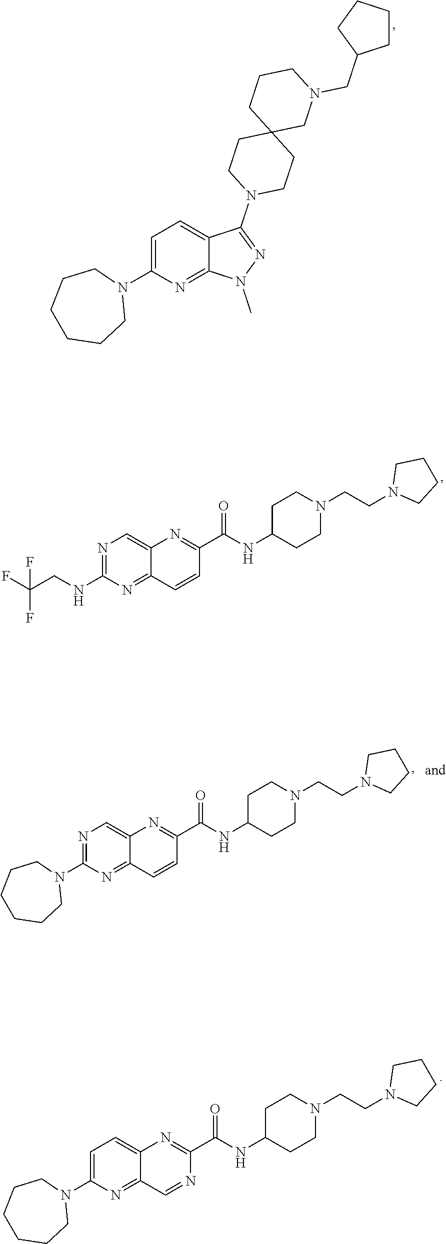 [SMILES: Cn1nc(N2CCC3(CCCN(CC4CCCC4)C3)CC2)c2ccc(N3CCCCCC3)nc21 ; O=C(NC1CCN(CCN2CCCC2)CC1)c1ccc2nc(NCC(F)(F)F)ncc2n1 ; O=C(NC1CCN(CCN2CCCC2)CC1)c1ccc2nc(N3CCCCCC3)ncc2n1 ; O=C(NC1CCN(CCN2CCCC2)CC1)c1ncc2nc(N3CCCCCC3)ccc2n1]

Also provided are compositions, e.g., a composition that includes: (a) a pharmaceutically acceptable carrier, excipient, or diluent; and (b) a compound, or pharmaceutically acceptable salt thereof, selected from the group consisting of:

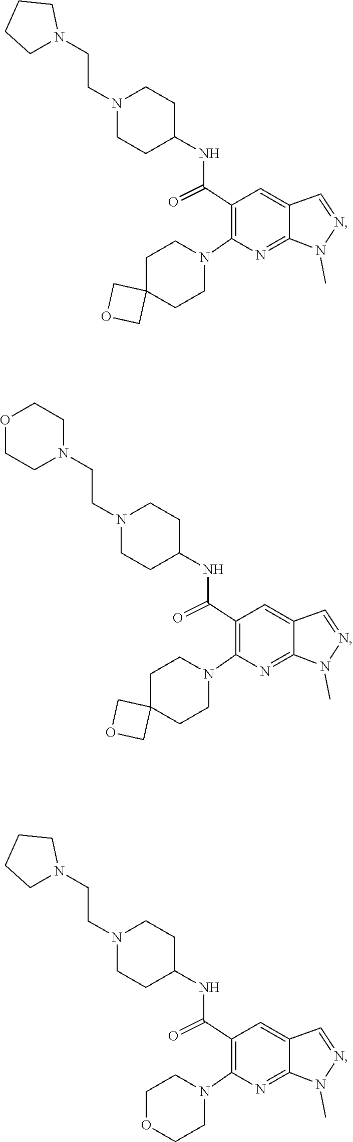

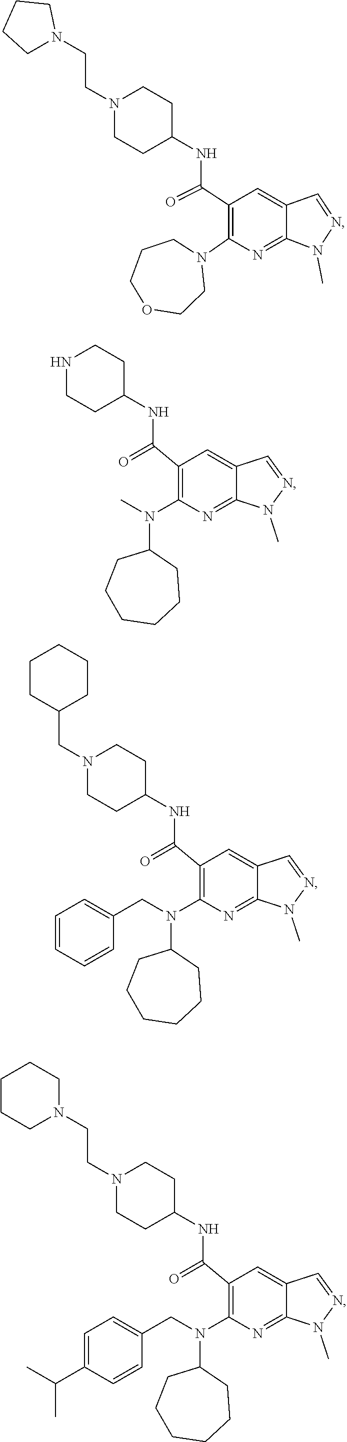

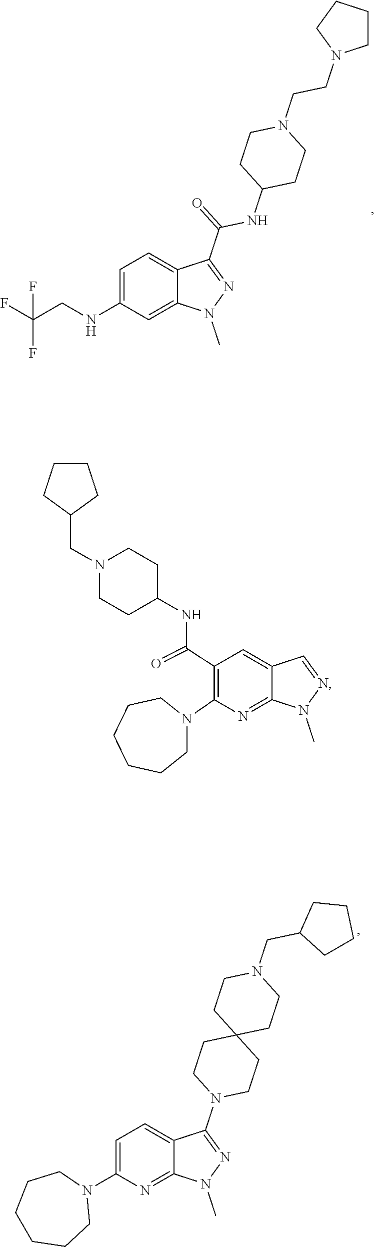 [SMILES: Cn1nc(C(=O)NC2CCN(CCN3CCCC3)CC2)c2ccc(NCC(F)(F)F)cc21 ; Cn1ncc2cc(C(=O)NC3CCN(CC4CCCC4)CC3)c(N3CCCCCC3)nc21 ; Cn1nc(N2CCC3(CCN(CC4CCCC4)CC3)CC2)c2ccc(N3CCCCCC3)nc21]

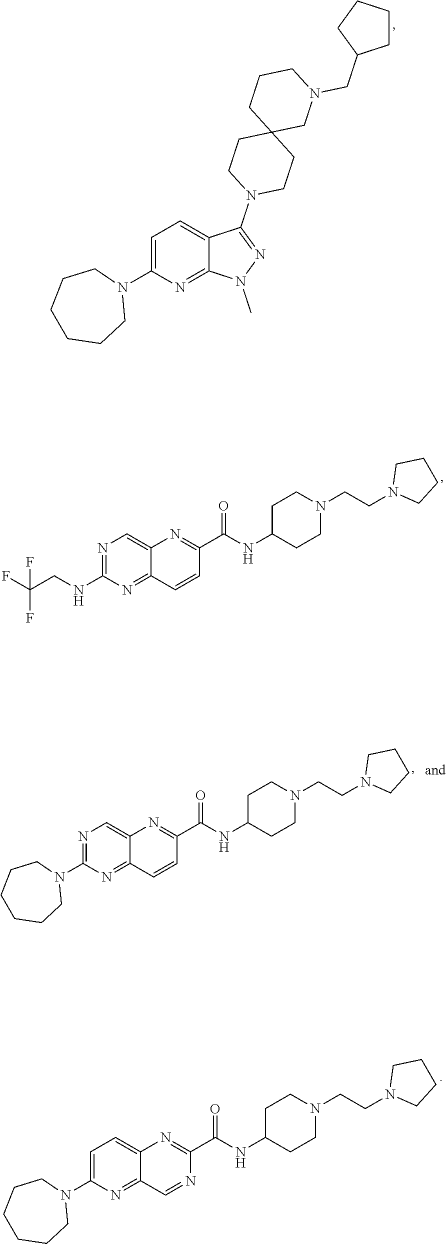 [SMILES: Cn1nc(N2CCC3(CCCN(CC4CCCC4)C3)CC2)c2ccc(N3CCCCCC3)nc21 ; O=C(NC1CCN(CCN2CCCC2)CC1)c1ccc2nc(NCC(F)(F)F)ncc2n1 ; O=C(NC1CCN(CCN2CCCC2)CC1)c1ccc2nc(N3CCCCCC3)ncc2n1 ; O=C(NC1CCN(CCN2CCCC2)CC1)c1ncc2nc(N3CCCCCC3)ccc2n1]

In some cases, the composition is a medicament for the treatment of fatty liver- and in some cases for a viral infection, e.g., a non-flaviviridae viral infection (e.g., an hepadnaviridae virus infection such as HBV, an alphavirus infection such as ONNV, SFV, VEEV, or CHIKV, a coronaviridae infection such as SARS-CoV-2, a paramyxoviridae infection such as RSV, an orthomyxoviridae infection such as influenza, and the like.

### BRIEF DESCRIPTION OF THE FIGURES

FIG. 1: A27 inhibits fat accumulation in a liver cell line cultured in the presence of supplemented fatty acids. HepG2 cells were cultured in media supplemented with oleic acid and palmitic acid to induce steatosis and treated with A27 or vehicle at the indicated concentrations for 3 days.

FIG. 2: A27 inhibits steatosis development in mice on high fat diet as measured by ultrasound. Representative liver ultrasound images of mice on high fat diet treated with vehicle (top panel) or A27 (bottom panel) at 100 mg/kg/day for 4 weeks.

FIG. 3: Molecular modeling of different amphipathic peptides with KxL motif and their interaction with A27.

FIG. 4: A27 reduces HBsAg production in engineered human liver tissue infected with HBV.

FIG. 5: A27 inhibits SARS-CoV-2 in cell culture.

FIG. 6: A27 inhibits VEEV in cell culture.

FIG. 7: Proposed scheme to synthesize A27 derivatives finding use in embodiments of the invention.

FIG. 8: Table showing functional properties for compounds of Formulae I-VI (see, e.g., Examples 2 and 3).

### DEFINITIONS

The term “specific binding” refers to a direct association between two molecules, due to, for example, covalent, electrostatic, hydrophobic, and ionic and/or hydrogen-bond interactions, including interactions such as salt bridges and water bridges. A specific binding member describes a member of a pair of molecules which have binding specificity for one another. The members of a specific binding pair may be naturally derived or wholly or partially synthetically produced. One member of the pair of molecules has an area on its surface, or a cavity, which specifically binds to and is therefore complementary to a particular spatial and polar organization of the other member of the pair of molecules. Thus, the members of the pair have the property of binding specifically to each other. Examples of pairs of specific binding members are antigen-antibody, biotin-avidin, hormone-hormone receptor, receptor-ligand, enzyme-substrate. Specific binding members of a binding pair exhibit high affinity and binding specificity for binding with each other. Typically, affinity between the specific binding members of a pair is characterized by a K_{d} (dissociation constant) of 10⁻⁶ M or less, such as 10⁻⁷ M or less, including 10⁻⁸ M or less, e.g., 10⁻⁹ M or less, 10⁻¹⁰ M or less, 10⁻¹¹ M or less, 10⁻¹² M or less, 10⁻¹³ M or less, 10⁻¹⁴ M or less, including 10⁻¹⁵ M or less. “Affinity” refers to the strength of binding, increased binding affinity being correlated with a lower KD. In an embodiment, affinity is determined by surface plasmon resonance (SPR), e.g., as used by Biacore systems. The affinity of one molecule for another molecule is determined by measuring the binding kinetics of the interaction, e.g., at 25° C. “Affinity” refers to the strength of binding, increased binding affinity being correlated with a lower KD. In an embodiment, affinity is determined by surface plasmon resonance (SPR), e.g., as used by Biacore systems. The affinity of one molecule for another molecule is determined by measuring the binding kinetics of the interaction, e.g., at 25° C.

The methods described herein may include multiple steps. Each step may be performed after a predetermined amount of time has elapsed between steps, as desired. As such, the time between performing each step may be 1 second or more, 10 seconds or more, 30 seconds or more, 60 seconds or more, 5 minutes or more, 10 minutes or more, 60 minutes or more and including 5 hours or more. In certain embodiments, each subsequent step is performed immediately after completion of the previous step. In other embodiments, a step may be performed after an incubation or waiting time after completion of the previous step, e.g., a few minutes to an overnight waiting time.

As used herein, the terms “evaluating”, “determining,” “measuring,” and “assessing,” and “assaying” are used interchangeably and include both quantitative and qualitative determinations.

The term “separating”, as used herein, refers to physical separation of two elements (e.g., by size or affinity, etc.) as well as degradation of one element, leaving the other intact.

### DETAILED DESCRIPTION

Methods and compositions for treating fatty liver and viral infections are provided. Aspects of the methods includes administering to a subject in need thereof an effective amount of a KxL motif binding agent, e.g., A27, to treat the subject. Also provided are compositions for use in practicing embodiments of the methods.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

Methods and compositions for treating fatty liver and viral infections are provided. Aspects of the methods includes administering to a subject in need thereof an effective amount of a KxL motif binding agent, e.g., A27, to treat the subject. Also provided are compositions for use in practicing embodiments of the methods.

By “treatment” it is meant that at least an amelioration of one or more symptoms associated with target condition afflicting the subject is achieved, where amelioration is used in a broad sense to refer to at least a reduction in the magnitude of a parameter, e.g., a symptom associated with the target condition being treated. As such, treatment also includes situations where a pathological condition, or at least symptoms associated therewith, are completely inhibited, e.g., prevented from happening, or stopped, e.g., terminated, such that the adult mammal no longer suffers from the target condition, or at least the symptoms that characterize the impairment. In some instances, “treatment”, “treating” and the like refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” may be any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. Treatment may result in a variety of different physical manifestations, e.g., modulation in gene expression, rejuvenation of tissue or organs, etc. Treatment of ongoing disease, where the treatment stabilizes or reduces the undesirable clinical symptoms of the patient, occurs in some embodiments. Such treatment may be performed prior to complete loss of function in the affected tissues. The subject therapy may be administered during the symptomatic stage of the disease, and in some cases after the symptomatic stage of the disease.

Methods described herein may be employed to treat any type of subject in need of treatment. Subject include mammalian species. Mammalian species that may be treated with the present methods include canines and felines; equines; bovines; ovines; etc., and primates, including humans. The subject methods, compositions, and reagents may also be applied to animal models, including small mammals, e.g., murine, lagomorpha, etc., for example, in experimental investigations.

The terms “recipient”, “individual”, “subject”, “host”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, etc. In some embodiments, the mammal is human. In some embodiments the individual is not human (e.g., a non-human mammal).

The inventors discovered that A27 and derivatives thereof can bind to the KxL motif of ApoB, which plays a role in fatty liver (steatosis, hepatic steatosis). A27 (and derivatives) can be used to reduce steatosis and can therefore be used to treat fatty liver. Thus, in some embodiments an individual to which A27 and/or an A27 derivative is delivered has a fatty liver.

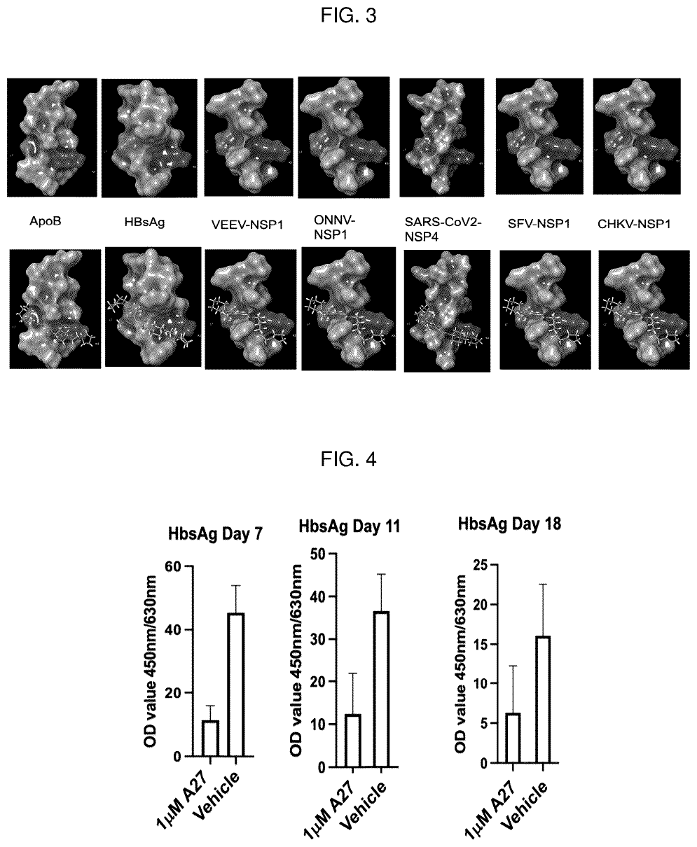

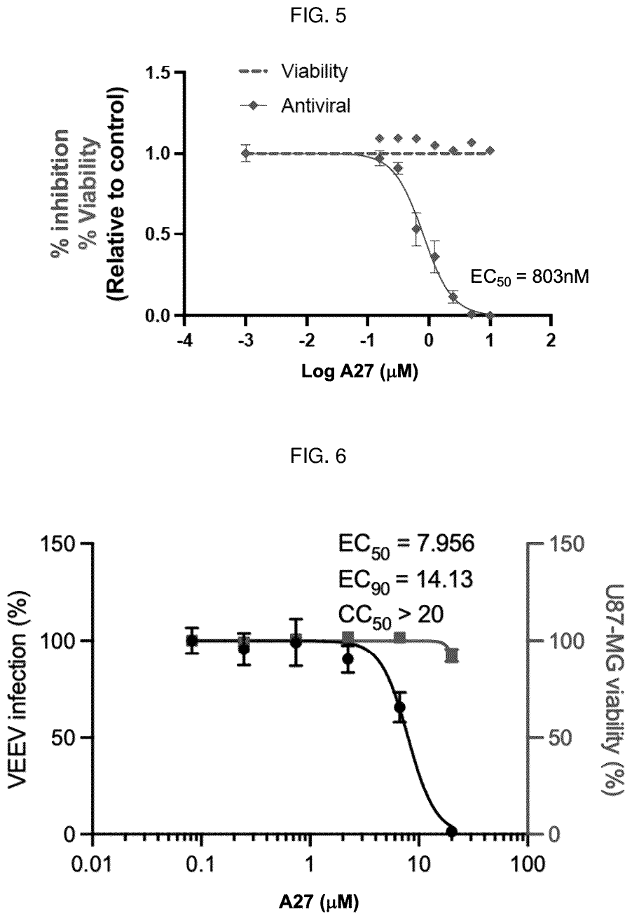

As noted above, the inventors discovered that A27 and derivatives thereof can bind to the KxL motif of ApoB, which they found to also be present in the amphipathic helical domains of viral proteins, including hepatitis B surface antigen (HBsAg), the nonstructural protein (NSP) 1 of Venezuelan equine encephalitis virus (VEEV), NSP1 of O'nyong nyong virus (ONNV), NSP1 of Semliki Forest virus (SFV), NSP1 of chikungunya virus (CHIKV or CHKV), and NSP4 of SARS-CoV-2, (FIG. 3). Thus, the compounds described herein (e.g., A27 and derivatives thereof) can be used to treat viral infections.

In some embodiments the viral infection is not a flaviviridae (e.g., HCV) virus infection (i.e., a non-flaviviridae infection). In some embodiments the viral infection is a Hepadnaviridae virus infection (e.g., hepatitis B virus (HBV)). In some cases the infection is an alphavirus infection (e.g., ONNV, SFV, VEEV, CHIKV, eastern equine encephalomyelitis (EEE), western equine encephalomyelitis (WEE), and the like). In some cases, the infection is a coronaviridae infection (e.g., severe respiratory acute respiratory syndrome (SARS)-CoV-2). In some cases, the infection is a paramyxoviridae infection (e.g., respiratory syncytial virus (RSV)). In some cases, the infection is an Orthomyxoviridae infection (e.g., influenza).

In some cases, the infection is a hepatitis B virus (HBV) infection, a venezuelan equine encephalitis virus (VEEV) infection, or a severe respiratory acute respiratory syndrome (SARS)-CoV-2 infection. In some cases, the infection is an HBV infection, a VEEV infection, a SARS-CoV-2 infection, an ONNV infection, an SFV infection, or a CHIKV infection. In some cases the infection is an alphavirus infection (e.g., ONNV, eastern equine encephalomyelitis (EEE), western equine encephalomyelitis (WEE), VEEV, CHIKV).

As summarized above, embodiments of the methods employ a KxL motif binding agent. For example, small molecules that bind to the KxL motif are of interest. Naturally occurring or synthetic small molecule compounds of interest include numerous chemical classes, such as organic molecules, e.g., small organic compounds having a molecular weight of more than 50 and less than about 2,500 daltons. Candidate agents comprise functional groups for structural interaction with proteins, particularly hydrogen bonding, and typically include at least an amine, carbonyl, hydroxyl or carboxyl group, preferably at least two of the functional chemical groups. The candidate agents may include cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Such molecules may be identified, among other ways, by employing the screening protocols described below.

In some embodiments, the agent is A27 or a derivative thereof (see, e.g., compounds of Formulae I-III, illustrated below). In some in some embodiments, the agent is as disclosed in United States Published Patent Application Publication No. US20120232062A1 (see, e.g., compounds of Formulae I-III); the disclosure of which is herein incorporated by reference.

In some in some embodiments, the agent is

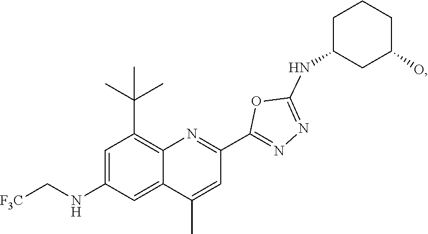

or a derivative thereof (see, e.g., compounds of Formulae IV-V, illustrated below). In some in some embodiments, the agent is as disclosed in international Patent Application Publication No. WO2013090929 (see, e.g., compounds of Formulae IV-V); the disclosure of which is herein incorporated by reference.

### Select Definition of Chemical Terminology

The following terms are referred to in their conventional sense, such as described below. Any undefined terms have their art recognized meanings.

As used herein, the term “alkyl” by itself or as part of another substituent refers to a saturated branched or straight-chain monovalent hydrocarbon radical derived by the removal of one hydrogen atom from a single carbon atom of a parent alkane. Typical alkyl groups include, but are not limited to, methyl; ethyl, propyls such as propan-1-yl or propan-2-yl; and butyls such as butan-1-yl, butan-2-yl, 2-methyl-propan-1-yl or 2-methyl-propan-2-yl. In some embodiments, an alkyl group comprises from 1 to 20 carbon atoms. In other embodiments, an alkyl group comprises from 1 to 10 carbon atoms. In still other embodiments, an alkyl group comprises from 1 to 6 carbon atoms, such as from 1 to 4 carbon atoms.

“Alkanyl” by itself or as part of another substituent refers to a saturated branched, straight-chain or cyclic alkyl radical derived by the removal of one hydrogen atom from a single carbon atom of an alkane. Typical alkanyl groups include, but are not limited to, methanyl; ethanyl; propanyls such as propan-1-yl, propan-2-yl (isopropyl), cyclopropan-1-yl, etc.; butanyls such as butan-1-yl, butan-2-yl (sec-butyl), 2-methyl-propan-1-yl (isobutyl), 2-methyl propan-2-yl (t-butyl), cyclobutan-1-yl, etc.; and the like.

“Alkylene” refers to a branched or unbranched saturated hydrocarbon chain, usually having from 1 to 40 carbon atoms, more usually 1 to 10 carbon atoms and even more usually 1 to 6 carbon atoms. This term is exemplified by groups such as methylene (—CH₂—), ethylene (—CH₂CH₂—), the propylene isomers (e.g., —CH₂CH₂CH₂— and —CH(CH₃)CH₂—) and the like.

“Alkenyl” by itself or as part of another substituent refers to an unsaturated branched, straight-chain or cyclic alkyl radical having at least one carbon-carbon double bond derived by the removal of one hydrogen atom from a single carbon atom of an alkene. The group may be in either the cis or trans conformation about the double bond(s). Typical alkenyl groups include, but are not limited to, ethenyl; propenyls such as prop-1-en-1-yl, prop-1-en-2-yl, prop-2-en-1-yl (allyl), prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like.

“Alkynyl” by itself or as part of another substituent refers to an unsaturated branched, straight-chain or cyclic alkyl radical having at least one carbon-carbon triple bond derived by the removal of one hydrogen atom from a single carbon atom of an alkyne. Typical alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like.

“Acyl” by itself or as part of another substituent refers to a radical —C(O)R³⁰, where R³⁰ is hydrogen, alkyl, cycloalkyl, cycloheteroalkyl, aryl, arylalkyl, heteroalkyl, heteroaryl, heteroarylalkyl as defined herein and substituted versions thereof. Representative examples include, but are not limited to formyl, acetyl, cyclohexylcarbonyl, cyclohexylmethylcarbonyl, benzoyl, benzylcarbonyl, piperonyl, propionyl, succinyl, and malonyl, and the like.

The term “aminoacyl” refers to the group —C(O)NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Alkoxy” by itself or as part of another substituent refers to a radical —OR³¹ where R³¹ represents an alkyl or cycloalkyl group as defined herein. Representative examples include, but are not limited to, methoxy, ethoxy, propoxy, butoxy, cyclohexyloxy and the like.

“Alkoxycarbonyl” by itself or as part of another substituent refers to a radical —C(O)OR³¹ where R³¹ represents an alkyl or cycloalkyl group as defined herein. Representative examples include, but are not limited to, methoxycarbonyl, ethoxycarbonyl, propoxycarbonyl, butoxycarbonyl, cyclohexyloxycarbonyl and the like.

“Aryl” by itself or as part of another substituent refers to a monovalent aromatic hydrocarbon radical derived by the removal of one hydrogen atom from a single carbon atom of an aromatic ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like. In certain embodiments, an aryl group comprises from 6 to 20 carbon atoms. In certain embodiments, an aryl group comprises from 6 to 12 carbon atoms. Examples of an aryl group are phenyl and naphthyl.

“Arylalkyl” by itself or as part of another substituent refers to an acyclic alkyl radical in which one of the hydrogen atoms bonded to a carbon atom, typically a terminal or sp³ carbon atom, is replaced with an aryl group. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylalkenyl and/or arylalkynyl is used. In certain embodiments, an arylalkyl group is (C₇-C₃₀) arylalkyl, e.g., the alkanyl, alkenyl or alkynyl moiety of the arylalkyl group is (C₁-C₁₀) and the aryl moiety is (C₆-C₂₀). In certain embodiments, an arylalkyl group is (C₇-C₂₀) arylalkyl, e.g., the alkanyl, alkenyl or alkynyl moiety of the arylalkyl group is (C₁-C₈) and the aryl moiety is (C₆-C₁₂).

“Arylaryl” by itself or as part of another substituent, refers to a monovalent hydrocarbon group derived by the removal of one hydrogen atom from a single carbon atom of a ring system in which two or more identical or non-identical aromatic ring systems are joined directly together by a single bond, where the number of such direct ring junctions is one less than the number of aromatic ring systems involved. Typical arylaryl groups include, but are not limited to, biphenyl, triphenyl, phenyl-napthyl, binaphthyl, biphenyl-napthyl, and the like. When the number of carbon atoms in an arylaryl group are specified, the numbers refer to the carbon atoms comprising each aromatic ring. For example, (C₅-C₁₄) arylaryl is an arylaryl group in which each aromatic ring comprises from 5 to 14 carbons, e.g., biphenyl, triphenyl, binaphthyl, phenylnapthyl, etc. In certain embodiments, each aromatic ring system of an arylaryl group is independently a (C₅-C₁₄) aromatic. In certain embodiments, each aromatic ring system of an arylaryl group is independently a (C₅-C₁₀) aromatic. In certain embodiments, each aromatic ring system is identical, e.g., biphenyl, triphenyl, binaphthyl, trinaphthyl, etc.

“Cycloalkyl” by itself or as part of another substituent refers to a saturated or unsaturated cyclic alkyl radical. Where a specific level of saturation is intended, the nomenclature “cycloalkanyl” or “cycloalkenyl” is used. Typical cycloalkyl groups include, but are not limited to, groups derived from cyclopropane, cyclobutane, cyclopentane, cyclohexane and the like. In certain embodiments, the cycloalkyl group is (C₃-C₁₀) cycloalkyl. In certain embodiments, the cycloalkyl group is (C₃-C₇) cycloalkyl.

“Cycloheteroalkyl” or “heterocyclyl” by itself or as part of another substituent, refers to a saturated or unsaturated cyclic alkyl radical in which one or more carbon atoms (and any associated hydrogen atoms) are independently replaced with the same or different heteroatom. Typical heteroatoms to replace the carbon atom(s) include, but are not limited to, N, P, O, S, Si, etc. Where a specific level of saturation is intended, the nomenclature “cycloheteroalkanyl” or “cycloheteroalkenyl” is used. Typical cycloheteroalkyl groups include, but are not limited to, groups derived from epoxides, azirines, thiiranes, imidazolidine, morpholine, piperazine, piperidine, pyrazolidine, pyrrolidine, quinuclidine and the like.

“Heteroalkyl, Heteroalkanyl, Heteroalkenyl and Heteroalkynyl” by themselves or as part of another substituent refer to alkyl, alkanyl, alkenyl and alkynyl groups, respectively, in which one or more of the carbon atoms (and any associated hydrogen atoms) are independently replaced with the same or different heteroatomic groups. Typical heteroatomic groups which can be included in these groups include, but are not limited to, —O—, —S—, —S—S—, —O—S—, —NR³⁷R³⁸—, ═N—N═, —N═N—, —N═N—NR³⁹R⁴⁰, —PR⁴¹—, —P(O)₂—, —POR⁴²—, —O—P(O)₂—, —S—O—, —S—(O)—, —SO₂—, —SnR⁴³R⁴⁴— and the like, where R³⁷, R³⁸, R³⁹, R⁴⁰, R⁴¹, R⁴², R⁴³ and R⁴⁴ are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl or substituted heteroarylalkyl.

“Heteroaryl” by itself or as part of another substituent, refers to a monovalent heteroaromatic radical derived by the removal of one hydrogen atom from a single atom of a heteroaromatic ring system. Typical heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, benzodioxole and the like. In certain embodiments, the heteroaryl group is from 5-20 membered heteroaryl. In certain embodiments, the heteroaryl group is from 5-10 membered heteroaryl. In certain embodiments, heteroaryl groups are those derived from thiophene, pyrrole, benzothiophene, benzofuran, indole, pyridine, quinoline, imidazole, oxazole and pyrazine.

“Heteroarylalkyl” by itself or as part of another substituent, refers to an acyclic alkyl radical in which one of the hydrogen atoms bonded to a carbon atom, typically a terminal or sp³ carbon atom, is replaced with a heteroaryl group. Where specific alkyl moieties are intended, the nomenclature heteroarylalkanyl, heteroarylalkenyl and/or heterorylalkynyl is used. In certain embodiments, the heteroarylalkyl group is a 6-30 membered heteroarylalkyl, e.g., the alkanyl, alkenyl or alkynyl moiety of the heteroarylalkyl is 1-10 membered and the heteroaryl moiety is a 5-20-membered heteroaryl. In certain embodiments, the heteroarylalkyl group is 6-20 membered heteroarylalkyl, e.g., the alkanyl, alkenyl or alkynyl moiety of the heteroarylalkyl is 1-8 membered and the heteroaryl moiety is a 5-12-membered heteroaryl.

“Aromatic Ring System” by itself or as part of another substituent, refers to an unsaturated cyclic or polycyclic ring system having a conjugated TT electron system. Specifically included within the definition of “aromatic ring system” are fused ring systems in which one or more of the rings are aromatic and one or more of the rings are saturated or unsaturated, such as, for example, fluorene, indane, indene, phenalene, etc. Typical aromatic ring systems include, but are not limited to, aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like.

“Heteroaromatic Ring System’ by itself or as part of another substituent, refers to an aromatic ring system in which one or more carbon atoms (and any associated hydrogen atoms) are independently replaced with the same or different heteroatom. Typical heteroatoms to replace the carbon atoms include, but are not limited to, N, P, O, S, Si, etc. Specifically included within the definition of “heteroaromatic ring systems” are fused ring systems in which one or more of the rings are aromatic and one or more of the rings are saturated or unsaturated, such as, for example, arsindole, benzodioxan, benzofuran, chromane, chromene, indole, indoline, xanthene, etc. Typical heteroaromatic ring systems include, but are not limited to, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene and the like.

“Substituted” refers to a group in which one or more hydrogen atoms are independently replaced with the same or different substituent(s). Typical substituents include, but are not limited to, alkylenedioxy (such as methylenedioxy), -M, —R⁶⁰, —O, ═O, —OR⁶⁰, —SR⁶⁰, —S⁻, ═S, —NR⁶⁰R⁶¹, ═NR⁶⁰, —CF₃, —CN, —OCN, —SCN, —NO, —NO₂, ═N₂, —N₃, —S(O)₂O⁻, —S(O)₂OH, —S(O)₂R⁶⁰, —OS(O)₂O⁻, —OS(O)₂R⁶⁰, —P(O)(O⁻)₂, —P(O)(OR⁶⁰)(O⁻), —OP(O)(OR⁶⁰)(OR⁶¹), —C(O)R⁶⁰, —C(S)R⁶⁰, —C(O)OR⁶⁰, —C(O)NR⁶⁰R⁶¹, —C(O)O⁻, —C(S)OR⁶⁰, —NR⁶²C(O)NR⁶⁰R⁶¹, —NR⁶²C(S)NR⁶⁰R⁶¹, —NR⁶²C(NR⁶³)NR⁶⁰R⁶¹ and —C(NR⁶²)NR⁶⁰R⁶¹ where M is halogen; R⁶⁰, R⁶¹, R⁶² and R⁶³ are independently hydrogen, alkyl, substituted alkyl, alkoxy, substituted alkoxy, cycloalkyl, substituted cycloalkyl, cycloheteroalkyl, substituted cycloheteroalkyl, aryl, substituted aryl, heteroaryl or substituted heteroaryl, or optionally R⁶⁰ and R⁶¹ together with the nitrogen atom to which they are bonded form a cycloheteroalkyl or substituted cycloheteroalkyl ring; and R⁶⁴ and R⁶⁵ are independently hydrogen, alkyl, substituted alkyl, aryl, cycloalkyl, substituted cycloalkyl, cycloheteroalkyl, substituted cycloheteroalkyl, aryl, substituted aryl, heteroaryl or substituted heteroaryl, or optionally R⁶⁴ and R⁶⁵ together with the nitrogen atom to which they are bonded form a cycloheteroalkyl or substituted cycloheteroalkyl ring. In certain embodiments, substituents include -M, —R⁶⁰, ═O, —OR⁶⁰, —SR⁶⁰, —S⁻, ═S, —NR⁶⁰R⁶¹, ═NR⁶⁰, —CF₃, —CN, —OCN, —SCN, —NO, —NO₂, ═N₂, —N₃, —S(O)₂R⁶⁰, —OS(O)₂O⁻, —OS(O)₂R⁶⁰, —P(O)(O⁻)₂, —P(O)(OR⁶⁰)(O⁻), —OP(O)(OR⁶⁰)(OR⁶¹), —C(O)R⁶⁰, —C(S)R⁶⁰, —C(O)OR⁶⁰, —C(O)NR⁶⁰R⁶¹, —C(O)O⁻, —NR⁶²C(O)NR⁶⁰R⁶¹. In certain embodiments, substituents include -M, —R⁶⁰, ═O, —OR⁶⁰, —SR⁶⁰, —NR⁶⁰R⁶¹, —CF₃, —CN, —NO₂, —S(O)₂R⁶⁰, —P(O)(OR⁶⁰)(O), —OP(O)(OR⁶⁰)(OR⁶¹), —C(O)R⁶⁰, —C(O)OR⁶⁰, —C(O)NR⁶⁰R⁶¹, —C(O)O. In certain embodiments, substituents include -M, —R⁶⁰, ═O, —OR⁶⁰, —SR⁶⁰, —NR⁶⁰R⁶¹, —CF₃, —CN, —NO₂, —S(O)₂R⁶⁰, —OP(O)(OR⁶⁰)(OR⁶¹), —C(O)R⁶⁰, —C(O)O R⁶⁰, —C(O)O⁻, where R⁶⁰, R⁶¹ and R⁶² are as defined above. For example, a substituted group may bear a methylenedioxy substituent or one, two, or three substituents selected from a halogen atom, a (1-4C)alkyl group and a (1-4C)alkoxy group.

### Compounds for Treating Fatty Liver and Viral Infections

As summarized above, aspects of the invention include methods for treating fatty liver and viral infections. In embodiments, compounds of the present disclosure are isolated compounds. In another embodiment, the isolated compounds are at least about 80%, at least about 90% pure, at least about 98% pure, or at least about 99% pure.

In embodiments, “salts” of the compounds of the present disclosure may include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3 (4 hydroxybenzoyl)benzoic acid, cinnamic acid, mandelic acid, methanesulfonic acid, ethanesulfonic acid, 1,2 ethane disulfonic acid, 2 hydroxyethanesulfonic acid, benzenesulfonic acid, 4 chlorobenzenesulfonic acid, 2 naphthalenesulfonic acid, 4 toluenesulfonic acid, camphorsulfonic acid, 4 methylbicyclo[2.2.2] oct 2 ene 1 carboxylic acid, glucoheptonic acid, 3 phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the compound is replaced by a metal ion, e.g., an alkali metal ion, an alkaline earth ion, or an aluminum ion; or coordinates with an organic base such as ethanolamine, diethanolamine, triethanolamine, N methylglucamine and the like.

The term “solvate” as used herein refers to a complex or aggregate formed by one or more molecules of a solute and one or more molecules of a solvent. Such solvates may be crystalline solids having a substantially fixed molar ratio of solute and solvent. Representative solvents include by way of example, water, methanol, ethanol, isopropanol, acetic acid, and the like. When the solvent is water, the solvate formed is a hydrate.

In some embodiments, compounds of interest include a KxL motif binding agent. Compounds for treating fatty liver and viral infections according to certain embodiments include an azaindazole. In some embodiments, the compound is a compound of Formula I:

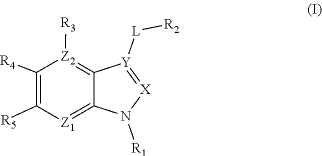

wherein:
X is C or N;
Y is C or N;
Z₁ is C or N;
Z₂ is C or N;
R₁ is hydrogen; C₁-C₆ alkyl; C₁-C₆ alkyl substituted with a substituted or unsubstituted C₃-C₈ cycloalkyl, 5-8 membered heterocyclyl, or a 6 membered aryl group; C₂-C₆ alkenyl; substituted or unsubstituted C₃-C₈ cycloalkyl, —CO—(C₃-C₈ cycloalkyl), —CO—(C₁-C₆ alkyl), —CO-aryl, —CO-heteroaryl, —CO-heterocyclyl, —SO₂—(C₁-C₆ alkyl), or —SO₂—(C₃-C₈ cycloalkyl) group; or R₁ and R₂ together form a 12-25 membered heterocycle, or R₁ and R₅ together form a 12-25 membered heterocycle;
L is a bond, —CONH—, —NH—CO—, substituted or unsubstituted C₁-C₅ alkylene, substituted or unsubstituted C₂-C₅ heteroalkylene, a substituted or unsubstituted 5 membered heteroaryl group, or a substituted or unsubstituted 5-7 membered heterocyclyl, C₅-C₇ cycloalkyl, 5-6 membered heteroaryl, or a 6 membered aryl group; or a combination thereof;
R₂ is —NH₂, —NHR′, —NR′R′, —NHCOR′, —NR′COR′, —NHSO₂R′, —NR′SO₂R′, —NHSO₂NH₂, —NHSO₂NHR′, —NHC(O)NH₂, —NHC(O)NHR′, —N(R′)SO₂NH₂, —N(R)SO₂NHR′, —N(R′)C(O)NH₂, and —N(R′)C(O)NHR′, or a substituted or unsubstituted 5-7 membered heterocyclyl, C₅-C₇ cycloalkyl, 5-6 membered heteroaryl, or a 6 membered aryl group;
R₃, R₄, and R₅ are independently hydrogen, halo, —CN, —OH, —OR′, —NH₂, —NHR′, —NR′R′, —NHCOR′, —NR′COR′, —NHSO₂R′, —NR′SO₂R′, —NHSO₂NH₂, —NHSO₂NHR′, —NHC(O)NH₂, —NHC(O)NHR′, —N(R′)SO₂NH₂, —N(R′)SO₂NHR′, —N(R′)C(O)NH₂, and N(R)C(O)NHR′, —SO₂R′, —SO₂NH₂, SO₂NHR′, SO₂NR′R′, —CONH₂, —CONHR′, —CONR′R′, —CO₂H, —CO₂R′, or a substituted or unsubstituted C₁-C₆ alkyl, C₃-C₈ cycloalkyl, aryl, heteroaryl, or heterocyclyl group; and
R′ is a substituted or unsubstituted C₁-C₆ alkyl, C₂-C₆ alkenyl, C₂-C₆ alkynyl, C₃-C₈ cycloalkyl, aryl, heteroaryl, or heterocyclyl group, or two R′ groups together with the nitrogen atom to which they are bonded form a heterocyclic ring.

In some embodiments, the compound is a compound of Formula Ia:

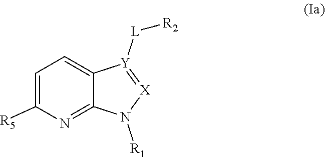

wherein:
X is C or N;
Y is C or N;
wherein L is —CO—NH—, —NH—CO—, —CO—NH—CH₂—, and —CH₂—Y—(CH₂)ₚ— wherein p is 0 or an integer from 1 to 4 and Y is a bond, —O— or —NH—, wherein the right hand side of each L moiety is attached to R₂. In one embodiment, L is —CO—NH—. In one embodiment, L is —NH—CO—. In one embodiment, L is —CO—NH—CH₂—. In one embodiment, L is —CH₂—Y—(CH₂)ₚ— wherein p is 1 or an integer from 1 to 4 and Y is —O— or —NH—. In one embodiment, Y is —O—. In one embodiment, Y is —NH—. In one embodiment, p is 0. In one embodiment, p is 1. In one embodiment, p is 2. In one embodiment, p is 3. In one embodiment, p is 4.

In some embodiments, the compound is a compound of Formula Ib:

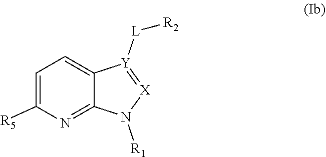

wherein:
X is C or N;
Y is C or N;
R₁ is hydrogen; C₁-C₆ alkyl; C₁-C₆ alkyl substituted with a substituted or unsubstituted C₃-C₈ cycloalkyl, 5-8 membered heterocyclyl, or a 6 membered aryl group; C₂-C₆ alkenyl; substituted or unsubstituted C₃-C₈ cycloalkyl, —CO—(C₃-C₈ cycloalkyl), —CO—(C₁-C₆ alkyl), —CO—(C₃-C₈ cycloheteroalkyl), —CO—(C₁-C₆ heteroalkyl), —SO₂—(C₁-C₆ cycloalkyl), or —SO₂—(C₃-C₈ cycloalkyl) group;
L is a bond, —CONH—, —NH—CO—, substituted or unsubstituted C₁-C₅ alkylene, substituted or unsubstituted C₂-C₅ heteroalkylene, or a combination thereof;
R₂ is a substituted or unsubstituted 5-7 membered heterocyclyl, C₅-C₇ cycloalkyl, 5-6 membered heteroaryl, or a 6 membered aryl group;
R₅ is R₅₁R₅₂N—, R₅₃ (MeSO₂)N—, R₅₄O—, or substituted or unsubstituted C₁-C₆ alkyl;
R₅₁ is hydrogen or C₁-C₃ alkyl;
R₅₂ is C₁-C₃ alkyl, substituted or unsubstituted cycloalkyl, aryl, heterocyclyl, or heteroaryl group, wherein each cycloalkyl, aryl, heterocyclyl, or heteroaryl group contains 6-8 ring atoms, or R₅₁ and R₅₂ together with the nitrogen atom to which they are bonded form a 6, 7, 8, or 9-membered heterocyclyl ring containing up to 3 heteroatoms substituted by a substituted or unsubstituted benzyl, acyl, or sulfonyl group;
R₅₃ is substituted and unsubstituted C₁-C₆ alkyl; and
R₅₄ is hydrogen, substituted or unsubstituted benzyl group, branched C₃-C₈ alkyl, unsubstituted C₅-C₈ cycloalkyl, or C₅-C₈ cycloalkyl substituted with one or more linear or branched C₁-C₄ alkyl groups.

In some embodiments, the compound is a compound of Formula Ic or Id:

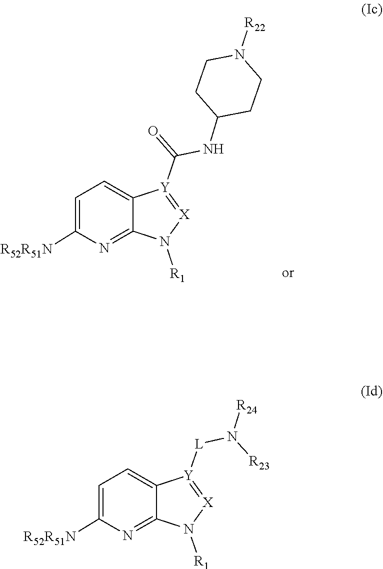

wherein:
X is C or N;
Y is C or N;
R₁, R₂₂, R₂₃, R₂₄, R₅₁, and R₅₂ are defined as in any aspect or embodiment above (or below).

In another embodiment, R₁ is hydrogen, C₁-C₅ alkyl, or —(CH₂)ₖ—R₁₁; k is 1 or 2; and R₁₁ is C₃-C₈ cycloalkyl or a substituted or unsubstituted aryl or heteroaryl group. In another embodiment, R₁ is C₁-C₅ alkyl. In another embodiment, R₁ is hydrogen, methyl, ethyl, propyl, isopropyl, isobutyl, cyclopropylmethyl, or 4-chlorobenzyl. In another embodiment, R₁ is methyl. In another embodiment, R₁ is 4-chlorobenzyl. In another embodiment, R₁ is hydrogen, methyl, or 4-chlorobenzyl. In another embodiment, R₁ is hydrogen or methyl. In another embodiment, R₁ is hydrogen.

In another embodiment, L is —CONH— and the carbon atom of the —CO—NH— is bonded to the azaindazole ring.

In another embodiment, L is a substituted or unsubstituted C₁-C₅ alkylene or C₂-C₅ heteroalkylene group. In another embodiment, L is —(CH₂)ₙ—, —O—(CH₂)ₙ—, or —CH₂—O—(CH₂)ₙ— wherein, the left hand side of the L is bonded to the azaindazole moiety; and n is 1, 2, 3, or 4. In another embodiment, L is —(CH₂)ₙ—. In another embodiment, L is —O—(CH₂)ₙ—. In another embodiment, L is —CH₂—O—(CH₂)ₙ—. In another embodiment, n is 3 or 4. In another embodiment, n is 3 wherein L is —(CH₂)ₙ—. In another embodiment, R₁ is 4-chlorobenzyl, wherein L is —CH₂—O—(CH₂)ₙ— and n is 2 or 3.

In another embodiment, R₂ is substituted or unsubstituted piperidinyl, pyrrolidinyl, piperazinyl, or azepanyl group. In another embodiment, R₂ is a substituted or unsubstituted piperidin-3-yl or piperidin-4-yl group. In another embodiment, the substituted piperidin-4-yl group is:

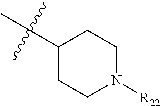

wherein R₂₂ is a substituted or unsubstituted C₂-C₃ alkyl. In another embodiment, R₂₂ is C₂-C₃ alkyl. In another embodiment, R₂₂ is a substituted ethyl group. In another embodiment, R₂₂ is —CH₂CH₂—NR₂₃R₂₄ and R₂₃ and R₂₄ are independently C₁-C₃ alkyl or C₁-C₃ alkyl substituted with a C₃-C₄ cycloalkyl ring, or R₂₃ and R₂₄ together with the nitrogen atom to which they are bonded form a substituted or unsubstituted 5-8 membered heterocyclic ring. Suitable substituents for the 5-8 membered heterocyclic rings include, without limitation, 1 or 2 methyl, hydroxymethyl, methoxymethyl, or hydroxyl groups. In another embodiment, the 5-8 membered heterocyclic ring is a pyrrolidinyl, piperidinyl, or azepanyl ring, which is substituted or unsubstituted. Within this embodiment, in one embodiment, L is —CO—NH—, wherein the NH moiety is bonded to the piperidinyl moiety. In another embodiment, R₂ is —NR₂₃R₂₄ and R₂₃ and R₂₄ are independently C₁-C₃ alkyl or C₁-C₃ alkyl substituted with a C₃-C₄ cycloalkyl ring, or R₂₃ and R₂₄ together with the nitrogen atom to which they are bonded form a substituted or unsubstituted 5-8 membered heterocyclic ring. Suitable substituents for the 5-8 membered heterocyclic rings include, without limitation, 1 or 2 methyl, hydroxymethyl, methoxymethyl, or hydroxyl groups. In another embodiment, the 5-8 membered heterocyclic ring is a pyrrolidinyl, piperidinyl, or azepanyl ring, which is substituted or unsubstituted. Within this embodiment, in one embodiment, L is —(CH₂)ₙ—, —O—(CH₂)ₙ—, or —CH₂—O—(CH₂)ₙ— wherein, the left hand side of the L is bonded to the azaindazole moiety and n is 1, 2, 3, or 4.

In other embodiments, R₂ may be a 4-piperidinyl group that is:

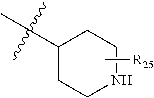

wherein R₂₅ is H or a substituent that is substituted or unsubstituted C₁-C₃ alkyl substituting a carbon or the nitrogen atom.

In some embodiments, the compound is a compound of Formula II:

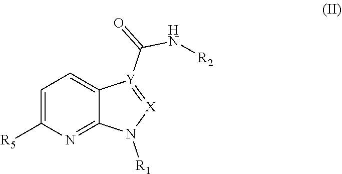

wherein:
X is C or N;
Y is C or N;
R₁ is hydrogen, branched or linear C₁-C₅ alkyl, C₂-C₁₅ alkenyl, unsubstituted or substituted cycloalkyl, —CO-(cycloalkyl), —SO₂-(cycloalkyl) group, or —(CH₂)ₙ— R₁₁, or R₅ and R₁ together form a 12-18 membered heterocycle; n is 1 or 2;
R₂ is substituted or unsubstituted piperidinyl, 4-pyridyl, pyrrolidinyl, piperazinyl, benzyl, substituted phenyl, or pirazolyl group; R₅ is R₅₁R₅₂N— or R₅₄O—;
R₅₁ is H or substituted or unsubstituted C₁-C₃ alkyl; R₅₂ is C₆-C₈ cycloalkyl, substituted or unsubstituted linear C₁-C₃ alkyl, or branched C₄-C₅ alkyl or R₅₁ and R₅₂ together with the nitrogen atom to which they are bonded form a 6, 7, 8, or 9-membered heterocyclyl ring containing up to 3 heteroatoms optionally substituted, other than the azaindazole moiety to which it is already attached, by a substituted or unsubstituted benzyl acyl, or sulfonyl group; R₅₄ is H, substituted or unsubstituted benzyl group, branched C₃-C₈ alkyl, unsubstituted C₅-C₈ cycloalkyl, or C₅-C₈ cycloalkyl substituted with one or more linear or branched C₁-C₄ alkyl groups; R₁₁ is C₅-C₈ cycloalkyl or substituted or unsubstituted aryl, or substituted or unsubstituted heteroaryl. In various embodiments, the compound of Formula II can have R₁, R₂, and R₃ groups as defined below.

In another embodiment of Formula II, R₁ is C₁-C₃ alkyl. In one embodiment, R₁ is methyl. In one embodiment, Ru is cyclohexyl. In one embodiment, R₁₁ is halo substituted phenyl. In one embodiment, R₁₁ is 2-chlorophenyl or 3-chlorophenyl.

In another embodiment of Formula II, R₁ is substituted or unsubstituted 4-piperidinyl or 3-piperidinyl group. In various embodiments, R₂ is a 4-piperidinyl group that is:

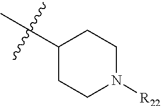

wherein R₂₂ is a substituted or unsubstituted C₂-C₃ alkyl. In another embodiment, R₂₂ is C₂-C₃ alkyl. In another embodiment, R₂₂ is a substituted ethyl group. In another embodiment, R₂₂ is —CH₂CH₂—NR₂₃R₂₄ and R₂₃ and R₂₄ are independently C₁-C₃ alkyl or C₁-C₃ alkyl substituted with a C₃-C₄ cycloalkyl ring, or R₂₃ and R₂₄ together with the nitrogen atom to which they are bonded form a substituted or unsubstituted 5-8 membered heterocyclic ring. Suitable substituents for the 5-8 membered heterocyclic rings include, without limitation, 1 or 2 methyl, hydroxymethyl, methoxymethyl, or hydroxyl groups. In another embodiment, the 5-8 membered heterocyclic ring is a pyrrolidinyl, piperidinyl, or azepanyl ring, which is substituted or unsubstituted. Within this embodiment, in one embodiment, L is —CO—NH—, wherein the NH moiety is bonded to the piperidinyl moiety. In another embodiment, R₂ is —NR₂₃R₂₄ and R₂₃ and R₂₄ are independently C₁-C₃ alkyl or C₁-C₃ alkyl substituted with a C₃-C₄ cycloalkyl ring, or R₂₃ and R₂₄ together with the nitrogen atom to which they are bonded form a substituted or unsubstituted 5-8 membered heterocyclic ring. Suitable substituents for the 5-8 membered heterocyclic rings include, without limitation, 1 or 2 methyl, hydroxymethyl, methoxymethyl, or hydroxyl groups. In another embodiment, the 5-8 membered heterocyclic ring is a pyrrolidinyl, piperidinyl, or azepanyl ring, which is substituted or unsubstituted. Within this embodiment, in one embodiment, L is —(CH₂)ₙ—, —O—(CH₂)ₙ—, or —CH₂—O—(CH₂)ₙ— wherein, the left hand side of the L is bonded to the azaindazole moiety and n is 1, 2, 3, or 4.

In other embodiments, R₂ may be a 4-piperidinyl group that is:

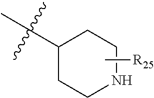

wherein R₂₅ is H or a substituent that is substituted or unsubstituted C₁-C₃ alkyl substituting a carbon or the nitrogen atom.

In some embodiments, the compound is a compound selected from:

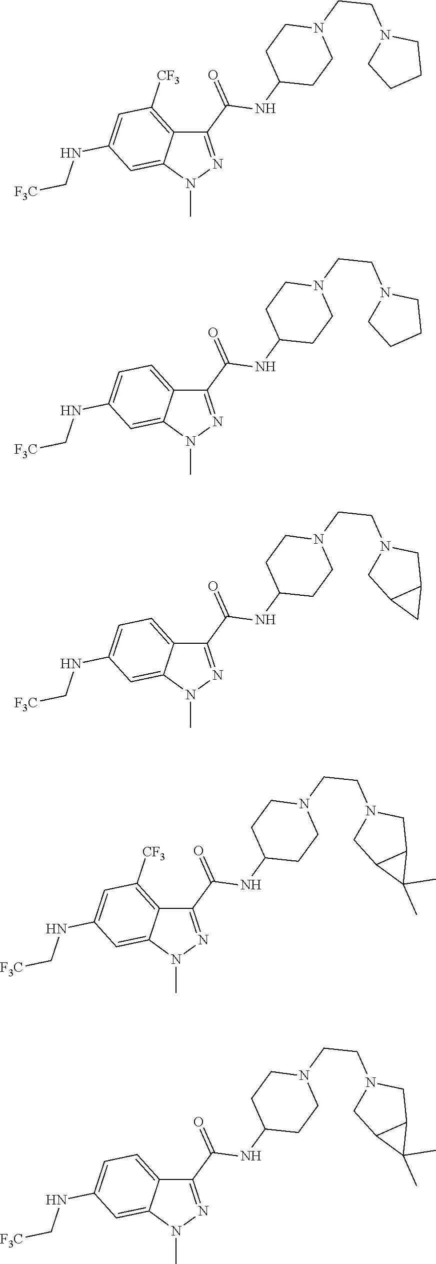 [SMILES: Cn1nc(C(=O)NC2CCN(CCN3CCCC3)CC2)c2c(C(F)(F)F)cc(NCC(F)(F)F)cc21 ; Cn1nc(C(=O)NC2CCN(CCN3CCCC3)CC2)c2ccc(NCC(F)(F)F)cc21 ; Cn1nc(C(=O)NC2CCN(CCN3CC4CC4C3)CC2)c2ccc(NCC(F)(F)F)cc21 ; Cn1nc(C(=O)NC2CCN(CCN3CC4C(C3)C4(C)C)CC2)c2c(C(F)(F)F)cc(NCC(F)(F)F)cc21 ; Cn1nc(C(=O)NC2CCN(CCN3CC4C(C3)C4(C)C)CC2)c2ccc(NCC(F)(F)F)cc21]

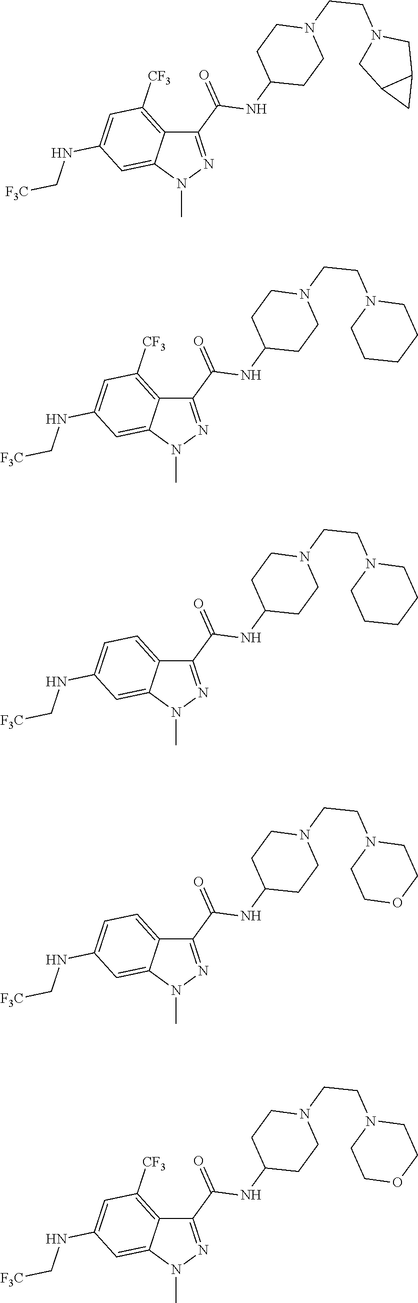 [SMILES: Cn1nc(C(=O)NC2CCN(CCN3CC4CC4C3)CC2)c2c(C(F)(F)F)cc(NCC(F)(F)F)cc21 ; Cn1nc(C(=O)NC2CCN(CCN3CCCCC3)CC2)c2c(C(F)(F)F)cc(NCC(F)(F)F)cc21 ; Cn1nc(C(=O)NC2CCN(CCN3CCCCC3)CC2)c2ccc(NCC(F)(F)F)cc21 ; Cn1nc(C(=O)NC2CCN(CCN3CCOCC3)CC2)c2ccc(NCC(F)(F)F)cc21 ; Cn1nc(C(=O)NC2CCN(CCN3CCOCC3)CC2)c2c(C(F)(F)F)cc(NCC(F)(F)F)cc21]

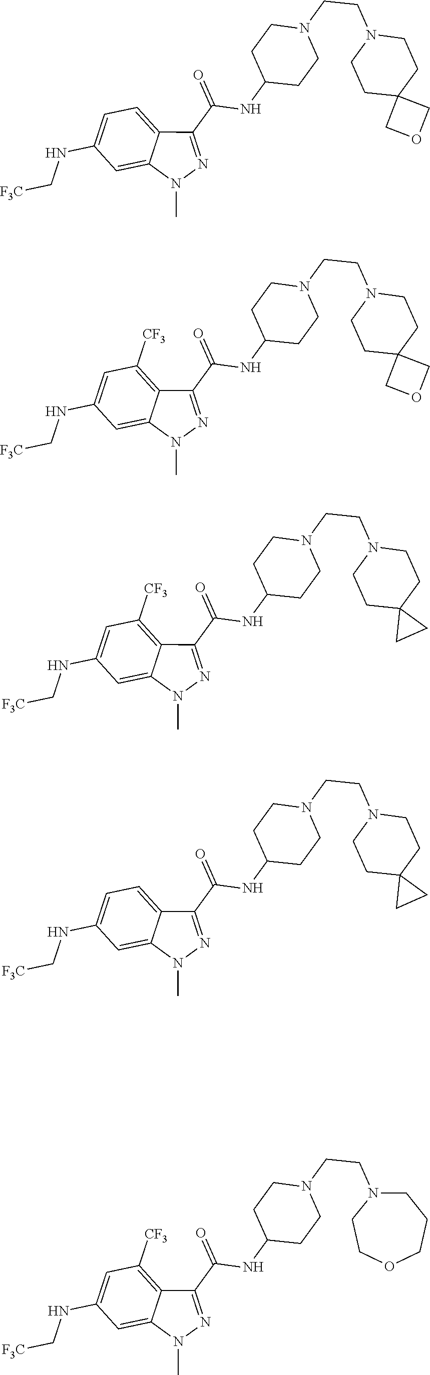 [SMILES: Cn1nc(C(=O)NC2CCN(CCN3CCC4(CC3)COC4)CC2)c2ccc(NCC(F)(F)F)cc21 ; Cn1nc(C(=O)NC2CCN(CCN3CCC4(CC3)COC4)CC2)c2c(C(F)(F)F)cc(NCC(F)(F)F)cc21 ; Cn1nc(C(=O)NC2CCN(CCN3CCC4(CC3)CC4)CC2)c2c(C(F)(F)F)cc(NCC(F)(F)F)cc21 ; Cn1nc(C(=O)NC2CCN(CCN3CCC4(CC3)CC4)CC2)c2ccc(NCC(F)(F)F)cc21 ; Cn1nc(C(=O)NC2CCN(CCN3CCCOCC3)CC2)c2c(C(F)(F)F)cc(NCC(F)(F)F)cc21]

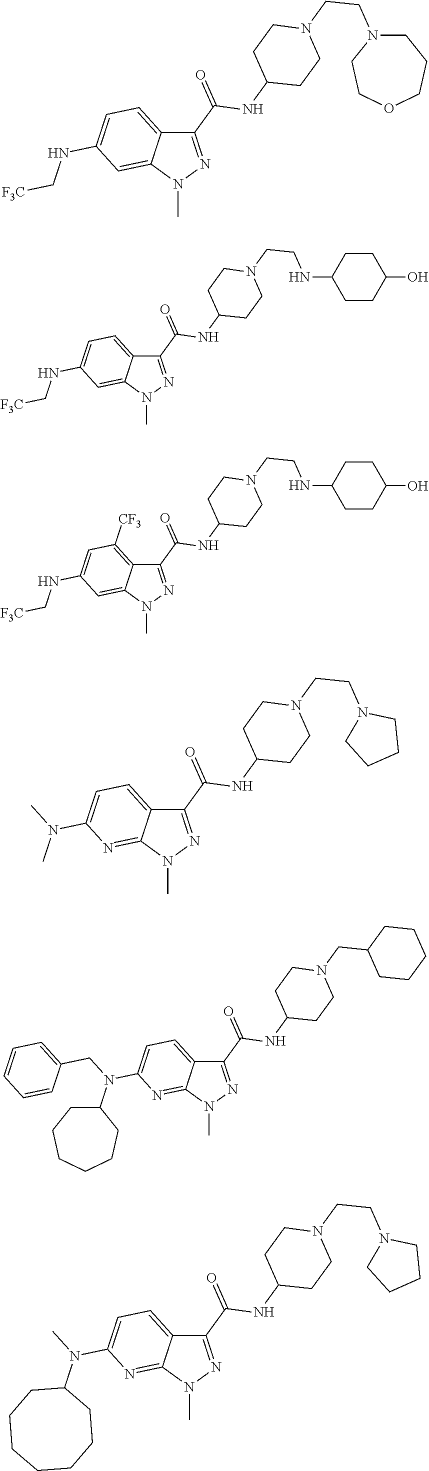 [SMILES: Cn1nc(C(=O)NC2CCN(CCN3CCCOCC3)CC2)c2ccc(NCC(F)(F)F)cc21 ; Cn1nc(C(=O)NC2CCN(CCNC3CCC(O)CC3)CC2)c2ccc(NCC(F)(F)F)cc21 ; Cn1nc(C(=O)NC2CCN(CCNC3CCC(O)CC3)CC2)c2c(C(F)(F)F)cc(NCC(F)(F)F)cc21 ; CN(C)c1ccc2c(C(=O)NC3CCN(CCN4CCCC4)CC3)nn(C)c2n1 ; Cn1nc(C(=O)NC2CCN(CC3CCCCC3)CC2)c2ccc(N(Cc3ccccc3)C3CCCCCC3)nc21 ; CN(c1ccc2c(C(=O)NC3CCN(CCN4CCCC4)CC3)nn(C)c2n1)C1CCCCCCC1]

In some embodiments, the compound is a compound of Formula IIa:

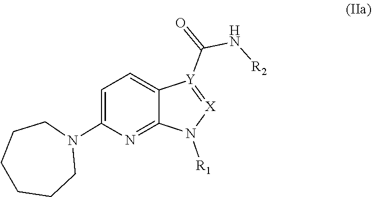

wherein:
X is C or N;
Y is C or N;
R₁ is hydrogen; C₁-C₆ alkyl; C₁-C₆ alkyl substituted with a substituted or unsubstituted C₃-C₈ cycloalkyl, 5-8 membered heterocyclyl, or a 6 membered aryl group; C₂-C₆ alkenyl; substituted or unsubstituted C₃-C₈ cycloalkyl, —CO—(C₃-C₈ cycloalkyl), —CO—(C₁-C₆ alkyl), —CO-aryl, —CO-heteroaryl, —CO-heterocyclyl, —SO₂—(C₁-C₆ alkyl), or —SO₂ (C₃-C₈ cycloalkyl) group; or R₁ and R₂ together form a 12-25 membered heterocycle, or R₁ and R₅ together form a 12-25 membered heterocycle;
R₂ is —NH₂, —NHR′, —NR′R′, —NHCOR′, —NR′COR′, —NHSO₂R′, —NR′SO₂R′, —NHSO₂NH₂, —NHSO₂NHR′, —NHC(O)NH₂, —NHC(O)NHR′, —N(R′)SO₂NH₂, —N(R)SO₂NHR′, —N(R′)C(O)NH₂, and —N(R′)C(O)NHR′, or a substituted or unsubstituted 5-7 membered heterocyclyl, C₅-C₇ cycloalkyl, 5-6 membered heteroaryl, or a 6 membered aryl group.

In some embodiments, the compound is a compound of Formula IIb:

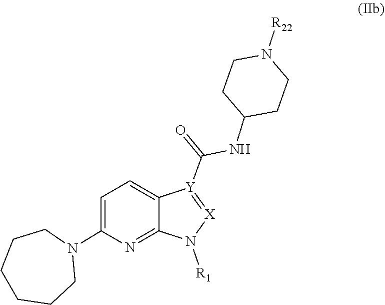

wherein:
X is C or N;
Y is C or N;
R₁ is hydrogen; C₁-C₆ alkyl; C₁-C₆ alkyl substituted with a substituted or unsubstituted C₃-C₈ cycloalkyl, 5-8 membered heterocyclyl, or a 6 membered aryl group; C₂-C₆ alkenyl; substituted or unsubstituted C₃-C₈ cycloalkyl, —CO—(C₃-C₈ cycloalkyl), —CO—(C₁-C₆ alkyl), —CO-aryl, —CO-heteroaryl, —CO-heterocyclyl, —SO₂—(C₁-C₆ alkyl), or —SO₂—(C₃-C₈ cycloalkyl) group; or R₁ and R₂ together form a 12-25 membered heterocycle, or R₁ and R₅ together form a 12-25 membered heterocycle;
wherein R₂₂ is hydrogen, a substituted or unsubstituted C₂-C₃ alkyl. In another embodiment, R₂₂ is C₂-C₃ alkyl. In another embodiment, R₂₂ is a substituted ethyl group. In another embodiment, R₂₂ is —CH₂CH₂—NR₂₃R₂₄ and R₂₃ and R₂₄ are independently C₁-C₃ alkyl or C₁-C₃ alkyl substituted with a C₃-C₄ cycloalkyl ring, or R₂₃ and R₂₄ together with the nitrogen atom to which they are bonded form a substituted or unsubstituted 5-8 membered heterocyclic ring. Suitable substituents for the 5-8 membered heterocyclic rings include, without limitation, 1 or 2 methyl, hydroxymethyl, methoxymethyl, or hydroxyl groups. In another embodiment, the 5-8 membered heterocyclic ring is a pyrrolidinyl, piperidinyl, or azepanyl ring, which is substituted or unsubstituted. Within this embodiment, in one embodiment, L is —CO—NH—, wherein the NH moiety is bonded to the piperidinyl moiety. In another embodiment, R₂ is —NR₂₃R₂₄ and R₂₃ and R₂₄ are independently C₁-C₃ alkyl or C₁-C₃ alkyl substituted with a C₃-C₄ cycloalkyl ring, or R₂₃ and R₂₄ together with the nitrogen atom to which they are bonded form a substituted or unsubstituted 5-8 membered heterocyclic ring. Suitable substituents for the 5-8 membered heterocyclic rings include, without limitation, 1 or 2 methyl, hydroxymethyl, methoxymethyl, or hydroxyl groups. In another embodiment, the 5-8 membered heterocyclic ring is a pyrrolidinyl, piperidinyl, or azepanyl ring, which is substituted or unsubstituted. Within this embodiment, in one embodiment, L is —(CH₂)ₙ—, —O—(CH₂)ₙ—, or —CH₂—O—(CH₂)ₙ— wherein, the left hand side of the L is bonded to the azaindazole moiety and n is 1, 2, 3, or 4.

In some embodiments, the compound is a compound of Formula IIc:

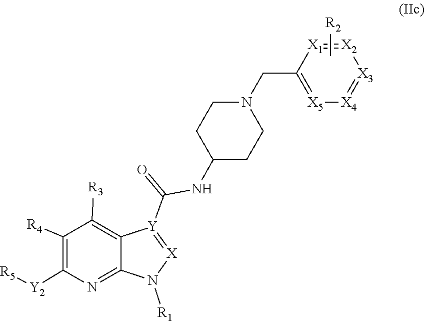

wherein:
X is C or N;
Y is C or N;
X₁, X₂, X₃, X₄ and X₅ are independently selected from CH or N;
Y₂ is selected from O or NR′;
R₁ is hydrogen; C₁-C₆ alkyl; C₁-C₆ alkyl substituted with a substituted or unsubstituted C₃-C₈ cycloalkyl, 5-8 membered heterocyclyl, or a 6 membered aryl group; C₂-C₆ alkenyl; substituted or unsubstituted C₃-C₈ cycloalkyl, —CO—(C₃-C₈ cycloalkyl), —CO—(C₁-C₆ alkyl), —CO-aryl, —CO-heteroaryl, —CO-heterocyclyl, —SO₂—(C₁-C₆ alkyl), or —SO₂—(C₃-C₈ cycloalkyl) group
R₂ is hydrogen; C₁-C₆ alkyl; C₁-C₆ alkyl substituted with a substituted or unsubstituted C₃-C₈ cycloalkyl, 5-8 membered heterocyclyl, or a 6 membered aryl group; C₂-C₆ alkenyl; substituted or unsubstituted C₃-C₈ cycloalkyl, —CO—(C₃-C₈ cycloalkyl), —CO—(C₁-C₆ alkyl), —CO-aryl, —CO-heteroaryl, —CO-heterocyclyl, —SO₂—(C₁-C₆ alkyl), or —SO₂—(C₃-C₈ cycloalkyl) group.
R₃, R₄, and R₅ are independently hydrogen, hydrogen; C₁-C₆ alkyl; substituted C₁-C₆ alkyl, CF₃, halo, —OH, —OR′, —NH₂, —NHR′, —NR′R′, —NHCOR′, —NR′COR′, —NHSO₂R′, —NR′SO₂R′, —NHSO₂NH₂, —NHSO₂NHR′, —NHC(O)NH₂, —NHC(O)NHR′, —N(R′)SO₂NH₂, —N(R′)SO₂NHR′, —N(R′)C(O)NH₂, and N(R)C(O)NHR′, —SO₂R′, —SO₂NH₂, SO₂NHR′, SO₂NR′R′, —CONH₂, —CONHR′, —CONR′R′, —CO₂H, —CO₂R′, or a substituted or unsubstituted C₁-C₆ alkyl, C₃-C₈ cycloalkyl, aryl, heteroaryl, or heterocyclyl group or together with Y₂ form a substituted or unsubstituted C₃-C₈ cycloalkyl, 5-8 membered substituted or unsubstituted heterocyclyl, a substituted or unsubstituted C₃-C₈ aryl or a 5-8 membered substituted or unsubstituted heteroheteroaryl; and
R′ is a substituted or unsubstituted C₁-C₆ alkyl, C₂-C₆ alkenyl, C₂-C₆ alkynyl, C₃-C₈ cycloalkyl, aryl, heteroaryl, or heterocyclyl group, or two R′ groups together with the nitrogen atom to which they are bonded form a heterocyclic ring.

In some embodiments, the compound is a compound selected from:

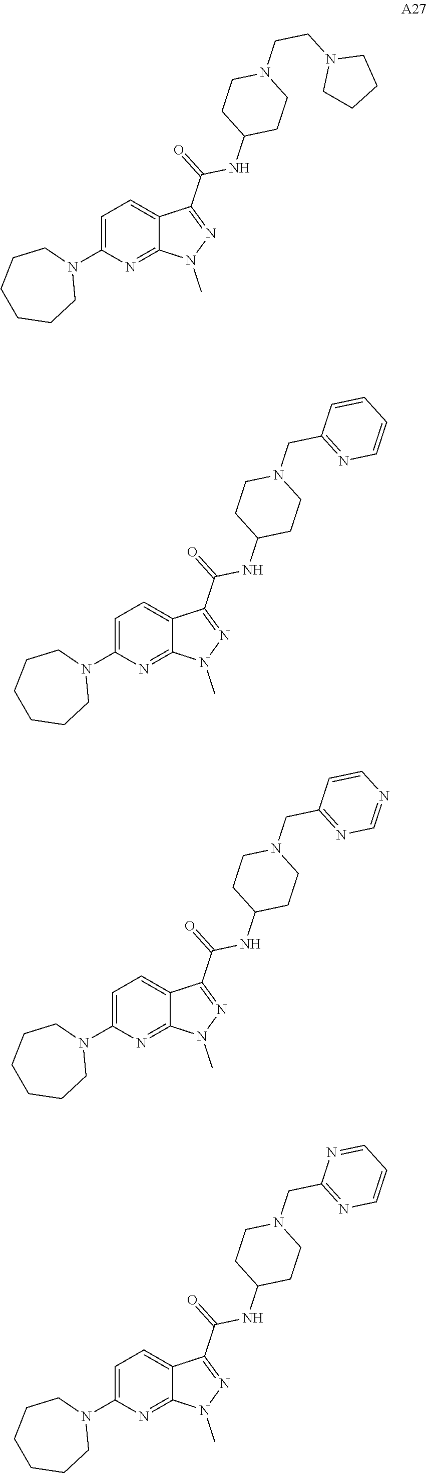 [SMILES: Cn1nc(C(=O)NC2CCN(CCN3CCCC3)CC2)c2ccc(N3CCCCCC3)nc21 ; Cn1nc(C(=O)NC2CCN(Cc3ccccn3)CC2)c2ccc(N3CCCCCC3)nc21 ; Cn1nc(C(=O)NC2CCN(Cc3ccncn3)CC2)c2ccc(N3CCCCCC3)nc21 ; Cn1nc(C(=O)NC2CCN(Cc3ncccn3)CC2)c2ccc(N3CCCCCC3)nc21]

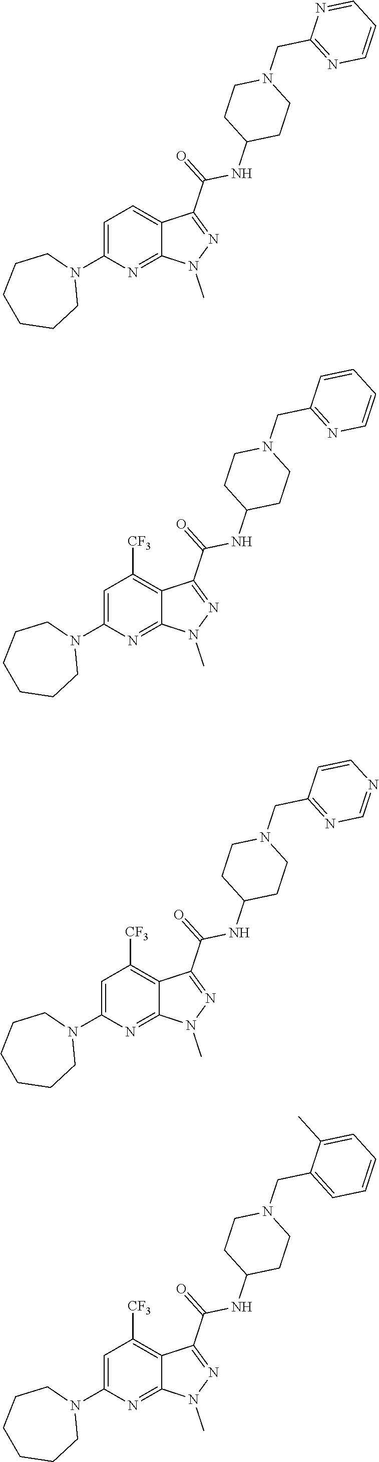 [SMILES: Cn1nc(C(=O)NC2CCN(Cc3ncccn3)CC2)c2ccc(N3CCCCCC3)nc21 ; Cn1nc(C(=O)NC2CCN(Cc3ccccn3)CC2)c2c(C(F)(F)F)cc(N3CCCCCC3)nc21 ; Cn1nc(C(=O)NC2CCN(Cc3ccncn3)CC2)c2c(C(F)(F)F)cc(N3CCCCCC3)nc21 ; Cc1ccccc1CN1CCC(NC(=O)c2nn(C)c3nc(N4CCCCCC4)cc(C(F)(F)F)c23)CC1]

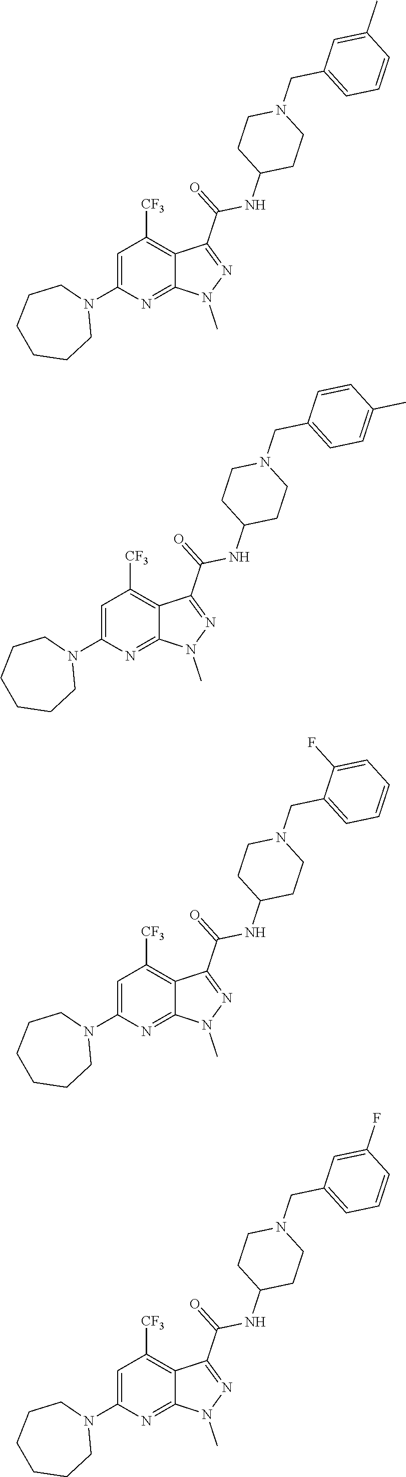 [SMILES: Cc1cccc(CN2CCC(NC(=O)c3nn(C)c4nc(N5CCCCCC5)cc(C(F)(F)F)c34)CC2)c1 ; Cc1ccc(CN2CCC(NC(=O)c3nn(C)c4nc(N5CCCCCC5)cc(C(F)(F)F)c34)CC2)cc1 ; Cn1nc(C(=O)NC2CCN(Cc3ccccc3F)CC2)c2c(C(F)(F)F)cc(N3CCCCCC3)nc21 ; Cn1nc(C(=O)NC2CCN(Cc3cccc(F)c3)CC2)c2c(C(F)(F)F)cc(N3CCCCCC3)nc21]

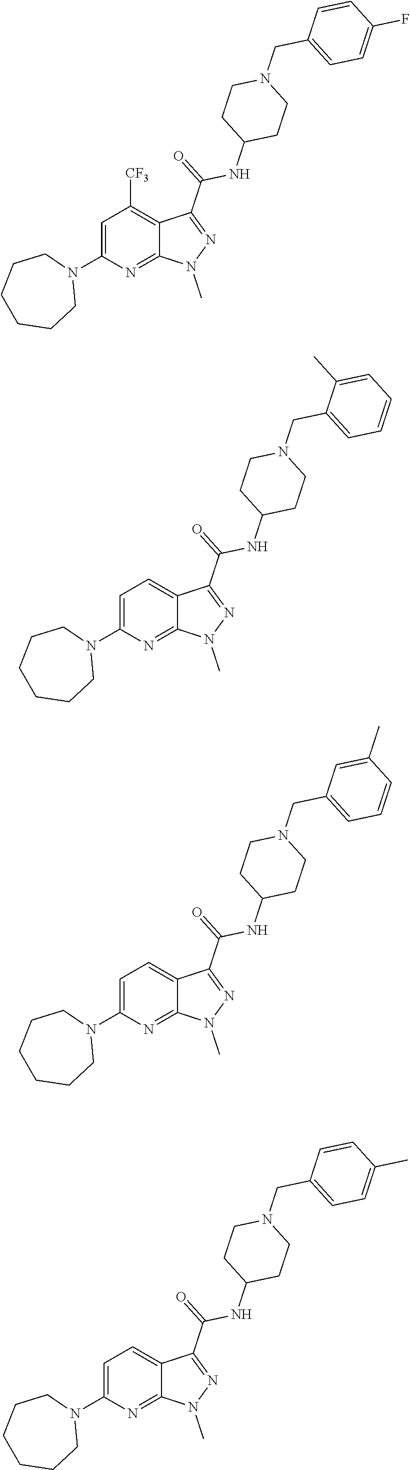 [SMILES: Cn1nc(C(=O)NC2CCN(Cc3ccc(F)cc3)CC2)c2c(C(F)(F)F)cc(N3CCCCCC3)nc21 ; Cc1ccccc1CN1CCC(NC(=O)c2nn(C)c3nc(N4CCCCCC4)ccc23)CC1 ; Cc1cccc(CN2CCC(NC(=O)c3nn(C)c4nc(N5CCCCCC5)ccc34)CC2)c1 ; Cc1ccc(CN2CCC(NC(=O)c3nn(C)c4nc(N5CCCCCC5)ccc34)CC2)cc1]

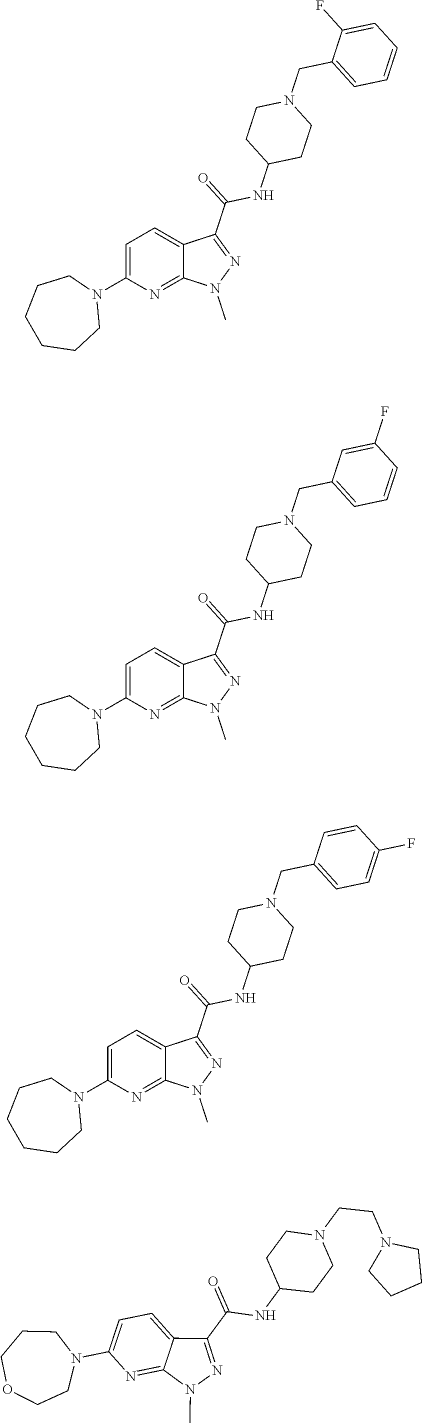 [SMILES: Cn1nc(C(=O)NC2CCN(Cc3ccccc3F)CC2)c2ccc(N3CCCCCC3)nc21 ; Cn1nc(C(=O)NC2CCN(Cc3cccc(F)c3)CC2)c2ccc(N3CCCCCC3)nc21 ; Cn1nc(C(=O)NC2CCN(Cc3ccc(F)cc3)CC2)c2ccc(N3CCCCCC3)nc21 ; Cn1nc(C(=O)NC2CCN(CCN3CCCC3)CC2)c2ccc(N3CCCOCC3)nc21]

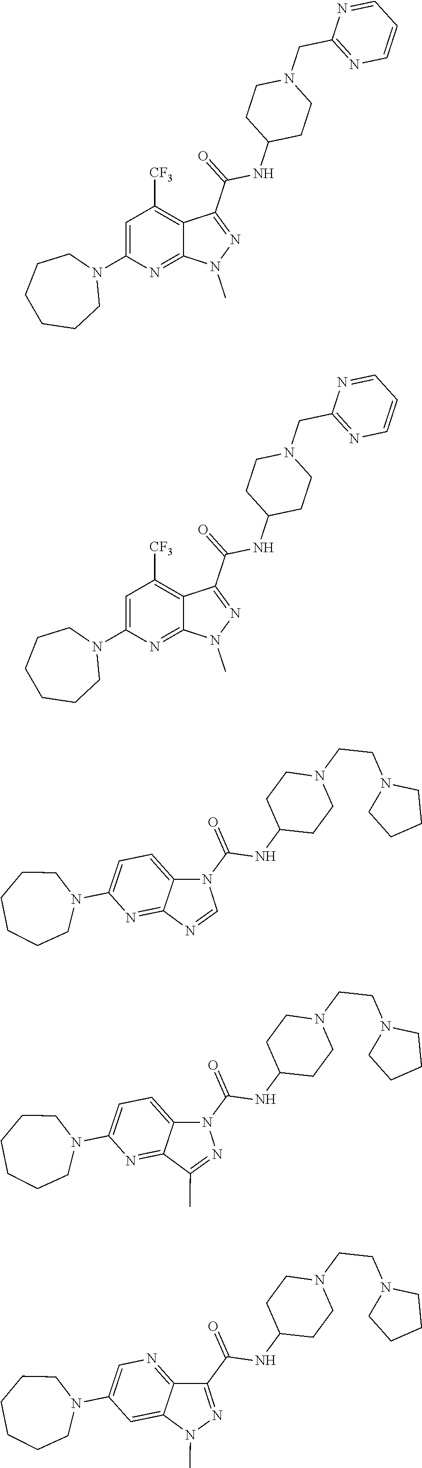 [SMILES: Cn1nc(C(=O)NC2CCN(Cc3ncccn3)CC2)c2c(C(F)(F)F)cc(N3CCCCCC3)nc21 ; Cn1nc(C(=O)NC2CCN(Cc3ncccn3)CC2)c2c(C(F)(F)F)cc(N3CCCCCC3)nc21 ; O=C(NC1CCN(CCN2CCCC2)CC1)n1cnc2nc(N3CCCCCC3)ccc21 ; Cc1nn(C(=O)NC2CCN(CCN3CCCC3)CC2)c2ccc(N3CCCCCC3)nc12 ; Cn1nc(C(=O)NC2CCN(CCN3CCCC3)CC2)c2ncc(N3CCCCCC3)cc21]

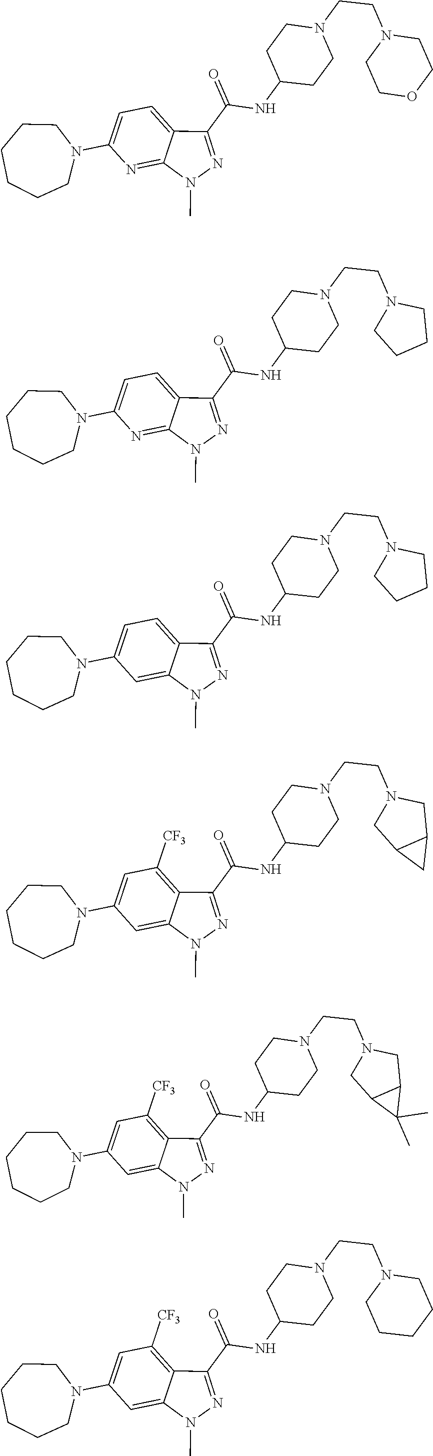 [SMILES: Cn1nc(C(=O)NC2CCN(CCN3CCOCC3)CC2)c2ccc(N3CCCCCC3)nc21 ; Cn1nc(C(=O)NC2CCN(CCN3CCCC3)CC2)c2ccc(N3CCCCCC3)nc21 ; Cn1nc(C(=O)NC2CCN(CCN3CCCC3)CC2)c2ccc(N3CCCCCC3)cc21 ; Cn1nc(C(=O)NC2CCN(CCN3CC4CC4C3)CC2)c2c(C(F)(F)F)cc(N3CCCCCC3)cc21 ; Cn1nc(C(=O)NC2CCN(CCN3CC4C(C3)C4(C)C)CC2)c2c(C(F)(F)F)cc(N3CCCCCC3)cc21 ; Cn1nc(C(=O)NC2CCN(CCN3CCCCC3)CC2)c2c(C(F)(F)F)cc(N3CCCCCC3)cc21]

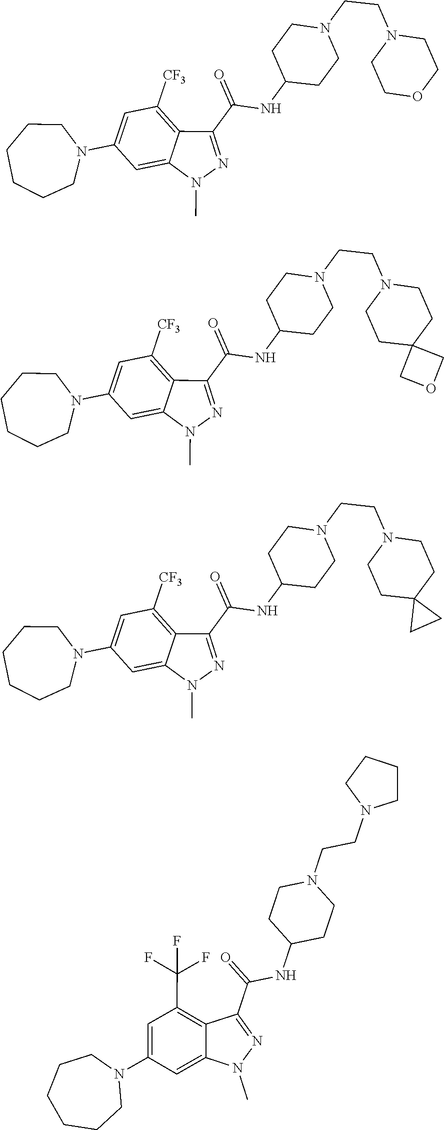 [SMILES: Cn1nc(C(=O)NC2CCN(CCN3CCOCC3)CC2)c2c(C(F)(F)F)cc(N3CCCCCC3)cc21 ; Cn1nc(C(=O)NC2CCN(CCN3CCC4(CC3)COC4)CC2)c2c(C(F)(F)F)cc(N3CCCCCC3)cc21 ; Cn1nc(C(=O)NC2CCN(CCN3CCC4(CC3)CC4)CC2)c2c(C(F)(F)F)cc(N3CCCCCC3)cc21 ; Cn1nc(C(=O)NC2CCN(CCN3CCCC3)CC2)c2c(C(F)(F)F)cc(N3CCCCCC3)cc21]

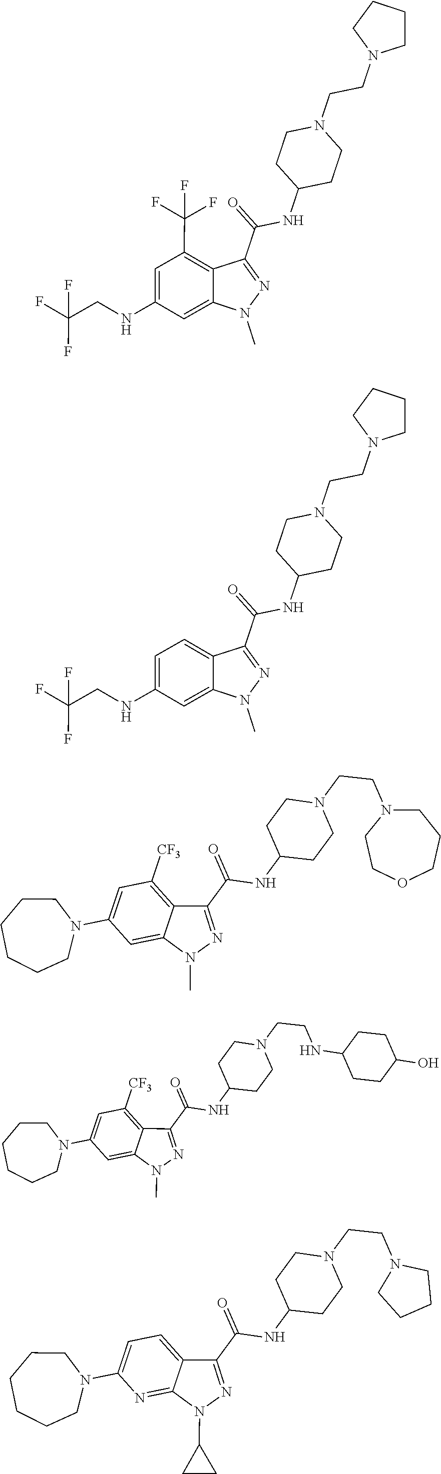 [SMILES: Cn1nc(C(=O)NC2CCN(CCN3CCCC3)CC2)c2c(C(F)(F)F)cc(NCC(F)(F)F)cc21 ; Cn1nc(C(=O)NC2CCN(CCN3CCCC3)CC2)c2ccc(NCC(F)(F)F)cc21 ; Cn1nc(C(=O)NC2CCN(CCN3CCCOCC3)CC2)c2c(C(F)(F)F)cc(N3CCCCCC3)cc21 ; Cn1nc(C(=O)NC2CCN(CCNC3CCC(O)CC3)CC2)c2c(C(F)(F)F)cc(N3CCCCCC3)cc21 ; O=C(NC1CCN(CCN2CCCC2)CC1)c1nn(C2CC2)c2nc(N3CCCCCC3)ccc12]

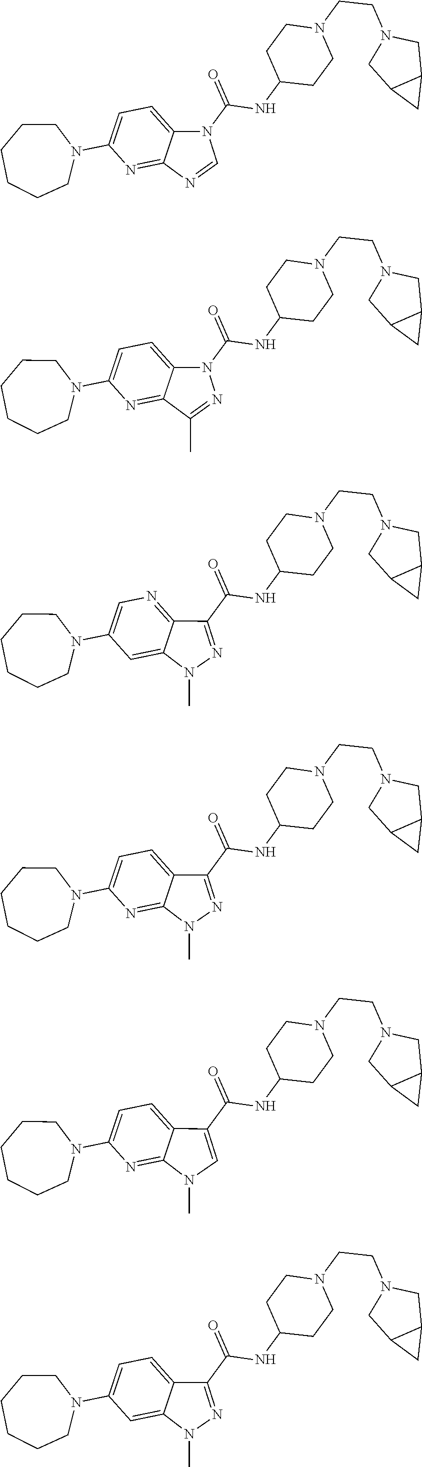 [SMILES: O=C(NC1CCN(CCN2CC3CC3C2)CC1)n1cnc2nc(N3CCCCCC3)ccc21 ; Cc1nn(C(=O)NC2CCN(CCN3CC4CC4C3)CC2)c2ccc(N3CCCCCC3)nc12 ; Cn1nc(C(=O)NC2CCN(CCN3CC4CC4C3)CC2)c2ncc(N3CCCCCC3)cc21 ; Cn1nc(C(=O)NC2CCN(CCN3CC4CC4C3)CC2)c2ccc(N3CCCCCC3)nc21 ; Cn1cc(C(=O)NC2CCN(CCN3CC4CC4C3)CC2)c2ccc(N3CCCCCC3)nc21 ; Cn1nc(C(=O)NC2CCN(CCN3CC4CC4C3)CC2)c2ccc(N3CCCCCC3)cc21]

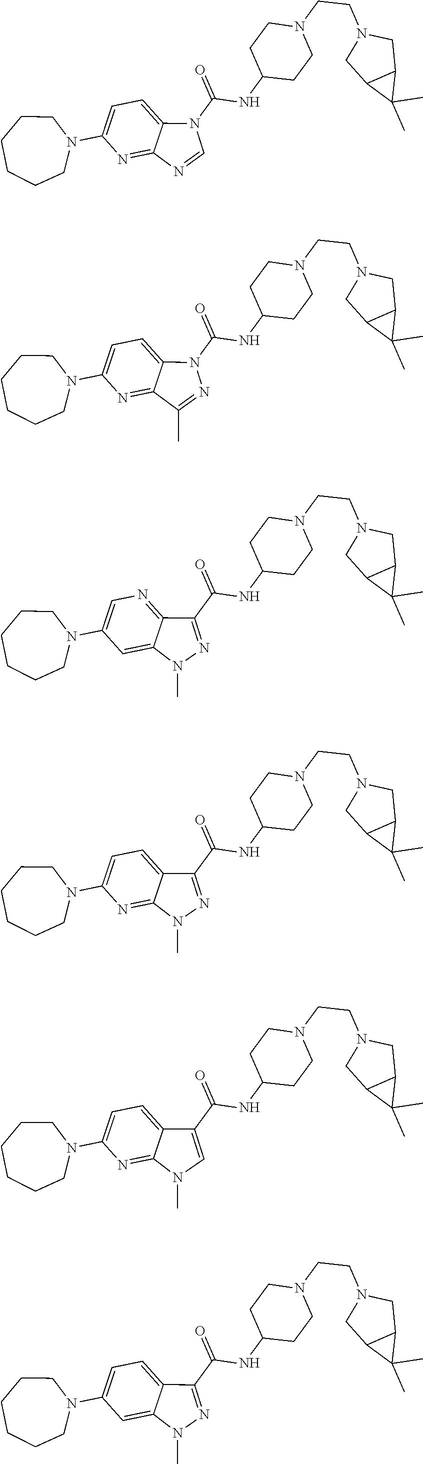 [SMILES: CC1(C)C2CN(CCN3CCC(NC(=O)n4cnc5nc(N6CCCCCC6)ccc54)CC3)CC21 ; Cc1nn(C(=O)NC2CCN(CCN3CC4C(C3)C4(C)C)CC2)c2ccc(N3CCCCCC3)nc12 ; Cn1nc(C(=O)NC2CCN(CCN3CC4C(C3)C4(C)C)CC2)c2ncc(N3CCCCCC3)cc21 ; Cn1nc(C(=O)NC2CCN(CCN3CC4C(C3)C4(C)C)CC2)c2ccc(N3CCCCCC3)nc21 ; Cn1cc(C(=O)NC2CCN(CCN3CC4C(C3)C4(C)C)CC2)c2ccc(N3CCCCCC3)nc21 ; Cn1nc(C(=O)NC2CCN(CCN3CC4C(C3)C4(C)C)CC2)c2ccc(N3CCCCCC3)cc21]

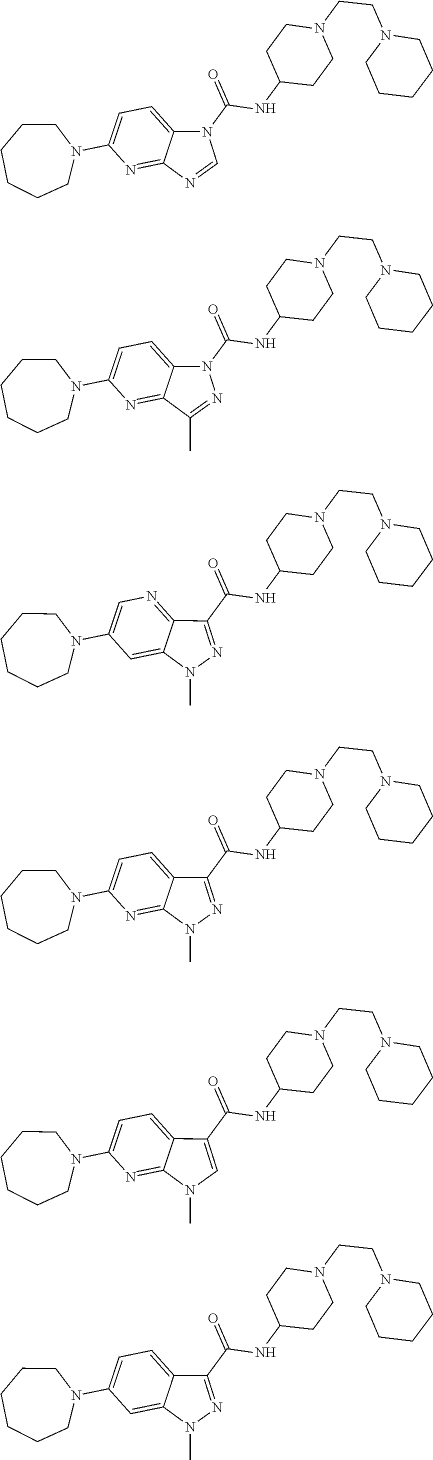 [SMILES: O=C(NC1CCN(CCN2CCCCC2)CC1)n1cnc2nc(N3CCCCCC3)ccc21 ; Cc1nn(C(=O)NC2CCN(CCN3CCCCC3)CC2)c2ccc(N3CCCCCC3)nc12 ; Cn1nc(C(=O)NC2CCN(CCN3CCCCC3)CC2)c2ncc(N3CCCCCC3)cc21 ; Cn1nc(C(=O)NC2CCN(CCN3CCCCC3)CC2)c2ccc(N3CCCCCC3)nc21 ; Cn1cc(C(=O)NC2CCN(CCN3CCCCC3)CC2)c2ccc(N3CCCCCC3)nc21 ; Cn1nc(C(=O)NC2CCN(CCN3CCCCC3)CC2)c2ccc(N3CCCCCC3)cc21]

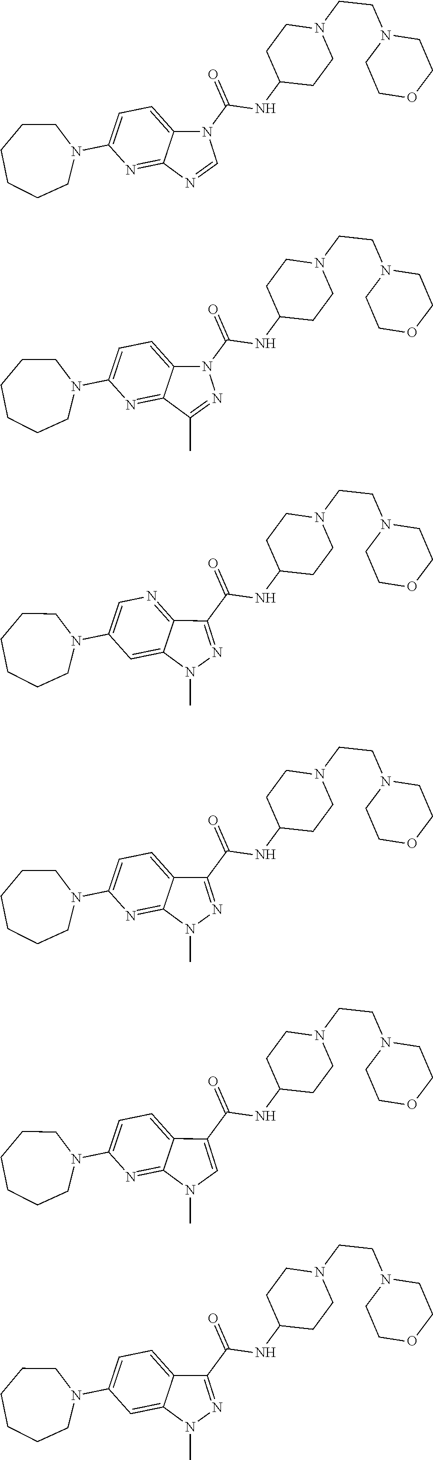 [SMILES: O=C(NC1CCN(CCN2CCOCC2)CC1)n1cnc2nc(N3CCCCCC3)ccc21 ; Cc1nn(C(=O)NC2CCN(CCN3CCOCC3)CC2)c2ccc(N3CCCCCC3)nc12 ; Cn1nc(C(=O)NC2CCN(CCN3CCOCC3)CC2)c2ncc(N3CCCCCC3)cc21 ; Cn1nc(C(=O)NC2CCN(CCN3CCOCC3)CC2)c2ccc(N3CCCCCC3)nc21 ; Cn1cc(C(=O)NC2CCN(CCN3CCOCC3)CC2)c2ccc(N3CCCCCC3)nc21 ; Cn1nc(C(=O)NC2CCN(CCN3CCOCC3)CC2)c2ccc(N3CCCCCC3)cc21]

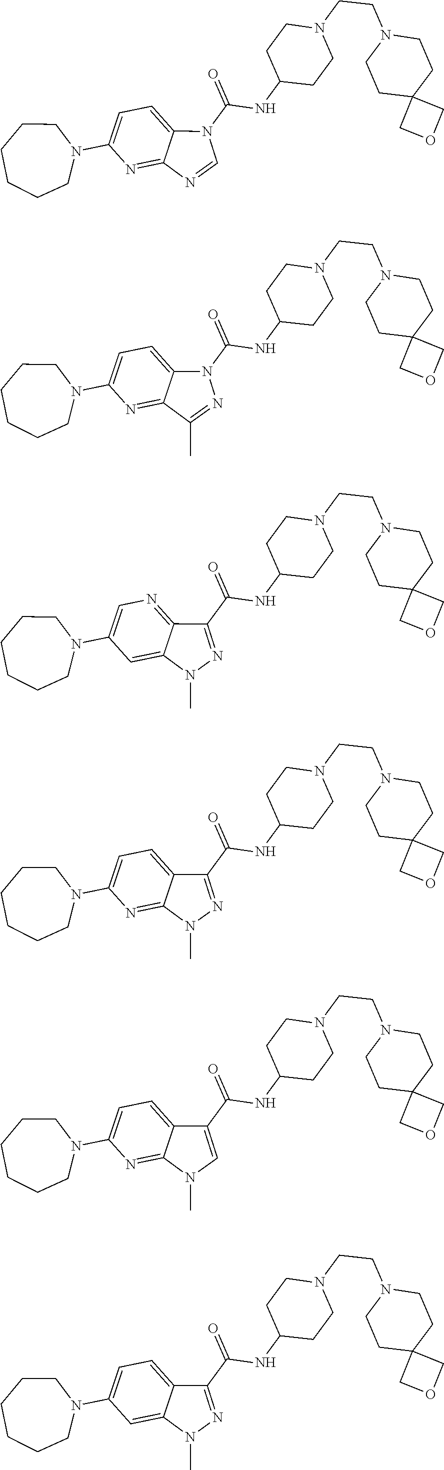 [SMILES: O=C(NC1CCN(CCN2CCC3(CC2)COC3)CC1)n1cnc2nc(N3CCCCCC3)ccc21 ; Cc1nn(C(=O)NC2CCN(CCN3CCC4(CC3)COC4)CC2)c2ccc(N3CCCCCC3)nc12 ; Cn1nc(C(=O)NC2CCN(CCN3CCC4(CC3)COC4)CC2)c2ncc(N3CCCCCC3)cc21 ; Cn1nc(C(=O)NC2CCN(CCN3CCC4(CC3)COC4)CC2)c2ccc(N3CCCCCC3)nc21 ; Cn1cc(C(=O)NC2CCN(CCN3CCC4(CC3)COC4)CC2)c2ccc(N3CCCCCC3)nc21 ; Cn1nc(C(=O)NC2CCN(CCN3CCC4(CC3)COC4)CC2)c2ccc(N3CCCCCC3)cc21]

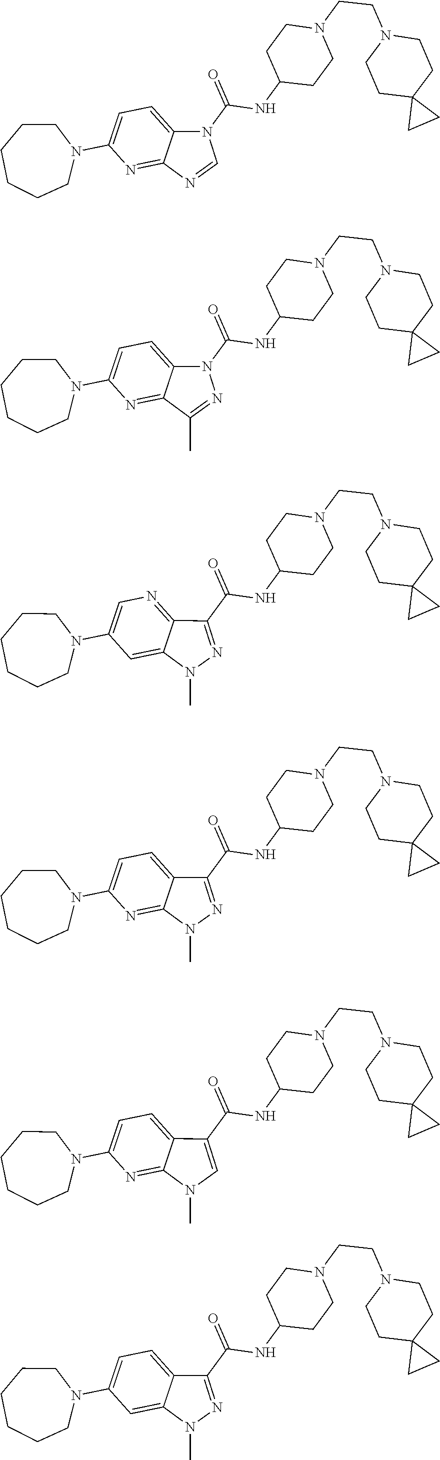 [SMILES: O=C(NC1CCN(CCN2CCC3(CC2)CC3)CC1)n1cnc2nc(N3CCCCCC3)ccc21 ; Cc1nn(C(=O)NC2CCN(CCN3CCC4(CC3)CC4)CC2)c2ccc(N3CCCCCC3)nc12 ; Cn1nc(C(=O)NC2CCN(CCN3CCC4(CC3)CC4)CC2)c2ncc(N3CCCCCC3)cc21 ; Cn1nc(C(=O)NC2CCN(CCN3CCC4(CC3)CC4)CC2)c2ccc(N3CCCCCC3)nc21 ; Cn1cc(C(=O)NC2CCN(CCN3CCC4(CC3)CC4)CC2)c2ccc(N3CCCCCC3)nc21 ; Cn1nc(C(=O)NC2CCN(CCN3CCC4(CC3)CC4)CC2)c2ccc(N3CCCCCC3)cc21]

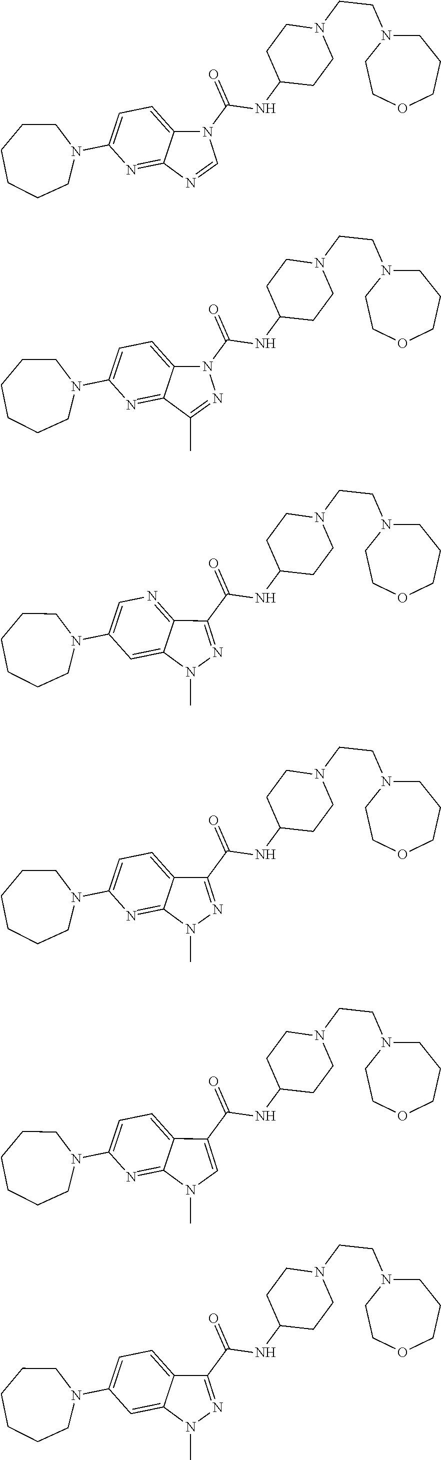 [SMILES: O=C(NC1CCN(CCN2CCCOCC2)CC1)n1cnc2nc(N3CCCCCC3)ccc21 ; Cc1nn(C(=O)NC2CCN(CCN3CCCOCC3)CC2)c2ccc(N3CCCCCC3)nc12 ; Cn1nc(C(=O)NC2CCN(CCN3CCCOCC3)CC2)c2ncc(N3CCCCCC3)cc21 ; Cn1nc(C(=O)NC2CCN(CCN3CCCOCC3)CC2)c2ccc(N3CCCCCC3)nc21 ; Cn1cc(C(=O)NC2CCN(CCN3CCCOCC3)CC2)c2ccc(N3CCCCCC3)nc21 ; Cn1nc(C(=O)NC2CCN(CCN3CCCOCC3)CC2)c2ccc(N3CCCCCC3)cc21]

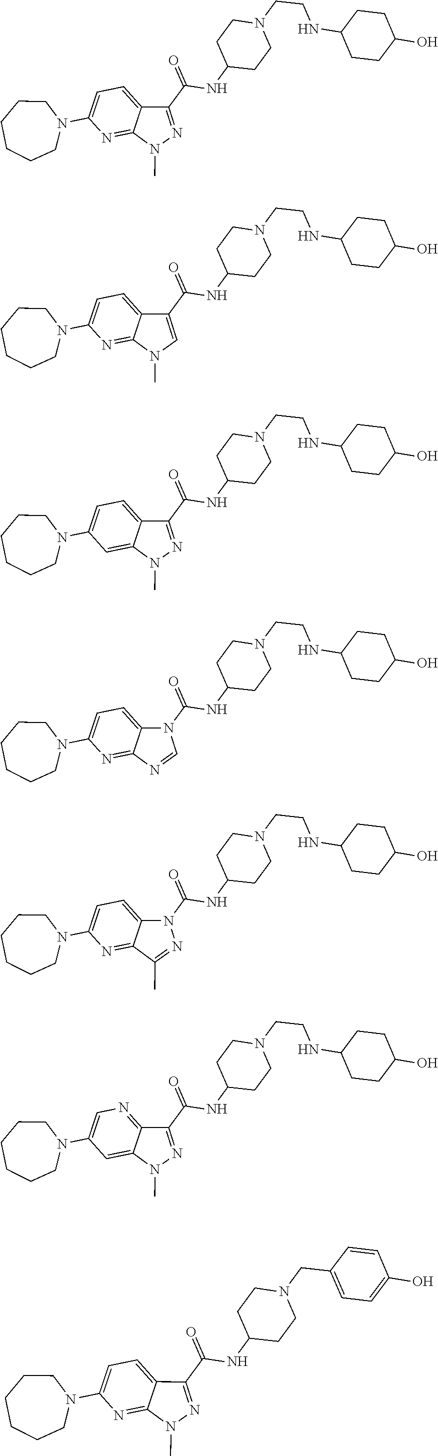

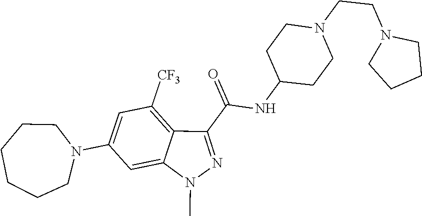 [SMILES: Cn1nc(C(=O)NC2CCN(CCN3CCCC3)CC2)c2c(C(F)(F)F)cc(N3CCCCCC3)cc21]

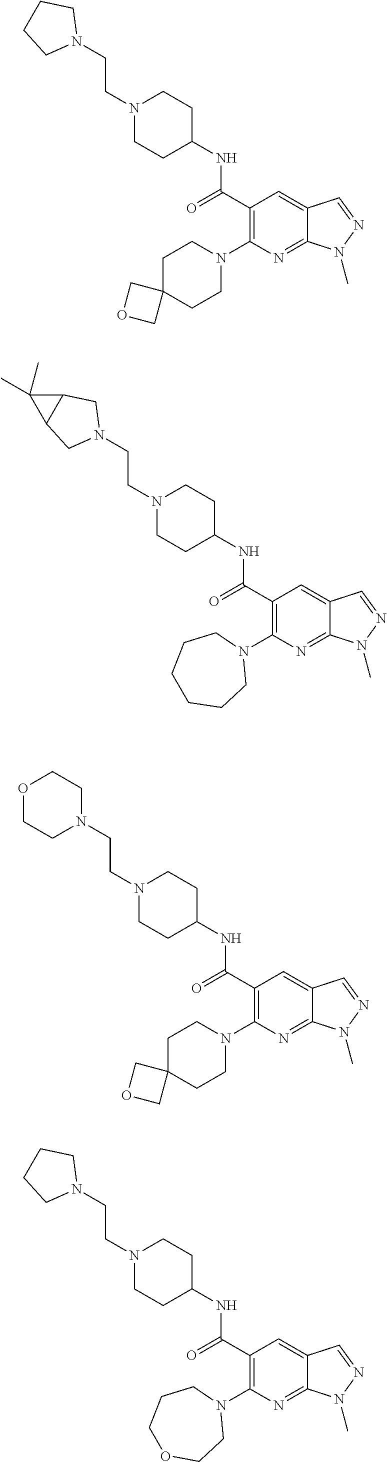 [SMILES: Cn1ncc2cc(C(=O)NC3CCN(CCN4CCCC4)CC3)c(N3CCC4(CC3)COC4)nc21 ; Cn1ncc2cc(C(=O)NC3CCN(CCN4CC5C(C4)C5(C)C)CC3)c(N3CCCCCC3)nc21 ; Cn1ncc2cc(C(=O)NC3CCN(CCN4CCOCC4)CC3)c(N3CCC4(CC3)COC4)nc21 ; Cn1ncc2cc(C(=O)NC3CCN(CCN4CCCC4)CC3)c(N3CCCOCC3)nc21]

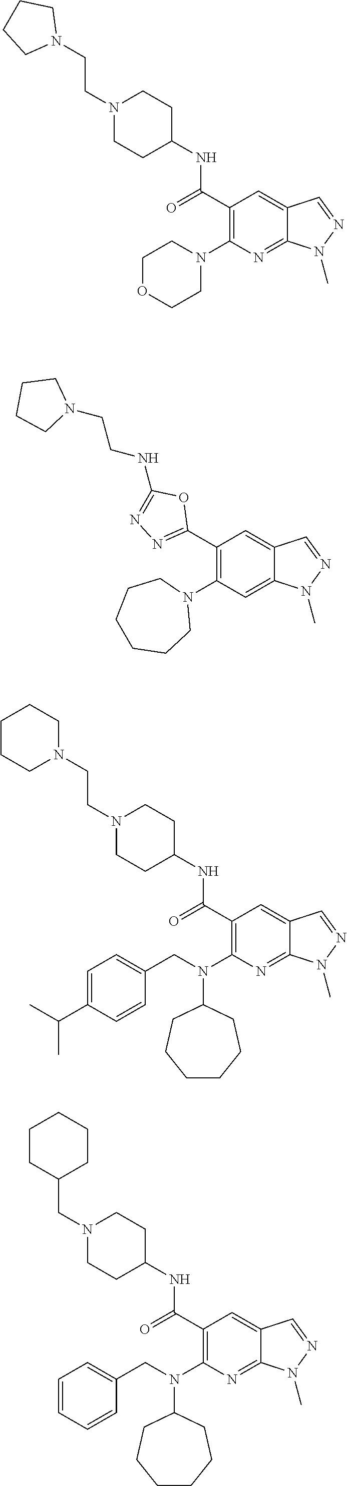 [SMILES: Cn1ncc2cc(C(=O)NC3CCN(CCN4CCCC4)CC3)c(N3CCOCC3)nc21 ; Cn1ncc2cc(-c3nnc(NCCN4CCCC4)o3)c(N3CCCCCC3)cc21 ; CC(C)c1ccc(CN(c2nc3c(cnn3C)cc2C(=O)NC2CCN(CCN3CCCCC3)CC2)C2CCCCCC2)cc1 ; Cn1ncc2cc(C(=O)NC3CCN(CC4CCCCC4)CC3)c(N(Cc3ccccc3)C3CCCCCC3)nc21]

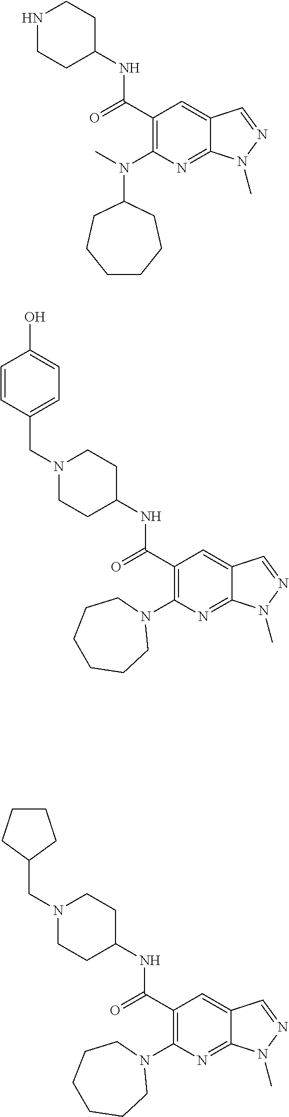 [SMILES: CN(c1nc2c(cnn2C)cc1C(=O)NC1CCNCC1)C1CCCCCC1 ; Cn1ncc2cc(C(=O)NC3CCN(Cc4ccc(O)cc4)CC3)c(N3CCCCCC3)nc21 ; Cn1ncc2cc(C(=O)NC3CCN(CC4CCCC4)CC3)c(N3CCCCCC3)nc21]

In some embodiments, the compound is a compound of Formula IId:

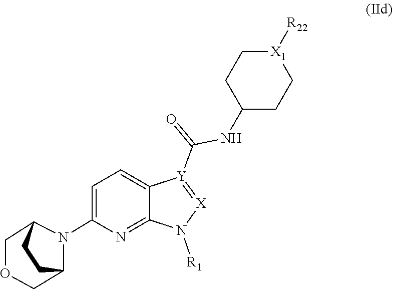

wherein:
X is C or N;
Y is C or N;
X₁ is C or N;
R₁ is hydrogen; C₁-C₆ alkyl; C₁-C₆ alkyl substituted with a substituted or unsubstituted C₃-C₈ cycloalkyl, 5-8 membered heterocyclyl, or a 6 membered aryl group; C₂-C₆ alkenyl; substituted or unsubstituted C₃-C₈ cycloalkyl, —CO—(C₃-C₈ cycloalkyl), —CO—(C₁-C₆ alkyl), —CO-aryl, —CO-heteroaryl, —CO-heterocyclyl, —SO₂—(C₁-C₆ alkyl), or —SO₂ (C₃-C₈ cycloalkyl) group; or R₁ and R₂ together form a 12-25 membered heterocycle, or R₁ and R₅ together form a 12-25 membered heterocycle;
wherein R₂₂ is hydrogen, a substituted or unsubstituted C₂-C₃ alkyl. In another embodiment, R₂₂ is C₂-C₃ alkyl. In another embodiment, R₂₂ is a substituted ethyl group. In another embodiment, R₂₂ is —CH₂CH₂—NR₂₃R₂₄ and R₂₃ and R₂₄ are independently C₁-C₃ alkyl or C₁-C₃ alkyl substituted with a C₃-C₄ cycloalkyl ring, or R₂₃ and R₂₄ together with the nitrogen atom to which they are bonded form a substituted or unsubstituted 5-8 membered heterocyclic ring. Suitable substituents for the 5-8 membered heterocyclic rings include, without limitation, 1 or 2 methyl, hydroxymethyl, methoxymethyl, or hydroxyl groups. In another embodiment, the 5-8 membered heterocyclic ring is a pyrrolidinyl, piperidinyl, or azepanyl ring, which is substituted or unsubstituted. Within this embodiment, in one embodiment, L is —CO—NH—, wherein the NH moiety is bonded to the piperidinyl moiety. In another embodiment, R₂ is —NR₂₃R₂₄ and R₂₃ and R₂₄ are independently C₁-C₃ alkyl or C₁-C₃ alkyl substituted with a C₃-C₄ cycloalkyl ring, or R₂₃ and R₂₄ together with the nitrogen atom to which they are bonded form a substituted or unsubstituted 5-8 membered heterocyclic ring. Suitable substituents for the 5-8 membered heterocyclic rings include, without limitation, 1 or 2 methyl, hydroxymethyl, methoxymethyl, or hydroxyl groups. In another embodiment, the 5-8 membered heterocyclic ring is a pyrrolidinyl, piperidinyl, or azepanyl ring, which is substituted or unsubstituted. Within this embodiment, in one embodiment, L is —(CH₂)ₙ—, —O—(CH₂)ₙ—, or —CH₂—O—(CH₂)ₙ— wherein, the left hand side of the L is bonded to the azaindazole moiety and n is 1, 2, 3, or 4.

In some embodiments, the compound is a compound selected from:

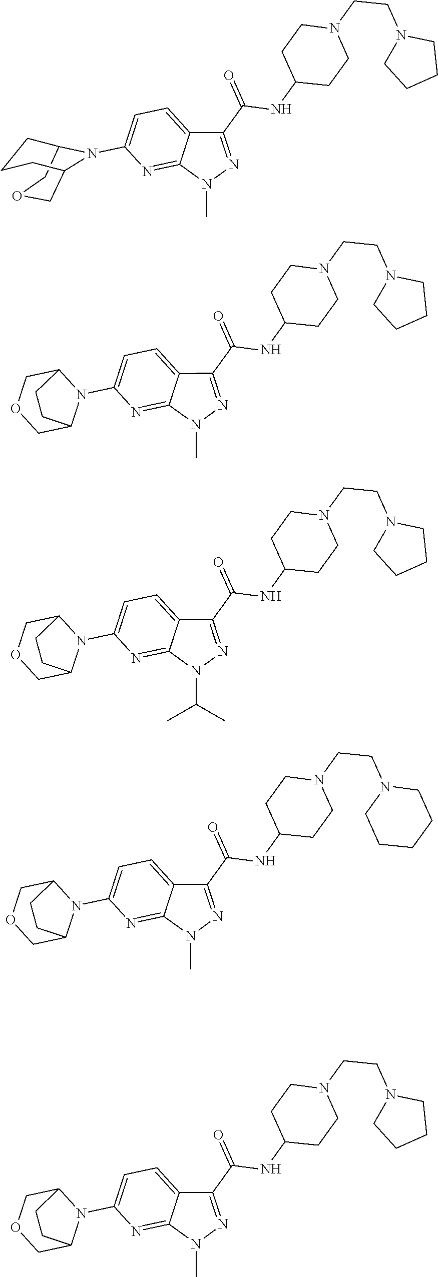 [SMILES: Cn1nc(C(=O)NC2CCN(CCN3CCCC3)CC2)c2ccc(N3C4CCCC3COC4)nc21 ; Cn1nc(C(=O)NC2CCN(CCN3CCCC3)CC2)c2ccc(N3C4CCC3COC4)nc21 ; CC(C)n1nc(C(=O)NC2CCN(CCN3CCCC3)CC2)c2ccc(N3C4CCC3COC4)nc21 ; Cn1nc(C(=O)NC2CCN(CCN3CCCCC3)CC2)c2ccc(N3C4CCC3COC4)nc21 ; Cn1nc(C(=O)NC2CCN(CCN3CCCC3)CC2)c2ccc(N3C4CCC3COC4)nc21]

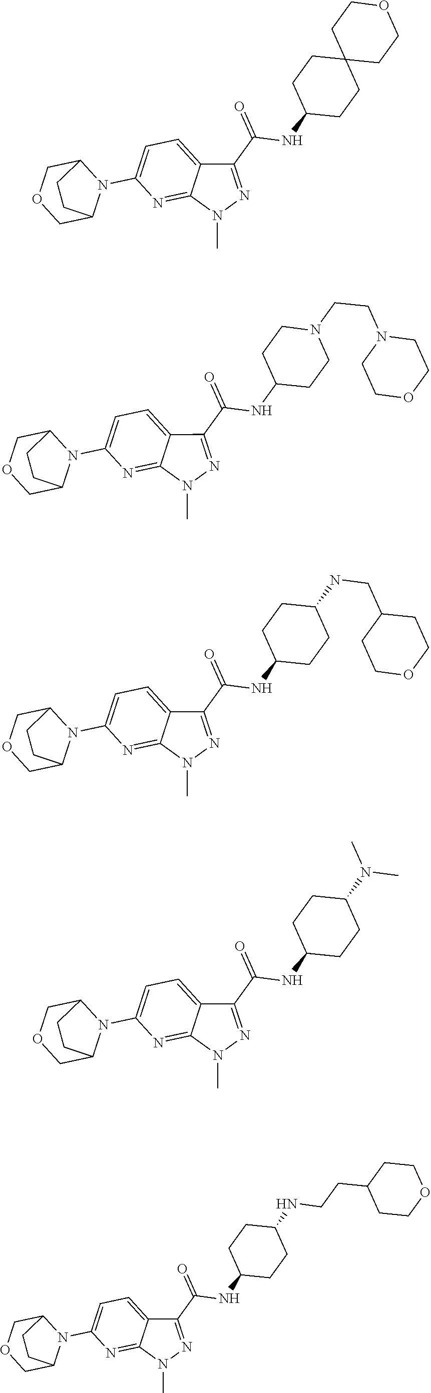

In some embodiments, the compound is a compound of Formula IIe:

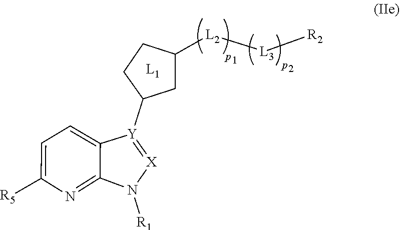

wherein:
X is C or N;
Y is C or N;
R₁ is hydrogen, branched or linear C₁-C₅ alkyl, C₂-C₁₅ alkenyl, unsubstituted or substituted cycloalkyl, —CO-(cycloalkyl), —SO₂-(cycloalkyl) group, or —(CH₂)ₙ— R₁₁, or R₅ and R₁ together form a 12-18 membered heterocycle; n is 1 or 2;
wherein L₁ is 5 membered heteroaryl containing up to 3 heteroatoms selected from the group consisting of O, N, and S; L₂ is —CO—NH— wherein the carbon atom is attached to L₁; or is 6-membered heteroaryl containing up to 3 heteroatoms selected from the group consisting of O, N, and S; or is a 5 membered cycloalkyl containing up to 3 heteroatoms selected from the group consisting of O, N, and S; L₂ is —CO—NH— wherein the carbon atom is attached to L₁; or is a 6 membered cycloalkyl containing up to 3 heteroatoms selected from the group consisting of O, N, and S; L₂ is —CO—NH— wherein the carbon atom is attached to L₁;
L₂ is —CO—NH— wherein the carbon atom is attached to L₁
L₃ is substituted or unsubstituted C₁-C₃ alkylene; p₁ is 0 or 1; p₂ is 0, 1, or 2; R₂ is 3- or 4-piperidinyl;
R₅ is —NR₅₁R₅₂ or —OR₅₄ wherein R₅₁, R₅₂, and R₅₂ is defined as in any one of the formula above; and R₁ is methyl. In another embodiment, P₁ is 1 and P₂ is 0 or 1. In another embodiment, P₁ is 0 and P₂ is 0 or 1. In another embodiment, P₂ is 0. In another embodiment, P₂ is 1. In another embodiment, L₃ is —CH₂—. In other embodiments, the compounds of Formula IIe have the following Formulas, wherein R₇, R₈, and Ry are independently hydrogen or substituted or unsubstituted C₁-C₃ alkyl:

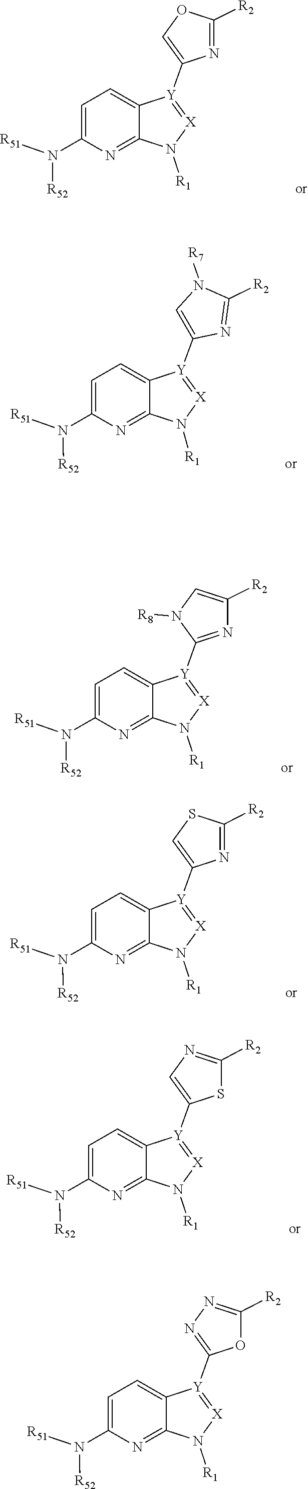

wherein R₅₁, R₅₂, and R₅₂ is defined as in any one of the formula above; and R₁ is methyl. In another embodiment, P₁ is 1 and P₂ is 0 or 1. In another embodiment, P₁ is 0 and P₂ is 0 or 1. In another embodiment, P₂ is 0. In another embodiment, P₂ is 1. In another embodiment, L₃ is —CH₂—. In other embodiments, the compounds of Formula IIe have the following Formulas, wherein R₇, R₈, and Ry are independently hydrogen or substituted or unsubstituted C₁-C₃ alkyl.

In some embodiments, the compound is a compound of Formula IIf:

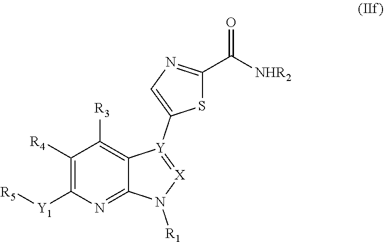

X is C or N;
Y is C or N;
R₁ is hydrogen, branched or linear C₁-C₅ alkyl, C₂-C₁₅ alkenyl, unsubstituted or substituted cycloalkyl, —CO-(cycloalkyl), —SO₂-(cycloalkyl) group, or —(CH₂)ₙ— R₁₁, or R₅ and R₁ together form a 12-18 membered heterocycle; n is 1 or 2;
wherein R₅₁, R₅₂, and R₅₂ is defined as in any one of the formula above; and R₁ is methyl. In another embodiment, P₁ is 1 and P₂ is 0 or 1. In another embodiment, P₁ is 0 and P₂ is 0 or 1. In another embodiment, P₂ is 0. In another embodiment, P₂ is 1. In another embodiment, L₃ is —CH₂—. In other embodiments, the compounds of Formula IIf have the following Formulas, wherein R₇, R₈, and Ry are independently hydrogen or substituted or unsubstituted C₁-C₃ alkyl.

In some embodiments, the compound is a compound selected from:

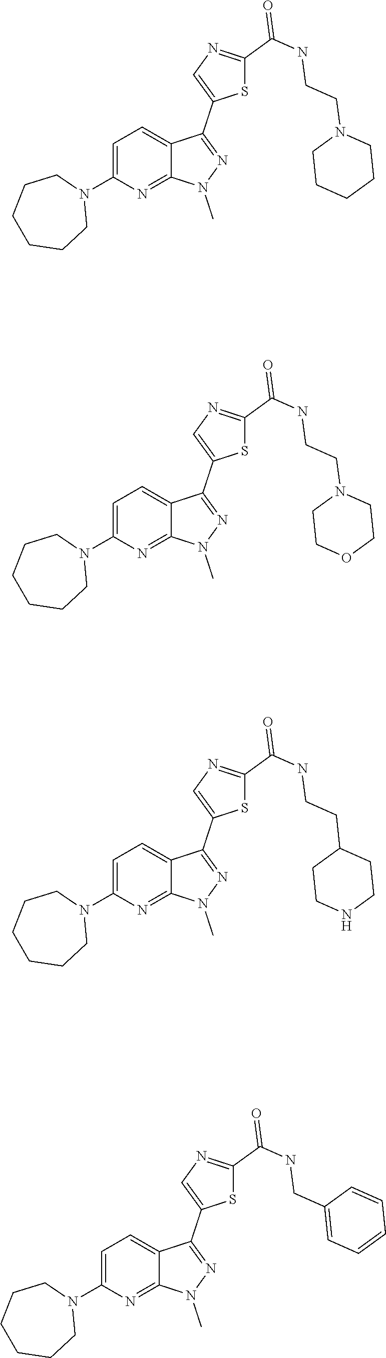

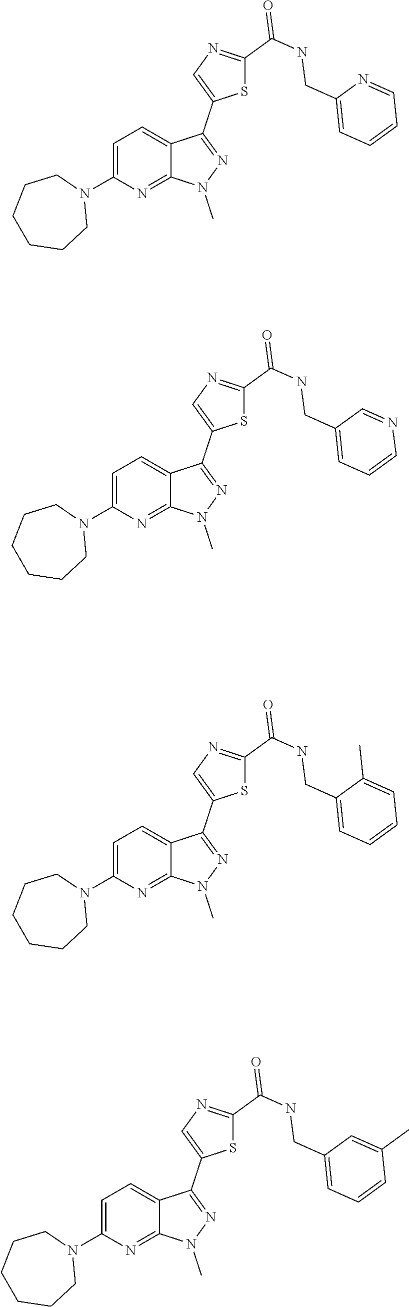

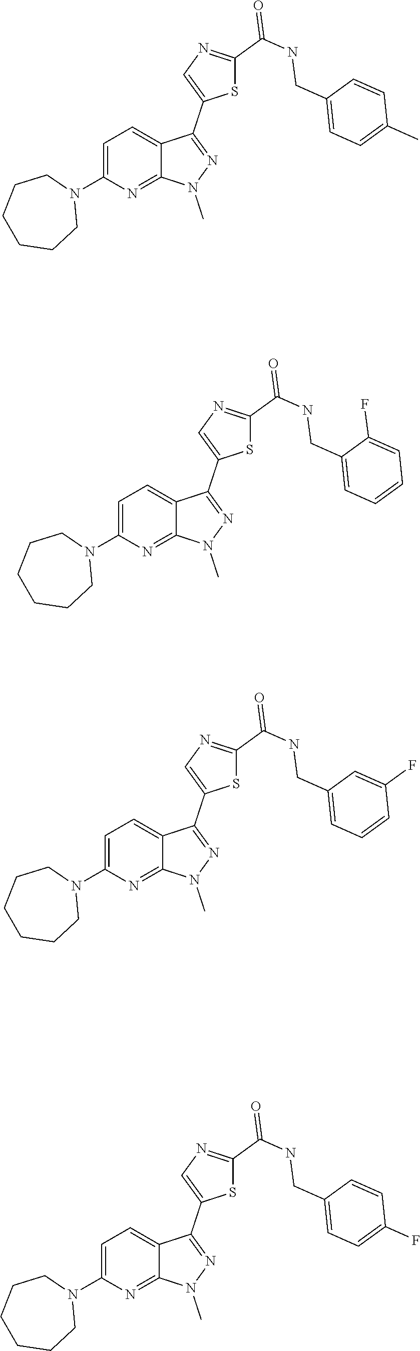

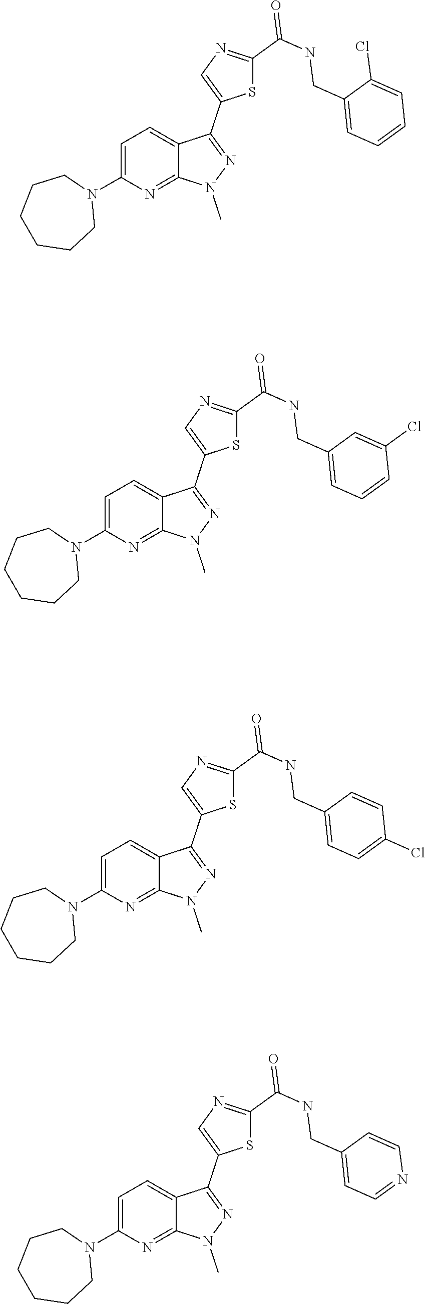

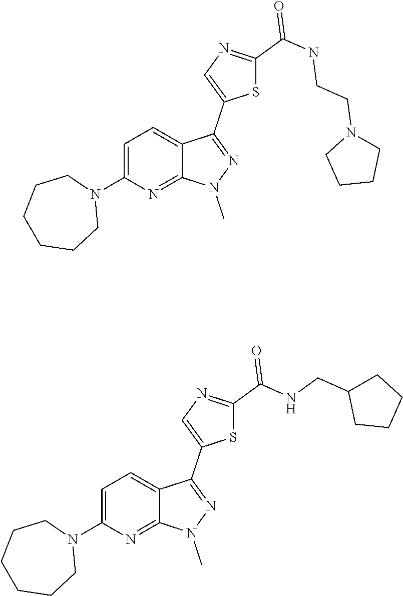

In some embodiments, the compound is a compound of Formula IIg:

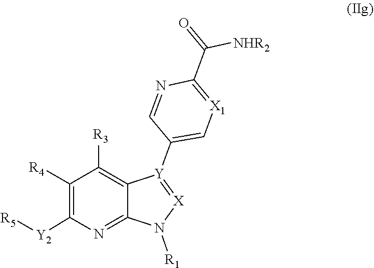

wherein:
X is C or N;
Y is C or N;
X₁ is selected from CH or N;
Y₂ is selected from O or NR′;
R₁ is hydrogen; C₁-C₆ alkyl; C₁-C₆ alkyl substituted with a substituted or unsubstituted C₃-C₈ cycloalkyl, 5-8 membered heterocyclyl, or a 6 membered aryl group; C₂-C₆ alkenyl; substituted or unsubstituted C₃-C₈ cycloalkyl, —CO—(C₃-C₈ cycloalkyl), —CO—(C₁-C₆ alkyl), —CO-aryl, —CO-heteroaryl, —CO-heterocyclyl, —SO₂—(C₁-C₆ alkyl), or —SO₂ (C₃-C₈ cycloalkyl) group; or R₁ and R₂ together form a 12-25 membered heterocycle, or R₁ and R₅ together form a 12-25 membered heterocycle;
R₂ is hydrogen; C₁-C₆ alkyl; C₁-C₆ alkyl substituted with a substituted or unsubstituted C₃-C₈ cycloalkyl, 5-8 membered heterocyclyl, or a 6 membered aryl group; C₂-C₆ alkenyl; substituted or unsubstituted C₃-C₈ cycloalkyl, —CO—(C₃-C₈ cycloalkyl), —CO—(C₁-C₆ alkyl), —CO-aryl, —CO-heteroaryl, —CO-heterocyclyl, —SO₂—(C₁-C₆ alkyl), or —SO₂ (C₃-C₈ cycloalkyl) group.
R₃, R₄, and R₅ are independently hydrogen, hydrogen; C₁-C₆ alkyl; substituted C₁-C₆ alkyl, CF₃, halo, —OH, —OR′, —NH₂, —NHR′, —NR′R′, —NHCOR′, —NR′COR′, —NHSO₂R′, —NR′SO₂R′, —NHSO₂NH₂, —NHSO₂NHR′, —NHC(O)NH₂, —NHC(O)NHR′, —N(R′)SO₂NH₂, —N(R′)SO₂NHR′, —N(R′)C(O)NH₂, and N(R)C(O)NHR′, —SO₂R′, —SO₂NH₂, SO₂NHR′, SO₂NR′R′, —CONH₂, —CONHR′, —CONR′R′, —CO₂H, —CO₂R′, or a substituted or unsubstituted C₁-C₆ alkyl, C₃-C₈ cycloalkyl, aryl, heteroaryl, or heterocyclyl group; and
R′ is a substituted or unsubstituted C₁-C₆ alkyl, C₂-C₆ alkenyl, C₂-C₆ alkynyl, C₃-C₈ cycloalkyl, aryl, heteroaryl, or heterocyclyl group, or two R′ groups together with the nitrogen atom to which they are bonded form a heterocyclic ring.

In some embodiments, the compound is a compound selected from:

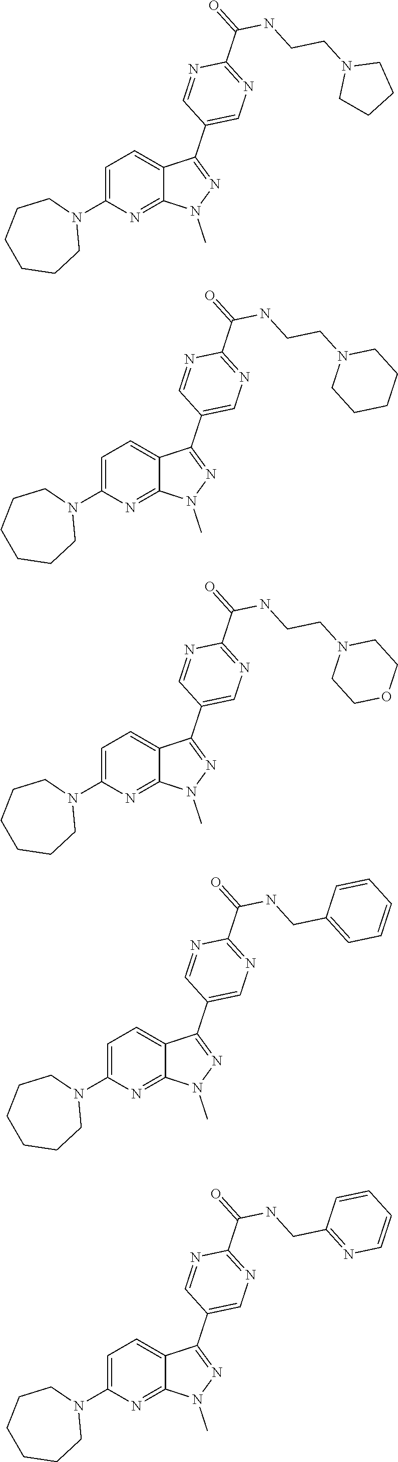

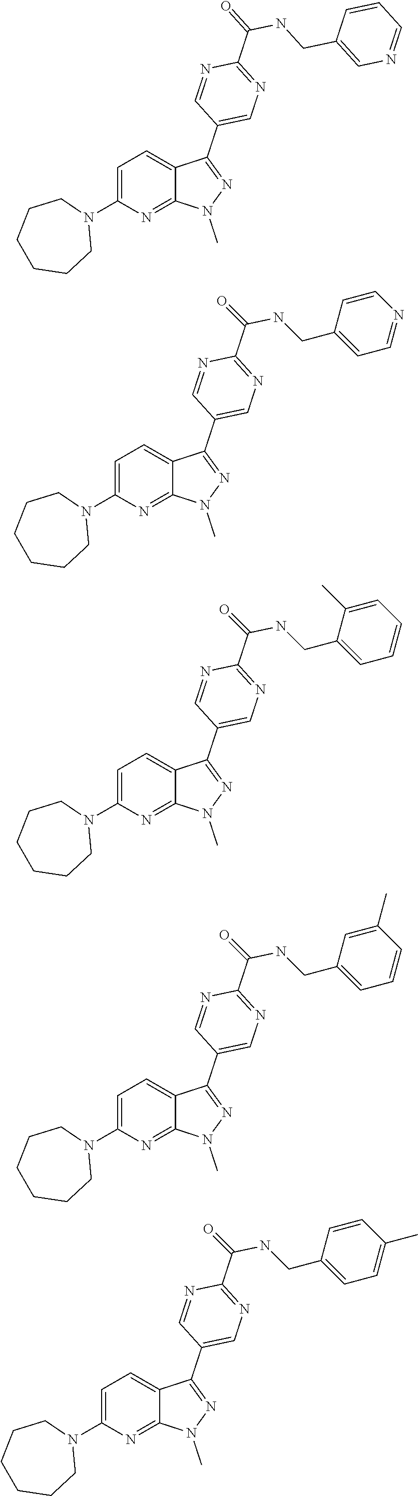

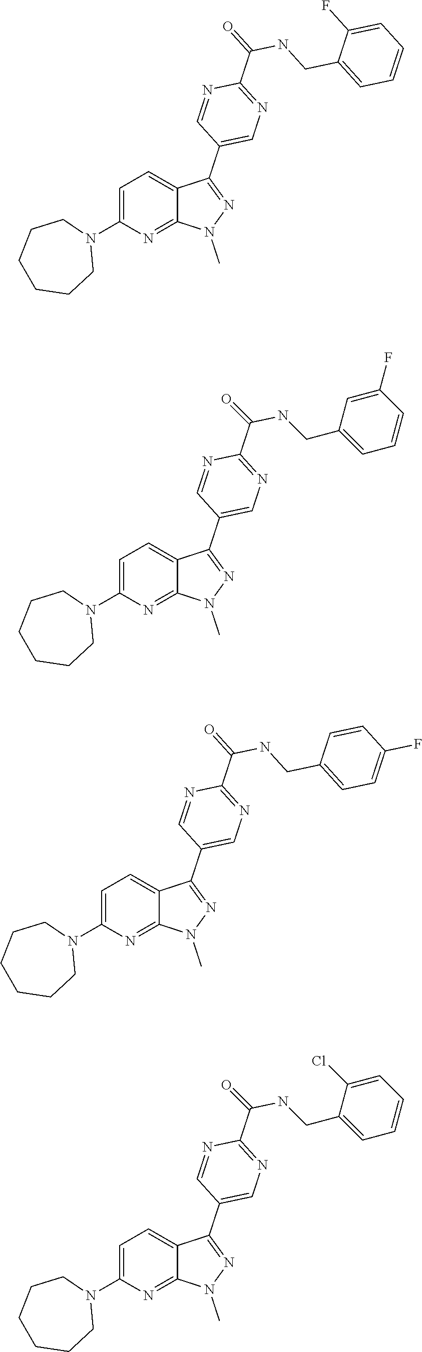

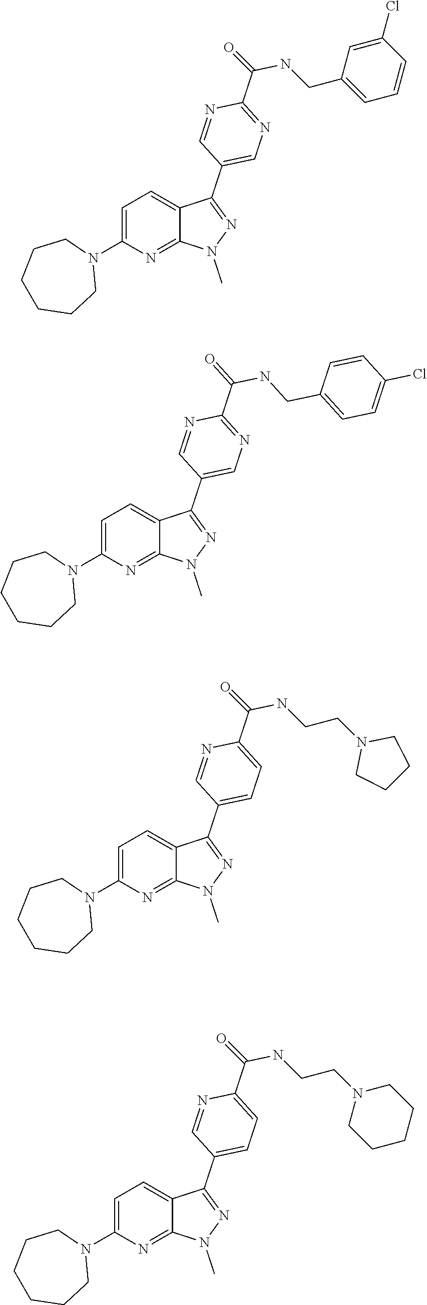

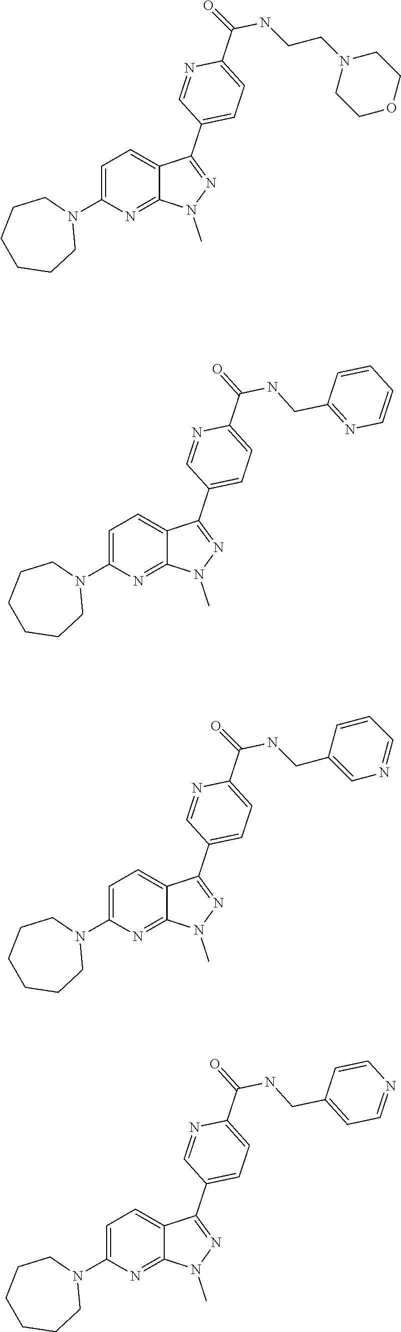

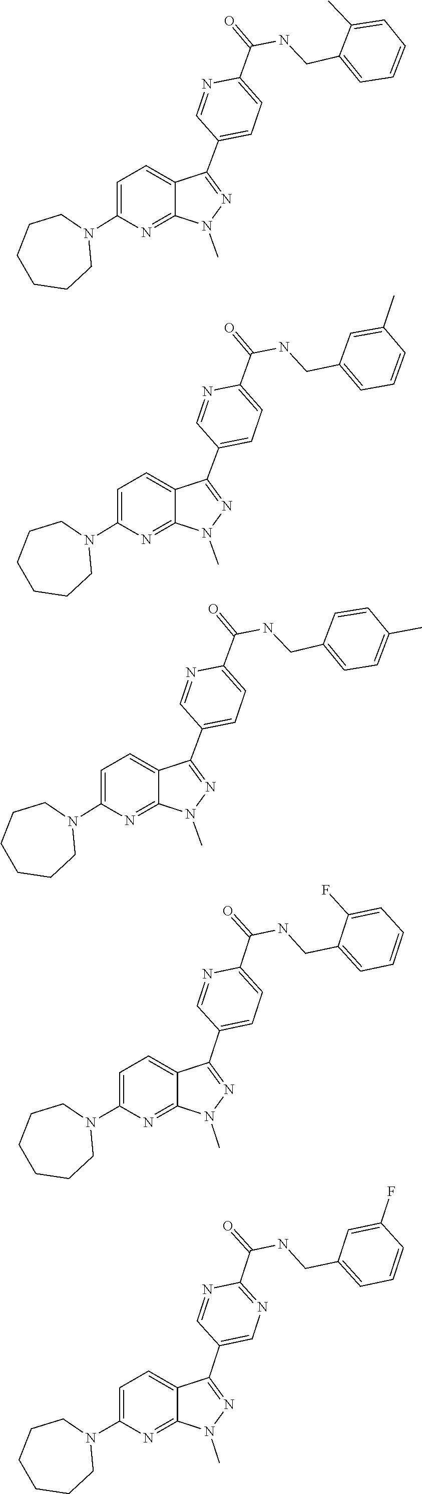

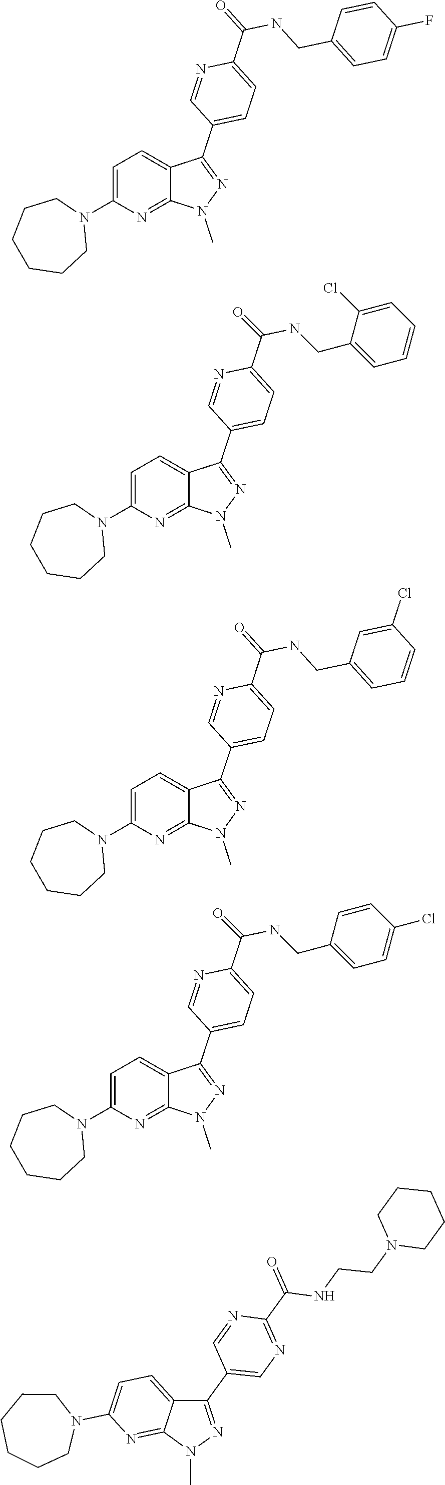

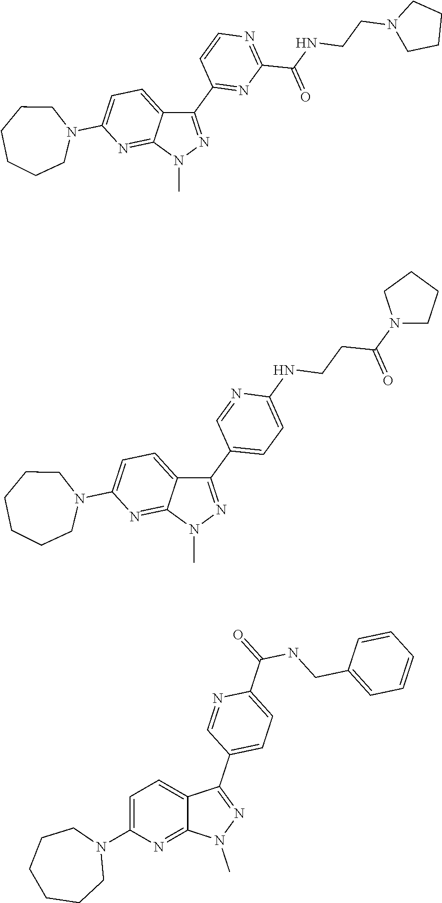

In some embodiments, the compound is a compound of Formula IIh:

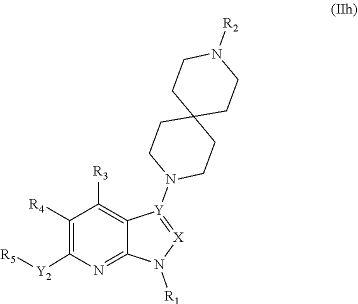

wherein:
X is C or N;
Y is C or N;
X₁ is selected from CH or N;
Y₂ is selected from O or NR;
R₁ is hydrogen; C₁-C₆ alkyl; C₁-C₆ alkyl substituted with a substituted or unsubstituted C₃-C₈ cycloalkyl, 5-8 membered heterocyclyl, or a 6 membered aryl group; C₂-C₆ alkenyl; substituted or unsubstituted C₃-C₈ cycloalkyl, —CO—(C₃-C₈ cycloalkyl), —CO—(C₁-C₆ alkyl), —CO-aryl, —CO-heteroaryl, —CO-heterocyclyl, —SO₂—(C₁-C₆ alkyl), or —SO₂—(C₃-C₈ cycloalkyl) group; or R₁ and R₂ together form a 12-25 membered heterocycle, or R₁ and R₅ together form a 12-25 membered heterocycle;
R₂ is hydrogen; C₁-C₆ alkyl; C₁-C₆ alkyl substituted with a substituted or unsubstituted C₃-C₈ cycloalkyl, 5-8 membered heterocyclyl, or a 6 membered aryl group; C₂-C₆ alkenyl; substituted or unsubstituted C₃-C₈ cycloalkyl, —CO—(C₃-C₈ cycloalkyl), —CO—(C₁-C₆ alkyl), —CO-aryl, —CO-heteroaryl, —CO-heterocyclyl, —SO₂—(C₁-C₆ alkyl), or —SO₂—(C₃-C₈ cycloalkyl) group.
R₃, R₄, and R₅ are independently hydrogen, hydrogen; C₁-C₆ alkyl; substituted C₁-C₆ alkyl, CF₃, halo, —OH, —OR′, —NH₂, —NHR′, —NR′R′, —NHCOR′, —NR′COR′, —NHSO₂R′, —NR′SO₂R′, —NHSO₂NH₂, —NHSO₂NHR′, —NHC(O)NH₂, —NHC(O)NHR′, —N(R′)SO₂NH₂, —N(R′)SO₂NHR′, —N(R′)C(O)NH₂, and N(R)C(O)NHR′, —SO₂R′, —SO₂NH₂, SO₂NHR′, SO₂NR′R′, —CONH₂, —CONHR′, —CONR′R′, —CO₂H, —CO₂R′, or a substituted or unsubstituted C₁-C₆ alkyl, C₃-C₈ cycloalkyl, aryl, heteroaryl, or heterocyclyl group; and
R′ is a substituted or unsubstituted C₁-C₆ alkyl, C₂-C₆ alkenyl, C₂-C₆ alkynyl, C₃-C₈ cycloalkyl, aryl, heteroaryl, or heterocyclyl group, or two R′ groups together with the nitrogen atom to which they are bonded form a heterocyclic ring.

In some embodiments, the compound is a compound selected from:

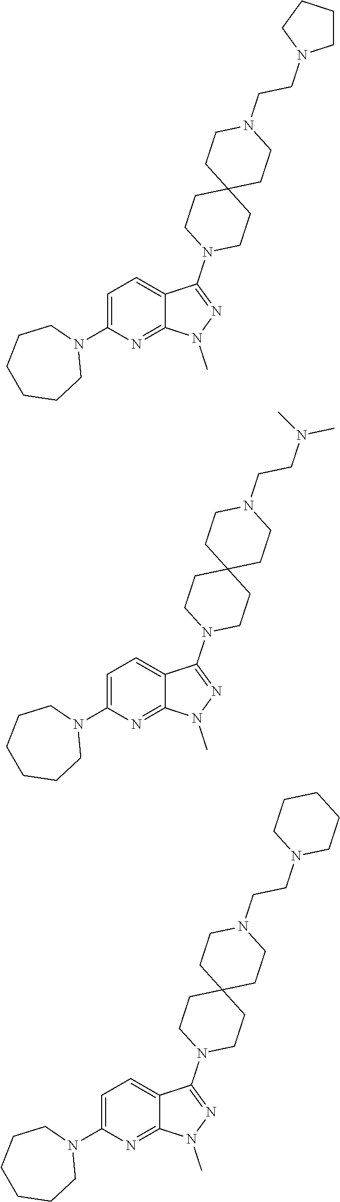 [SMILES: Cn1nc(N2CCC3(CCN(CCN4CCCC4)CC3)CC2)c2ccc(N3CCCCCC3)nc21 ; CN(C)CCN1CCC2(CC1)CCN(c1nn(C)c3nc(N4CCCCCC4)ccc13)CC2 ; Cn1nc(N2CCC3(CCN(CCN4CCCCC4)CC3)CC2)c2ccc(N3CCCCCC3)nc21]

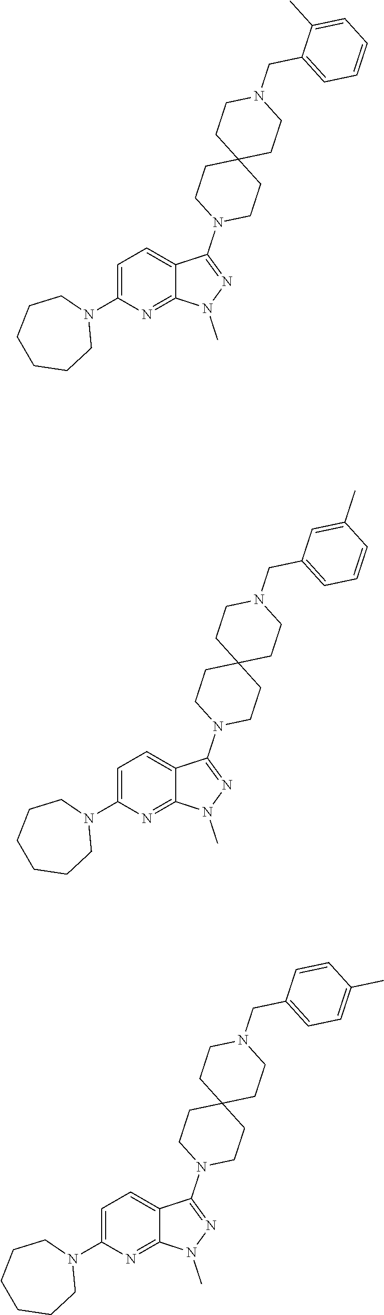 [SMILES: Cc1ccccc1CN1CCC2(CC1)CCN(c1nn(C)c3nc(N4CCCCCC4)ccc13)CC2 ; Cc1cccc(CN2CCC3(CC2)CCN(c2nn(C)c4nc(N5CCCCCC5)ccc24)CC3)c1 ; Cc1ccc(CN2CCC3(CC2)CCN(c2nn(C)c4nc(N5CCCCCC5)ccc24)CC3)cc1]

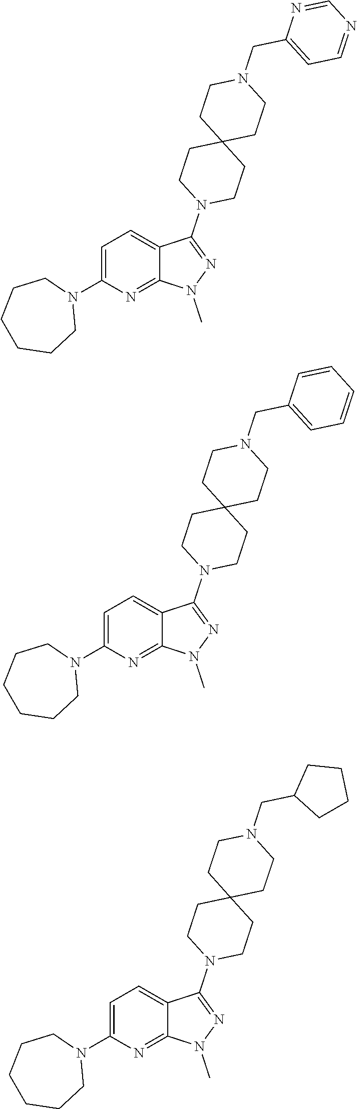 [SMILES: Cn1nc(N2CCC3(CCN(Cc4ccncn4)CC3)CC2)c2ccc(N3CCCCCC3)nc21 ; Cn1nc(N2CCC3(CCN(Cc4ccccc4)CC3)CC2)c2ccc(N3CCCCCC3)nc21 ; Cn1nc(N2CCC3(CCN(CC4CCCC4)CC3)CC2)c2ccc(N3CCCCCC3)nc21]

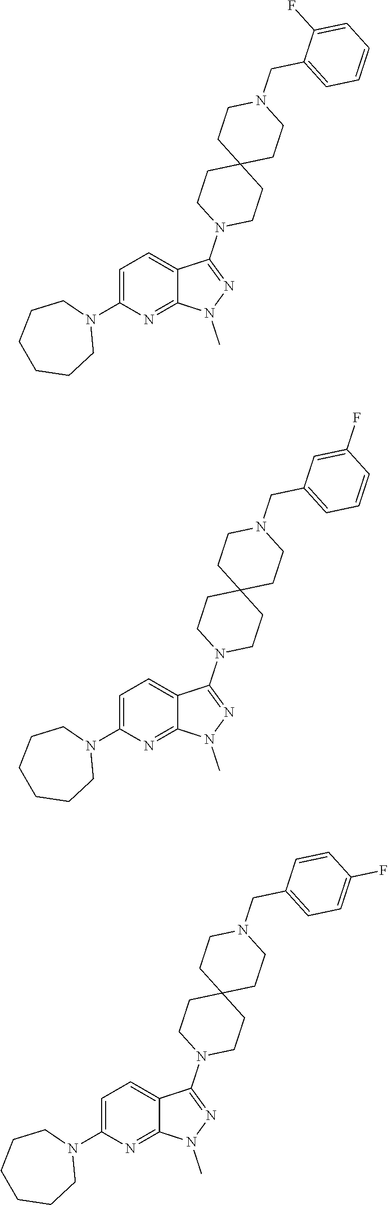 [SMILES: Cn1nc(N2CCC3(CCN(Cc4ccccc4F)CC3)CC2)c2ccc(N3CCCCCC3)nc21 ; Cn1nc(N2CCC3(CCN(Cc4cccc(F)c4)CC3)CC2)c2ccc(N3CCCCCC3)nc21 ; Cn1nc(N2CCC3(CCN(Cc4ccc(F)cc4)CC3)CC2)c2ccc(N3CCCCCC3)nc21]

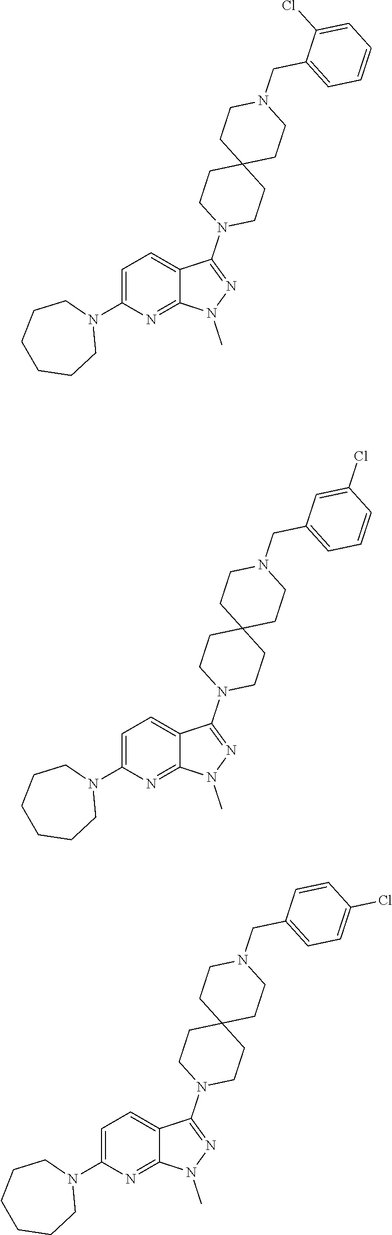 [SMILES: Cn1nc(N2CCC3(CCN(Cc4ccccc4Cl)CC3)CC2)c2ccc(N3CCCCCC3)nc21 ; Cn1nc(N2CCC3(CCN(Cc4cccc(Cl)c4)CC3)CC2)c2ccc(N3CCCCCC3)nc21 ; Cn1nc(N2CCC3(CCN(Cc4ccc(Cl)cc4)CC3)CC2)c2ccc(N3CCCCCC3)nc21]

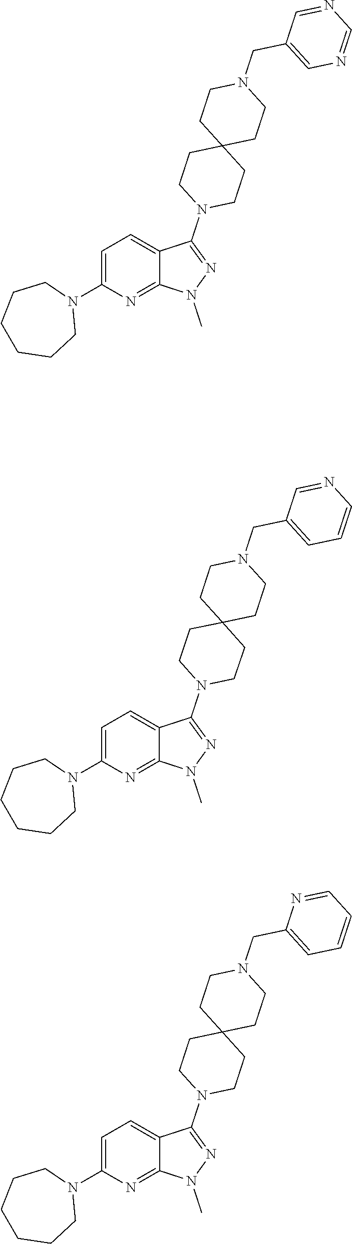 [SMILES: Cn1nc(N2CCC3(CCN(Cc4cncnc4)CC3)CC2)c2ccc(N3CCCCCC3)nc21 ; Cn1nc(N2CCC3(CCN(Cc4cccnc4)CC3)CC2)c2ccc(N3CCCCCC3)nc21 ; Cn1nc(N2CCC3(CCN(Cc4ccccn4)CC3)CC2)c2ccc(N3CCCCCC3)nc21]

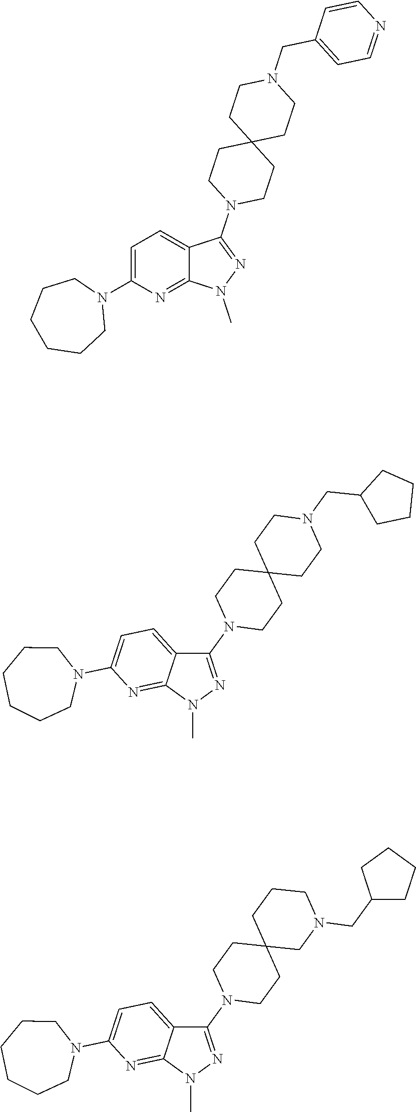 [SMILES: Cn1nc(N2CCC3(CCN(Cc4ccncc4)CC3)CC2)c2ccc(N3CCCCCC3)nc21 ; Cn1nc(N2CCC3(CCN(CC4CCCC4)CC3)CC2)c2ccc(N3CCCCCC3)nc21 ; Cn1nc(N2CCC3(CCCN(CC4CCCC4)C3)CC2)c2ccc(N3CCCCCC3)nc21]

In some embodiments, the compound is a compound of Formula III:

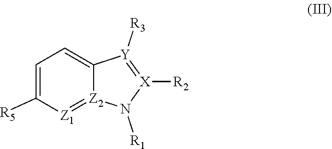

wherein:
X is C or N;
Y is C or N;
Z₁ is C or N;
Z₂ is C or N;
R₁ is hydrogen, branched or linear C₁-C₅ alkyl, C₂-C₁₅ alkenyl, unsubstituted or substituted cycloalkyl, —CO-(cycloalkyl), —SO₂-(cycloalkyl) group, or —(CH₂)ₙ— R₁₁, or R₅ and R₁ together form a 12-18 membered heterocycle; n is 1 or 2;
R₂ is substituted or unsubstituted phenyl, piperidinyl, 4-pyridyl, pyrrolidinyl, piperazinyl, benzyl, substituted phenyl, or pirazolyl group; R₅ is R₅₁R₅₂N— or R₅₄O—;
R₅₁ is H or substituted or unsubstituted C₁-C₃ alkyl; R₅₂ is C₆-C₈ cycloalkyl, substituted or unsubstituted linear C₁-C₃ alkyl, or branched C₄-C₅ alkyl or R₅₁ and R₅₂ together with the nitrogen atom to which they are bonded form a 6, 7, 8, or 9-membered heterocyclyl ring containing up to 3 heteroatoms optionally substituted, other than the azaindazole moiety to which it is already attached, by a substituted or unsubstituted benzyl acyl, or sulfonyl group; R₅₄ is H, substituted or unsubstituted benzyl group, branched C₃-C₈ alkyl, unsubstituted C₅-C₈ cycloalkyl, or C₅-C₈ cycloalkyl substituted with one or more linear or branched C₁-C₄ alkyl groups; R₁₁ is C₅-C₈ cycloalkyl or substituted or unsubstituted aryl, or substituted or unsubstituted heteroaryl;
R₃ and R₅ are independently hydrogen, halo, —CN, —OH, —OR′, —NH₂, —NHR′, —NR′R′, —NHCOR′, —NR′COR′, —NHSO₂R′, —NR′SO₂R′, —NHSO₂NH₂, —NHSO₂NHR′, —NHC(O)NH₂, —NHC(O)NHR′, —N(R′)SO₂NH₂, —N(R′)SO₂NHR′, —N(R′)C(O)NH₂, and N(R)C(O)NHR′, —SO₂R′, —SO₂NH₂, SO₂NHR′, SO₂NR′R′, —CONH₂, —CONHR′, —CONR′R′, —CO₂H, —CO₂R′, or a substituted or unsubstituted C₁-C₆ alkyl, C₃-C₈ cycloalkyl, aryl, heteroaryl, or heterocyclyl group.

In some embodiments, the compound is a compound selected from:

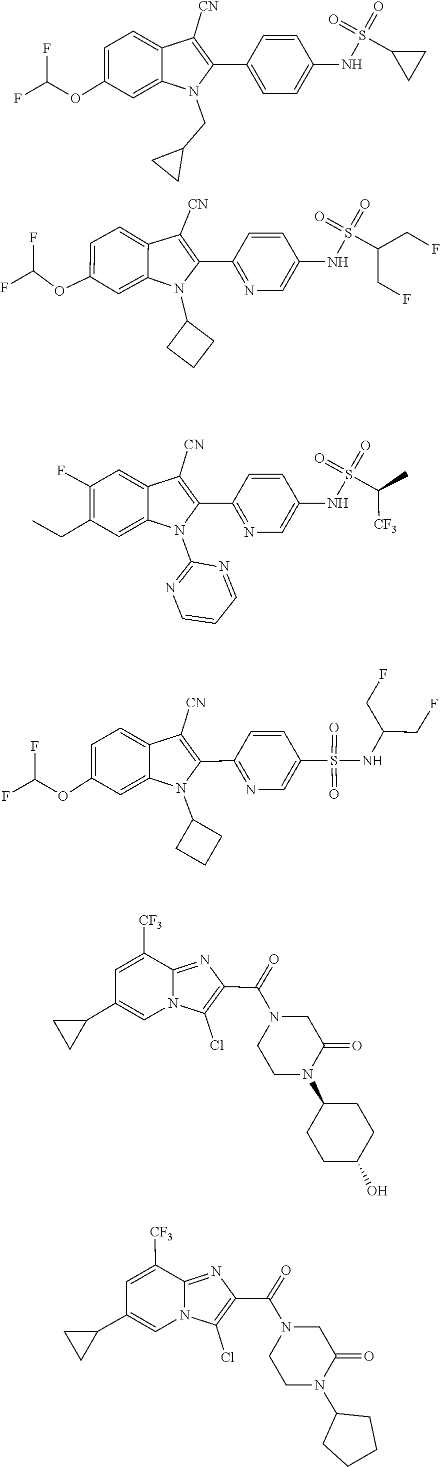

In some embodiments, the compound is a compound of Formula IV:

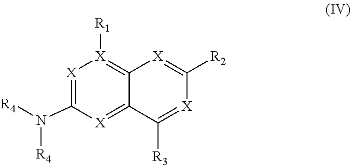

wherein:

each X is independently selected from C or N;
R¹ is absent, hydrogen, (C₁-C₈) alkyl, NR⁹R¹⁰, halo, amino, —C≡N, (C₂-C₈) alkenyl, (C₂-C₈) alkynyl, (C C₈) haloalkyl, (C₂-C₈) haloalkenyl, (C₂-C₈) haloalkynyl, (C₁-C₈) alkoxy, (C₁-C₈) haloalkoxy, (C₁-C₈) alkyl (C₁-C₆) alkoxy, 4-7 membered heterocyclyl, 5-6 membered heteroaryl, CH(O), C(O)OR⁸, SF₅, —OH, —SH, (C₁-C₆) hydroxyalkyl, (C₁-C₄) alkylsulfonyl, aminosulfonyl, amino (C₁-C₄) alkylsulfonyl or aryl;
R² is C(O)NR¹¹R¹², C(O)R¹³, 5-membered heterocycle, 5-membered substituted heterocycle, 5-membered heteroaryl, 5-membered substituted heteroaryl, 6-membered heterocycle, 6-membered substituted heterocycle, 6-membered heteroaryl, 6-membered substituted heteroaryl, 5-membered cycloalkyl, or 5-membered substituted cycloalkyl, 6-membered cycloalkyl, or 6-membered substituted cycloalkyl;
R³ is H, (C₁-C₈) alkyl, (C₁-C₈) alkenyl, (C₁-C₈) alkynyl, (C₁-C₈) alkoxy, hydroxyl, halo, amino, amido, amino (C₁-C₈) alkylamido, heterocyclyl, sulfonyl, aminosulfonyl, amino (C₁-C₈) alkysulfonyl, cyano, or (C₁-C₃) haloalkyl;
each R⁴ is independently selected from (C₁-C₈) alkyl, either unsubstituted or substituted with halo, (C₁-C₈) alkoxy, (C₁-C₈) haloaikoxy, S(O)—R⁶, S(O)₂, S(O)₂—R⁶, S(O)₂, C(O)R⁶, C(O)OR⁷ or (C₁-C₈) cycloalkyl; or both R⁴ together form a (C₁-C₈) cycloalkyl, a (C₁-C₈) substituted cycloalkyl, a (C₁-C₈) heterocycloalkyl, a (C₁-C₈) substituted heterocycloalkyl, a (C₁-C₈) aryl, a (C₁-C₈) substituted aryl, a (C₁-C₈) heteroaryl or a (C₁-C₈) substituted heteroaryl;
R⁶ is H, alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, halo, (C₁-C₄) haloalkyl and cyano;
R⁷ is H, alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, halo, (C₁-C₄) haloalkyl and cyano;
R⁸ is H, alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, halo, (C₁-C₄) haloalkyl and cyano;
R⁹ is H, (C₁-C₈) alkyl, said (C₁-C₈) alkyl being unsubstituted or substituted with one or more halo;
R¹⁰ is H, (C₁-C₈) alkyl, said (C₁-C₈) alkyl being unsubstituted or substituted with one or more halo, or R⁹ and R¹⁰, together with the nitrogen atom to which they are attached, form a 4 or 5 membered nitrogen containing heterocycle, said 4 or 5 membered nitrogen containing heterocycle being unsubstituted or substituted with one or more halo; R is H, (C₁-C₄) alkyl, (C₁-C₄) haloalkyl, 5-6 membered heterocycle or 5-6 membered heteroaryl;
R¹¹ is selected from H, (C₁-C₄) alkyl, (C₁-C₄) haloalkyl, a 5-membered heterocycle, 5-membered substituted heterocycle, 5-membered heteroaryl, 5-membered substituted heteroaryl, 6-membered heterocycle, 6-membered substituted heterocycle, 6-membered heteroaryl, 6-membered substituted heteroaryl, 5-membered cycloalkyl, or 5-membered substituted cycloalkyl, 6-membered cycloalkyl, or a 6-membered substituted cycloalkyl;
R¹² is selected from H, (C₁-C₄) alkyl, (C₁-C₄) haloalkyl, a 5-membered heterocycle, 5-membered substituted heterocycle, 5-membered heteroaryl, 5-membered substituted heteroaryl, 6-membered heterocycle, 6-membered substituted heterocycle, 6-membered heteroaryl, 6-membered substituted heteroaryl, 5-membered cycloalkyl, or 5-membered substituted cycloalkyl, 6-membered cycloalkyl, or a 6-membered substituted cycloalkyl;
R¹¹ and R¹², together with the nitrogen atom to which they are attached, form a 5-8 membered nitrogen containing heterocycle, said 5-8 membered nitrogen containing heterocycle being unsubstituted or substituted with OH, halo, ═O, or (C₁-C₈) alkyl;
R¹³ is OH, O—(C₁-C₄) alkyl;
R¹⁵ is H, (C₁-C₆) alkyl, (C₁-C) haloalkyl, 4-7 membered heterocycle, 5-6 membered heteroaryl, 3-7 membered cycloalkyl, and wherein each of said 4-7 membered heterocycle, 5-6 membered heteroaryl, 3-7 membered cycloalkyl is unsubstituted or substituted at a substitutable position with one or more ═O, OH, (C₁-C₈) alkyl, (C₁-C₈) haloalkyl, (C₁-C₈) alkoxy, amino, or, (C₁-C₈) aminoalkyl; and
R¹⁶ is H, (C₁-C₈) alkyl, or (C₁-C₈) haloalkyl.

In some embodiments, the compound is a compound of Formula IVa:

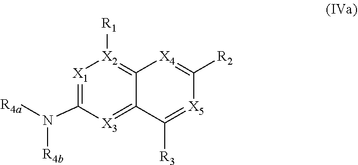

wherein:
X₁ is independently selected from C or N;
X₂ is independently selected from C or N;
X₃ is independently selected from C or N;
X₄ is independently selected from C or N;
X₅ is independently selected from C or N;
R¹ is absent, hydrogen, (C₁-C₈) alkyl, NR⁹R¹⁰, halo, amino, —C≡N, (C₂-C₈) alkenyl, (C₂-C₈) alkynyl, (C C₈) haloalkyl, (C₂-C₈) haloalkenyl, (C₂-C₈) haloalkynyl, (C₁-C₈) alkoxy, (C₁-C₈) haloalkoxy, (C₁-C₈) alkyl (C₁-C₆) alkoxy, 4-7 membered heterocyclyl, 5-6 membered heteroaryl, CH(O), C(O)OR⁸, SF₅, —OH, —SH, (C₁-C₆) hydroxyalkyl, (C₁-C₄) alkylsulfonyl, aminosulfonyl, amino (C₁-C₄) alkylsulfonyl or aryl;
R² is C(O)NR¹¹R¹², C(O)R¹³, 5-membered heterocycle, 5-membered substituted heterocycle, 5-membered heteroaryl, 5-membered substituted heteroaryl, 6-membered heterocycle, 6-membered substituted heterocycle, 6-membered heteroaryl, 6-membered substituted heteroaryl, 5-membered cycloalkyl, or 5-membered substituted cycloalkyl, 6-membered cycloalkyl, or 6-membered substituted cycloalkyl;
R³ is H, (C₁-C₈) alkyl, (C₁-C₈) alkenyl, (C₁-C₈) alkynyl, (C₁-C₈) alkoxy, hydroxyl, halo, amino, amido, amino (C₁-C₈) alkylamido, heterocyclyl, sulfonyl, aminosulfonyl, amino (C₁-C₈) alkysulfonyl, cyano, or (C₁-C₃) haloalkyl;
R^{4a} is selected from H, (C₁-C₈) alkyl, either unsubstituted or substituted with halo, (C₁-C₈) alkoxy, (C₁-C₈) haloalkoxy, S(O)—R⁶, S(O)₂, S(O)₂—R⁶, S(O)₂, C(O)R⁶, C(O)OR⁷ or (C₁-C₈) cycloalkyl;
R^{4b} is selected from H, (C₁-C₈) alkyl, either unsubstituted or substituted with halo, (C₁-C₈) alkoxy, (C₁-C₈) haloalkoxy, S(O)—R⁶, S(O)₂, S(O)₂—R⁶, S(O)₂, C(O)R⁶, C(O)OR⁷ or (C₁-C₈) cycloalkyl;
or R^{4a} and R^{4b} together form a (C₁-C₈) cycloalkyl, a (C₁-C₈) substituted cycloalkyl, a (C₁-C₈) heterocycloalkyl, a (C₁-C₈) substituted heterocycloalkyl, a (C₁-C₈) aryl, a (C₁-C₈) substituted aryl, a (C₁-C₈) heteroaryl or a (C₁-C₈) substituted heteroaryl;
R⁶ is H, alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, halo, (C₁-C₄) haloalkyl and cyano;
R⁷ is H, alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, halo, (C₁-C₄) haloalkyl and cyano;
R⁸ is H, alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, halo, (C₁-C₄) haloalkyl and cyano;
R⁹ is H, (C₁-C₈) alkyl, said (C₁-C₈) alkyl being unsubstituted or substituted with one or more halo;
R¹⁰ is H, (C₁-C₈) alkyl, said (C₁-C₈) alkyl being unsubstituted or substituted with one or more halo, or R⁹ and R¹⁰, together with the nitrogen atom to which they are attached, form a 4 or 5 membered nitrogen containing heterocycle, said 4 or 5 membered nitrogen containing heterocycle being unsubstituted or substituted with one or more halo; R is H, (C₁-C₄) alkyl, (C₁-C₄) haloalkyl, 5-6 membered heterocycle or 5-6 membered heteroaryl;
R¹¹ is selected from H, (C₁-C₄) alkyl, (C₁-C₄) haloalkyl, a 5-membered heterocycle, 5-membered substituted heterocycle, 5-membered heteroaryl, 5-membered substituted heteroaryl, 6-membered heterocycle, 6-membered substituted heterocycle, 6-membered heteroaryl, 6-membered substituted heteroaryl, 5-membered cycloalkyl, or 5-membered substituted cycloalkyl, 6-membered cycloalkyl, or a 6-membered substituted cycloalkyl;
R¹² is selected from H, (C₁-C₄) alkyl, (C₁-C₄) haloalkyl, a 5-membered heterocycle, 5-membered substituted heterocycle, 5-membered heteroaryl, 5-membered substituted heteroaryl, 6-membered heterocycle, 6-membered substituted heterocycle, 6-membered heteroaryl, 6-membered substituted heteroaryl, 5-membered cycloalkyl, or 5-membered substituted cycloalkyl, 6-membered cycloalkyl, or a 6-membered substituted cycloalkyl;
R¹¹ and R¹², together with the nitrogen atom to which they are attached, form a 5-8 membered nitrogen containing heterocycle, said 5-8 membered nitrogen containing heterocycle being unsubstituted or substituted with OH, halo, ═O, or (C₁-C₈) alkyl;
R¹³ is OH, O—(C₁-C₄) alkyl;
R¹⁵ is H, (C₁-C₆) alkyl, (C₁-C) haloalkyl, 4-7 membered heterocycle, 5-6 membered heteroaryl, 3-7 membered cycloalkyl, and wherein each of said 4-7 membered heterocycle, 5-6 membered heteroaryl, 3-7 membered cycloalkyl is unsubstituted or substituted at a substitutable position with one or more ═O, OH, (C₁-C₈) alkyl, (C₁-C₈) haloalkyl, (C₁-C₈) alkoxy, amino, or, (C₁-C₈) aminoalkyl; and
R¹⁶ is H, (C₁-C₈) alkyl, or (C₁-C₈) haloalkyl.

In some embodiments, X₁ is N. In some embodiments, X₁ is C. In some embodiments, X₂ is N. In some embodiments, X₂ is C. In some embodiments, X₃ is N. In some embodiments, X₃ is C. In some embodiments, X₄ is N. In some embodiments, X₄ is C. In some embodiments, X₅ is N. In some embodiments, X₅ is C.

In some embodiments, X₁ is N, X₂ is C, X₃ is N, X₄ is N and X₅ is C. In some embodiments, X₁ is N, X₂ is C, X₃ is C, X₄ is N and X₅ is N. In some embodiments, X₁ is C, X₂ is N, X₃ is C, X₄ is C and X₅ is N. In some embodiments, X₁ is C, X₂ is C, X₃ is N, X₄ is N and X₅ is C. In some embodiments, X₁ is N, X₂ is C, X₃ is N, X₄ is N and X₅ is N.

In some embodiments, R₁ is selected from hydrogen, (C₁-C₈) alkyl, halo or haloalkyl. In some instances, R₁ is hydrogen. In some instances, R₁ is a (C₁-C₈) alkyl, such as methyl, ethyl, propyl, i-propyl, butyl, t-butyl, i-butyl or pentyl. In certain instances, R₁ is methyl. In certain instances, R₁ is t-butyl. In some instances, R₁ is halo, such as fluoro, chloro or bromo. In certain instances, R₁ is fluoro. In some instances, R₁ is a haloalkyl, such as CF₃, CCl₃ or CBr₃. In certain instances, R₁ is CF₃.

In some embodiments, R² is C(O)NR¹¹R¹² where R¹¹ is selected from H, (C₁-C₄) alkyl, (C₁-C₄) haloalkyl, a 5-membered heterocycle, 5-membered substituted heterocycle, 5-membered heteroaryl, 5-membered substituted heteroaryl, 6-membered heterocycle, 6-membered substituted heterocycle, 6-membered heteroaryl, 6-membered substituted heteroaryl, 5-membered cycloalkyl, or 5-membered substituted cycloalkyl, 6-membered cycloalkyl, or a 6-membered substituted cycloalkyl; and R¹² is selected from H, (C₁-C₄) alkyl, (C₁-C₄) haloalkyl, a 5-membered heterocycle, 5-membered substituted heterocycle, 5-membered heteroaryl, 5-membered substituted heteroaryl, 6-membered heterocycle, 6-membered substituted heterocycle, 6-membered heteroaryl, 6-membered substituted heteroaryl, 5-membered cycloalkyl, or 5-membered substituted cycloalkyl, 6-membered cycloalkyl, or a 6-membered substituted cycloalkyl.

In some embodiments, R² is a 5-membered heteroaryl. In some instances, R² is:

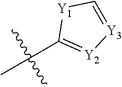

where
Y₁ is O, NH, CH₂ or S;
Y₂ is CH or N; and
Y₃ is CH or N.

In some embodiments, R¹¹ is H and R¹² is selected from a 5-membered heterocycle, 5-membered substituted heterocycle, 5-membered heteroaryl, 5-membered substituted heteroaryl, 6-membered heterocycle, 6-membered substituted heterocycle, 6-membered heteroaryl, 6-membered substituted heteroaryl, 5-membered cycloalkyl, or 5-membered substituted cycloalkyl, 6-membered cycloalkyl, or a 6-membered substituted cycloalkyl. In some instances, R¹¹ is H and R¹² is a 6-membered heterocycle. In some instances, R¹¹ is H and R¹² is a substituted 6-membered heterocycle.

In certain instances, R¹¹ is H and R¹² is substituted or unsubstituted piperidinyl, pyrrolidinyl, piperazinyl, or azepanyl group. In another embodiment, R¹² is a substituted or unsubstituted piperidin-3-yl or piperidin-4-yl group. In another embodiment, the substituted piperidin-4-yl group is:

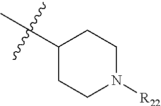

wherein R₂₂ is a substituted or unsubstituted C₂-C₃ alkyl. In another embodiment, R₂₂ is C₂-C₃ alkyl. In another embodiment, R₂₂ is a substituted ethyl group. In another embodiment, R₂₂ is —CH₂CH₂—NR₂₃R₂₄ and R₂₃ and R₂₄ are independently C₁-C₃ alkyl or C₁-C₃ alkyl substituted with a C₃-C₄ cycloalkyl ring, or R₂₃ and R₂₄ together with the nitrogen atom to which they are bonded form a substituted or unsubstituted 5-8 membered heterocyclic ring. Suitable substituents for the 5-8 membered heterocyclic rings include, without limitation, 1 or 2 methyl, hydroxymethyl, methoxymethyl, or hydroxyl groups. In another embodiment, the 5-8 membered heterocyclic ring is a pyrrolidinyl, piperidinyl, or azepanyl ring, which is substituted or unsubstituted. Within this embodiment, in one embodiment, L is —CO—NH—, wherein the NH moiety is bonded to the piperidinyl moiety. In another embodiment, R₂ is —NR₂₃R₂₄ and R₂₃ and R₂₄ are independently C₁-C₃ alkyl or C₁-C₃ alkyl substituted with a C₃-C₄ cycloalkyl ring, or R₂₃ and R₂₄ together with the nitrogen atom to which they are bonded form a substituted or unsubstituted 5-8 membered heterocyclic ring. Suitable substituents for the 5-8 membered heterocyclic rings include, without limitation, 1 or 2 methyl, hydroxymethyl, methoxymethyl, or hydroxyl groups. In another embodiment, the 5-8 membered heterocyclic ring is a pyrrolidinyl, piperidinyl, or azepanyl ring, which is substituted or unsubstituted. Within this embodiment, in one embodiment, L is —(CH₂)ₙ—, —O—(CH₂), or —CH₂—O—(CH₂)ₙ— wherein, the left hand side of the L is bonded to the azaindazole moiety and n is 1, 2, 3, or 4.

In certain instances, R¹¹ is H and R¹² is:

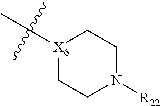

wherein X₆ is N or C; and
R₂₂ is a substituted or unsubstituted C₂-C₃ alkyl. In another embodiment, R₂₂ is C₂-C₃ alkyl. In another embodiment, R₂₂ is a substituted ethyl group. In another embodiment, R₂₂ is —CH₂CH₂—NR₂₃R₂₄ and R₂₃ and R₂₄ are independently C₁-C₃ alkyl or C₁-C₃ alkyl substituted with a C₃-C₄ cycloalkyl ring, or R₂₃ and R₂₄ together with the nitrogen atom to which they are bonded form a substituted or unsubstituted 5-8 membered heterocyclic ring. Suitable substituents for the 5-8 membered heterocyclic rings include, without limitation, 1 or 2 methyl, hydroxymethyl, methoxymethyl, or hydroxyl groups. In another embodiment, the 5-8 membered heterocyclic ring is a pyrrolidinyl, piperidinyl, or azepanyl ring, which is substituted or unsubstituted.

Within this embodiment, in one embodiment, L is —CO—NH—, wherein the NH moiety is bonded to the piperidinyl moiety. In another embodiment, R₂ is —NR₂₃R₂₄ and R₂₃ and R₂₄ are independently C₁-C₃ alkyl or C₁-C₃ alkyl substituted with a C₃-C₄ cycloalkyl ring, or R₂₃ and R₂₄ together with the nitrogen atom to which they are bonded form a substituted or unsubstituted 5-8 membered heterocyclic ring. Suitable substituents for the 5-8 membered heterocyclic rings include, without limitation, 1 or 2 methyl, hydroxymethyl, methoxymethyl, or hydroxyl groups. In another embodiment, the 5-8 membered heterocyclic ring is a pyrrolidinyl, piperidinyl, or azepanyl ring, which is substituted or unsubstituted. Within this embodiment, in one embodiment, L is —(CH₂) n, —O—(CH₂)ₙ—, or —CH₂—O—(CH₂)ₙ— wherein, the left hand side of the L is bonded to the azaindazole moiety and n is 1, 2, 3, or 4.

In some embodiments, R₄ₐ is H, (C₁-C₈) alkyl, either unsubstituted or substituted with halo, (C₁-C₈) alkoxy, (C₁-C₈) haloalkoxy or (C₁-C₈) cycloalkyl. In some embodiments, R_{4b} is H (C₁-C₈) alkyl, either unsubstituted or substituted with halo, (C₁-C₈) alkoxy, (C₁-C₈) haloaikoxy, S(O)—R₆, S(O)₂, S(O)₂—R₆, S(O)₂, C(O)R₆, C(O)OR₇ or (C₁-C₈) cycloalkyl. In some embodiments, R₄ₐ is (C₁-C₈) alkyl. In some embodiments, R₄ₐ is (C₁-C₈) alkyl substituted with halo. In some instances, R₄ₐ is ethyl substituted with halo.

In some embodiments, R^{4a} and R^{4b} together form a (C₁-C₈) cycloalkyl, a (C₁-C₈) substituted cycloalkyl, a (C₁-C₈) heterocycloalkyl, a (C₁-C₈) substituted heterocycloalkyl, a (C₁-C₈) aryl, a (C₁-C₈) substituted aryl, a (C₁-C₈) heteroaryl or a (C₁-C₈) substituted heteroaryl. In some instances, R^{4a} and R^{4b} together with the nitrogen to which they are bonded form a (C₁-C₈) heterocycloalkyl. In certain instances, R^{4a} and R^{4b} together form together with the nitrogen form a seven-membered heterocycloalkyl.

In some embodiments, the compound is a compound of Formula IVb:

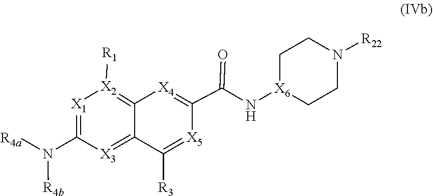

wherein:
X₁ is independently selected from C or N;
X₂ is independently selected from C or N;
X₃ is independently selected from C or N;
X₄ is independently selected from C or N;
X₅ is independently selected from C or N;
X₆ is independently selected from C or N;
R¹ is absent, hydrogen, (C₁-C₈) alkyl, NR⁹R¹⁰, halo, amino, —C≡N, (C₂-C₈) alkenyl, (C₂-C₈) alkynyl, (C C₈) haloalkyl, (C₂-C₈) haloalkenyl, (C₂-C₈) haloalkynyl, (C₁-C₈) alkoxy, (C₁-C₈) haloalkoxy, (C₁-C₈) alkyl (C₁-C₆) alkoxy, 4-7 membered heterocyclyl, 5-6 membered heteroaryl, CH(O), C(O)OR⁸, SF₅, —OH, —SH, (C₁-C₆) hydroxyalkyl, (C₁-C₄) alkylsulfonyl, aminosulfonyl, amino (C₁-C₄) alkylsulfonyl or aryl;
R² is C(O)NR¹¹R¹², C(O)R¹³, 5-membered heterocycle, 5-membered substituted heterocycle, 5-membered heteroaryl, 5-membered substituted heteroaryl, 6-membered heterocycle, 6-membered substituted heterocycle, 6-membered heteroaryl, 6-membered substituted heteroaryl, 5-membered cycloalkyl, or 5-membered substituted cycloalkyl, 6-membered cycloalkyl, or 6-membered substituted cycloalkyl;
R³ is H, (C₁-C₈) alkyl, (C₁-C₈) alkenyl, (C₁-C₈) alkynyl, (C₁-C₈) alkoxy, hydroxyl, halo, amino, amido, amino (C₁-C₈) alkylamido, heterocyclyl, sulfonyl, aminosulfonyl, amino (C₁-C₈) alkysulfonyl, cyano, or (C₁-C₃) haloalkyl;
R^{4a} is selected from H, (C₁-C₈) alkyl, either unsubstituted or substituted with halo, (C₁-C₈) alkoxy, (C₁-C₈) haloalkoxy, S(O)—R⁶, S(O)₂, S(O)₂—R⁶, S(O)₂, C(O)R⁶, C(O)OR⁷ or (C₁-C₈) cycloalkyl;
R^{4b} is selected from H, (C₁-C₈) alkyl, either unsubstituted or substituted with halo, (C₁-C₈) alkoxy, (C₁-C₈) haloalkoxy, S(O)—R⁶, S(O)₂, S(O)₂—R⁶, S(O)₂, C(O)R⁶, C(O)OR⁷ or (C₁-C₈) cycloalkyl;
or R^{4a} and R^{4b} together form a (C₁-C₈) cycloalkyl, a (C₁-C₈) substituted cycloalkyl, a (C₁-C₈) heterocycloalkyl, a (C₁-C₈) substituted heterocycloalkyl, a (C₁-C₈) aryl, a (C₁-C₈) substituted aryl, a (C₁-C₈) heteroaryl or a (C₁-C₈) substituted heteroaryl;
R⁶ is H, alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, halo, (C₁-C₄) haloalkyl and cyano;
R⁷ is H, alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, halo, (C₁-C₄) haloalkyl and cyano;
R⁸ is H, alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, halo, (C₁-C₄) haloalkyl and cyano;
R⁹ is H, (C₁-C₈) alkyl, said (C₁-C₈) alkyl being unsubstituted or substituted with one or more halo;
R¹⁰ is H, (C₁-C₈) alkyl, said (C₁-C₈) alkyl being unsubstituted or substituted with one or more halo, or R⁹ and R¹⁰, together with the nitrogen atom to which they are attached, form a 4 or 5 membered nitrogen containing heterocycle, said 4 or 5 membered nitrogen containing heterocycle being unsubstituted or substituted with one or more halo; R is H, (C₁-C₄) alkyl, (C₁-C₄) haloalkyl, 5-6 membered heterocycle or 5-6 membered heteroaryl;
R¹¹ is selected from H, (C₁-C₄) alkyl, (C₁-C₄) haloalkyl, a 5-membered heterocycle, 5-membered substituted heterocycle, 5-membered heteroaryl, 5-membered substituted heteroaryl, 6-membered heterocycle, 6-membered substituted heterocycle, 6-membered heteroaryl, 6-membered substituted heteroaryl, 5-membered cycloalkyl, or 5-membered substituted cycloalkyl, 6-membered cycloalkyl, or a 6-membered substituted cycloalkyl;
R¹² is selected from H, (C₁-C₄) alkyl, (C₁-C₄) haloalkyl, a 5-membered heterocycle, 5-membered substituted heterocycle, 5-membered heteroaryl, 5-membered substituted heteroaryl, 6-membered heterocycle, 6-membered substituted heterocycle, 6-membered heteroaryl, 6-membered substituted heteroaryl, 5-membered cycloalkyl, or 5-membered substituted cycloalkyl, 6-membered cycloalkyl, or a 6-membered substituted cycloalkyl;
R¹¹ and R¹², together with the nitrogen atom to which they are attached, form a 5-8 membered nitrogen containing heterocycle, said 5-8 membered nitrogen containing heterocycle being unsubstituted or substituted with OH, halo, ═O, or (C₁-C₈) alkyl;
R¹³ is OH, O—(C₁-C₄) alkyl;
R¹⁵ is H, (C₁-C₆) alkyl, (C₁-C) haloalkyl, 4-7 membered heterocycle, 5-6 membered heteroaryl, 3-7 membered cycloalkyl, and wherein each of said 4-7 membered heterocycle, 5-6 membered heteroaryl, 3-7 membered cycloalkyl is unsubstituted or substituted at a substitutable position with one or more ═O, OH, (C₁-C₈) alkyl, (C₁-C₈) haloalkyl, (C₁-C₈) alkoxy, amino, or, (C₁-C₈) aminoalkyl; and
R¹⁶ is H, (C₁-C₈) alkyl, or (C₁-C₈) haloalkyl; and
R₂₂ is a substituted or unsubstituted C₂-C₃ alkyl. In another embodiment, R₂₂ is C₂-C₃ alkyl. In another embodiment, R₂₂ is a substituted ethyl group. In another embodiment, R₂₂ is —CH₂CH₂—NR₂₃R₂₄ and R₂₃ and R₂₄ are independently C₁-C₃ alkyl or C₁-C₃ alkyl substituted with a C₃-C₄ cycloalkyl ring, or R₂₃ and R₂₄ together with the nitrogen atom to which they are bonded form a substituted or unsubstituted 5-8 membered heterocyclic ring. Suitable substituents for the 5-8 membered heterocyclic rings include, without limitation, 1 or 2 methyl, hydroxymethyl, methoxymethyl, or hydroxyl groups. In another embodiment, the 5-8 membered heterocyclic ring is a pyrrolidinyl, piperidinyl, or azepanyl ring, which is substituted or unsubstituted. Within this embodiment, in one embodiment, L is —CO—NH—, wherein the NH moiety is bonded to the piperidinyl moiety. In another embodiment, R₂ is —NR₂₃R₂₄ and R₂₃ and R₂₄ are independently C₁-C₃ alkyl or C₁-C₃ alkyl substituted with a C₃-C₄ cycloalkyl ring, or R₂₃ and R₂₄ together with the nitrogen atom to which they are bonded form a substituted or unsubstituted 5-8 membered heterocyclic ring. Suitable substituents for the 5-8 membered heterocyclic rings include, without limitation, 1 or 2 methyl, hydroxymethyl, methoxymethyl, or hydroxyl groups. In another embodiment, the 5-8 membered heterocyclic ring is a pyrrolidinyl, piperidinyl, or azepanyl ring, which is substituted or unsubstituted. Within this embodiment, in one embodiment, L is —(CH₂)ₙ—, —O—(CH₂) n, or —CH₂—O—(CH₂)ₙ— wherein, the left hand side of the L is bonded to the azaindazole moiety and n is 1, 2, 3, or 4.

In some embodiments, the compound is a compound selected from:

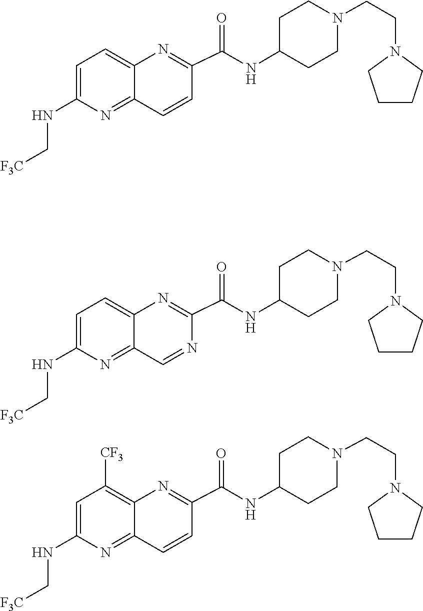 [SMILES: O=C(NC1CCN(CCN2CCCC2)CC1)c1ccc2nc(NCC(F)(F)F)ccc2n1 ; O=C(NC1CCN(CCN2CCCC2)CC1)c1ncc2nc(NCC(F)(F)F)ccc2n1 ; O=C(NC1CCN(CCN2CCCC2)CC1)c1ccc2nc(NCC(F)(F)F)cc(C(F)(F)F)c2n1]

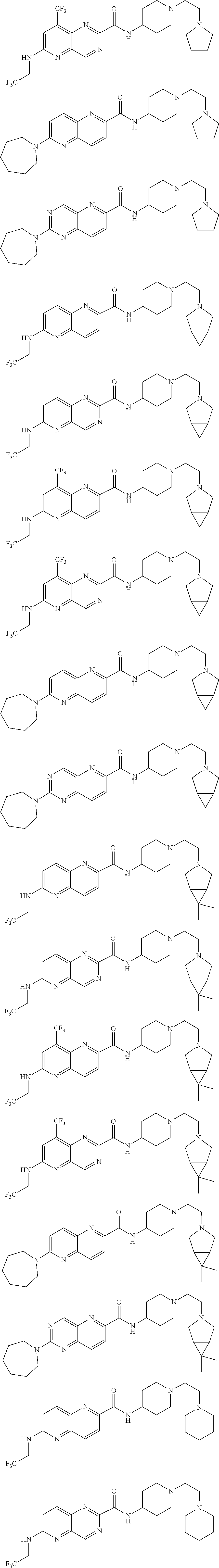

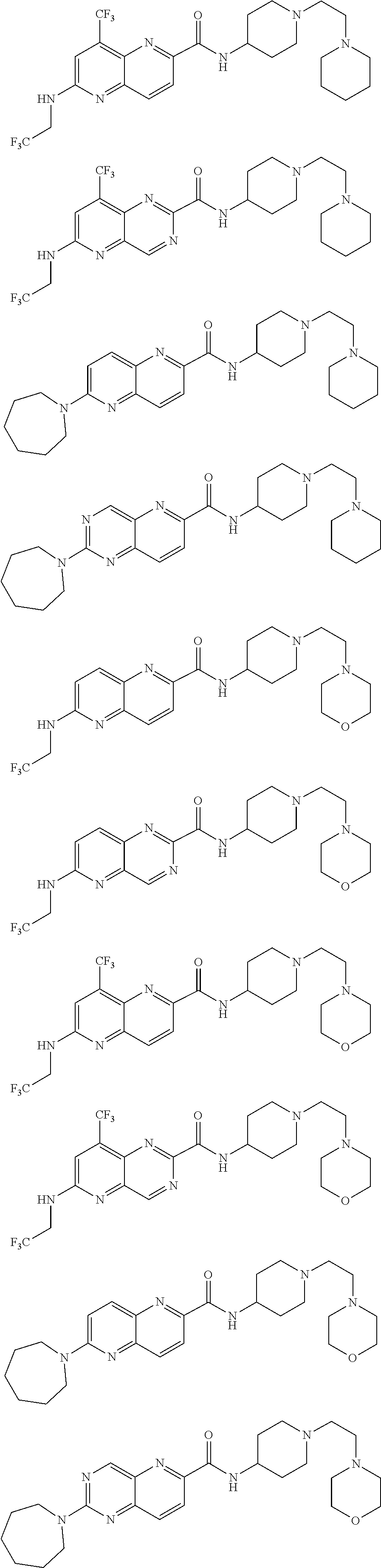

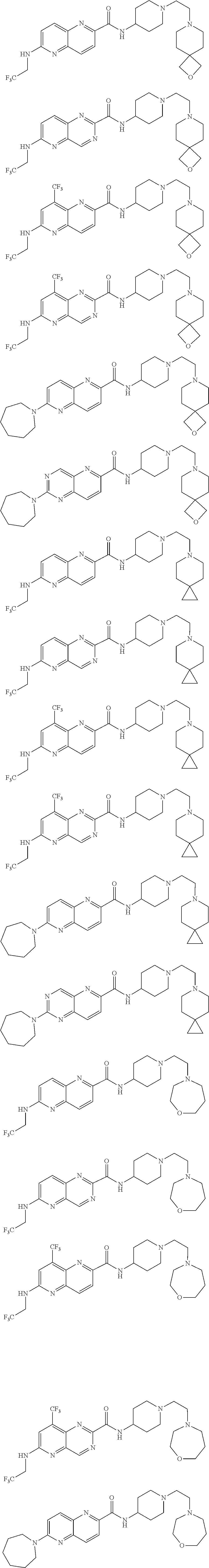

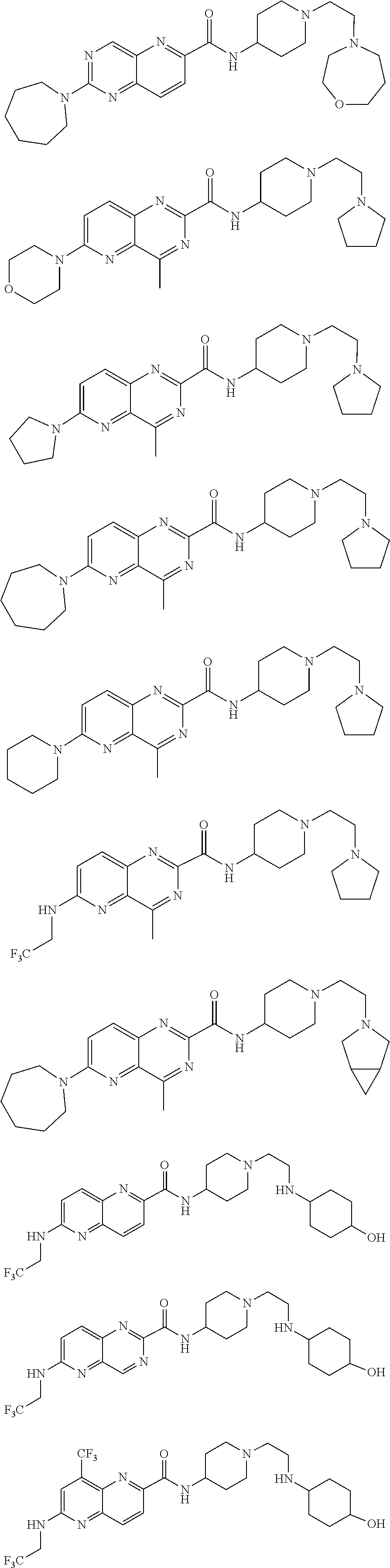

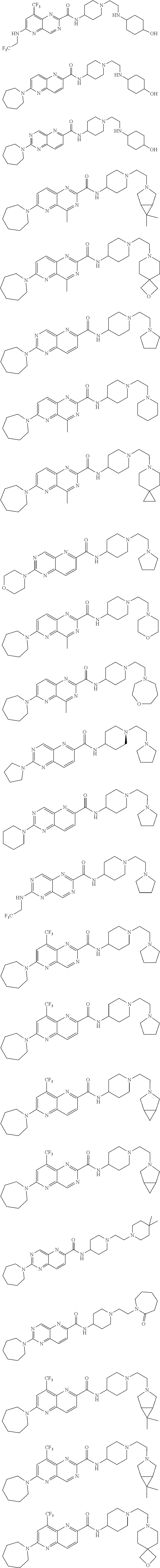

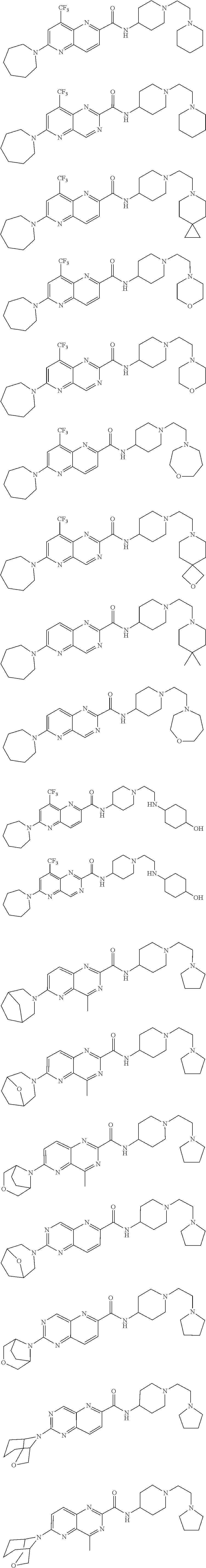

In some embodiments, the compound is a compound of Formula IVc:

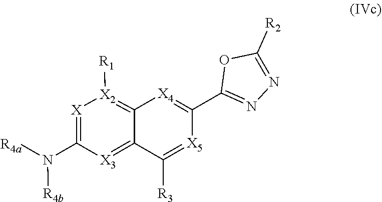

wherein:
X₁ is independently selected from C or N;
X₂ is independently selected from C or N;
X₃ is independently selected from C or N;
X₄ is independently selected from C or N;
X₅ is independently selected from C or N;
R¹ is absent, hydrogen, (C₁-C₈) alkyl, NR⁹R¹⁰, halo, amino, —C≡N, (C₂-C₈) alkenyl, (C₂-C₈) alkynyl, (C C₈) haloalkyl, (C₂-C₈) haloalkenyl, (C₂-C₈) haloalkynyl, (C₁-C₈) alkoxy, (C₁-C₈) haloalkoxy, (C₁-C₈) alkyl (C₁-C₆) alkoxy, 4-7 membered heterocyclyl, 5-6 membered heteroaryl, CH(O), C(O)OR⁸, SF₅, —OH, —SH, (C₁-C₆) hydroxyalkyl, (C₁-C₄) alkylsulfonyl, aminosulfonyl, amino (C₁-C₄) alkylsulfonyl or aryl;
R² is hydrogen, (C₁-C₈) alkyl, NR⁹R¹⁰, halo, amino, —C≡N, (C₂-C₈) alkenyl, (C₂-C₈) alkynyl, (C C₈) haloalkyl, (C₂-C₈) haloalkenyl, (C₂-C₈) haloalkynyl, (C₁-C₈) alkoxy, (C₁-C₈) haloalkoxy, (C₁-C₈) alkyl (C₁-C₆) alkoxy, 4-7 membered heterocyclyl, 5-6 membered heteroaryl, CH(O), C(O)OR⁸, SF₅, —OH, —SH, (C₁-C₆) hydroxyalkyl, (C₁-C₄) alkylsulfonyl, aminosulfonyl, amino (C₁-C₄) alkylsulfonyl or aryl;
R³ is H, (C₁-C₈) alkyl, (C₁-C₈) alkenyl, (C₁-C₈) alkynyl, (C₁-C₈) alkoxy, hydroxyl, halo, amino, amido, amino (C₁-C₈) alkylamido, heterocyclyl, sulfonyl, aminosulfonyl, amino (C₁-C₈) alkysulfonyl, cyano, or (C₁-C₃) haloalkyl;
R^{4a} is selected from H, (C₁-C₈) alkyl, either unsubstituted or substituted with halo, (C₁-C₈) alkoxy, (C₁-C₈) haloalkoxy, S(O)—R⁶, S(O)₂, S(O)₂—R⁶, S(O)₂, C(O)R⁶, C(O)OR⁷ or (C₁-C₈) cycloalkyl;
R^{4b} is selected from H, (C₁-C₈) alkyl, either unsubstituted or substituted with halo, (C₁-C₈) alkoxy, (C₁-C₈) haloalkoxy, S(O)—R⁶, S(O)₂, S(O)₂—R⁶, S(O)₂, C(O)R⁶, C(O)OR⁷ or (C₁-C₈) cycloalkyl;
or R^{4a} and R^{4b} together form a (C₁-C₈) cycloalkyl, a (C₁-C₈) substituted cycloalkyl, a (C₁-C₈) heterocycloalkyl, a (C₁-C₈) substituted heterocycloalkyl, a (C₁-C₈) aryl, a (C₁-C₈) substituted aryl, a (C₁-C₈) heteroaryl or a (C₁-C₈) substituted heteroaryl;
R⁶ is H, alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, halo, (C₁-C₄) haloalkyl and cyano;
R⁷ is H, alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, halo, (C₁-C₄) haloalkyl and cyano;
R⁸ is H, alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, halo, (C₁-C₄) haloalkyl and cyano;
R⁹ is H, (C₁-C₈) alkyl, said (C₁-C₈) alkyl being unsubstituted or substituted with one or more halo;
R¹⁰ is H, (C₁-C₈) alkyl, said (C₁-C₈) alkyl being unsubstituted or substituted with one or more halo, or R⁹ and R¹⁰, together with the nitrogen atom to which they are attached, form a 4 or 5 membered nitrogen containing heterocycle, said 4 or 5 membered nitrogen containing heterocycle being unsubstituted or substituted with one or more halo; R is H, (C₁-C₄) alkyl, (C₁-C₄) haloalkyl, 5-6 membered heterocycle or 5-6 membered heteroaryl;
R¹¹ is selected from H, (C₁-C₄) alkyl, (C₁-C₄) haloalkyl, a 5-membered heterocycle, 5-membered substituted heterocycle, 5-membered heteroaryl, 5-membered substituted heteroaryl, 6-membered heterocycle, 6-membered substituted heterocycle, 6-membered heteroaryl, 6-membered substituted heteroaryl, 5-membered cycloalkyl, or 5-membered substituted cycloalkyl, 6-membered cycloalkyl, or a 6-membered substituted cycloalkyl;
R¹² is selected from H, (C₁-C₄) alkyl, (C₁-C₄) haloalkyl, a 5-membered heterocycle, 5-membered substituted heterocycle, 5-membered heteroaryl, 5-membered substituted heteroaryl, 6-membered heterocycle, 6-membered substituted heterocycle, 6-membered heteroaryl, 6-membered substituted heteroaryl, 5-membered cycloalkyl, or 5-membered substituted cycloalkyl, 6-membered cycloalkyl, or a 6-membered substituted cycloalkyl;
R¹¹ and R¹², together with the nitrogen atom to which they are attached, form a 5-8 membered nitrogen containing heterocycle, said 5-8 membered nitrogen containing heterocycle being unsubstituted or substituted with OH, halo, ═O, or (C₁-C₈) alkyl;
R¹³ is OH, O—(C₁-C₄) alkyl;
R¹⁵ is H, (C₁-C₆) alkyl, (C₁-C) haloalkyl, 4-7 membered heterocycle, 5-6 membered heteroaryl, 3-7 membered cycloalkyl, and wherein each of said 4-7 membered heterocycle, 5-6 membered heteroaryl, 3-7 membered cycloalkyl is unsubstituted or substituted at a substitutable position with one or more ═O, OH, (C₁-C₈) alkyl, (C₁-C₈) haloalkyl, (C₁-C₈) alkoxy, amino, or, (C₁-C₈) aminoalkyl; and
R¹⁶ is H, (C₁-C₈) alkyl, or (C₁-C₈) haloalkyl.

In some embodiments, the compound is a compound selected from:

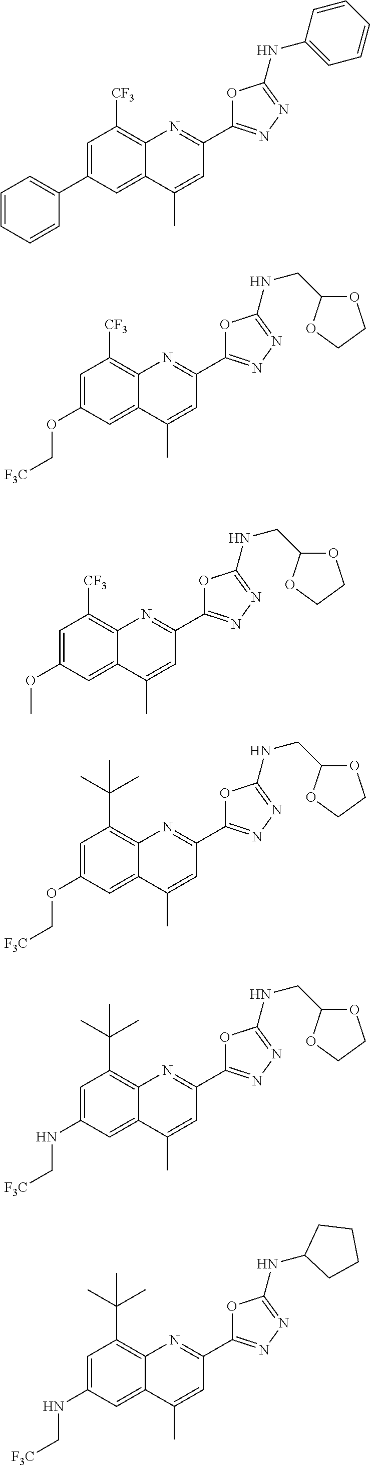 [SMILES: Cc1cc(-c2nnc(Nc3ccccc3)o2)nc2c(C(F)(F)F)cc(-c3ccccc3)cc12 ; Cc1cc(-c2nnc(NCC3OCCO3)o2)nc2c(C(F)(F)F)cc(OCC(F)(F)F)cc12 ; COc1cc(C(F)(F)F)c2nc(-c3nnc(NCC4OCCO4)o3)cc(C)c2c1 ; Cc1cc(-c2nnc(NCC3OCCO3)o2)nc2c(C(C)(C)C)cc(OCC(F)(F)F)cc12 ; Cc1cc(-c2nnc(NCC3OCCO3)o2)nc2c(C(C)(C)C)cc(NCC(F)(F)F)cc12 ; Cc1cc(-c2nnc(NC3CCCC3)o2)nc2c(C(C)(C)C)cc(NCC(F)(F)F)cc12]

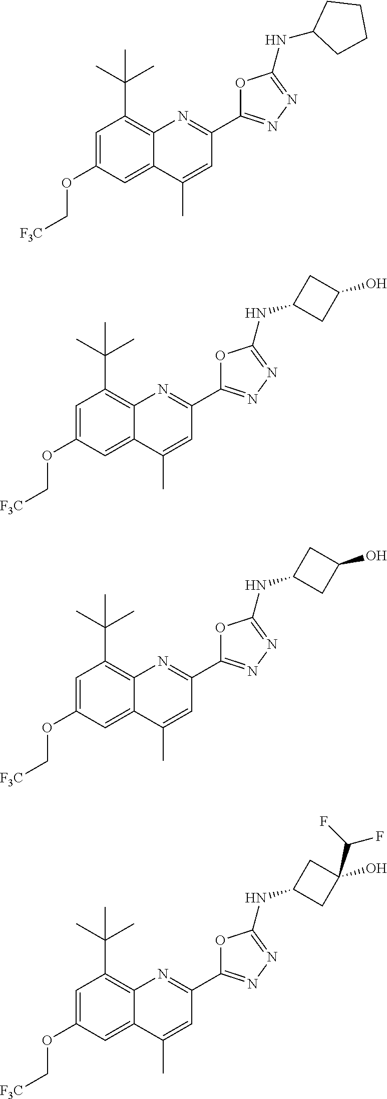 [SMILES: Cc1cc(-c2nnc(NC3CCCC3)o2)nc2c(C(C)(C)C)cc(OCC(F)(F)F)cc12 ; Cc1cc(-c2nnc(N[C@H]3C[C@@H](O)C3)o2)nc2c(C(C)(C)C)cc(OCC(F)(F)F)cc12 ; Cc1cc(-c2nnc(N[C@H]3C[C@H](O)C3)o2)nc2c(C(C)(C)C)cc(OCC(F)(F)F)cc12 ; Cc1cc(-c2nnc(N[C@H]3C[C@](O)(C(F)F)C3)o2)nc2c(C(C)(C)C)cc(OCC(F)(F)F)cc12]

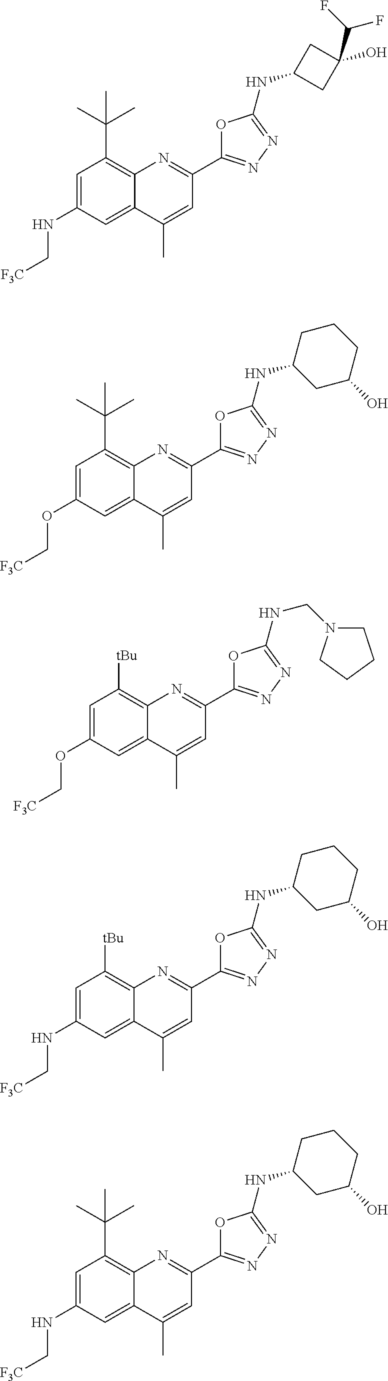 [SMILES: Cc1cc(-c2nnc(N[C@H]3C[C@](O)(C(F)F)C3)o2)nc2c(C(C)(C)C)cc(NCC(F)(F)F)cc12 ; Cc1cc(-c2nnc(N[C@@H]3CCC[C@H](O)C3)o2)nc2c(C(C)(C)C)cc(OCC(F)(F)F)cc12 ; Cc1cc(-c2nnc(NCN3CCCC3)o2)nc2c(C(C)(C)C)cc(OCC(F)(F)F)cc12 ; Cc1cc(-c2nnc(N[C@@H]3CCC[C@H](O)C3)o2)nc2c(C(C)(C)C)cc(NCC(F)(F)F)cc12 ; Cc1cc(-c2nnc(N[C@@H]3CCC[C@H](O)C3)o2)nc2c(C(C)(C)C)cc(NCC(F)(F)F)cc12]

In certain embodiments, the compound is:

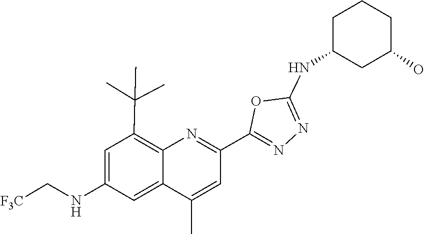

In some embodiments, the compound is a compound of Formula V:

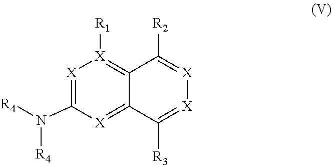

wherein:
each X is independently selected from C or N;
R¹ is absent, hydrogen, (C₁-C₈) alkyl, NR⁹R¹⁰, halo, amino, —C≡N, (C₂-C₈) alkenyl, (C₂-C₈) alkynyl, (C C₈) haloalkyl, (C₂-C₈) haloalkenyl, (C₂-C₈) haloalkynyl, (C₁-C₈) alkoxy, (C₁-C₈) haloalkoxy, (C₁-C₈) alkyl (C₁-C₆) alkoxy, 4-7 membered heterocyclyl, 5-6 membered heteroaryl, CH(O), C(O)OR⁸, SF₅, —OH, —SH, (C₁-C₆) hydroxyalkyl, (C₁-C₄) alkylsulfonyl, aminosulfonyl, amino (C₁-C₄) alkylsulfonyl or aryl;
R² is C(O)NR¹¹R¹², C(O)R¹³, 5-membered heterocycle, 5-membered substituted heterocycle, 5-membered heteroaryl, 5-membered substituted heteroaryl, 6-membered heterocycle, 6-membered substituted heterocycle, 6-membered heteroaryl, 6-membered substituted heteroaryl, 5-membered cycloalkyl, or 5-membered substituted cycloalkyl, 6-membered cycloalkyl, or 6-membered substituted cycloalkyl;
R³ is H, (C₁-C₈) alkyl, (C₁-C₈) alkenyl, (C₁-C₈) alkynyl, (C₁-C₈) alkoxy, hydroxyl, halo, amino, amido, amino (C₁-C₈) alkylamido, heterocyclyl, sulfonyl, aminosulfonyl, amino (C₁-C₈) alkysulfonyl, cyano, or (C₁-C₃) haloalkyl;
each R⁴ is independently selected from (C₁-C₈) alkyl, either unsubstituted or substituted with halo, (C₁-C₈) alkoxy, (C₁-C₈) haloaikoxy, S(O)—R⁶, S(O)₂, S(O)₂—R⁶, S(O)₂, C(O)R⁶, C(O)OR⁷ or (C₁-C₈) cycloalkyl; or both R⁴ together form a (C₁-C₈) cycloalkyl, a (C₁-C₈) substituted cycloalkyl, a (C₁-C₈) heterocycloalkyl, a (C₁-C₈) substituted heterocycloalkyl, a (C₁-C₈) aryl, a (C₁-C₈) substituted aryl, a (C₁-C₈) heteroaryl or a (C₁-C₈) substituted heteroaryl;
R⁶ is H, alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, halo, (C₁-C₄) haloalkyl and cyano;
R⁷ is H, alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, halo, (C₁-C₄) haloalkyl and cyano;
R⁸ is H, alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, halo, (C₁-C₄) haloalkyl and cyano;
R⁹ is H, (C₁-C₈) alkyl, said (C₁-C₈) alkyl being unsubstituted or substituted with one or more halo;
R¹⁰ is H, (C₁-C₈) alkyl, said (C₁-C₈) alkyl being unsubstituted or substituted with one or more halo, or R⁹ and R¹⁰, together with the nitrogen atom to which they are attached, form a 4 or 5 membered nitrogen containing heterocycle, said 4 or 5 membered nitrogen containing heterocycle being unsubstituted or substituted with one or more halo; R is H, (C₁-C₄) alkyl, (C₁-C₄) haloalkyl, 5-6 membered heterocycle or 5-6 membered heteroaryl;
R¹¹ is selected from H, (C₁-C₄) alkyl, (C₁-C₄) haloalkyl, a 5-membered heterocycle, 5-membered substituted heterocycle, 5-membered heteroaryl, 5-membered substituted heteroaryl, 6-membered heterocycle, 6-membered substituted heterocycle, 6-membered heteroaryl, 6-membered substituted heteroaryl, 5-membered cycloalkyl, or 5-membered substituted cycloalkyl, 6-membered cycloalkyl, or a 6-membered substituted cycloalkyl;
R¹² is selected from H, (C₁-C₄) alkyl, (C₁-C₄) haloalkyl, a 5-membered heterocycle, 5-membered substituted heterocycle, 5-membered heteroaryl, 5-membered substituted heteroaryl, 6-membered heterocycle, 6-membered substituted heterocycle, 6-membered heteroaryl, 6-membered substituted heteroaryl, 5-membered cycloalkyl, or 5-membered substituted cycloalkyl, 6-membered cycloalkyl, or a 6-membered substituted cycloalkyl;
R¹¹ and R¹², together with the nitrogen atom to which they are attached, form a 5-8 membered nitrogen containing heterocycle, said 5-8 membered nitrogen containing heterocycle being unsubstituted or substituted with OH, halo, ═O, or (C₁-C₈) alkyl;
R¹³ is OH, O—(C₁-C₄) alkyl;
R¹⁵ is H, (C₁-C₆) alkyl, (C₁-C) haloalkyl, 4-7 membered heterocycle, 5-6 membered heteroaryl, 3-7 membered cycloalkyl, and wherein each of said 4-7 membered heterocycle, 5-6 membered heteroaryl, 3-7 membered cycloalkyl is unsubstituted or substituted at a substitutable position with one or more ═O, OH, (C₁-C₈) alkyl, (C₁-C₈) haloalkyl, (C₁-C₈) alkoxy, amino, or, (C₁-C₈) aminoalkyl; and
R¹⁶ is H, (C₁-C₈) alkyl, or (C₁-C₈) haloalkyl.

In some embodiments, the compound is a compound of Formula Va:

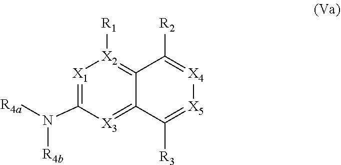

wherein:
X₁ is independently selected from C or N;
X₂ is independently selected from C or N;
X₃ is independently selected from C or N;
X₄ is independently selected from C or N;
X₅ is independently selected from C or N;
R¹ is absent, hydrogen, (C₁-C₈) alkyl, NR⁹R¹⁰, halo, amino, —C≡N, (C₂-C₈) alkenyl, (C₂-C₈) alkynyl, (C C₈) haloalkyl, (C₂-C₈) haloalkenyl, (C₂-C₈) haloalkynyl, (C₁-C₈) alkoxy, (C₁-C₈) haloalkoxy, (C₁-C₈) alkyl (C₁-C₆) alkoxy, 4-7 membered heterocyclyl, 5-6 membered heteroaryl, CH(O), C(O)OR⁸, SF₅, —OH, —SH, (C₁-C₆) hydroxyalkyl, (C₁-C₄) alkylsulfonyl, aminosulfonyl, amino (C₁-C₄) alkylsulfonyl or aryl;
R² is C(O)NR¹¹R¹², C(O)R¹³, 5-membered heterocycle, 5-membered substituted heterocycle, 5-membered heteroaryl, 5-membered substituted heteroaryl, 6-membered heterocycle, 6-membered substituted heterocycle, 6-membered heteroaryl, 6-membered substituted heteroaryl, 5-membered cycloalkyl, or 5-membered substituted cycloalkyl, 6-membered cycloalkyl, or 6-membered substituted cycloalkyl;
R³ is H, (C₁-C₈) alkyl, (C₁-C₈) alkenyl, (C₁-C₈) alkynyl, (C₁-C₈) alkoxy, hydroxyl, halo, amino, amido, amino (C₁-C₈) alkylamido, heterocyclyl, sulfonyl, aminosulfonyl, amino (C₁-C₈) alkysulfonyl, cyano, or (C₁-C₃) haloalkyl;
R^{4a} is selected from H, (C₁-C₈) alkyl, either unsubstituted or substituted with halo, (C₁-C₈) alkoxy, (C₁-C₈) haloalkoxy, S(O)—R⁶, S(O)₂, S(O)₂—R⁶, S(O)₂, C(O)R⁶, C(O)OR⁷ or (C₁-C₈) cycloalkyl;
R^{4b} is selected from H, (C₁-C₈) alkyl, either unsubstituted or substituted with halo, (C₁-C₈) alkoxy, (C₁-C₈) haloalkoxy, S(O)—R⁶, S(O)₂, S(O)₂—R⁶, S(O)₂, C(O)R⁶, C(O)OR⁷ or (C₁-C₈) cycloalkyl;
or R^{4a} and R^{4b} together form a (C₁-C₈) cycloalkyl, a (C₁-C₈) substituted cycloalkyl, a (C₁-C₈) heterocycloalkyl, a (C₁-C₈) substituted heterocycloalkyl, a (C₁-C₈) aryl, a (C₁-C₈) substituted aryl, a (C₁-C₈) heteroaryl or a (C₁-C₈) substituted heteroaryl;
R⁶ is H, alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, halo, (C₁-C₄) haloalkyl and cyano;
R⁷ is H, alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, halo, (C₁-C₄) haloalkyl and cyano;
R⁸ is H, alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, halo, (C₁-C₄) haloalkyl and cyano;
R⁹ is H, (C₁-C₈) alkyl, said (C₁-C₈) alkyl being unsubstituted or substituted with one or more halo;
R¹⁰ is H, (C₁-C₈) alkyl, said (C₁-C₈) alkyl being unsubstituted or substituted with one or more halo, or R⁹ and R¹⁰, together with the nitrogen atom to which they are attached, form a 4 or 5 membered nitrogen containing heterocycle, said 4 or 5 membered nitrogen containing heterocycle being unsubstituted or substituted with one or more halo; R is H, (C₁-C₄) alkyl, (C₁-C₄) haloalkyl, 5-6 membered heterocycle or 5-6 membered heteroaryl;
R¹¹ is selected from H, (C₁-C₄) alkyl, (C₁-C₄) haloalkyl, a 5-membered heterocycle, 5-membered substituted heterocycle, 5-membered heteroaryl, 5-membered substituted heteroaryl, 6-membered heterocycle, 6-membered substituted heterocycle, 6-membered heteroaryl, 6-membered substituted heteroaryl, 5-membered cycloalkyl, or 5-membered substituted cycloalkyl, 6-membered cycloalkyl, or a 6-membered substituted cycloalkyl;
R¹² is selected from H, (C₁-C₄) alkyl, (C₁-C₄) haloalkyl, a 5-membered heterocycle, 5-membered substituted heterocycle, 5-membered heteroaryl, 5-membered substituted heteroaryl, 6-membered heterocycle, 6-membered substituted heterocycle, 6-membered heteroaryl, 6-membered substituted heteroaryl, 5-membered cycloalkyl, or 5-membered substituted cycloalkyl, 6-membered cycloalkyl, or a 6-membered substituted cycloalkyl;
R¹¹ and R¹², together with the nitrogen atom to which they are attached, form a 5-8 membered nitrogen containing heterocycle, said 5-8 membered nitrogen containing heterocycle being unsubstituted or substituted with OH, halo, ═O, or (C₁-C₈) alkyl;
R¹³ is OH, O—(C₁-C₄) alkyl;
R¹⁵ is H, (C₁-C₆) alkyl, (C₁-C) haloalkyl, 4-7 membered heterocycle, 5-6 membered heteroaryl, 3-7 membered cycloalkyl, and wherein each of said 4-7 membered heterocycle, 5-6 membered heteroaryl, 3-7 membered cycloalkyl is unsubstituted or substituted at a substitutable position with one or more ═O, OH, (C₁-C₈) alkyl, (C₁-C₈) haloalkyl, (C₁-C₈) alkoxy, amino, or, (C₁-C₈) aminoalkyl; and
R¹⁶ is H, (C₁-C₈) alkyl, or (C₁-C₈) haloalkyl.

In some embodiments, X₁ is N. In some embodiments, X₁ is C. In some embodiments, X₂ is N. In some embodiments, X₂ is C. In some embodiments, X₃ is N. In some embodiments, X₃ is C. In some embodiments, X₄ is N. In some embodiments, X₄ is C. In some embodiments, X₅ is N. In some embodiments, X₅ is C.

In some embodiments, X₁ is N, X₂ is C, X₃ is N, X₄ is N and X₅ is C. In some embodiments, X₁ is N, X₂ is C, X₃ is C, X₄ is N and X₅ is N. In some embodiments, X₁ is C, X₂ is N, X₃ is C, X₄ is C and X₅ is N. In some embodiments, X₁ is C, X₂ is C, X₃ is N, X₄ is N and X₅ is C. In some embodiments, X₁ is N, X₂ is C, X₃ is N, X₄ is N and X₅ is N.

In some embodiments, R₁ is selected from hydrogen, (C₁-C₈) alkyl, halo or haloalkyl. In some instances, R₁ is hydrogen. In some instances, R₁ is a (C₁-C₈) alkyl, such as methyl, ethyl, propyl, i-propyl, butyl, t-butyl, i-butyl or pentyl. In certain instances, R₁ is methyl. In certain instances, R₁ is t-butyl. In some instances, R₁ is halo, such as fluoro, chloro or bromo. In certain instances, R₁ is fluoro. In some instances, R₁ is a haloalkyl, such as CF₃, CCl₃ or CBr₃. In certain instances, R₁ is CF₃.

In some embodiments, R² is C(O)NR¹¹R¹² where R¹¹ is selected from H, (C₁-C₄) alkyl, (C₁-C₄) haloalkyl, a 5-membered heterocycle, 5-membered substituted heterocycle, 5-membered heteroaryl, 5-membered substituted heteroaryl, 6-membered heterocycle, 6-membered substituted heterocycle, 6-membered heteroaryl, 6-membered substituted heteroaryl, 5-membered cycloalkyl, or 5-membered substituted cycloalkyl, 6-membered cycloalkyl, or a 6-membered substituted cycloalkyl; and R¹² is selected from H, (C₁-C₄) alkyl, (C₁-C₄) haloalkyl, a 5-membered heterocycle, 5-membered substituted heterocycle, 5-membered heteroaryl, 5-membered substituted heteroaryl, 6-membered heterocycle, 6-membered substituted heterocycle, 6-membered heteroaryl, 6-membered substituted heteroaryl, 5-membered cycloalkyl, or 5-membered substituted cycloalkyl, 6-membered cycloalkyl, or a 6-membered substituted cycloalkyl.

In some embodiments, R² is a 5-membered heteroaryl. In some instances, R² is:

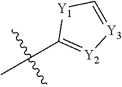

where
Y₁ is O, NH, CH₂ or S;
Y₂ is CH or N; and
Y₃ is CH or N.

In some embodiments, R¹¹ is H and R¹² is selected from a 5-membered heterocycle, 5-membered substituted heterocycle, 5-membered heteroaryl, 5-membered substituted heteroaryl, 6-membered heterocycle, 6-membered substituted heterocycle, 6-membered heteroaryl, 6-membered substituted heteroaryl, 5-membered cycloalkyl, or 5-membered substituted cycloalkyl, 6-membered cycloalkyl, or a 6-membered substituted cycloalkyl. In some instances, R¹¹ is H and R¹² is a 6-membered heterocycle. In some instances, R¹¹ is H and R¹² is a substituted 6-membered heterocycle.

In certain instances, R¹¹ is H and R¹² is substituted or unsubstituted piperidinyl, pyrrolidinyl, piperazinyl, or azepanyl group. In another embodiment, R¹² is a substituted or unsubstituted piperidin-3-yl or piperidin-4-yl group. In another embodiment, the substituted piperidin-4-yl group is:

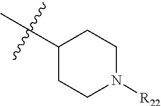

wherein R₂₂ is a substituted or unsubstituted C₂-C₃ alkyl. In another embodiment, R₂₂ is C₂-C₃ alkyl. In another embodiment, R₂₂ is a substituted ethyl group. In another embodiment, R₂₂ is —CH₂CH₂—NR₂₃R₂₄ and R₂₃ and R₂₄ are independently C₁-C₃ alkyl or C₁-C₃ alkyl substituted with a C₃-C₄ cycloalkyl ring, or R₂₃ and R₂₄ together with the nitrogen atom to which they are bonded form a substituted or unsubstituted 5-8 membered heterocyclic ring. Suitable substituents for the 5-8 membered heterocyclic rings include, without limitation, 1 or 2 methyl, hydroxymethyl, methoxymethyl, or hydroxyl groups. In another embodiment, the 5-8 membered heterocyclic ring is a pyrrolidinyl, piperidinyl, or azepanyl ring, which is substituted or unsubstituted. Within this embodiment, in one embodiment, L is —CO—NH—, wherein the NH moiety is bonded to the piperidinyl moiety. In another embodiment, R₂ is —NR₂₃R₂₄ and R₂₃ and R₂₄ are independently C₁-C₃ alkyl or C₁-C₃ alkyl substituted with a C₃-C₄ cycloalkyl ring, or R₂₃ and R₂₄ together with the nitrogen atom to which they are bonded form a substituted or unsubstituted 5-8 membered heterocyclic ring. Suitable substituents for the 5-8 membered heterocyclic rings include, without limitation, 1 or 2 methyl, hydroxymethyl, methoxymethyl, or hydroxyl groups. In another embodiment, the 5-8 membered heterocyclic ring is a pyrrolidinyl, piperidinyl, or azepanyl ring, which is substituted or unsubstituted. Within this embodiment, in one embodiment, L is —(CH₂)ₙ—, —O—(CH₂) n, or —CH₂—O—(CH₂)ₙ— wherein, the left hand side of the L is bonded to the azaindazole moiety and n is 1, 2, 3, or 4.

In certain instances, R¹¹ is H and R¹² is:

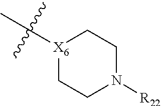

wherein X₆ is N or C; and
R₂₂ is a substituted or unsubstituted C₂-C₃ alkyl. In another embodiment, R₂₂ is C₂-C₃ alkyl. In another embodiment, R₂₂ is a substituted ethyl group. In another embodiment, R₂₂ is —CH₂CH₂—NR₂₃R₂₄ and R₂₃ and R₂₄ are independently C₁-C₃ alkyl or C₁-C₃ alkyl substituted with a C₃-C₄ cycloalkyl ring, or R₂₃ and R₂₄ together with the nitrogen atom to which they are bonded form a substituted or unsubstituted 5-8 membered heterocyclic ring. Suitable substituents for the 5-8 membered heterocyclic rings include, without limitation, 1 or 2 methyl, hydroxymethyl, methoxymethyl, or hydroxyl groups. In another embodiment, the 5-8 membered heterocyclic ring is a pyrrolidinyl, piperidinyl, or azepanyl ring, which is substituted or unsubstituted.

Within this embodiment, in one embodiment, L is —CO—NH—, wherein the NH moiety is bonded to the piperidinyl moiety. In another embodiment, R₂ is —NR₂₃R₂₄ and R₂₃ and R₂₄ are independently C₁-C₃ alkyl or C₁-C₃ alkyl substituted with a C₃-C₄ cycloalkyl ring, or R₂₃ and R₂₄ together with the nitrogen atom to which they are bonded form a substituted or unsubstituted 5-8 membered heterocyclic ring. Suitable substituents for the 5-8 membered heterocyclic rings include, without limitation, 1 or 2 methyl, hydroxymethyl, methoxymethyl, or hydroxyl groups. In another embodiment, the 5-8 membered heterocyclic ring is a pyrrolidinyl, piperidinyl, or azepanyl ring, which is substituted or unsubstituted. Within this embodiment, in one embodiment, L is —(CH₂) n, —O—(CH₂)ₙ—, or —CH₂—O—(CH₂)ₙ— wherein, the left hand side of the L is bonded to the azaindazole moiety and n is 1, 2, 3, or 4.

In some embodiments, R₄ₐ is H, (C₁-C₈) alkyl, either unsubstituted or substituted with halo, (C₁-C₈) alkoxy, (C₁-C₈) haloalkoxy or (C₁-C₈) cycloalkyl. In some embodiments, R_{4b} is H (C₁-C₈) alkyl, either unsubstituted or substituted with halo, (C₁-C₈) alkoxy, (C₁-C₈) haloaikoxy, S(O)—R₆, S(O)₂, S(O)₂—R₆, S(O)₂, C(O)R₆, C(O)OR₇ or (C₁-C₈) cycloalkyl. In some embodiments, R₄ₐ is (C₁-C₈) alkyl. In some embodiments, R₄ₐ is (C₁-C₈) alkyl substituted with halo. In some instances, R₄ₐ is ethyl substituted with halo.

In some embodiments, R^{4a} and R^{4b} together form a (C₁-C₈) cycloalkyl, a (C₁-C₈) substituted cycloalkyl, a (C₁-C₈) heterocycloalkyl, a (C₁-C₈) substituted heterocycloalkyl, a (C₁-C₈) aryl, a (C₁-C₈) substituted aryl, a (C₁-C₈) heteroaryl or a (C₁-C₈) substituted heteroaryl. In some instances, R^{4a} and R^{4b} together with the nitrogen to which they are bonded form a (C₁-C₈) heterocycloalkyl. In certain instances, R^{4a} and R^{4b} together form together with the nitrogen form a seven-membered heterocycloalkyl.

In some embodiments, the compound is a compound of Formula Vb:

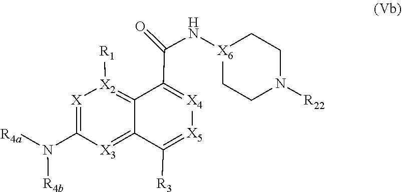

wherein:
X₁ is independently selected from C or N;
X₂ is independently selected from C or N;
X₃ is independently selected from C or N;
X₄ is independently selected from C or N;
X₅ is independently selected from C or N;
X₆ is independently selected from C or N;
R¹ is absent, hydrogen, (C₁-C₈) alkyl, NR⁹R¹⁰, halo, amino, —C≡N, (C₂-C₈) alkenyl, (C₂-C₈) alkynyl, (C C₈) haloalkyl, (C₂-C₈) haloalkenyl, (C₂-C₈) haloalkynyl, (C₁-C₈) alkoxy, (C₁-C₈) haloalkoxy, (C₁-C₈) alkyl (C₁-C₆) alkoxy, 4-7 membered heterocyclyl, 5-6 membered heteroaryl, CH(O), C(O)OR⁸, SF₅, —OH, —SH, (C₁-C₆) hydroxyalkyl, (C₁-C₄) alkylsulfonyl, aminosulfonyl, amino (C₁-C₄) alkylsulfonyl or aryl;
R² is C(O)NR¹¹R¹², C(O)R¹³, 5-membered heterocycle, 5-membered substituted heterocycle, 5-membered heteroaryl, 5-membered substituted heteroaryl, 6-membered heterocycle, 6-membered substituted heterocycle, 6-membered heteroaryl, 6-membered substituted heteroaryl, 5-membered cycloalkyl, or 5-membered substituted cycloalkyl, 6-membered cycloalkyl, or 6-membered substituted cycloalkyl;
R³ is H, (C₁-C₈) alkyl, (C₁-C₈) alkenyl, (C₁-C₈) alkynyl, (C₁-C₈) alkoxy, hydroxyl, halo, amino, amido, amino (C₁-C₈) alkylamido, heterocyclyl, sulfonyl, aminosulfonyl, amino (C₁-C₈) alkysulfonyl, cyano, or (C₁-C₃) haloalkyl;
R^{4a} is selected from H, (C₁-C₈) alkyl, either unsubstituted or substituted with halo, (C₁-C₈) alkoxy, (C₁-C₈) haloalkoxy, S(O)—R⁶, S(O)₂, S(O)₂—R⁶, S(O)₂, C(O)R⁶, C(O)OR⁷ or (C₁-C₈) cycloalkyl;
R^{4b} is selected from H, (C₁-C₈) alkyl, either unsubstituted or substituted with halo, (C₁-C₈) alkoxy, (C₁-C₈) haloalkoxy, S(O)—R⁶, S(O)₂, S(O)₂—R⁶, S(O)₂, C(O)R⁶, C(O)OR⁷ or (C₁-C₈) cycloalkyl;
or R^{4a} and R^{4b} together form a (C₁-C₈) cycloalkyl, a (C₁-C₈) substituted cycloalkyl, a (C₁-C₈) heterocycloalkyl, a (C₁-C₈) substituted heterocycloalkyl, a (C₁-C₈) aryl, a (C₁-C₈) substituted aryl, a (C₁-C₈) heteroaryl or a (C₁-C₈) substituted heteroaryl;
R⁶ is H, alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, halo, (C₁-C₄) haloalkyl and cyano;
R⁷ is H, alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, halo, (C₁-C₄) haloalkyl and cyano;
R⁸ is H, alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, halo, (C₁-C₄) haloalkyl and cyano;
R⁹ is H, (C₁-C₈) alkyl, said (C₁-C₈) alkyl being unsubstituted or substituted with one or more halo;
R¹⁰ is H, (C₁-C₈) alkyl, said (C₁-C₈) alkyl being unsubstituted or substituted with one or more halo, or R⁹ and R¹⁰, together with the nitrogen atom to which they are attached, form a 4 or 5 membered nitrogen containing heterocycle, said 4 or 5 membered nitrogen containing heterocycle being unsubstituted or substituted with one or more halo; R is H, (C₁-C₄) alkyl, (C₁-C₄) haloalkyl, 5-6 membered heterocycle or 5-6 membered heteroaryl;
R¹¹ is selected from H, (C₁-C₄) alkyl, (C₁-C₄) haloalkyl, a 5-membered heterocycle, 5-membered substituted heterocycle, 5-membered heteroaryl, 5-membered substituted heteroaryl, 6-membered heterocycle, 6-membered substituted heterocycle, 6-membered heteroaryl, 6-membered substituted heteroaryl, 5-membered cycloalkyl, or 5-membered substituted cycloalkyl, 6-membered cycloalkyl, or a 6-membered substituted cycloalkyl;
R¹² is selected from H, (C₁-C₄) alkyl, (C₁-C₄) haloalkyl, a 5-membered heterocycle, 5-membered substituted heterocycle, 5-membered heteroaryl, 5-membered substituted heteroaryl, 6-membered heterocycle, 6-membered substituted heterocycle, 6-membered heteroaryl, 6-membered substituted heteroaryl, 5-membered cycloalkyl, or 5-membered substituted cycloalkyl, 6-membered cycloalkyl, or a 6-membered substituted cycloalkyl;
R¹¹ and R¹², together with the nitrogen atom to which they are attached, form a 5-8 membered nitrogen containing heterocycle, said 5-8 membered nitrogen containing heterocycle being unsubstituted or substituted with OH, halo, ═O, or (C₁-C₈) alkyl;
R¹³ is OH, O—(C₁-C₄) alkyl;
R¹⁵ is H, (C₁-C₆) alkyl, (C₁-C) haloalkyl, 4-7 membered heterocycle, 5-6 membered heteroaryl, 3-7 membered cycloalkyl, and wherein each of said 4-7 membered heterocycle, 5-6 membered heteroaryl, 3-7 membered cycloalkyl is unsubstituted or substituted at a substitutable position with one or more ═O, OH, (C₁-C₈) alkyl, (C₁-C₈) haloalkyl, (C₁-C₈) alkoxy, amino, or, (C₁-C₈) aminoalkyl; and
R¹⁶ is H, (C₁-C₈) alkyl, or (C₁-C₈) haloalkyl; and
R₂₂ is a substituted or unsubstituted C₂-C₃ alkyl. In another embodiment, R₂₂ is C₂-C₃ alkyl. In another embodiment, R₂₂ is a substituted ethyl group. In another embodiment, R₂₂ is —CH₂CH₂—NR₂₃R₂₄ and R₂₃ and R₂₄ are independently C₁-C₃ alkyl or C₁-C₃ alkyl substituted with a C₃-C₄ cycloalkyl ring, or R₂₃ and R₂₄ together with the nitrogen atom to which they are bonded form a substituted or unsubstituted 5-8 membered heterocyclic ring. Suitable substituents for the 5-8 membered heterocyclic rings include, without limitation, 1 or 2 methyl, hydroxymethyl, methoxymethyl, or hydroxyl groups. In another embodiment, the 5-8 membered heterocyclic ring is a pyrrolidinyl, piperidinyl, or azepanyl ring, which is substituted or unsubstituted. Within this embodiment, in one embodiment, L is —CO—NH—, wherein the NH moiety is bonded to the piperidinyl moiety. In another embodiment, R₂ is —NR₂₃R₂₄ and R₂₃ and R₂₄ are independently C₁-C₃ alkyl or C₁-C₃ alkyl substituted with a C₃-C₄ cycloalkyl ring, or R₂₃ and R₂₄ together with the nitrogen atom to which they are bonded form a substituted or unsubstituted 5-8 membered heterocyclic ring. Suitable substituents for the 5-8 membered heterocyclic rings include, without limitation, 1 or 2 methyl, hydroxymethyl, methoxymethyl, or hydroxyl groups. In another embodiment, the 5-8 membered heterocyclic ring is a pyrrolidinyl, piperidinyl, or azepanyl ring, which is substituted or unsubstituted. Within this embodiment, in one embodiment, L is —(CH₂)ₙ—, —O—(CH₂)ₙ—, or —CH₂—O—(CH₂)ₙ— wherein, the left hand side of the L is bonded to the azaindazole moiety and n is 1, 2, 3, or 4.

In some embodiments, the compound is a compound selected from:

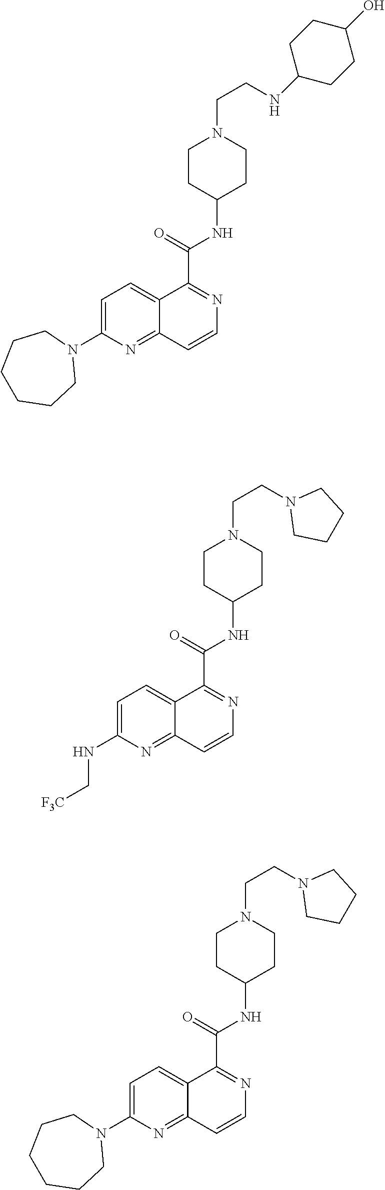 [SMILES: O=C(NC1CCN(CCNC2CCC(O)CC2)CC1)c1nccc2nc(N3CCCCCC3)ccc12 ; O=C(NC1CCN(CCN2CCCC2)CC1)c1nccc2nc(NCC(F)(F)F)ccc12 ; O=C(NC1CCN(CCN2CCCC2)CC1)c1nccc2nc(N3CCCCCC3)ccc12]

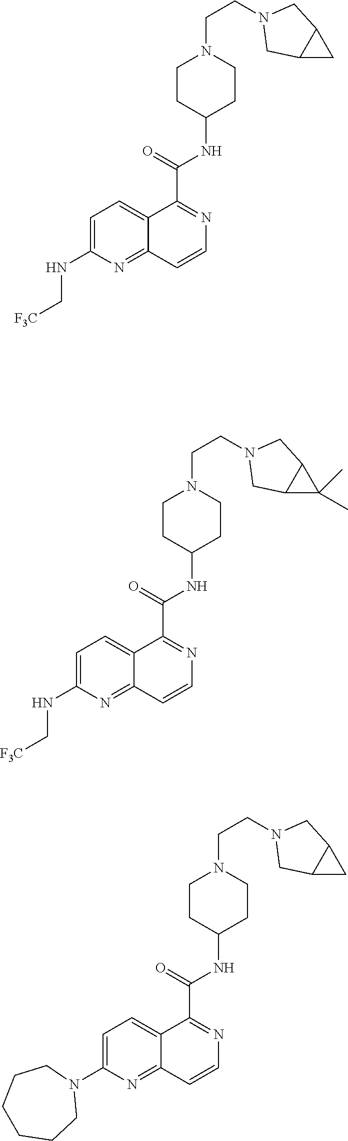 [SMILES: O=C(NC1CCN(CCN2CC3CC3C2)CC1)c1nccc2nc(NCC(F)(F)F)ccc12 ; CC1(C)C2CN(CCN3CCC(NC(=O)c4nccc5nc(NCC(F)(F)F)ccc45)CC3)CC21 ; O=C(NC1CCN(CCN2CC3CC3C2)CC1)c1nccc2nc(N3CCCCCC3)ccc12]

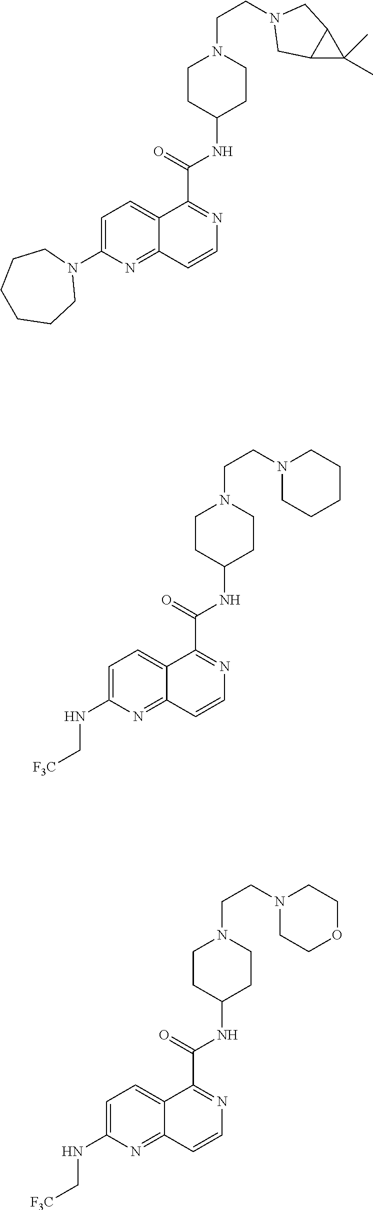 [SMILES: CC1(C)C2CN(CCN3CCC(NC(=O)c4nccc5nc(N6CCCCCC6)ccc45)CC3)CC21 ; O=C(NC1CCN(CCN2CCCCC2)CC1)c1nccc2nc(NCC(F)(F)F)ccc12 ; O=C(NC1CCN(CCN2CCOCC2)CC1)c1nccc2nc(NCC(F)(F)F)ccc12]

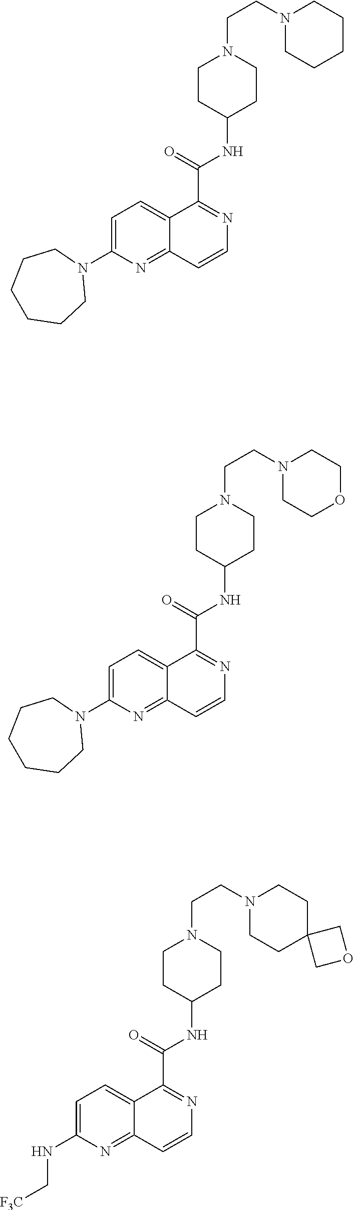 [SMILES: O=C(NC1CCN(CCN2CCCCC2)CC1)c1nccc2nc(N3CCCCCC3)ccc12 ; O=C(NC1CCN(CCN2CCOCC2)CC1)c1nccc2nc(N3CCCCCC3)ccc12 ; O=C(NC1CCN(CCN2CCC3(CC2)COC3)CC1)c1nccc2nc(NCC(F)(F)F)ccc12]

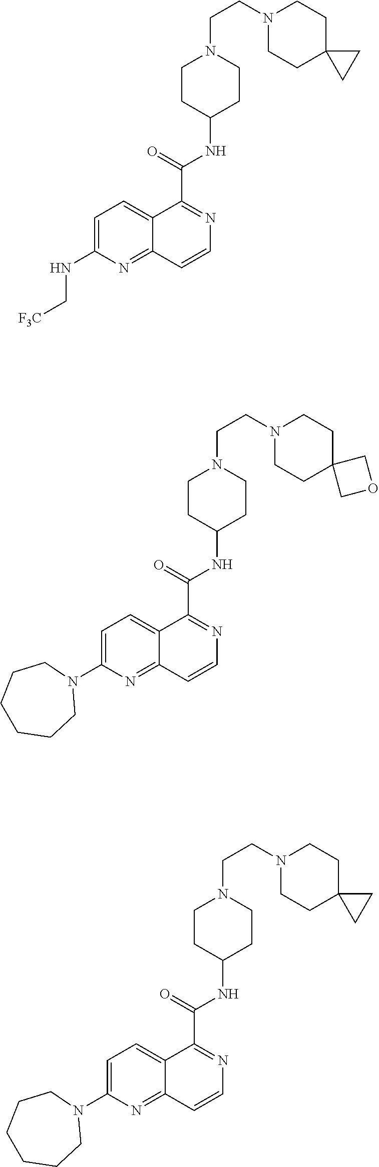 [SMILES: O=C(NC1CCN(CCN2CCC3(CC2)CC3)CC1)c1nccc2nc(NCC(F)(F)F)ccc12 ; O=C(NC1CCN(CCN2CCC3(CC2)COC3)CC1)c1nccc2nc(N3CCCCCC3)ccc12 ; O=C(NC1CCN(CCN2CCC3(CC2)CC3)CC1)c1nccc2nc(N3CCCCCC3)ccc12]

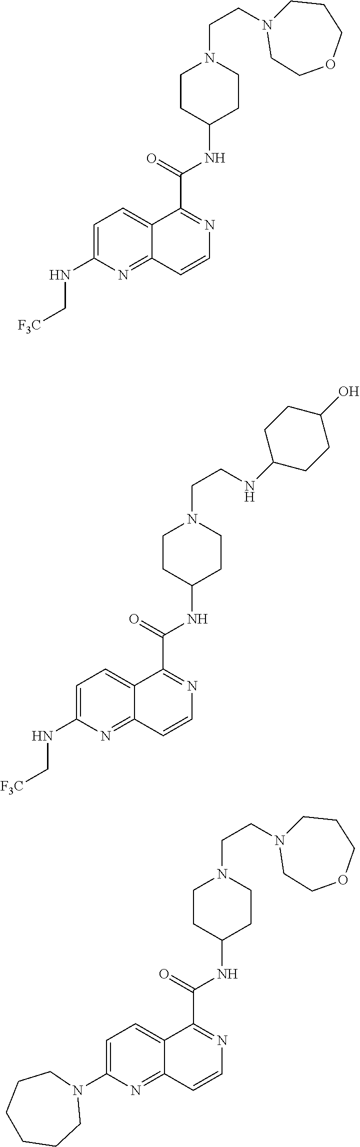 [SMILES: O=C(NC1CCN(CCN2CCCOCC2)CC1)c1nccc2nc(NCC(F)(F)F)ccc12 ; O=C(NC1CCN(CCNC2CCC(O)CC2)CC1)c1nccc2nc(NCC(F)(F)F)ccc12 ; O=C(NC1CCN(CCN2CCCOCC2)CC1)c1nccc2nc(N3CCCCCC3)ccc12]

In some embodiments, the compound is a compound of Formula Vc:

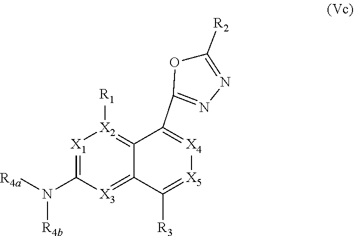

wherein:
X₁ is independently selected from C or N;
X₂ is independently selected from C or N;
X₃ is independently selected from C or N;
X₄ is independently selected from C or N;
X₅ is independently selected from C or N;
R¹ is absent, hydrogen, (C₁-C₈) alkyl, NR⁹R¹⁰, halo, amino, —C≡N, (C₂-C₈) alkenyl, (C₂-C₈) alkynyl, (C C₈) haloalkyl, (C₂-C₈) haloalkenyl, (C₂-C₈) haloalkynyl, (C₁-C₈) alkoxy, (C₁-C₈) haloalkoxy, (C₁-C₈) alkyl (C₁-C₆) alkoxy, 4-7 membered heterocyclyl, 5-6 membered heteroaryl, CH(O), C(O)OR⁸, SF₅, —OH, —SH, (C₁-C₆) hydroxyalkyl, (C₁-C₄) alkylsulfonyl, aminosulfonyl, amino (C₁-C₄) alkylsulfonyl or aryl;
R² is hydrogen, (C₁-C₈) alkyl, NR⁹R¹⁰, halo, amino, —C≡N, (C₂-C₈) alkenyl, (C₂-C₈) alkynyl, (C C₈) haloalkyl, (C₂-C₈) haloalkenyl, (C₂-C₈) haloalkynyl, (C₁-C₈) alkoxy, (C₁-C₈) haloalkoxy, (C₁-C₈) alkyl (C₁-C₆) alkoxy, 4-7 membered heterocyclyl, 5-6 membered heteroaryl, CH(O), C(O)OR⁸, SF₅, —OH, —SH, (C₁-C₆) hydroxyalkyl, (C₁-C₄) alkylsulfonyl, aminosulfonyl, amino (C₁-C₄) alkylsulfonyl or aryl;
R³ is H, (C₁-C₈) alkyl, (C₁-C₈) alkenyl, (C₁-C₈) alkynyl, (C₁-C₈) alkoxy, hydroxyl, halo, amino, amido, amino (C₁-C₈) alkylamido, heterocyclyl, sulfonyl, aminosulfonyl, amino (C₁-C₈) alkysulfonyl, cyano, or (C₁-C₃) haloalkyl;
R^{4a} is selected from H, (C₁-C₈) alkyl, either unsubstituted or substituted with halo, (C₁-C₈) alkoxy, (C₁-C₈) haloalkoxy, S(O)—R⁶, S(O)₂, S(O)₂—R⁶, S(O)₂, C(O)R⁶, C(O)OR⁷ or (C₁-C₈) cycloalkyl;
R^{4b} is selected from H, (C₁-C₈) alkyl, either unsubstituted or substituted with halo, (C₁-C₈) alkoxy, (C₁-C₈) haloalkoxy, S(O)—R⁶, S(O)₂, S(O)₂—R⁶, S(O)₂, C(O)R⁶, C(O)OR⁷ or (C₁-C₈) cycloalkyl;
or R^{4a} and R^{4b} together form a (C₁-C₈) cycloalkyl, a (C₁-C₈) substituted cycloalkyl, a (C₁-C₈) heterocycloalkyl, a (C₁-C₈) substituted heterocycloalkyl, a (C₁-C₈) aryl, a (C₁-C₈) substituted aryl, a (C₁-C₈) heteroaryl or a (C₁-C₈) substituted heteroaryl;
R⁶ is H, alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, halo, (C₁-C₄) haloalkyl and cyano;
R⁷ is H, alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, halo, (C₁-C₄) haloalkyl and cyano;
R⁸ is H, alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, halo, (C₁-C₄) haloalkyl and cyano;
R⁹ is H, (C₁-C₈) alkyl, said (C₁-C₈) alkyl being unsubstituted or substituted with one or more halo;
R¹⁰ is H, (C₁-C₈) alkyl, said (C₁-C₈) alkyl being unsubstituted or substituted with one or more halo, or R⁹ and R¹⁰, together with the nitrogen atom to which they are attached, form a 4 or 5 membered nitrogen containing heterocycle, said 4 or 5 membered nitrogen containing heterocycle being unsubstituted or substituted with one or more halo; R is H, (C₁-C₄) alkyl, (C₁-C₄) haloalkyl, 5-6 membered heterocycle or 5-6 membered heteroaryl;
R¹¹ is selected from H, (C₁-C₄) alkyl, (C₁-C₄) haloalkyl, a 5-membered heterocycle, 5-membered substituted heterocycle, 5-membered heteroaryl, 5-membered substituted heteroaryl, 6-membered heterocycle, 6-membered substituted heterocycle, 6-membered heteroaryl, 6-membered substituted heteroaryl, 5-membered cycloalkyl, or 5-membered substituted cycloalkyl, 6-membered cycloalkyl, or a 6-membered substituted cycloalkyl;
R¹² is selected from H, (C₁-C₄) alkyl, (C₁-C₄) haloalkyl, a 5-membered heterocycle, 5-membered substituted heterocycle, 5-membered heteroaryl, 5-membered substituted heteroaryl, 6-membered heterocycle, 6-membered substituted heterocycle, 6-membered heteroaryl, 6-membered substituted heteroaryl, 5-membered cycloalkyl, or 5-membered substituted cycloalkyl, 6-membered cycloalkyl, or a 6-membered substituted cycloalkyl;
R¹¹ and R¹², together with the nitrogen atom to which they are attached, form a 5-8 membered nitrogen containing heterocycle, said 5-8 membered nitrogen containing heterocycle being unsubstituted or substituted with OH, halo, ═O, or (C₁-C₈) alkyl;
R¹³ is OH, O—(C₁-C₄) alkyl;
R¹⁵ is H, (C₁-C₆) alkyl, (C₁-C) haloalkyl, 4-7 membered heterocycle, 5-6 membered heteroaryl, 3-7 membered cycloalkyl, and wherein each of said 4-7 membered heterocycle, 5-6 membered heteroaryl, 3-7 membered cycloalkyl is unsubstituted or substituted at a substitutable position with one or more ═O, OH, (C₁-C₈) alkyl, (C₁-C₈) haloalkyl, (C₁-C₈) alkoxy, amino, or, (C₁-C₈) aminoalkyl; and
R¹⁶ is H, (C₁-C₈) alkyl, or (C₁-C₈) haloalkyl.

In some embodiments, the compound is a compound of Formula VI:

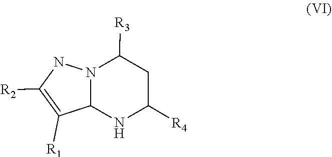

where:
R₁ is hydrogen; halo, haloalkyl C₁-C₆ alkyl; C₁-C₆ alkyl substituted with a substituted or unsubstituted C₃-C₈ cycloalkyl, 5-8 membered heterocyclyl, or a 6 membered aryl group; C₂-C₆ alkenyl; substituted or unsubstituted C₃-C₈ cycloalkyl, —CO—(C₃-C₈ cycloalkyl), —CO—(C₁-C₆ alkyl), —CO-aryl, —CO-heteroaryl, —CO-heterocyclyl, —SO₂—(C₁-C₆ alkyl), or —SO₂—(C₃-C₈ cycloalkyl) group;
R² is C(O)NR¹¹R¹², C(O)R¹³, 5-membered heterocycle, 5-membered substituted heterocycle, 5-membered heteroaryl, 5-membered substituted heteroaryl, 6-membered heterocycle, 6-membered substituted heterocycle, 6-membered heteroaryl, 6-membered substituted heteroaryl, 5-membered cycloalkyl, or 5-membered substituted cycloalkyl, 6-membered cycloalkyl, or 6-membered substituted cycloalkyl;
R³ is H, (C₁-C₈) alkyl, (C₁-C₈) alkenyl, (C₁-C₈) alkynyl, (C₁-C₈) alkoxy, hydroxyl, halo, amino, amido, amino (C₁-C₈) alkylamido, heterocyclyl, sulfonyl, aminosulfonyl, amino (C₁-C₈) alkysulfonyl, cyano, or (C₁-C₃) haloalkyl;
R¹¹ is selected from H, (C₁-C₄) alkyl, (C₁-C₄) haloalkyl, a 5-membered heterocycle, 5-membered substituted heterocycle, 5-membered heteroaryl, 5-membered substituted heteroaryl, 6-membered heterocycle, 6-membered substituted heterocycle, 6-membered heteroaryl, 6-membered substituted heteroaryl, 5-membered cycloalkyl, or 5-membered substituted cycloalkyl, 6-membered cycloalkyl, or a 6-membered substituted cycloalkyl or a alkyl-substituted with one or more of a 5-membered heterocycle, 5-membered substituted heterocycle, 5-membered heteroaryl, 5-membered substituted heteroaryl, 6-membered heterocycle, 6-membered substituted heterocycle, 6-membered heteroaryl, 6-membered substituted heteroaryl, 5-membered cycloalkyl, or 5-membered substituted cycloalkyl, 6-membered cycloalkyl, or a 6-membered substituted cycloalkyl
R¹² is selected from H, (C₁-C₄) alkyl, (C₁-C₄) haloalkyl, a 5-membered heterocycle, 5-membered substituted heterocycle, 5-membered heteroaryl, 5-membered substituted heteroaryl, 6-membered heterocycle, 6-membered substituted heterocycle, 6-membered heteroaryl, 6-membered substituted heteroaryl, 5-membered cycloalkyl, or 5-membered substituted cycloalkyl, 6-membered cycloalkyl, or a 6-membered substituted cycloalkyl or a alkyl-substituted with one or more of a 5-membered heterocycle, 5-membered substituted heterocycle, 5-membered heteroaryl, 5-membered substituted heteroaryl, 6-membered heterocycle, 6-membered substituted heterocycle, 6-membered heteroaryl, 6-membered substituted heteroaryl, 5-membered cycloalkyl, or 5-membered substituted cycloalkyl, 6-membered cycloalkyl, or a 6-membered substituted cycloalkyl
R¹¹ and R¹², together with the nitrogen atom to which they are attached, form a 5-8 membered nitrogen containing heterocycle, said 5-8 membered nitrogen containing heterocycle being unsubstituted or substituted with OH, halo, ═O, or (C₁-C₈) alkyl; and
R⁴ is hydrogen; halo, haloalkyl C₁-C₆ alkyl; C₁-C₆ alkyl substituted with a substituted or unsubstituted C₃-C₈ cycloalkyl, 5-8 membered heterocyclyl, a 5-8 membered aryl, a 5-8 membered heteroaryl, C₂-C₆ alkenyl; substituted or unsubstituted C₃-C₈ cycloalkyl, —CO—(C₃-C₈ cycloalkyl), —CO—(C₁-C₆ alkyl), —CO-aryl, —CO-heteroaryl, —CO-heterocyclyl, —SO₂—(C₁-C₆ alkyl), or —SO₂ (C₃-C₈ cycloalkyl) group.

In some embodiments, R₃ is selected from hydrogen, (C₁-C₈) alkyl, halo or haloalkyl. In some instances, R₃ is hydrogen. In some instances, R₃ is a (C₁-C₈) alkyl, such as methyl, ethyl, propyl, i-propyl, butyl, t-butyl, i-butyl or pentyl. In certain instances, R₃ is methyl. In certain instances, R₃ is t-butyl. In some instances, R₃ is halo, such as fluoro, chloro or bromo. In certain instances, R₃ is fluoro. In some instances, R₃ is a haloalkyl, such as CF₃, CCl₃ or CBr₃. In certain instances, R₃ is CF₃.

In some embodiments, R₄ is a 5-membered heteroaryl. In some instances, R₂ is:

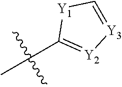

where
Y₁ is O, NH, CH₂ or S;
Y₂ is CH or N; and
Y₃ is CH or N.

In certain instances, R₄ is furan. In certain instances, R₄ is pyrrole. In certain instances, R₄ is thiophene. In certain instances, R₄ is imidazole. In certain instances, R₄ is pyrazole. In certain instances, R₄ is oxazole. In certain instances, R₄ is isoxazole.

In certain instances, the compound is:

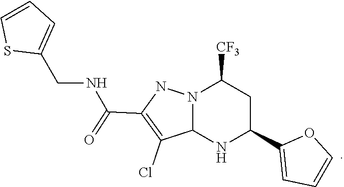

In certain instances, the compound is:

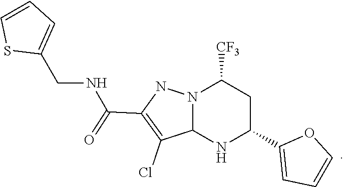

In some embodiments, compounds of interest include those set forth below, which are not to be construed as limitative.

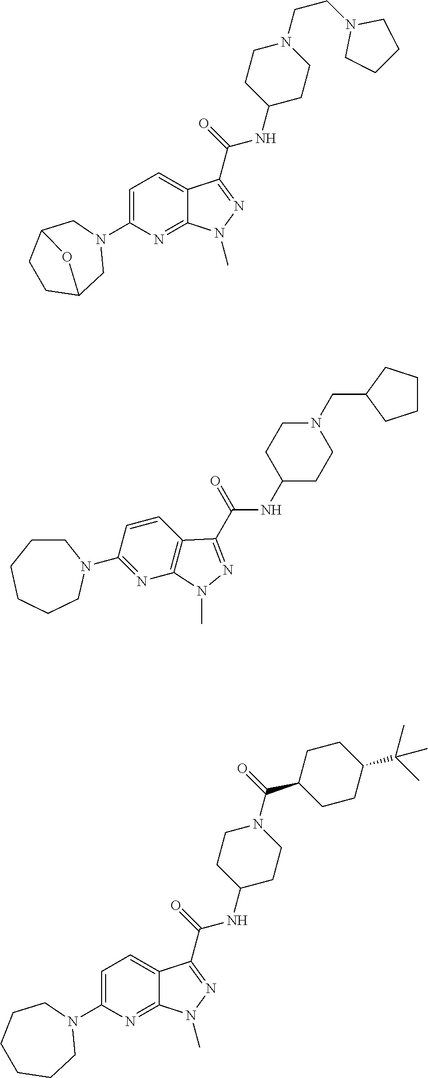 [SMILES: Cn1nc(C(=O)NC2CCN(CCN3CCCC3)CC2)c2ccc(N3CC4CCC(C3)O4)nc21 ; Cn1nc(C(=O)NC2CCN(CC3CCCC3)CC2)c2ccc(N3CCCCCC3)nc21 ; Cn1nc(C(=O)NC2CCN(C(=O)[C@H]3CC[C@H](C(C)(C)C)CC3)CC2)c2ccc(N3CCCCCC3)nc21]

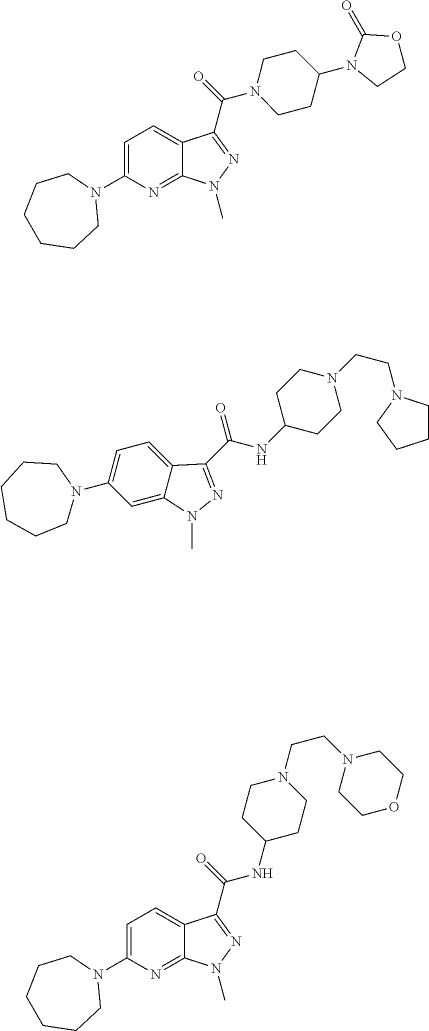 [SMILES: Cn1nc(C(=O)N2CCC(N3CCOC3=O)CC2)c2ccc(N3CCCCCC3)nc21 ; Cn1nc(C(=O)NC2CCN(CCN3CCCC3)CC2)c2ccc(N3CCCCCC3)cc21 ; Cn1nc(C(=O)NC2CCN(CCN3CCOCC3)CC2)c2ccc(N3CCCCCC3)nc21]

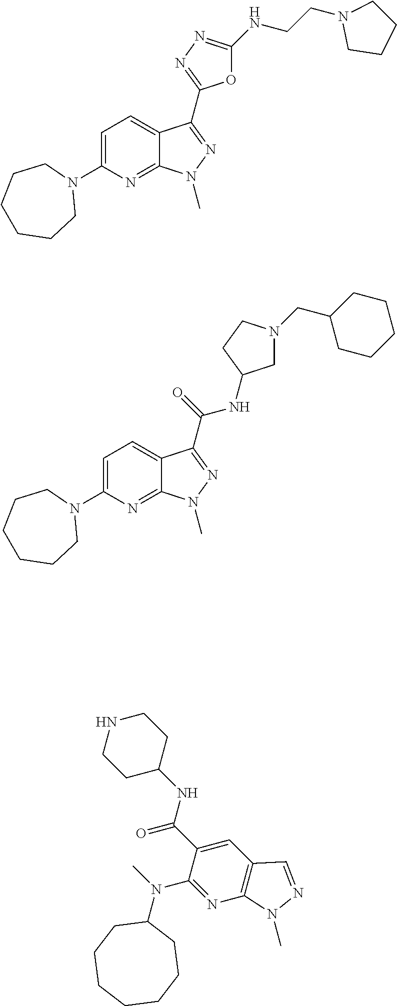 [SMILES: Cn1nc(-c2nnc(NCCN3CCCC3)o2)c2ccc(N3CCCCCC3)nc21 ; Cn1nc(C(=O)NC2CCN(CC3CCCCC3)C2)c2ccc(N3CCCCCC3)nc21 ; CN(c1nc2c(cnn2C)cc1C(=O)NC1CCNCC1)C1CCCCCCC1]

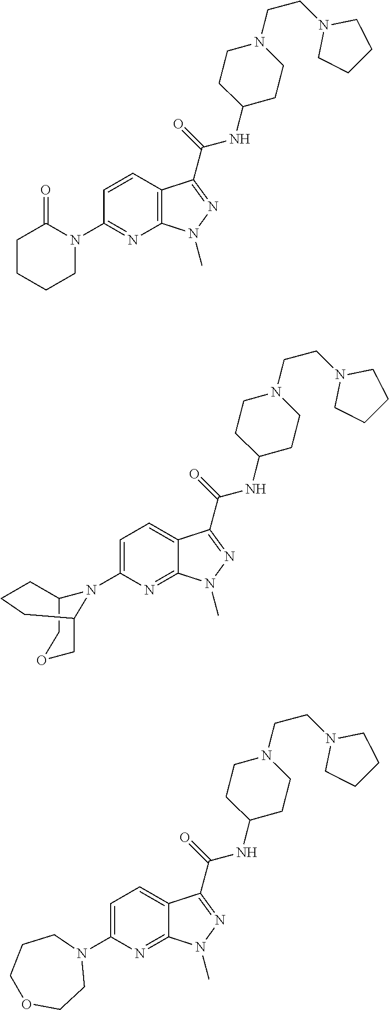 [SMILES: Cn1nc(C(=O)NC2CCN(CCN3CCCC3)CC2)c2ccc(N3CCCCC3=O)nc21 ; Cn1nc(C(=O)NC2CCN(CCN3CCCC3)CC2)c2ccc(N3C4CCCC3COC4)nc21 ; Cn1nc(C(=O)NC2CCN(CCN3CCCC3)CC2)c2ccc(N3CCCOCC3)nc21]

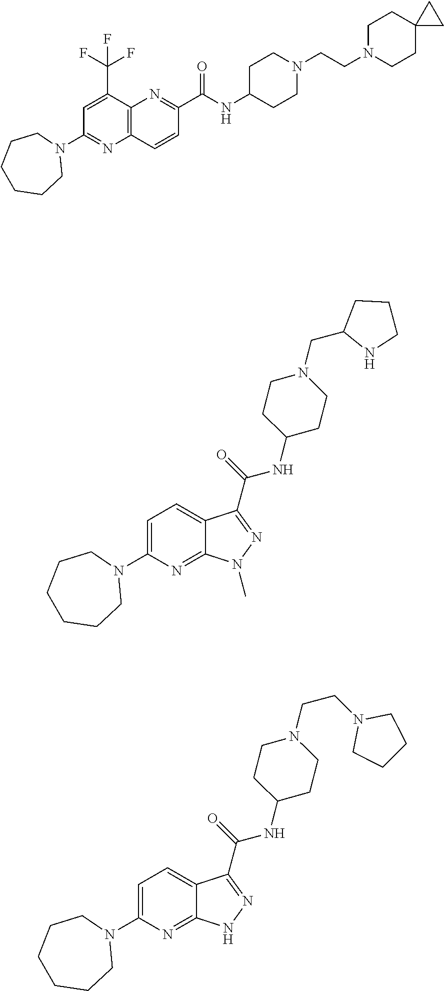 [SMILES: O=C(NC1CCN(CCN2CCC3(CC2)CC3)CC1)c1ccc2nc(N3CCCCCC3)cc(C(F)(F)F)c2n1 ; Cn1nc(C(=O)NC2CCN(CC3CCCN3)CC2)c2ccc(N3CCCCCC3)nc21 ; O=C(NC1CCN(CCN2CCCC2)CC1)c1n[nH]c2nc(N3CCCCCC3)ccc12]

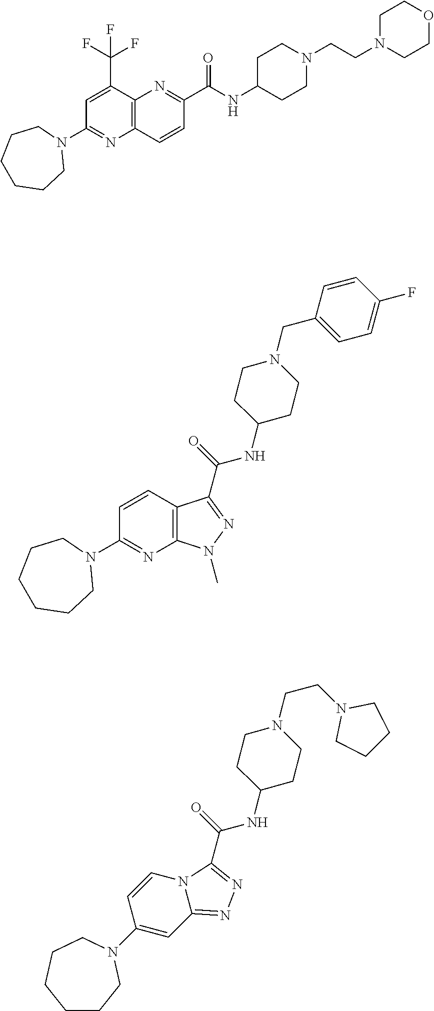 [SMILES: O=C(NC1CCN(CCN2CCOCC2)CC1)c1ccc2nc(N3CCCCCC3)cc(C(F)(F)F)c2n1 ; Cn1nc(C(=O)NC2CCN(Cc3ccc(F)cc3)CC2)c2ccc(N3CCCCCC3)nc21 ; O=C(NC1CCN(CCN2CCCC2)CC1)c1nnc2cc(N3CCCCCC3)ccn12]

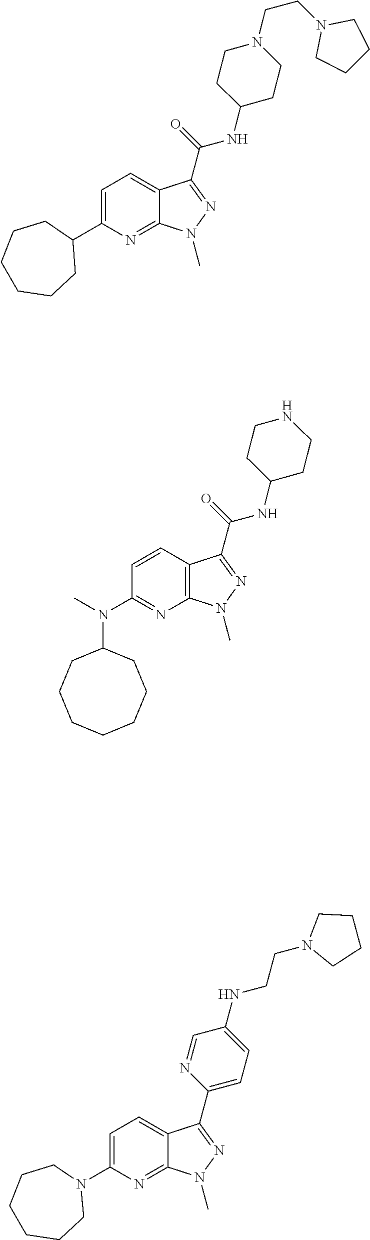 [SMILES: Cn1nc(C(=O)NC2CCN(CCN3CCCC3)CC2)c2ccc(C3CCCCCC3)nc21 ; CN(c1ccc2c(C(=O)NC3CCNCC3)nn(C)c2n1)C1CCCCCCC1 ; Cn1nc(-c2ccc(NCCN3CCCC3)cn2)c2ccc(N3CCCCCC3)nc21]

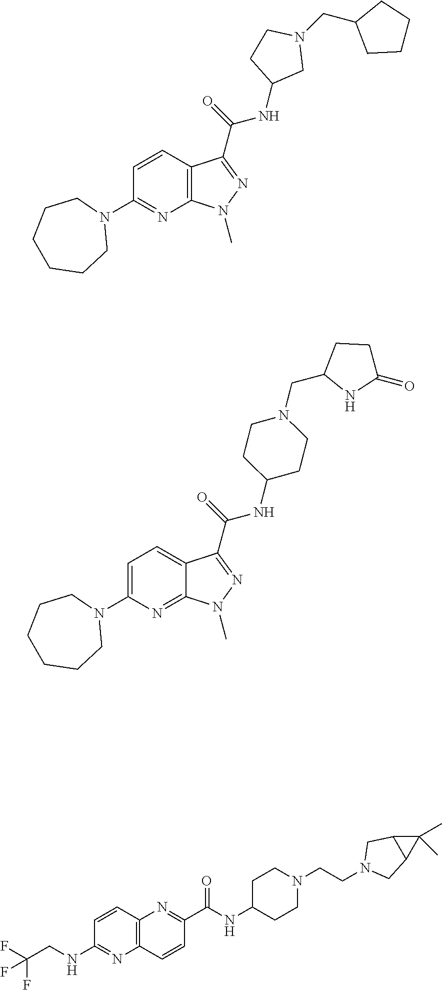 [SMILES: Cn1nc(C(=O)NC2CCN(CC3CCCC3)C2)c2ccc(N3CCCCCC3)nc21 ; Cn1nc(C(=O)NC2CCN(CC3CCC(=O)N3)CC2)c2ccc(N3CCCCCC3)nc21 ; CC1(C)C2CN(CCN3CCC(NC(=O)c4ccc5nc(NCC(F)(F)F)ccc5n4)CC3)CC21]

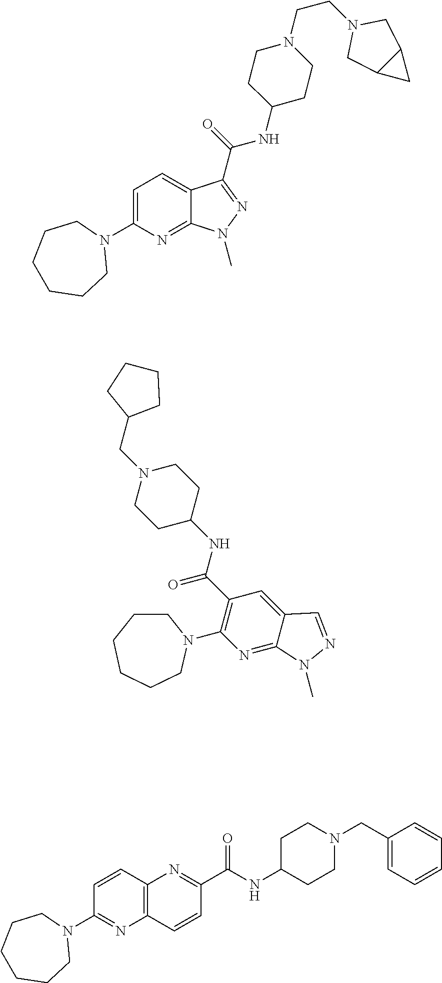 [SMILES: Cn1nc(C(=O)NC2CCN(CCN3CC4CC4C3)CC2)c2ccc(N3CCCCCC3)nc21 ; Cn1ncc2cc(C(=O)NC3CCN(CC4CCCC4)CC3)c(N3CCCCCC3)nc21 ; O=C(NC1CCN(Cc2ccccc2)CC1)c1ccc2nc(N3CCCCCC3)ccc2n1]

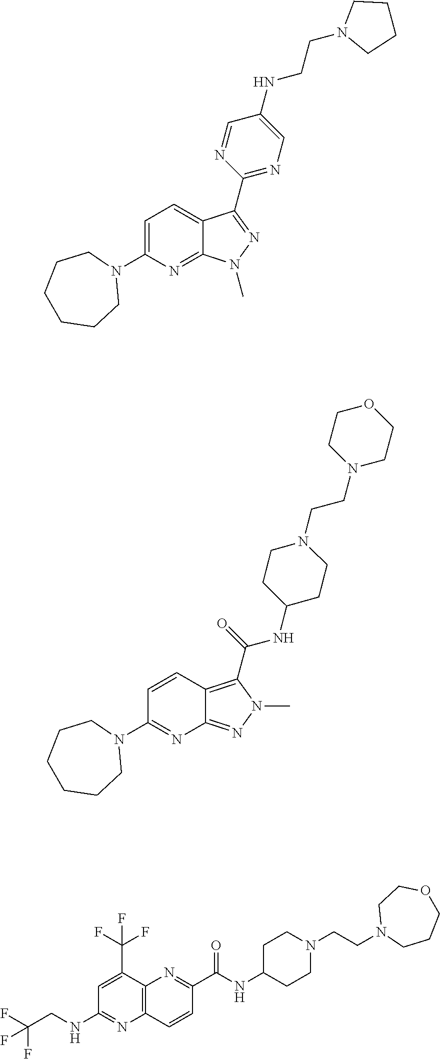 [SMILES: Cn1nc(-c2ncc(NCCN3CCCC3)cn2)c2ccc(N3CCCCCC3)nc21 ; Cn1nc2nc(N3CCCCCC3)ccc2c1C(=O)NC1CCN(CCN2CCOCC2)CC1 ; O=C(NC1CCN(CCN2CCCOCC2)CC1)c1ccc2nc(NCC(F)(F)F)cc(C(F)(F)F)c2n1]

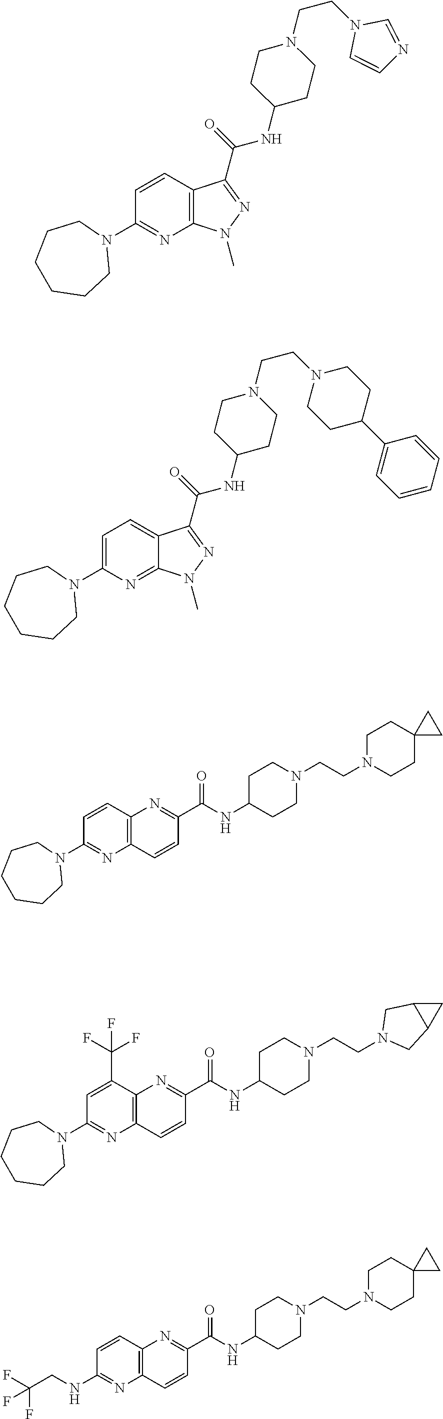 [SMILES: Cn1nc(C(=O)NC2CCN(CCn3ccnc3)CC2)c2ccc(N3CCCCCC3)nc21 ; Cn1nc(C(=O)NC2CCN(CCN3CCC(c4ccccc4)CC3)CC2)c2ccc(N3CCCCCC3)nc21 ; O=C(NC1CCN(CCN2CCC3(CC2)CC3)CC1)c1ccc2nc(N3CCCCCC3)ccc2n1 ; O=C(NC1CCN(CCN2CC3CC3C2)CC1)c1ccc2nc(N3CCCCCC3)cc(C(F)(F)F)c2n1 ; O=C(NC1CCN(CCN2CCC3(CC2)CC3)CC1)c1ccc2nc(NCC(F)(F)F)ccc2n1]

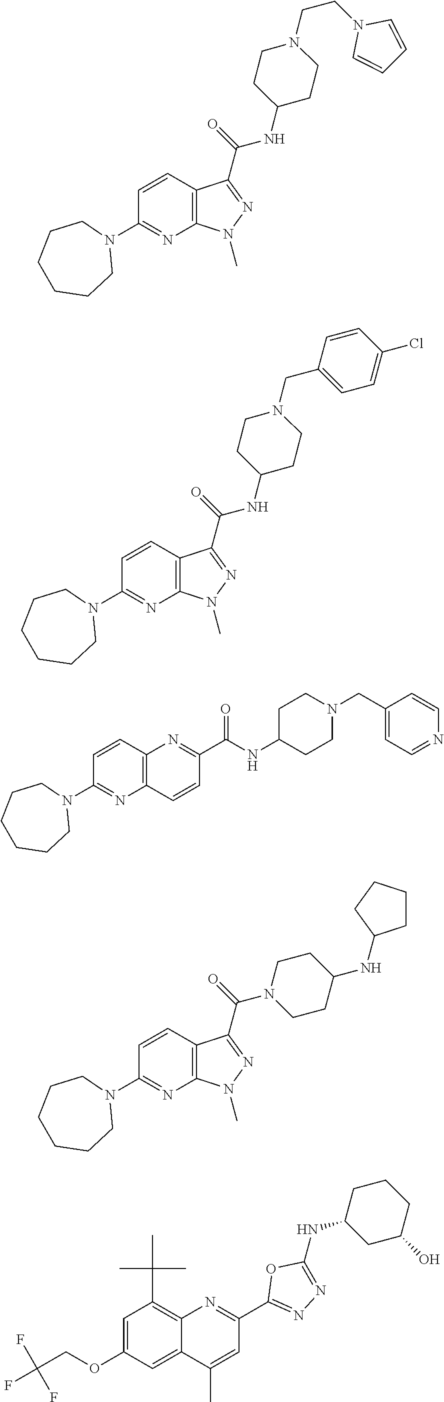 [SMILES: Cn1nc(C(=O)NC2CCN(CCn3cccc3)CC2)c2ccc(N3CCCCCC3)nc21 ; Cn1nc(C(=O)NC2CCN(Cc3ccc(Cl)cc3)CC2)c2ccc(N3CCCCCC3)nc21 ; O=C(NC1CCN(Cc2ccncc2)CC1)c1ccc2nc(N3CCCCCC3)ccc2n1 ; Cn1nc(C(=O)N2CCC(NC3CCCC3)CC2)c2ccc(N3CCCCCC3)nc21 ; Cc1cc(-c2nnc(N[C@@H]3CCC[C@H](O)C3)o2)nc2c(C(C)(C)C)cc(OCC(F)(F)F)cc12]

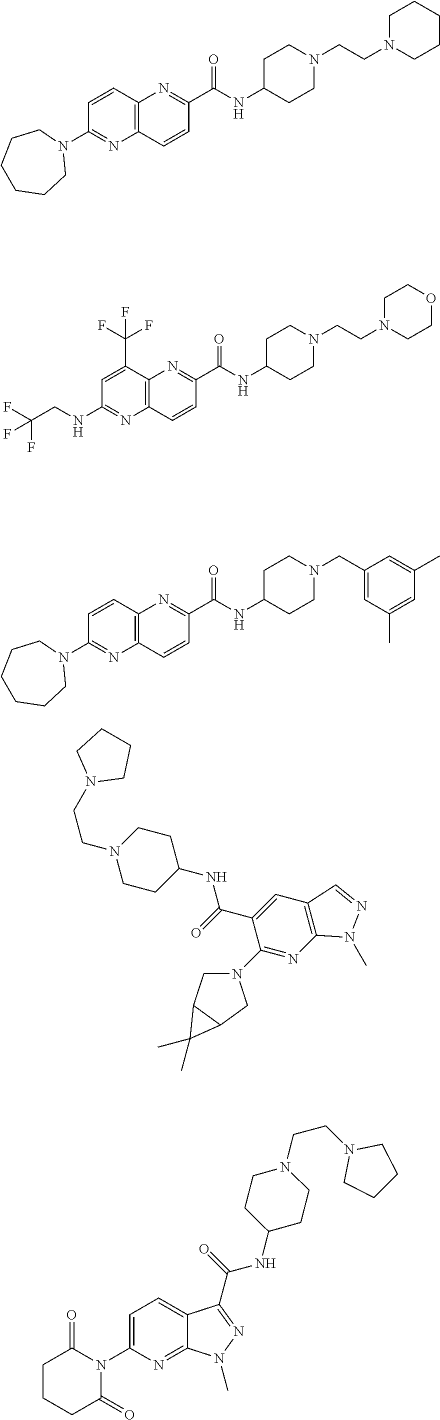 [SMILES: O=C(NC1CCN(CCN2CCCCC2)CC1)c1ccc2nc(N3CCCCCC3)ccc2n1 ; O=C(NC1CCN(CCN2CCOCC2)CC1)c1ccc2nc(NCC(F)(F)F)cc(C(F)(F)F)c2n1 ; Cc1cc(C)cc(CN2CCC(NC(=O)c3ccc4nc(N5CCCCCC5)ccc4n3)CC2)c1 ; Cn1ncc2cc(C(=O)NC3CCN(CCN4CCCC4)CC3)c(N3CC4C(C3)C4(C)C)nc21 ; Cn1nc(C(=O)NC2CCN(CCN3CCCC3)CC2)c2ccc(N3C(=O)CCCC3=O)nc21]

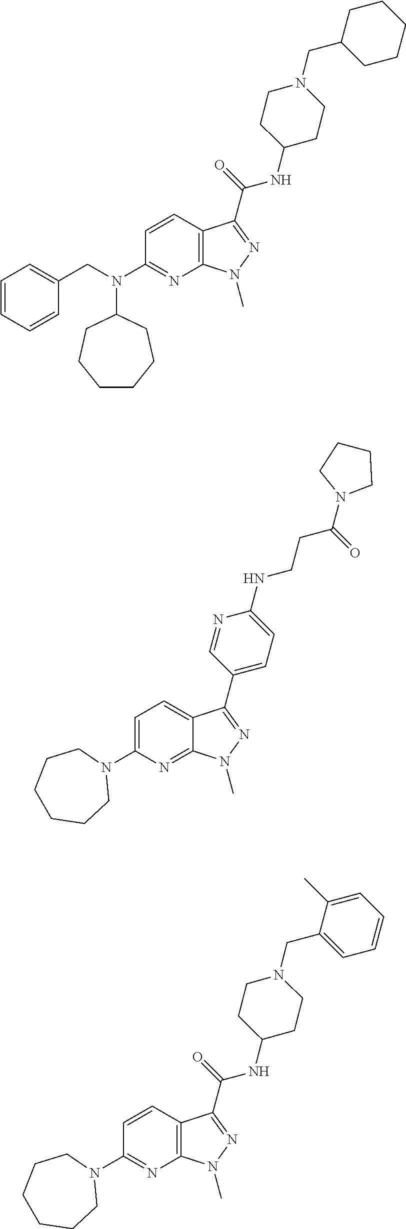 [SMILES: Cn1nc(C(=O)NC2CCN(CC3CCCCC3)CC2)c2ccc(N(Cc3ccccc3)C3CCCCCC3)nc21 ; Cn1nc(-c2ccc(NCCC(=O)N3CCCC3)nc2)c2ccc(N3CCCCCC3)nc21 ; Cc1ccccc1CN1CCC(NC(=O)c2nn(C)c3nc(N4CCCCCC4)ccc23)CC1]

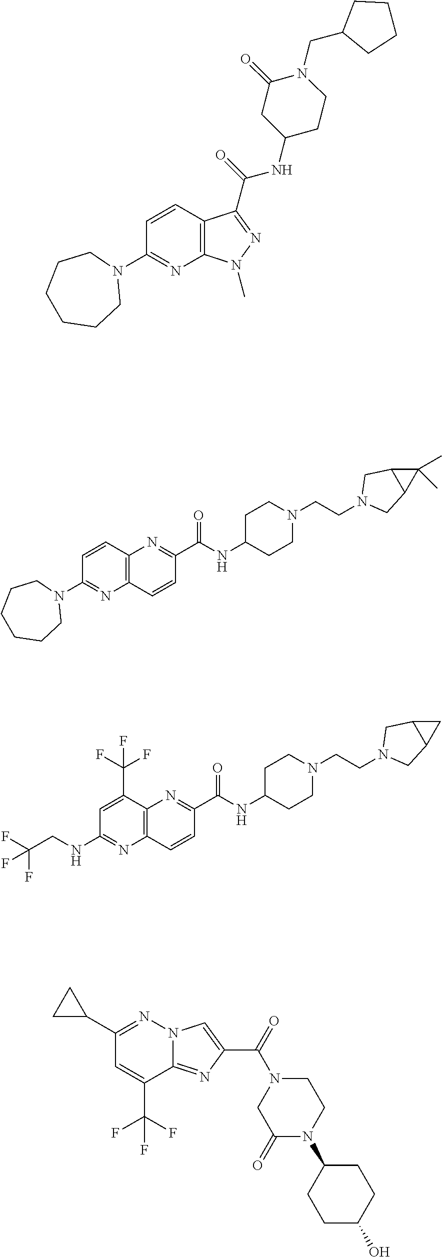 [SMILES: Cn1nc(C(=O)NC2CCN(CC3CCCC3)C(=O)C2)c2ccc(N3CCCCCC3)nc21 ; CC1(C)C2CN(CCN3CCC(NC(=O)c4ccc5nc(N6CCCCCC6)ccc5n4)CC3)CC21 ; O=C(NC1CCN(CCN2CC3CC3C2)CC1)c1ccc2nc(NCC(F)(F)F)cc(C(F)(F)F)c2n1 ; O=C(c1cn2nc(C3CC3)cc(C(F)(F)F)c2n1)N1CCN([C@H]2CC[C@H](O)CC2)C(=O)C1]

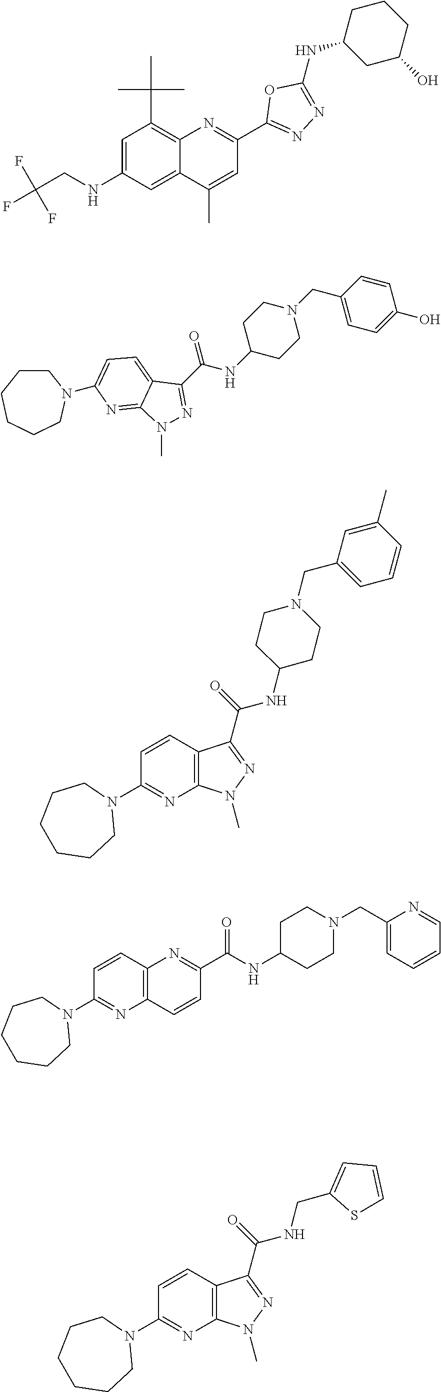 [SMILES: Cc1cc(-c2nnc(N[C@@H]3CCC[C@H](O)C3)o2)nc2c(C(C)(C)C)cc(NCC(F)(F)F)cc12 ; Cn1nc(C(=O)NC2CCN(Cc3ccc(O)cc3)CC2)c2ccc(N3CCCCCC3)nc21 ; Cc1cccc(CN2CCC(NC(=O)c3nn(C)c4nc(N5CCCCCC5)ccc34)CC2)c1 ; O=C(NC1CCN(Cc2ccccn2)CC1)c1ccc2nc(N3CCCCCC3)ccc2n1 ; Cn1nc(C(=O)NCc2cccs2)c2ccc(N3CCCCCC3)nc21]

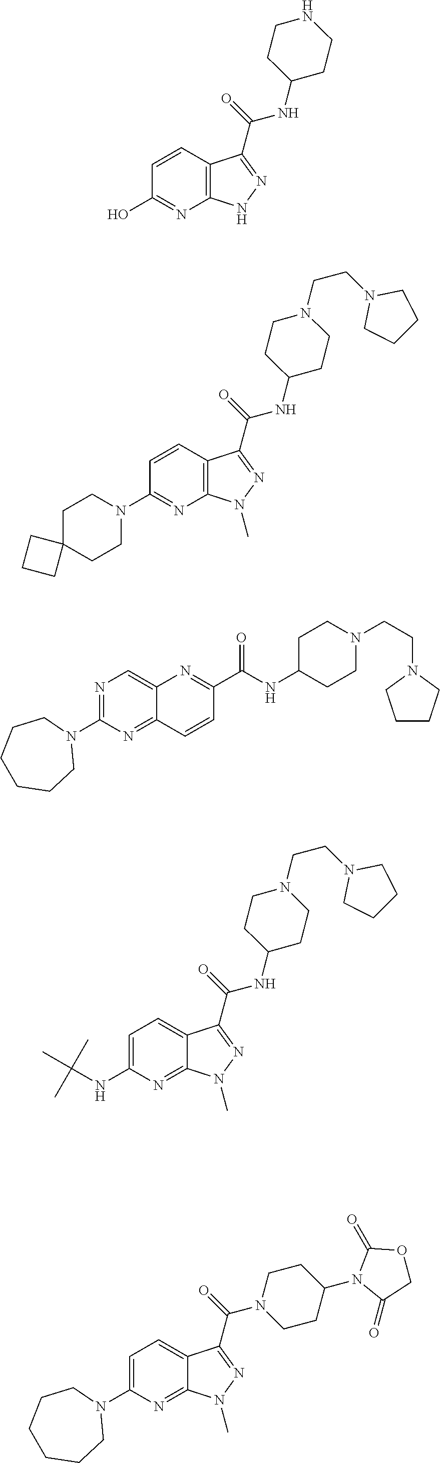 [SMILES: O=C(NC1CCNCC1)c1n[nH]c2nc(O)ccc12 ; Cn1nc(C(=O)NC2CCN(CCN3CCCC3)CC2)c2ccc(N3CCC4(CCC4)CC3)nc21 ; O=C(NC1CCN(CCN2CCCC2)CC1)c1ccc2nc(N3CCCCCC3)ncc2n1 ; Cn1nc(C(=O)NC2CCN(CCN3CCCC3)CC2)c2ccc(NC(C)(C)C)nc21 ; Cn1nc(C(=O)N2CCC(N3C(=O)COC3=O)CC2)c2ccc(N3CCCCCC3)nc21]

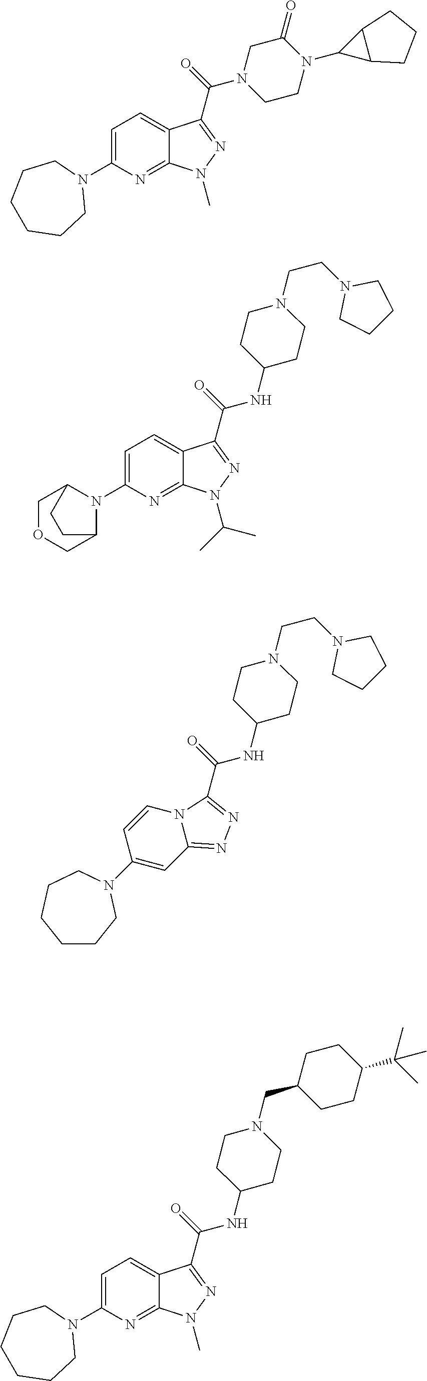 [SMILES: Cn1nc(C(=O)N2CCN(C3C4CCCC43)C(=O)C2)c2ccc(N3CCCCCC3)nc21 ; CC(C)n1nc(C(=O)NC2CCN(CCN3CCCC3)CC2)c2ccc(N3C4CCC3COC4)nc21 ; O=C(NC1CCN(CCN2CCCC2)CC1)c1nnc2cc(N3CCCCCC3)ccn12 ; Cn1nc(C(=O)NC2CCN(C[C@H]3CC[C@H](C(C)(C)C)CC3)CC2)c2ccc(N3CCCCCC3)nc21]

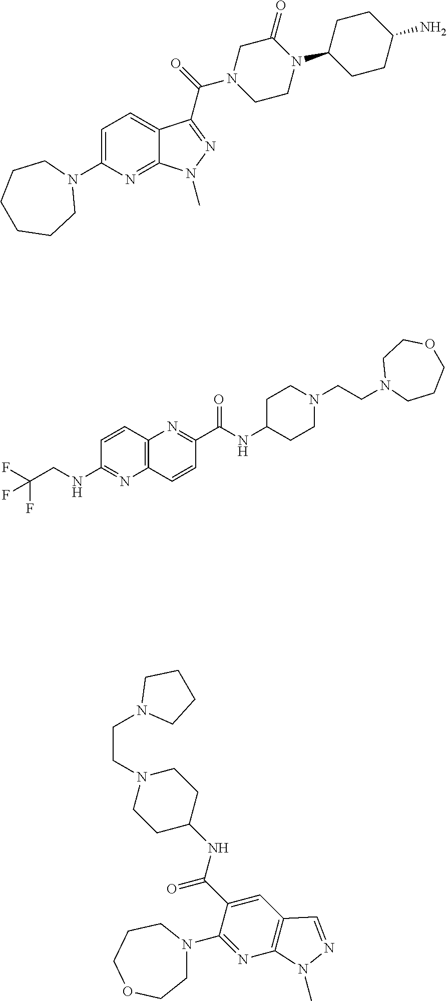 [SMILES: Cn1nc(C(=O)N2CCN([C@H]3CC[C@H](N)CC3)C(=O)C2)c2ccc(N3CCCCCC3)nc21 ; O=C(NC1CCN(CCN2CCCOCC2)CC1)c1ccc2nc(NCC(F)(F)F)ccc2n1 ; Cn1ncc2cc(C(=O)NC3CCN(CCN4CCCC4)CC3)c(N3CCCOCC3)nc21]

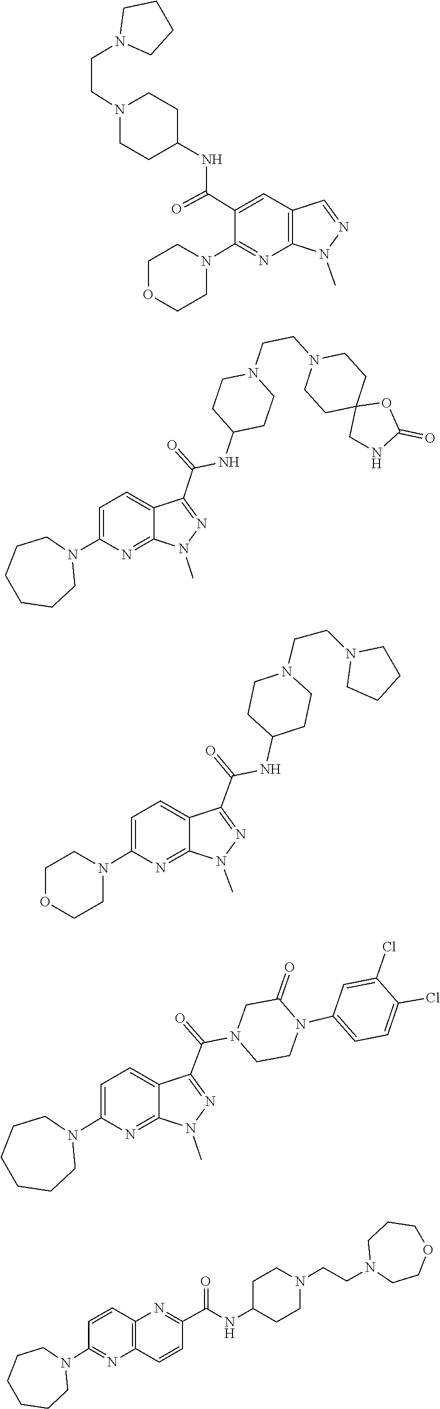 [SMILES: Cn1ncc2cc(C(=O)NC3CCN(CCN4CCCC4)CC3)c(N3CCOCC3)nc21 ; Cn1nc(C(=O)NC2CCN(CCN3CCC4(CC3)CNC(=O)O4)CC2)c2ccc(N3CCCCCC3)nc21 ; Cn1nc(C(=O)NC2CCN(CCN3CCCC3)CC2)c2ccc(N3CCOCC3)nc21 ; Cn1nc(C(=O)N2CCN(c3ccc(Cl)c(Cl)c3)C(=O)C2)c2ccc(N3CCCCCC3)nc21 ; O=C(NC1CCN(CCN2CCCOCC2)CC1)c1ccc2nc(N3CCCCCC3)ccc2n1]

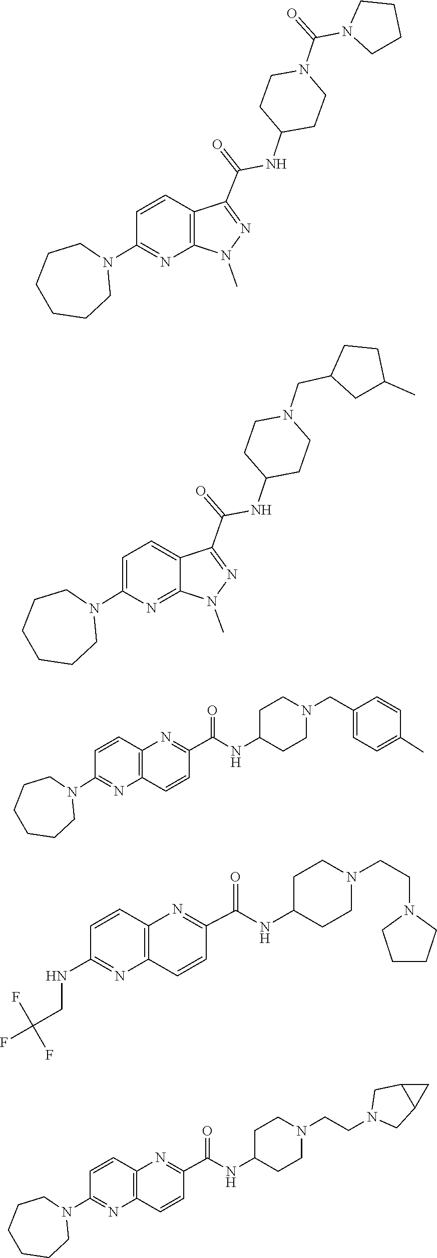 [SMILES: Cn1nc(C(=O)NC2CCN(C(=O)N3CCCC3)CC2)c2ccc(N3CCCCCC3)nc21 ; CC1CCC(CN2CCC(NC(=O)c3nn(C)c4nc(N5CCCCCC5)ccc34)CC2)C1 ; Cc1ccc(CN2CCC(NC(=O)c3ccc4nc(N5CCCCCC5)ccc4n3)CC2)cc1 ; O=C(NC1CCN(CCN2CCCC2)CC1)c1ccc2nc(NCC(F)(F)F)ccc2n1 ; O=C(NC1CCN(CCN2CC3CC3C2)CC1)c1ccc2nc(N3CCCCCC3)ccc2n1]

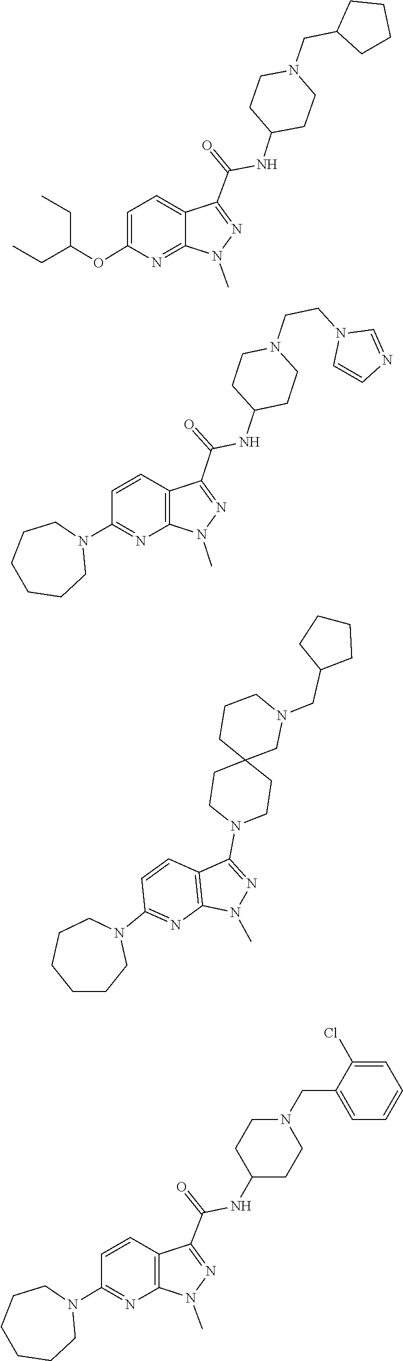 [SMILES: CCC(CC)Oc1ccc2c(C(=O)NC3CCN(CC4CCCC4)CC3)nn(C)c2n1 ; Cn1nc(C(=O)NC2CCN(CCn3ccnc3)CC2)c2ccc(N3CCCCCC3)nc21 ; Cn1nc(N2CCC3(CCCN(CC4CCCC4)C3)CC2)c2ccc(N3CCCCCC3)nc21 ; Cn1nc(C(=O)NC2CCN(Cc3ccccc3Cl)CC2)c2ccc(N3CCCCCC3)nc21]

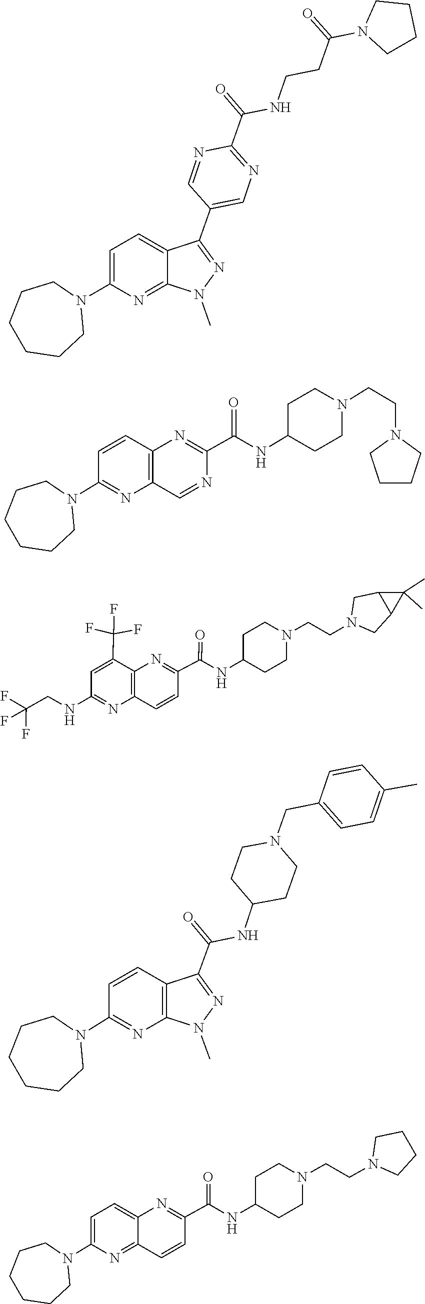 [SMILES: Cn1nc(-c2cnc(C(=O)NCCC(=O)N3CCCC3)nc2)c2ccc(N3CCCCCC3)nc21 ; O=C(NC1CCN(CCN2CCCC2)CC1)c1ncc2nc(N3CCCCCC3)ccc2n1 ; CC1(C)C2CN(CCN3CCC(NC(=O)c4ccc5nc(NCC(F)(F)F)cc(C(F)(F)F)c5n4)CC3)CC21 ; Cc1ccc(CN2CCC(NC(=O)c3nn(C)c4nc(N5CCCCCC5)ccc34)CC2)cc1 ; O=C(NC1CCN(CCN2CCCC2)CC1)c1ccc2nc(N3CCCCCC3)ccc2n1]

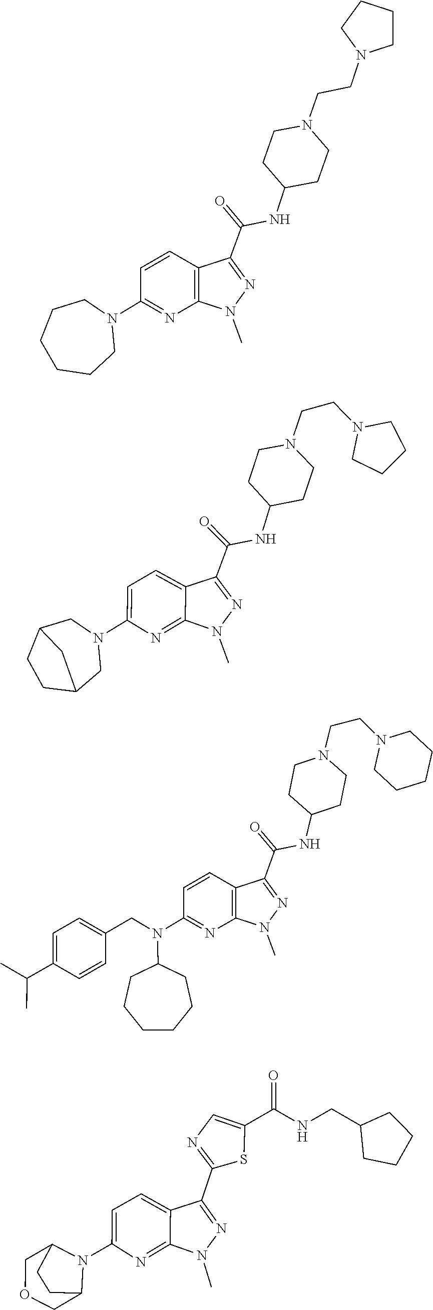 [SMILES: Cn1nc(C(=O)NC2CCN(CCN3CCCC3)CC2)c2ccc(N3CCCCCC3)nc21 ; Cn1nc(C(=O)NC2CCN(CCN3CCCC3)CC2)c2ccc(N3CC4CCC(C4)C3)nc21 ; CC(C)c1ccc(CN(c2ccc3c(C(=O)NC4CCN(CCN5CCCCC5)CC4)nn(C)c3n2)C2CCCCCC2)cc1 ; Cn1nc(-c2ncc(C(=O)NCC3CCCC3)s2)c2ccc(N3C4CCC3COC4)nc21]

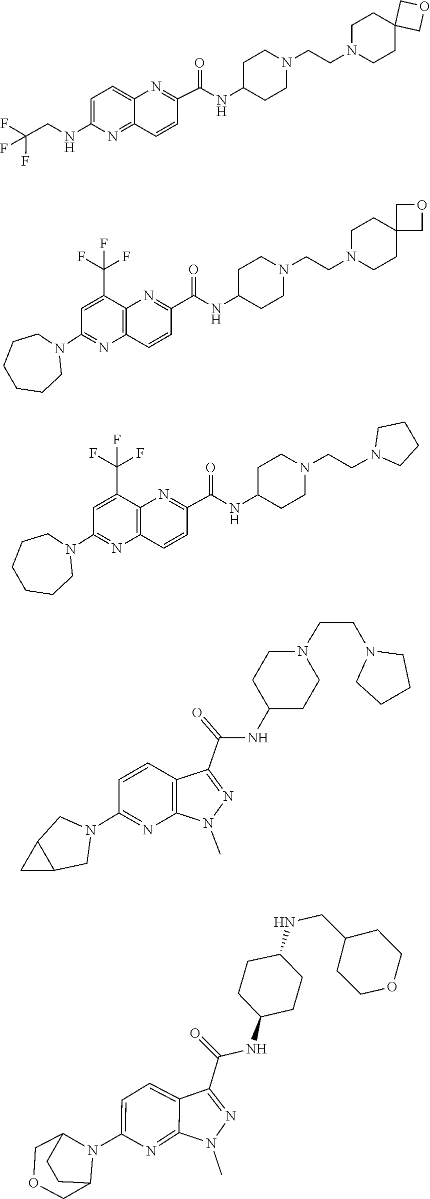 [SMILES: O=C(NC1CCN(CCN2CCC3(CC2)COC3)CC1)c1ccc2nc(NCC(F)(F)F)ccc2n1 ; O=C(NC1CCN(CCN2CCC3(CC2)COC3)CC1)c1ccc2nc(N3CCCCCC3)cc(C(F)(F)F)c2n1 ; O=C(NC1CCN(CCN2CCCC2)CC1)c1ccc2nc(N3CCCCCC3)cc(C(F)(F)F)c2n1 ; Cn1nc(C(=O)NC2CCN(CCN3CCCC3)CC2)c2ccc(N3CC4CC4C3)nc21 ; Cn1nc(C(=O)N[C@H]2CC[C@H](NCC3CCOCC3)CC2)c2ccc(N3C4CCC3COC4)nc21]

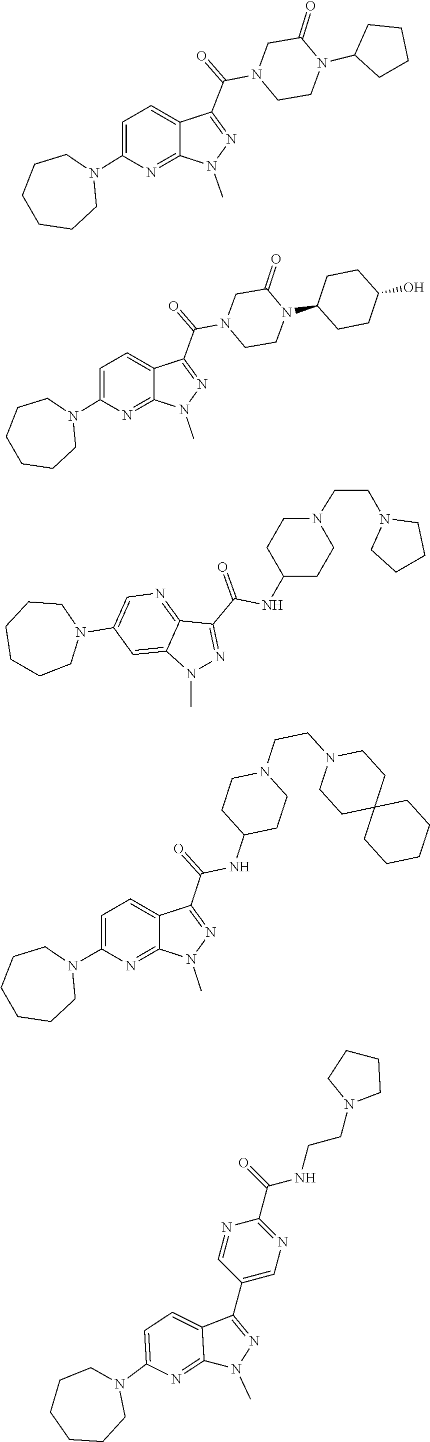 [SMILES: Cn1nc(C(=O)N2CCN(C3CCCC3)C(=O)C2)c2ccc(N3CCCCCC3)nc21 ; Cn1nc(C(=O)N2CCN([C@H]3CC[C@H](O)CC3)C(=O)C2)c2ccc(N3CCCCCC3)nc21 ; Cn1nc(C(=O)NC2CCN(CCN3CCCC3)CC2)c2ncc(N3CCCCCC3)cc21 ; Cn1nc(C(=O)NC2CCN(CCN3CCC4(CCCCC4)CC3)CC2)c2ccc(N3CCCCCC3)nc21 ; Cn1nc(-c2cnc(C(=O)NCCN3CCCC3)nc2)c2ccc(N3CCCCCC3)nc21]

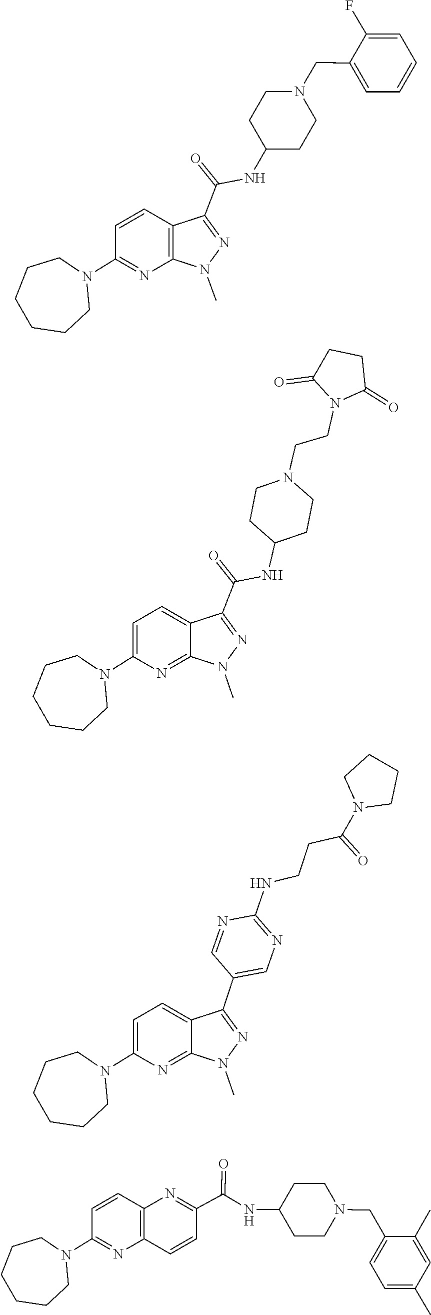 [SMILES: Cn1nc(C(=O)NC2CCN(Cc3ccccc3F)CC2)c2ccc(N3CCCCCC3)nc21 ; Cn1nc(C(=O)NC2CCN(CCN3C(=O)CCC3=O)CC2)c2ccc(N3CCCCCC3)nc21 ; Cn1nc(-c2cnc(NCCC(=O)N3CCCC3)nc2)c2ccc(N3CCCCCC3)nc21 ; Cc1ccc(CN2CCC(NC(=O)c3ccc4nc(N5CCCCCC5)ccc4n3)CC2)c(C)c1]

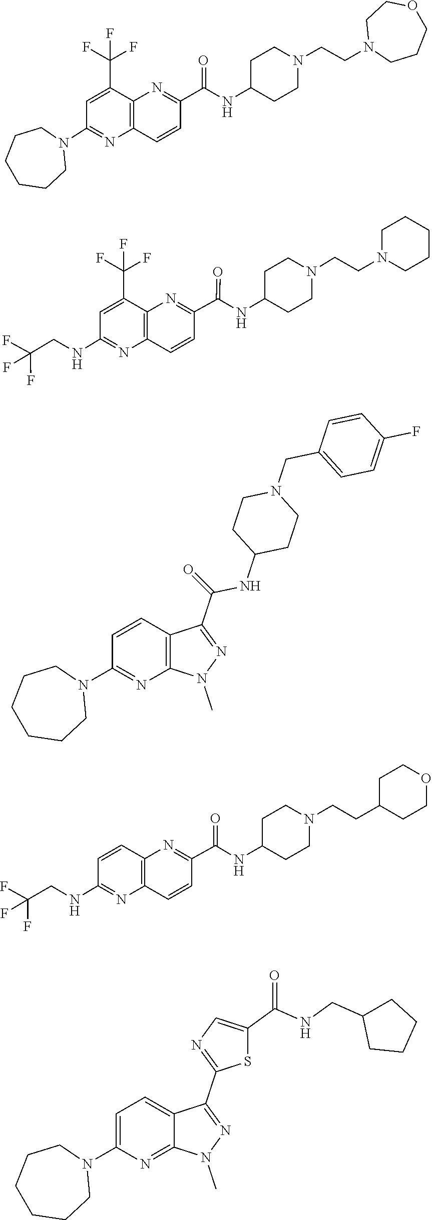 [SMILES: O=C(NC1CCN(CCN2CCCOCC2)CC1)c1ccc2nc(N3CCCCCC3)cc(C(F)(F)F)c2n1 ; O=C(NC1CCN(CCN2CCCCC2)CC1)c1ccc2nc(NCC(F)(F)F)cc(C(F)(F)F)c2n1 ; Cn1nc(C(=O)NC2CCN(Cc3ccc(F)cc3)CC2)c2ccc(N3CCCCCC3)nc21 ; O=C(NC1CCN(CCC2CCOCC2)CC1)c1ccc2nc(NCC(F)(F)F)ccc2n1 ; Cn1nc(-c2ncc(C(=O)NCC3CCCC3)s2)c2ccc(N3CCCCCC3)nc21]

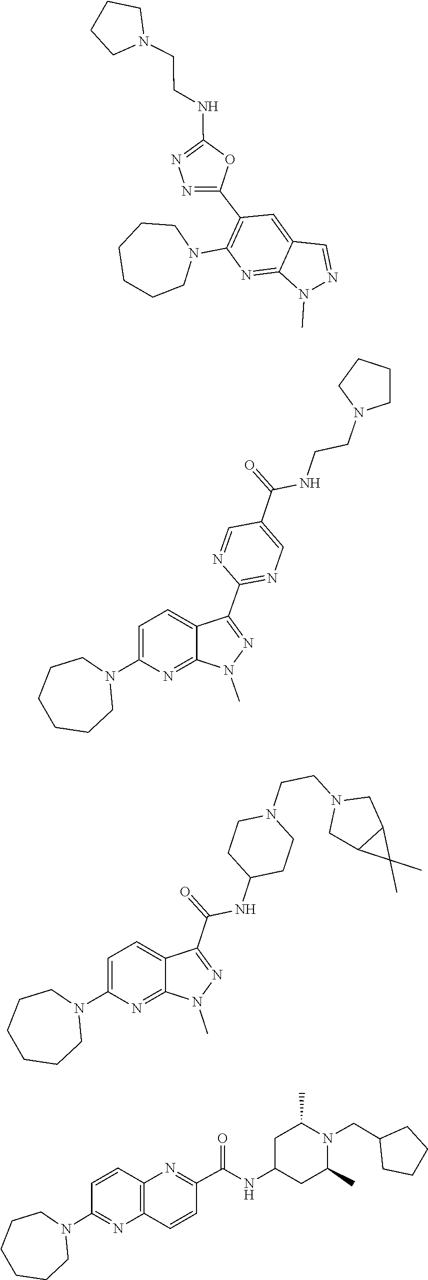 [SMILES: Cn1ncc2cc(-c3nnc(NCCN4CCCC4)o3)c(N3CCCCCC3)nc21 ; Cn1nc(-c2ncc(C(=O)NCCN3CCCC3)cn2)c2ccc(N3CCCCCC3)nc21 ; Cn1nc(C(=O)NC2CCN(CCN3CC4C(C3)C4(C)C)CC2)c2ccc(N3CCCCCC3)nc21 ; C[C@H]1CC(NC(=O)c2ccc3nc(N4CCCCCC4)ccc3n2)C[C@H](C)N1CC1CCCC1]

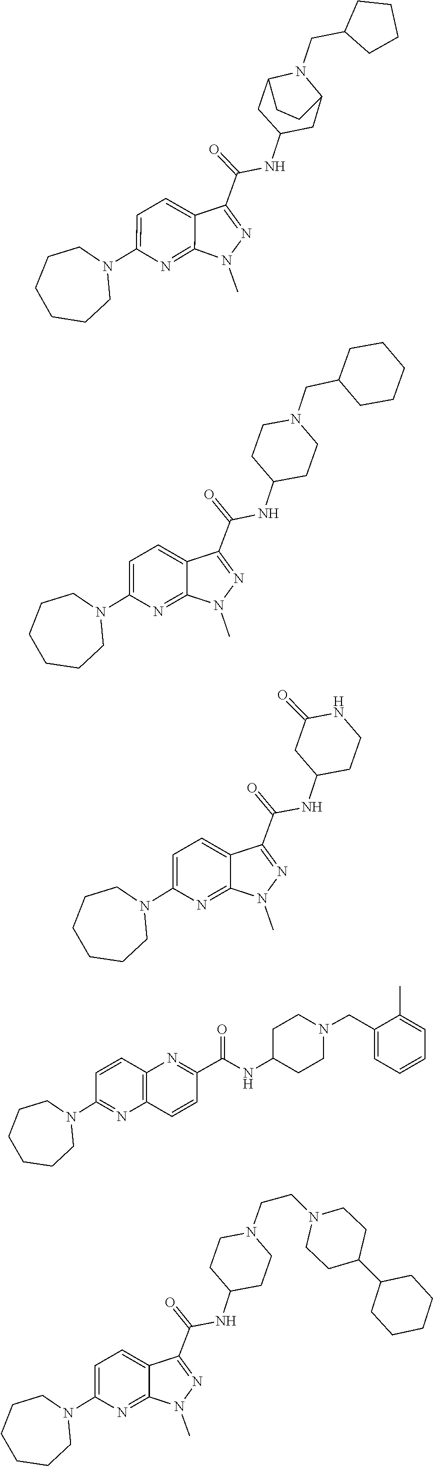 [SMILES: Cn1nc(C(=O)NC2CC3CCC(C2)N3CC2CCCC2)c2ccc(N3CCCCCC3)nc21 ; Cn1nc(C(=O)NC2CCN(CC3CCCCC3)CC2)c2ccc(N3CCCCCC3)nc21 ; Cn1nc(C(=O)NC2CCNC(=O)C2)c2ccc(N3CCCCCC3)nc21 ; Cc1ccccc1CN1CCC(NC(=O)c2ccc3nc(N4CCCCCC4)ccc3n2)CC1 ; Cn1nc(C(=O)NC2CCN(CCN3CCC(C4CCCCC4)CC3)CC2)c2ccc(N3CCCCCC3)nc21]

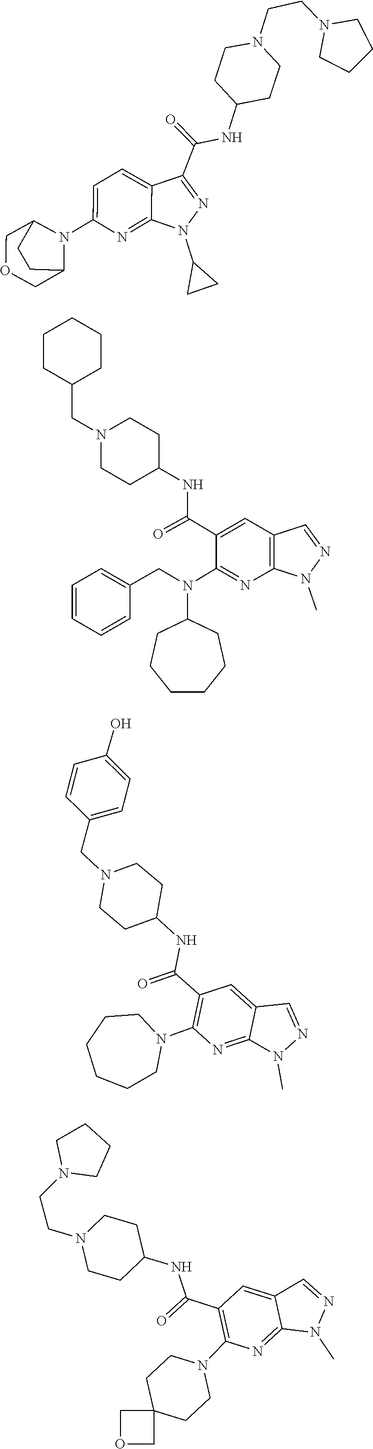 [SMILES: O=C(NC1CCN(CCN2CCCC2)CC1)c1nn(C2CC2)c2nc(N3C4CCC3COC4)ccc12 ; Cn1ncc2cc(C(=O)NC3CCN(CC4CCCCC4)CC3)c(N(Cc3ccccc3)C3CCCCCC3)nc21 ; Cn1ncc2cc(C(=O)NC3CCN(Cc4ccc(O)cc4)CC3)c(N3CCCCCC3)nc21 ; Cn1ncc2cc(C(=O)NC3CCN(CCN4CCCC4)CC3)c(N3CCC4(CC3)COC4)nc21]

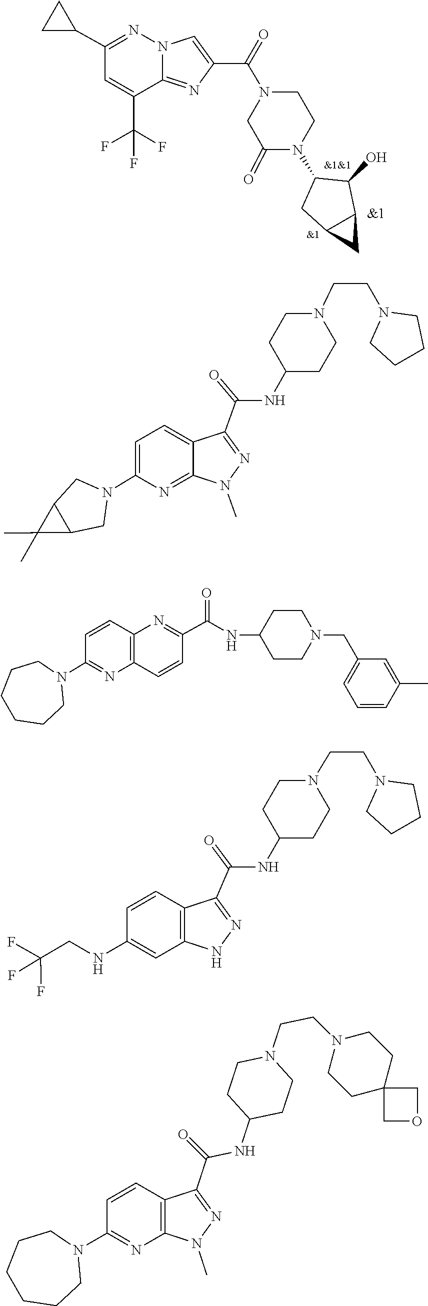

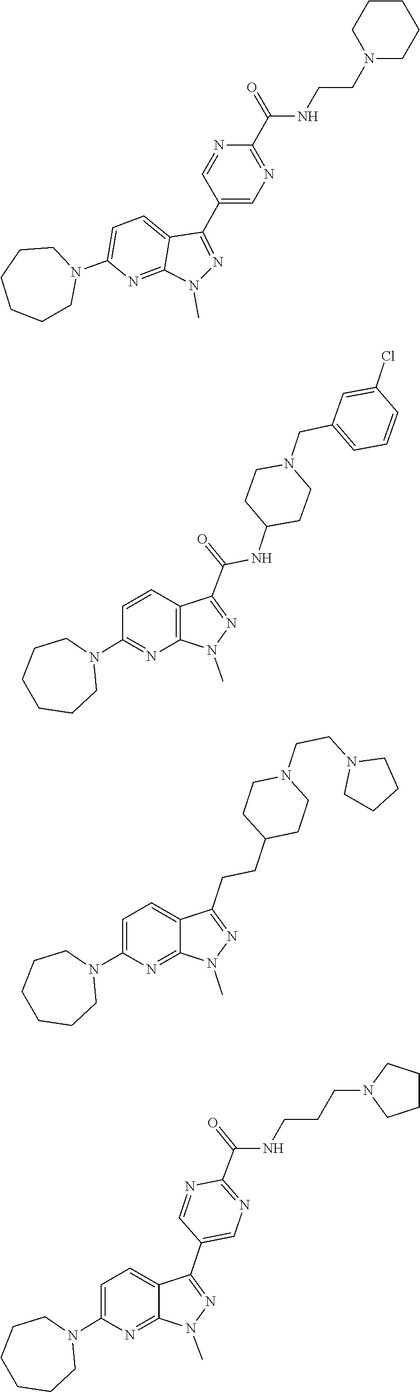 [SMILES: Cn1nc(-c2cnc(C(=O)NCCN3CCCCC3)nc2)c2ccc(N3CCCCCC3)nc21 ; Cn1nc(C(=O)NC2CCN(Cc3cccc(Cl)c3)CC2)c2ccc(N3CCCCCC3)nc21 ; Cn1nc(CCC2CCN(CCN3CCCC3)CC2)c2ccc(N3CCCCCC3)nc21 ; Cn1nc(-c2cnc(C(=O)NCCCN3CCCC3)nc2)c2ccc(N3CCCCCC3)nc21]

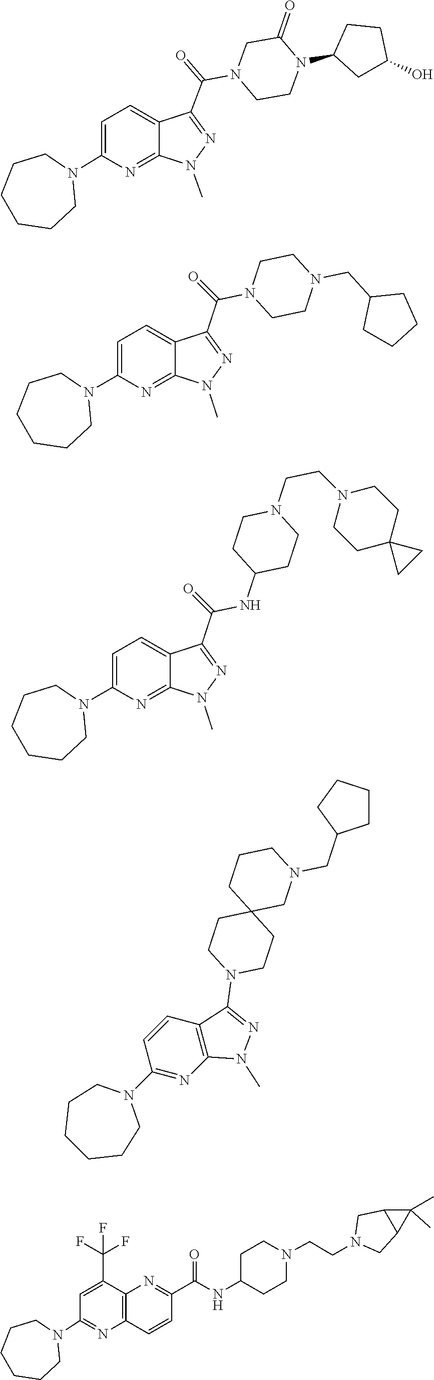 [SMILES: Cn1nc(C(=O)N2CCN([C@H]3CC[C@H](O)C3)C(=O)C2)c2ccc(N3CCCCCC3)nc21 ; Cn1nc(C(=O)N2CCN(CC3CCCC3)CC2)c2ccc(N3CCCCCC3)nc21 ; Cn1nc(C(=O)NC2CCN(CCN3CCC4(CC3)CC4)CC2)c2ccc(N3CCCCCC3)nc21 ; Cn1nc(N2CCC3(CCCN(CC4CCCC4)C3)CC2)c2ccc(N3CCCCCC3)nc21 ; CC1(C)C2CN(CCN3CCC(NC(=O)c4ccc5nc(N6CCCCCC6)cc(C(F)(F)F)c5n4)CC3)CC21]

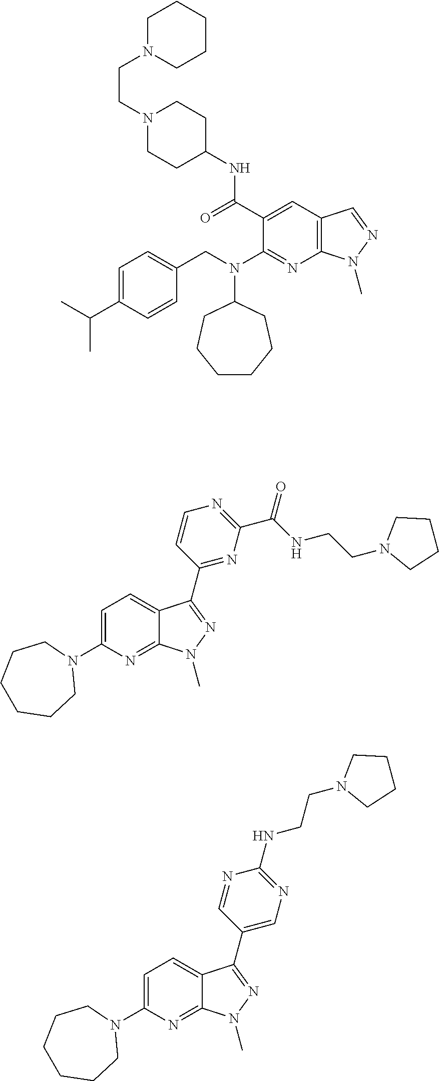 [SMILES: CC(C)c1ccc(CN(c2nc3c(cnn3C)cc2C(=O)NC2CCN(CCN3CCCCC3)CC2)C2CCCCCC2)cc1 ; Cn1nc(-c2ccnc(C(=O)NCCN3CCCC3)n2)c2ccc(N3CCCCCC3)nc21 ; Cn1nc(-c2cnc(NCCN3CCCC3)nc2)c2ccc(N3CCCCCC3)nc21]

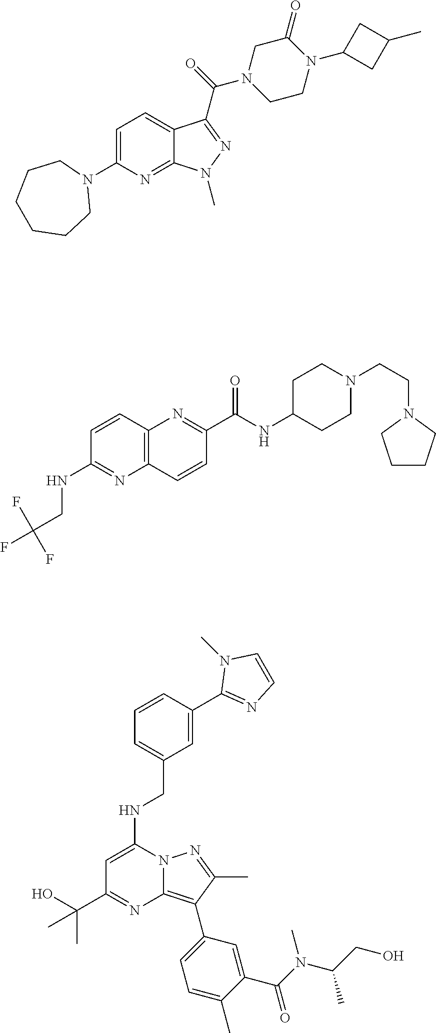 [SMILES: CC1CC(N2CCN(C(=O)c3nn(C)c4nc(N5CCCCCC5)ccc34)CC2=O)C1 ; O=C(NC1CCN(CCN2CCCC2)CC1)c1ccc2nc(NCC(F)(F)F)ccc2n1 ; Cc1ccc(-c2c(C)nn3c(NCc4cccc(-c5nccn5C)c4)cc(C(C)(C)O)nc23)cc1C(=O)N(C)[C@@H](C)CO]

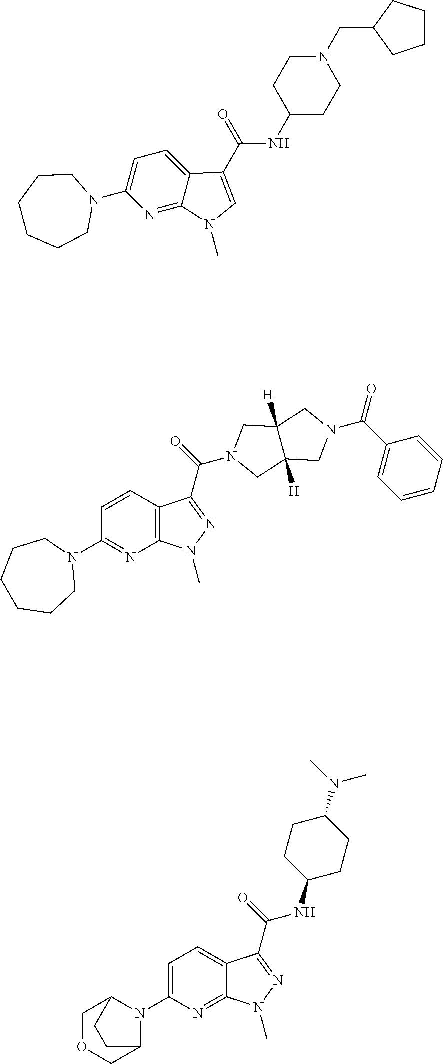 [SMILES: Cn1cc(C(=O)NC2CCN(CC3CCCC3)CC2)c2ccc(N3CCCCCC3)nc21 ; Cn1nc(C(=O)N2C[C@H]3CN(C(=O)c4ccccc4)C[C@H]3C2)c2ccc(N3CCCCCC3)nc21 ; Cn1nc(C(=O)N[C@H]2CC[C@H](N(C)C)CC2)c2ccc(N3C4CCC3COC4)nc21]

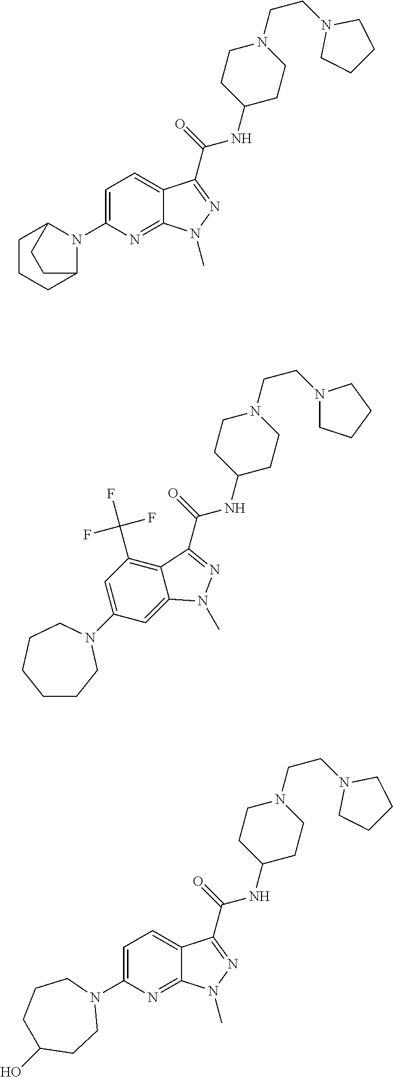 [SMILES: Cn1nc(C(=O)NC2CCN(CCN3CCCC3)CC2)c2ccc(N3C4CCCC3CC4)nc21 ; Cn1nc(C(=O)NC2CCN(CCN3CCCC3)CC2)c2c(C(F)(F)F)cc(N3CCCCCC3)cc21 ; Cn1nc(C(=O)NC2CCN(CCN3CCCC3)CC2)c2ccc(N3CCCC(O)CC3)nc21]

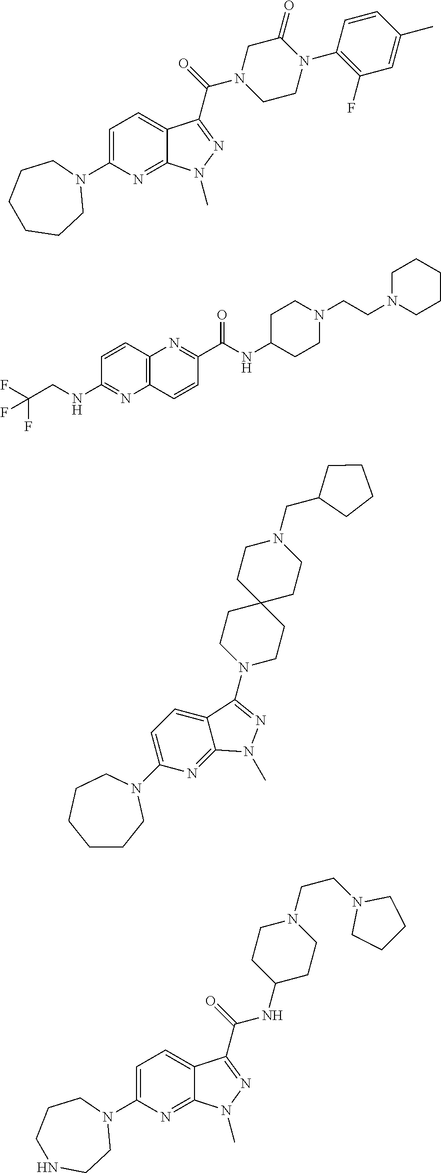 [SMILES: Cc1ccc(N2CCN(C(=O)c3nn(C)c4nc(N5CCCCCC5)ccc34)CC2=O)c(F)c1 ; O=C(NC1CCN(CCN2CCCCC2)CC1)c1ccc2nc(NCC(F)(F)F)ccc2n1 ; Cn1nc(N2CCC3(CCN(CC4CCCC4)CC3)CC2)c2ccc(N3CCCCCC3)nc21 ; Cn1nc(C(=O)NC2CCN(CCN3CCCC3)CC2)c2ccc(N3CCCNCC3)nc21]

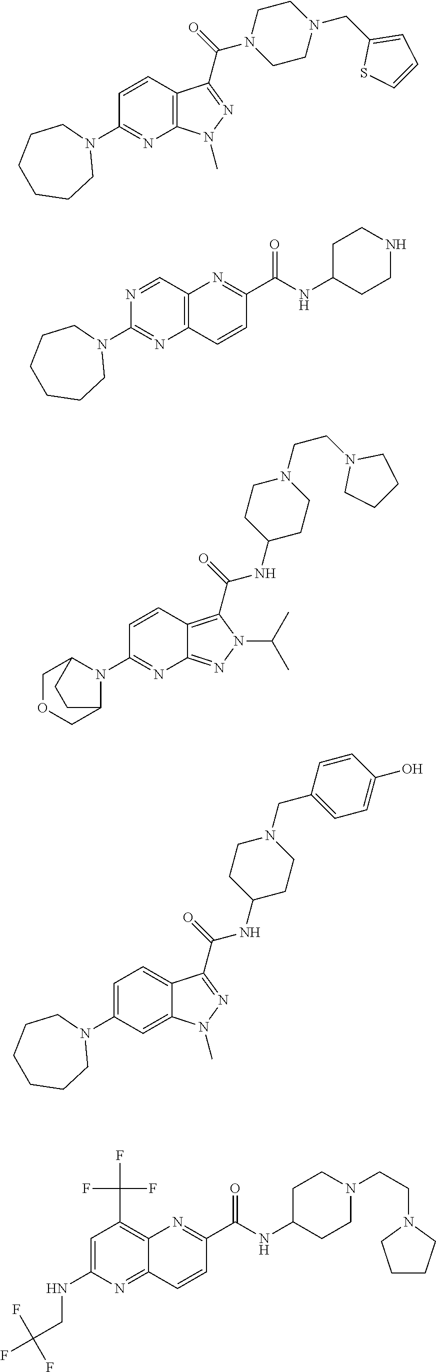 [SMILES: Cn1nc(C(=O)N2CCN(Cc3cccs3)CC2)c2ccc(N3CCCCCC3)nc21 ; O=C(NC1CCNCC1)c1ccc2nc(N3CCCCCC3)ncc2n1 ; CC(C)n1nc2nc(N3C4CCC3COC4)ccc2c1C(=O)NC1CCN(CCN2CCCC2)CC1 ; Cn1nc(C(=O)NC2CCN(Cc3ccc(O)cc3)CC2)c2ccc(N3CCCCCC3)cc21 ; O=C(NC1CCN(CCN2CCCC2)CC1)c1ccc2nc(NCC(F)(F)F)cc(C(F)(F)F)c2n1]

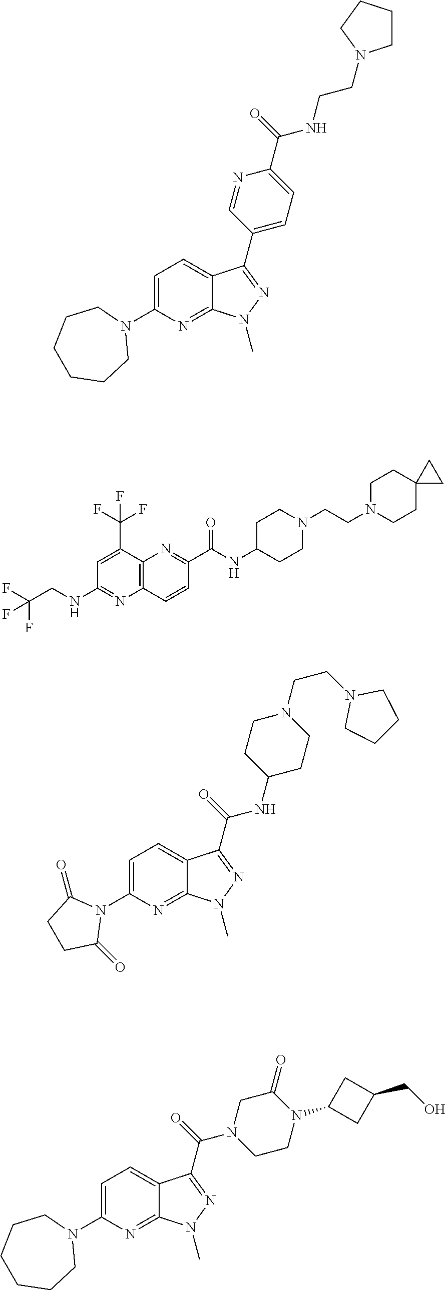 [SMILES: Cn1nc(-c2ccc(C(=O)NCCN3CCCC3)nc2)c2ccc(N3CCCCCC3)nc21 ; O=C(NC1CCN(CCN2CCC3(CC2)CC3)CC1)c1ccc2nc(NCC(F)(F)F)cc(C(F)(F)F)c2n1 ; Cn1nc(C(=O)NC2CCN(CCN3CCCC3)CC2)c2ccc(N3C(=O)CCC3=O)nc21 ; Cn1nc(C(=O)N2CCN([C@H]3C[C@H](CO)C3)C(=O)C2)c2ccc(N3CCCCCC3)nc21]

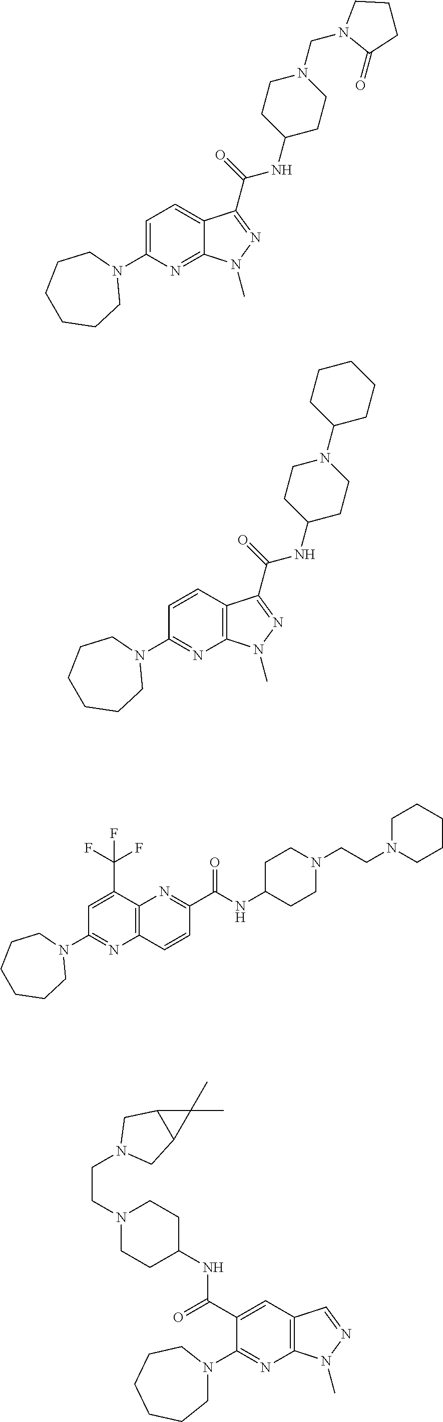 [SMILES: Cn1nc(C(=O)NC2CCN(CN3CCCC3=O)CC2)c2ccc(N3CCCCCC3)nc21 ; Cn1nc(C(=O)NC2CCN(C3CCCCC3)CC2)c2ccc(N3CCCCCC3)nc21 ; O=C(NC1CCN(CCN2CCCCC2)CC1)c1ccc2nc(N3CCCCCC3)cc(C(F)(F)F)c2n1 ; Cn1ncc2cc(C(=O)NC3CCN(CCN4CC5C(C4)C5(C)C)CC3)c(N3CCCCCC3)nc21]

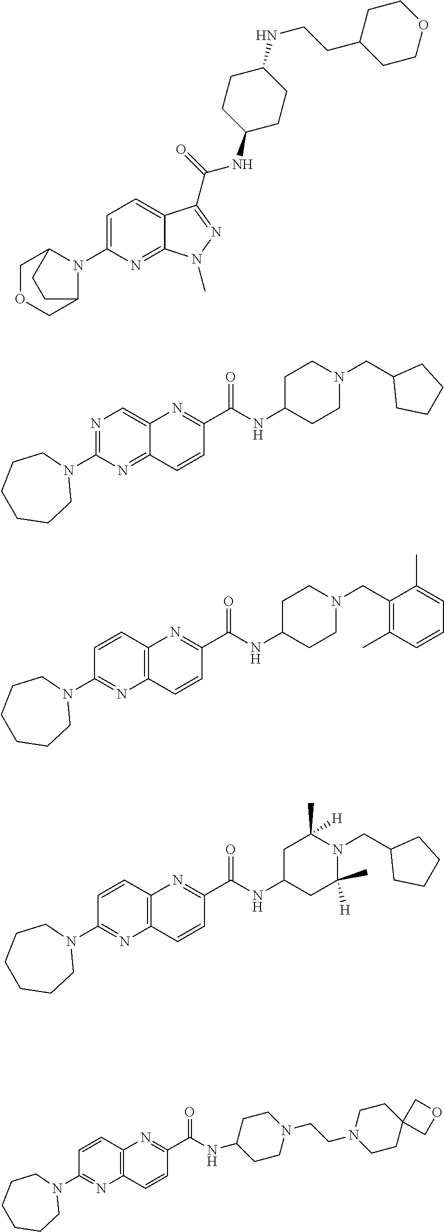 [SMILES: Cn1nc(C(=O)N[C@H]2CC[C@H](NCCC3CCOCC3)CC2)c2ccc(N3C4CCC3COC4)nc21 ; O=C(NC1CCN(CC2CCCC2)CC1)c1ccc2nc(N3CCCCCC3)ncc2n1 ; Cc1cccc(C)c1CN1CCC(NC(=O)c2ccc3nc(N4CCCCCC4)ccc3n2)CC1 ; C[C@@H]1CC(NC(=O)c2ccc3nc(N4CCCCCC4)ccc3n2)C[C@H](C)N1CC1CCCC1 ; O=C(NC1CCN(CCN2CCC3(CC2)COC3)CC1)c1ccc2nc(N3CCCCCC3)ccc2n1]

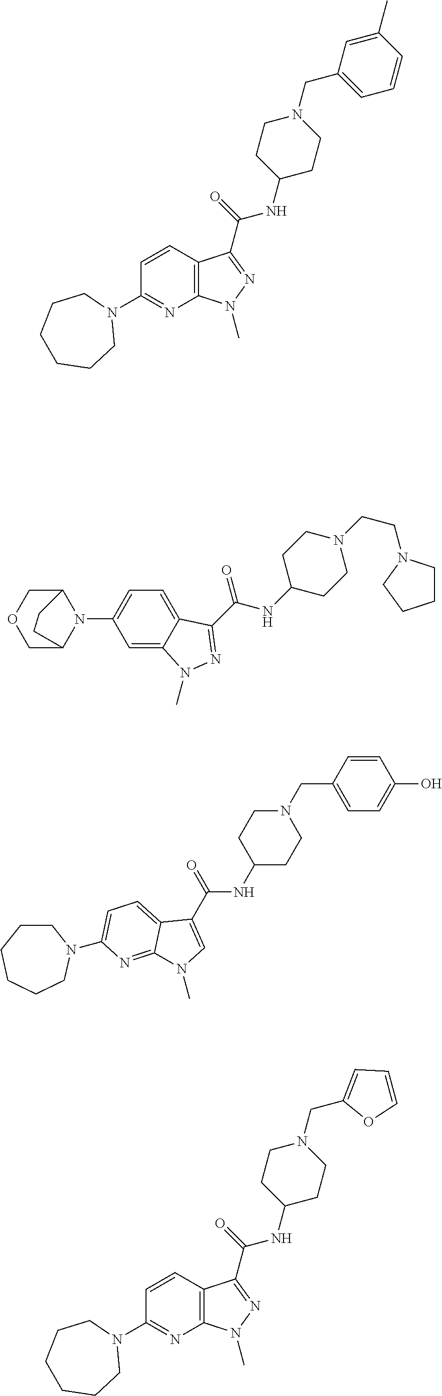 [SMILES: Cc1cccc(CN2CCC(NC(=O)c3nn(C)c4nc(N5CCCCCC5)ccc34)CC2)c1 ; Cn1nc(C(=O)NC2CCN(CCN3CCCC3)CC2)c2ccc(N3C4CCC3COC4)cc21 ; Cn1cc(C(=O)NC2CCN(Cc3ccc(O)cc3)CC2)c2ccc(N3CCCCCC3)nc21 ; Cn1nc(C(=O)NC2CCN(Cc3ccco3)CC2)c2ccc(N3CCCCCC3)nc21]

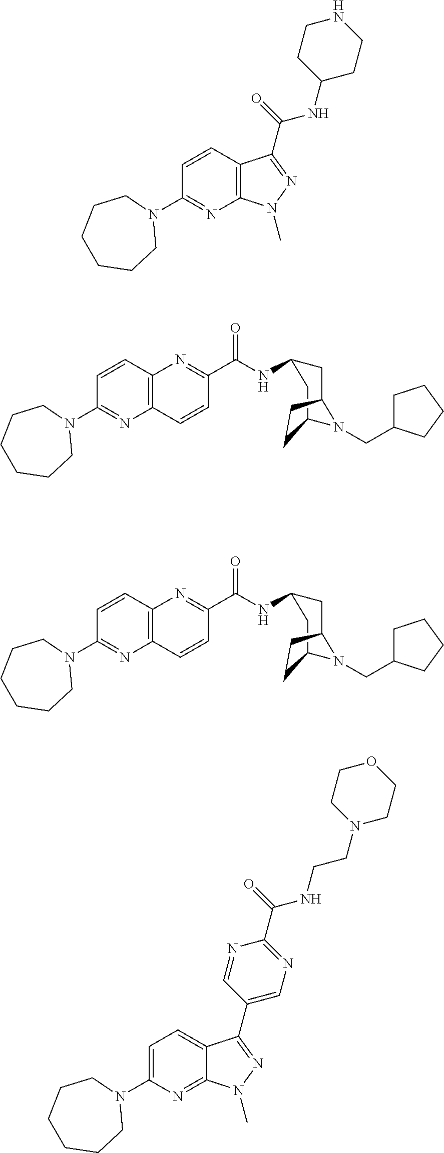

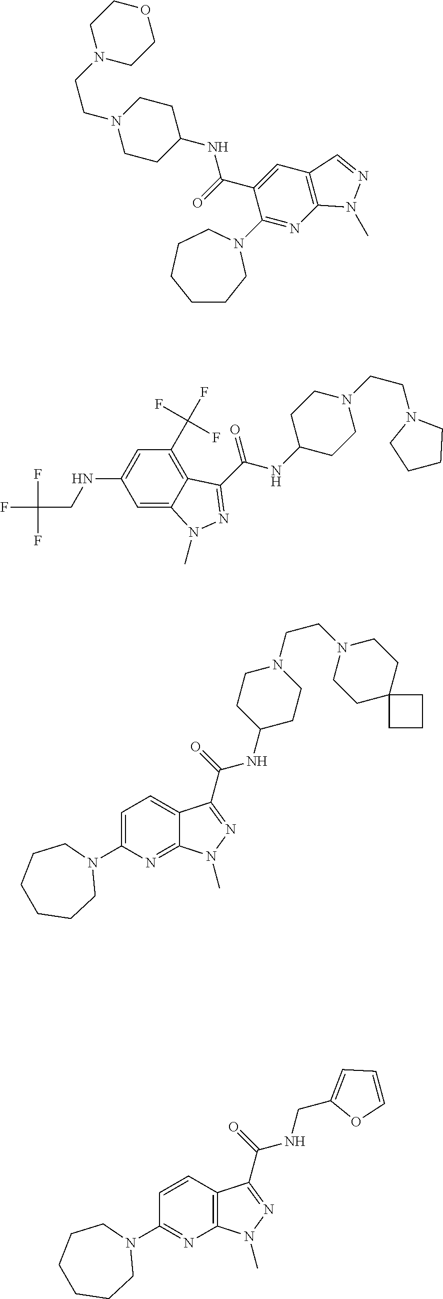 [SMILES: Cn1ncc2cc(C(=O)NC3CCN(CCN4CCOCC4)CC3)c(N3CCCCCC3)nc21 ; Cn1nc(C(=O)NC2CCN(CCN3CCCC3)CC2)c2c(C(F)(F)F)cc(NCC(F)(F)F)cc21 ; Cn1nc(C(=O)NC2CCN(CCN3CCC4(CCC4)CC3)CC2)c2ccc(N3CCCCCC3)nc21 ; Cn1nc(C(=O)NCc2ccco2)c2ccc(N3CCCCCC3)nc21]

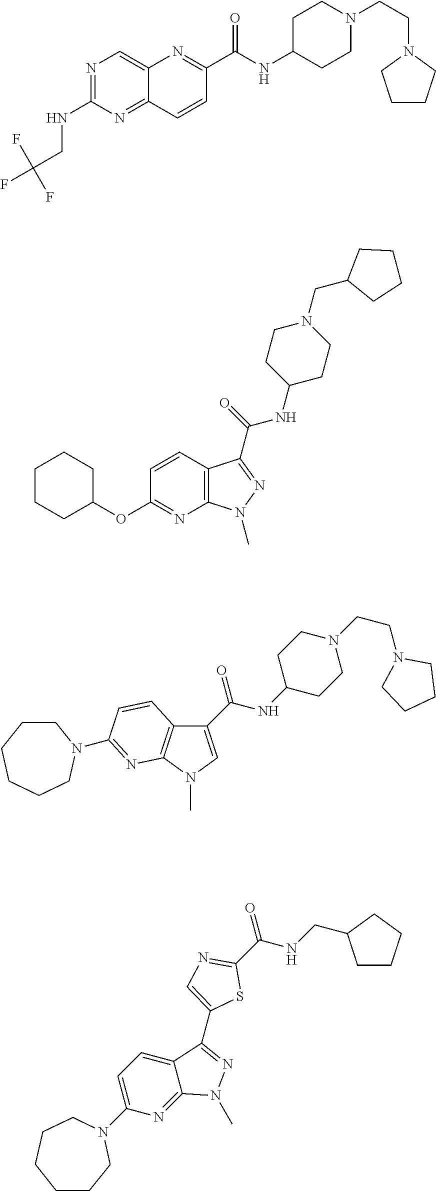 [SMILES: O=C(NC1CCN(CCN2CCCC2)CC1)c1ccc2nc(NCC(F)(F)F)ncc2n1 ; Cn1nc(C(=O)NC2CCN(CC3CCCC3)CC2)c2ccc(OC3CCCCC3)nc21 ; Cn1cc(C(=O)NC2CCN(CCN3CCCC3)CC2)c2ccc(N3CCCCCC3)nc21 ; Cn1nc(-c2cnc(C(=O)NCC3CCCC3)s2)c2ccc(N3CCCCCC3)nc21]

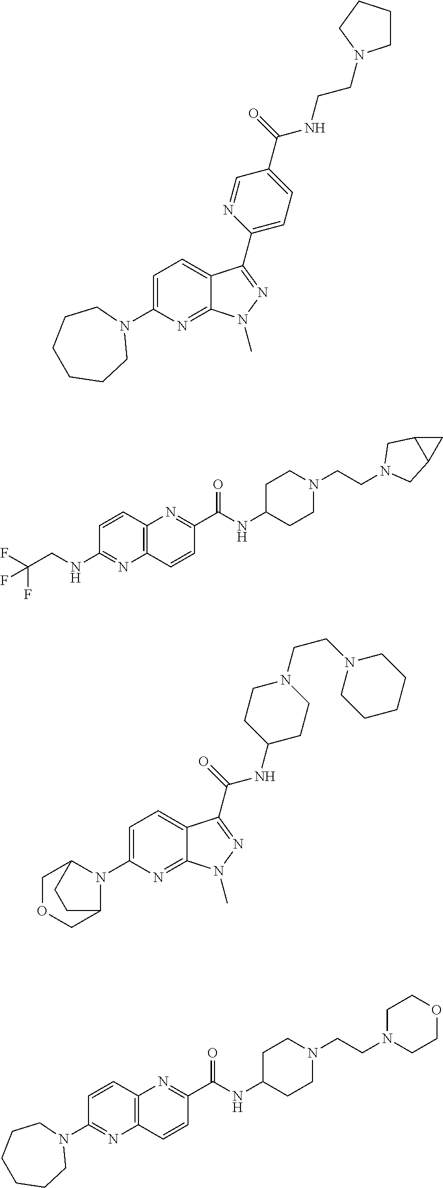 [SMILES: Cn1nc(-c2ccc(C(=O)NCCN3CCCC3)cn2)c2ccc(N3CCCCCC3)nc21 ; O=C(NC1CCN(CCN2CC3CC3C2)CC1)c1ccc2nc(NCC(F)(F)F)ccc2n1 ; Cn1nc(C(=O)NC2CCN(CCN3CCCCC3)CC2)c2ccc(N3C4CCC3COC4)nc21 ; O=C(NC1CCN(CCN2CCOCC2)CC1)c1ccc2nc(N3CCCCCC3)ccc2n1]

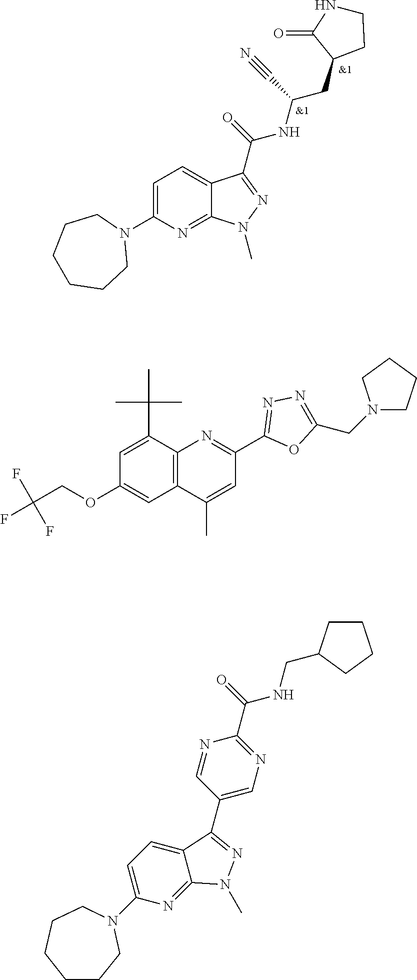 [SMILES: Cn1nc(C(=O)N[C@H](C#N)C[C@@H]2CCNC2=O)c2ccc(N3CCCCCC3)nc21 ; Cc1cc(-c2nnc(CN3CCCC3)o2)nc2c(C(C)(C)C)cc(OCC(F)(F)F)cc12 ; Cn1nc(-c2cnc(C(=O)NCC3CCCC3)nc2)c2ccc(N3CCCCCC3)nc21]

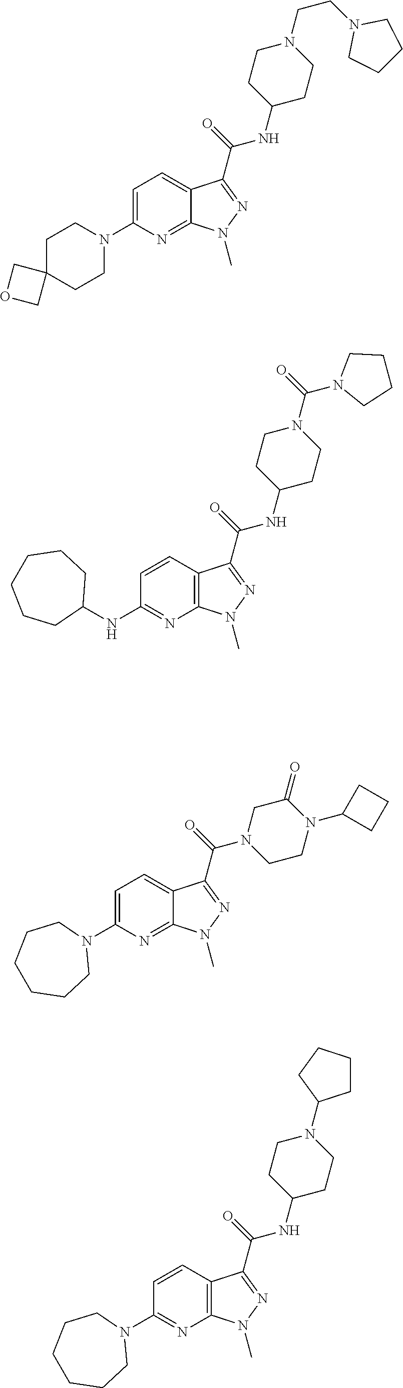 [SMILES: Cn1nc(C(=O)NC2CCN(CCN3CCCC3)CC2)c2ccc(N3CCC4(CC3)COC4)nc21 ; Cn1nc(C(=O)NC2CCN(C(=O)N3CCCC3)CC2)c2ccc(NC3CCCCCC3)nc21 ; Cn1nc(C(=O)N2CCN(C3CCC3)C(=O)C2)c2ccc(N3CCCCCC3)nc21 ; Cn1nc(C(=O)NC2CCN(C3CCCC3)CC2)c2ccc(N3CCCCCC3)nc21]

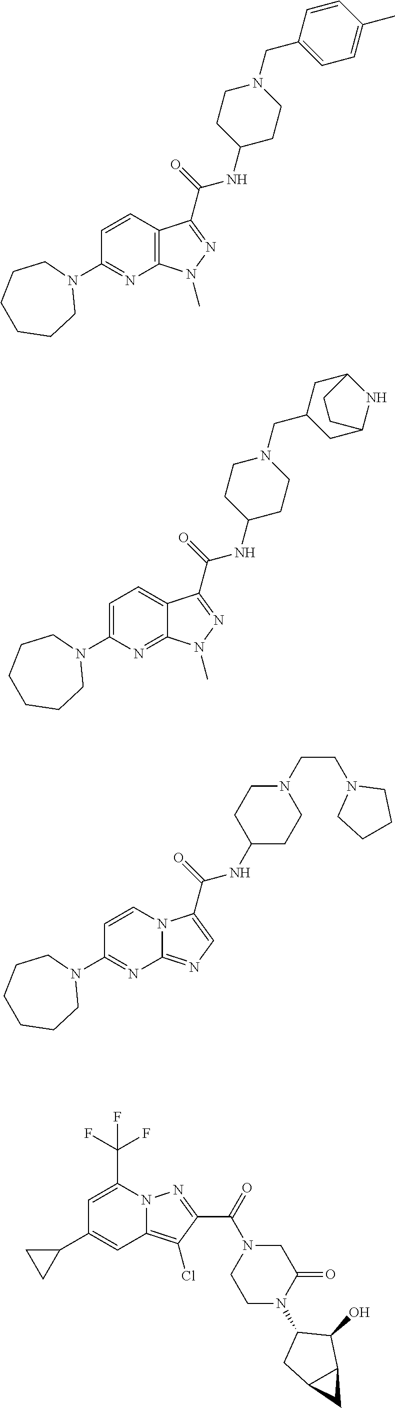 [SMILES: Cc1ccc(CN2CCC(NC(=O)c3nn(C)c4nc(N5CCCCCC5)ccc34)CC2)cc1 ; Cn1nc(C(=O)NC2CCN(CC3CC4CCC(C3)N4)CC2)c2ccc(N3CCCCCC3)nc21 ; O=C(NC1CCN(CCN2CCCC2)CC1)c1cnc2nc(N3CCCCCC3)ccn12 ; O=C(c1nn2c(C(F)(F)F)cc(C3CC3)cc2c1Cl)N1CCN([C@H]2C[C@@H]3C[C@@H]3[C@@H]2O)C(=O)C1]

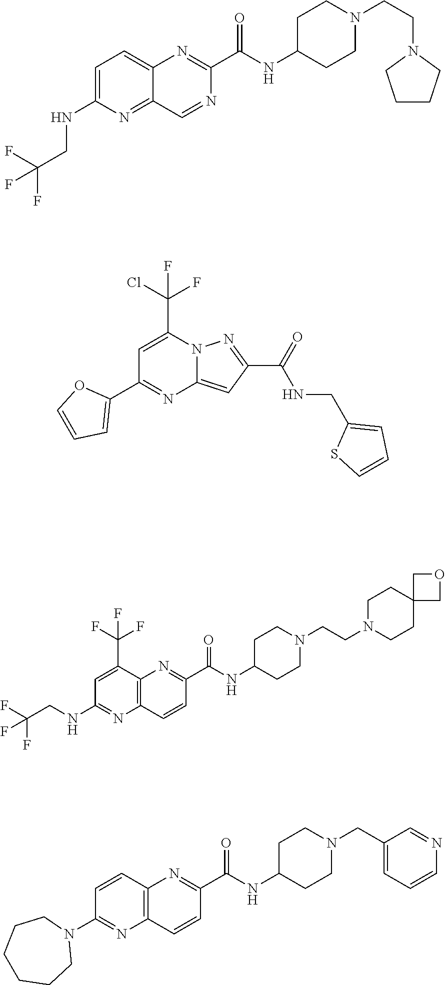 [SMILES: O=C(NC1CCN(CCN2CCCC2)CC1)c1ncc2nc(NCC(F)(F)F)ccc2n1 ; O=C(NCc1cccs1)c1cc2nc(-c3ccco3)cc(C(F)(F)Cl)n2n1 ; O=C(NC1CCN(CCN2CCC3(CC2)COC3)CC1)c1ccc2nc(NCC(F)(F)F)cc(C(F)(F)F)c2n1 ; O=C(NC1CCN(Cc2cccnc2)CC1)c1ccc2nc(N3CCCCCC3)ccc2n1]

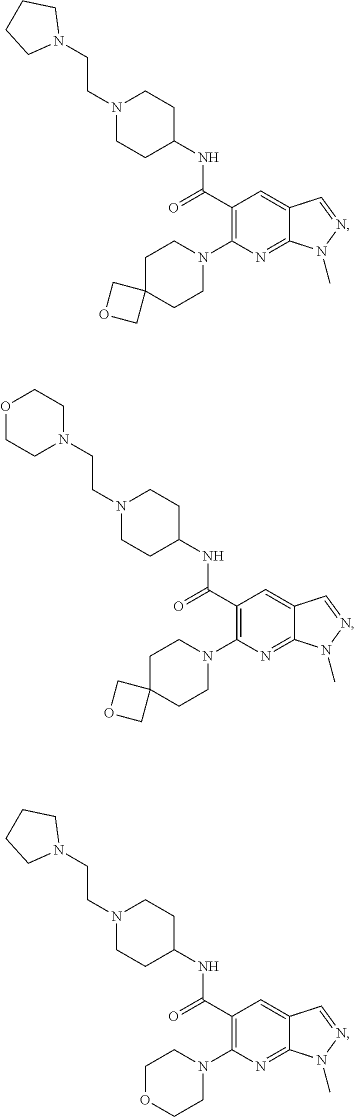 [SMILES: Cn1ncc2cc(C(=O)NC3CCN(CCN4CCCC4)CC3)c(N3CCC4(CC3)COC4)nc21 ; Cn1ncc2cc(C(=O)NC3CCN(CCN4CCOCC4)CC3)c(N3CCC4(CC3)COC4)nc21 ; Cn1ncc2cc(C(=O)NC3CCN(CCN4CCCC4)CC3)c(N3CCOCC3)nc21]

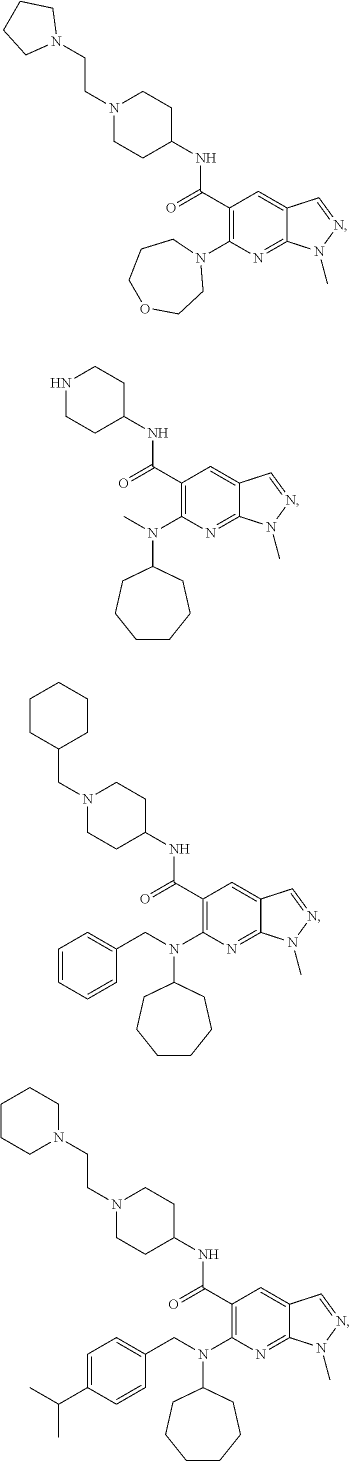 [SMILES: Cn1ncc2cc(C(=O)NC3CCN(CCN4CCCC4)CC3)c(N3CCCOCC3)nc21 ; CN(c1nc2c(cnn2C)cc1C(=O)NC1CCNCC1)C1CCCCCC1 ; Cn1ncc2cc(C(=O)NC3CCN(CC4CCCCC4)CC3)c(N(Cc3ccccc3)C3CCCCCC3)nc21 ; CC(C)c1ccc(CN(c2nc3c(cnn3C)cc2C(=O)NC2CCN(CCN3CCCCC3)CC2)C2CCCCCC2)cc1]

In some embodiments, a subject agent is a compound, or pharmaceutically acceptable salt thereof, disclosed herein. As an illustrative example, in some cases, a subject agent is a compound, or pharmaceutically acceptable salt thereof, selected from the group consisting of:

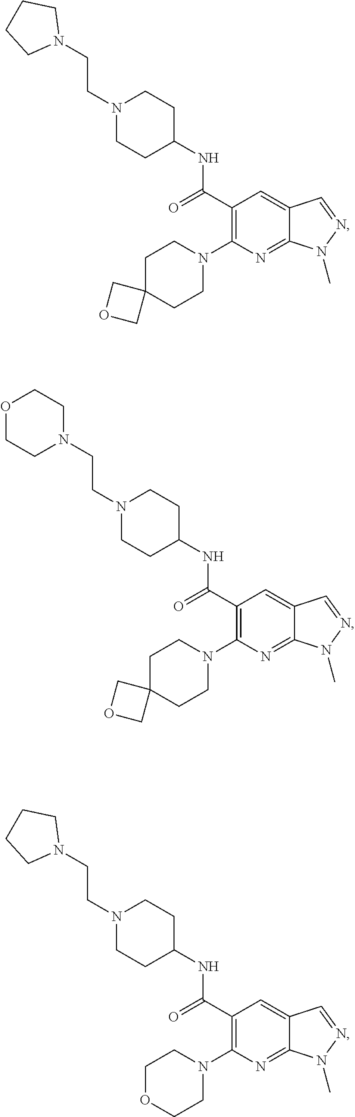 [SMILES: Cn1ncc2cc(C(=O)NC3CCN(CCN4CCCC4)CC3)c(N3CCC4(CC3)COC4)nc21 ; Cn1ncc2cc(C(=O)NC3CCN(CCN4CCOCC4)CC3)c(N3CCC4(CC3)COC4)nc21 ; Cn1ncc2cc(C(=O)NC3CCN(CCN4CCCC4)CC3)c(N3CCOCC3)nc21]

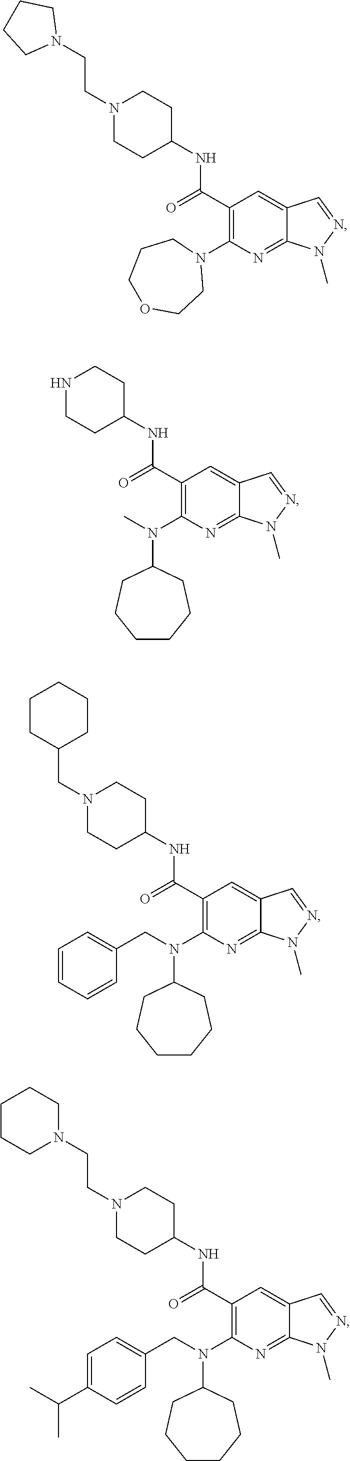 [SMILES: Cn1ncc2cc(C(=O)NC3CCN(CCN4CCCC4)CC3)c(N3CCCOCC3)nc21 ; CN(c1nc2c(cnn2C)cc1C(=O)NC1CCNCC1)C1CCCCCC1 ; Cn1ncc2cc(C(=O)NC3CCN(CC4CCCCC4)CC3)c(N(Cc3ccccc3)C3CCCCCC3)nc21 ; CC(C)c1ccc(CN(c2nc3c(cnn3C)cc2C(=O)NC2CCN(CCN3CCCCC3)CC2)C2CCCCCC2)cc1]

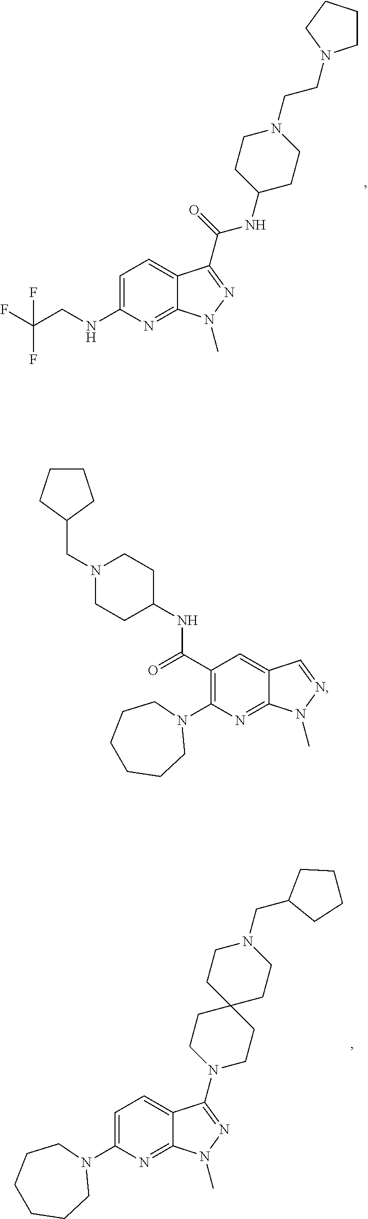 [SMILES: Cn1nc(C(=O)NC2CCN(CCN3CCCC3)CC2)c2ccc(NCC(F)(F)F)nc21 ; Cn1ncc2cc(C(=O)NC3CCN(CC4CCCC4)CC3)c(N3CCCCCC3)nc21 ; Cn1nc(N2CCC3(CCN(CC4CCCC4)CC3)CC2)c2ccc(N3CCCCCC3)nc21]

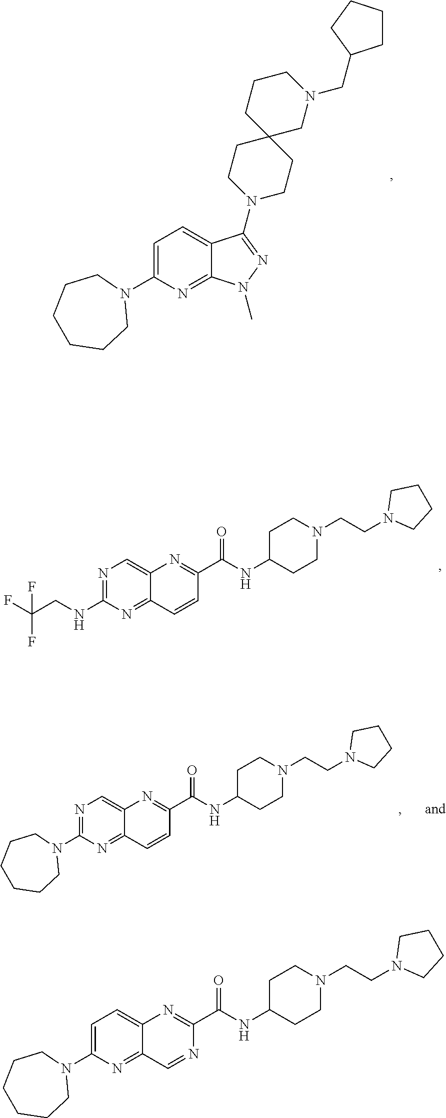 [SMILES: Cn1nc(N2CCC3(CCCN(CC4CCCC4)C3)CC2)c2ccc(N3CCCCCC3)nc21 ; O=C(NC1CCN(CCN2CCCC2)CC1)c1ccc2nc(NCC(F)(F)F)ncc2n1 ; O=C(NC1CCN(CCN2CCCC2)CC1)c1ccc2nc(N3CCCCCC3)ncc2n1 ; O=C(NC1CCN(CCN2CCCC2)CC1)c1ncc2nc(N3CCCCCC3)ccc2n1]

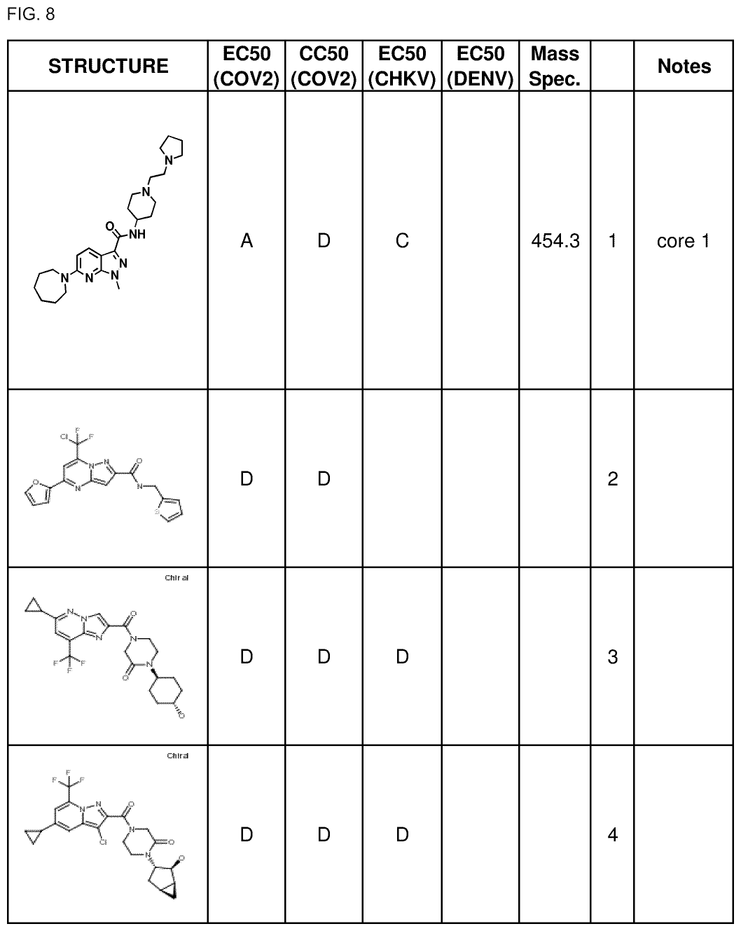

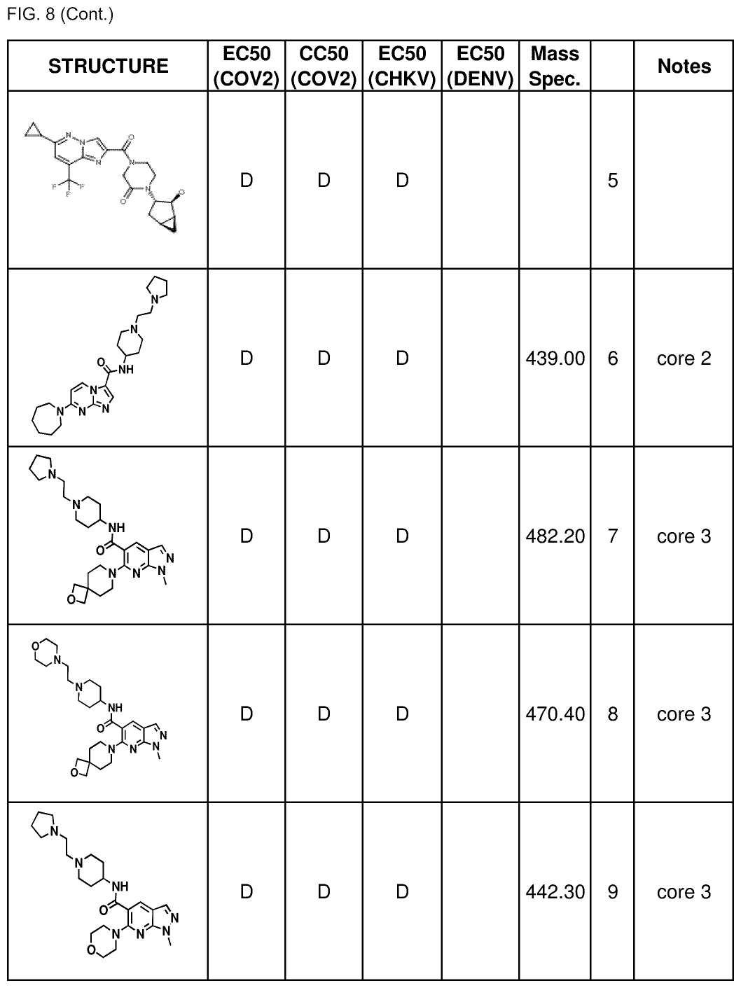

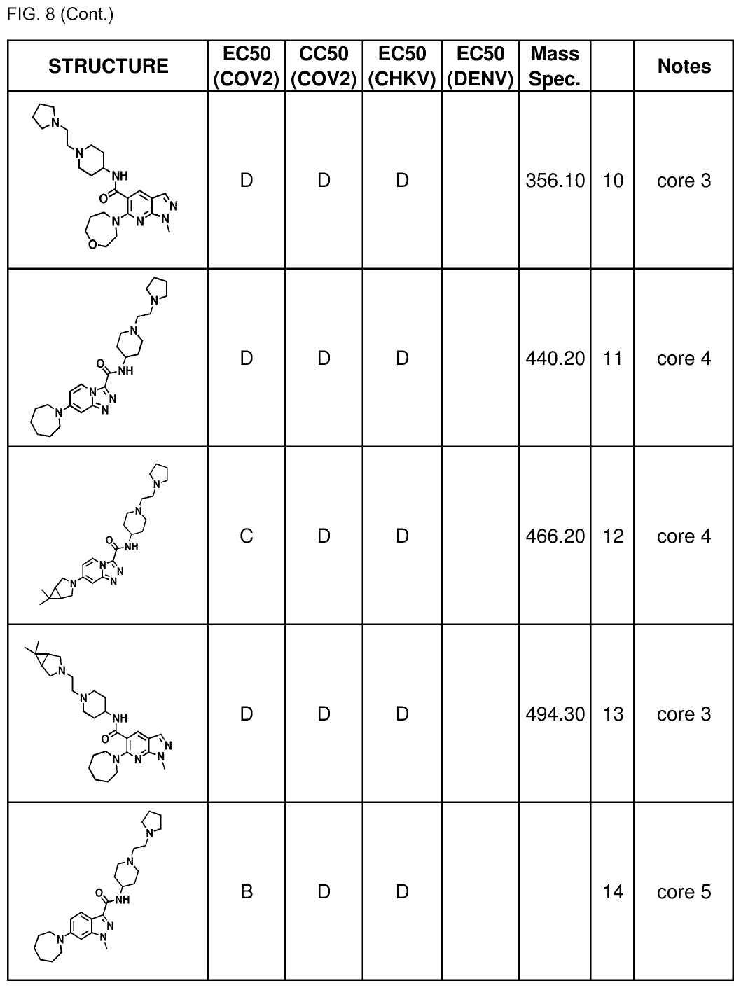

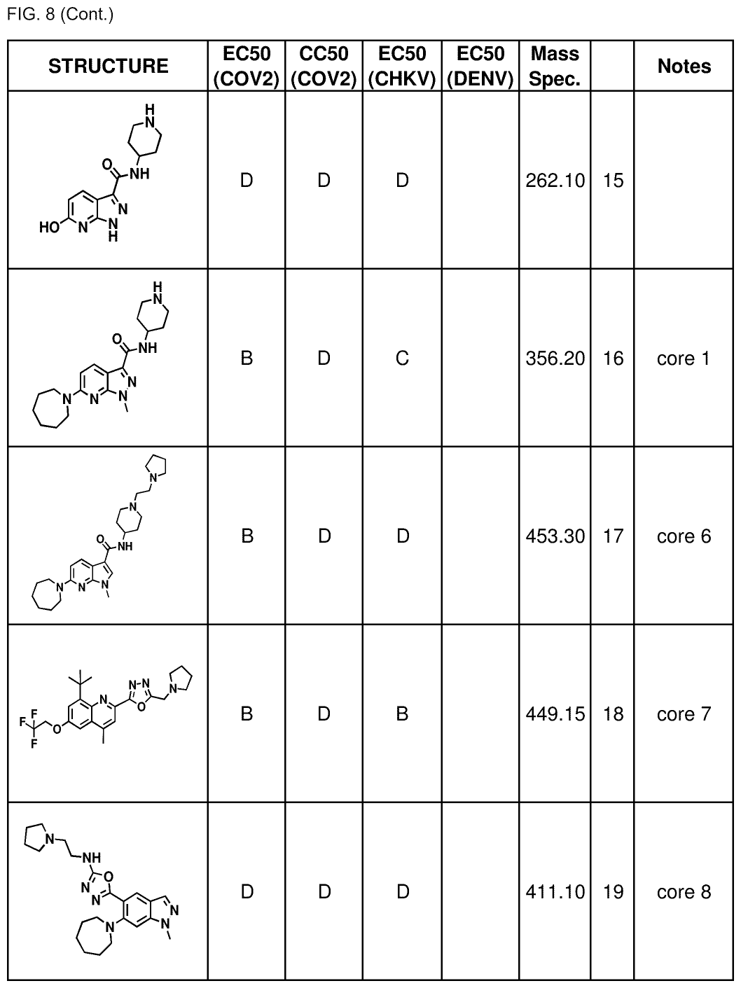

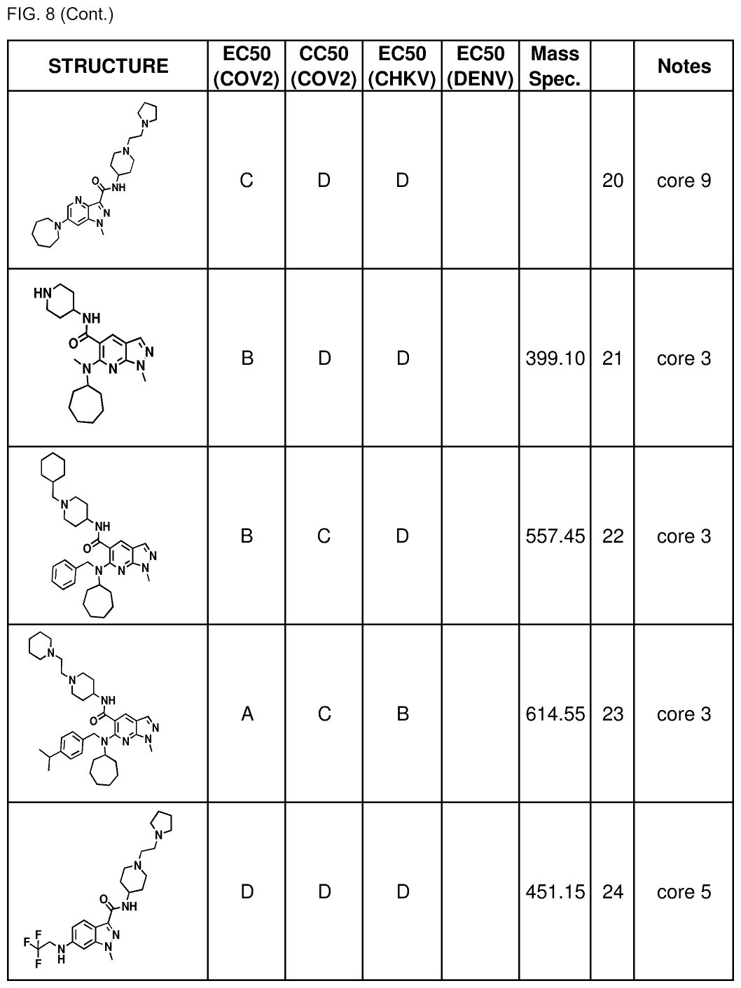

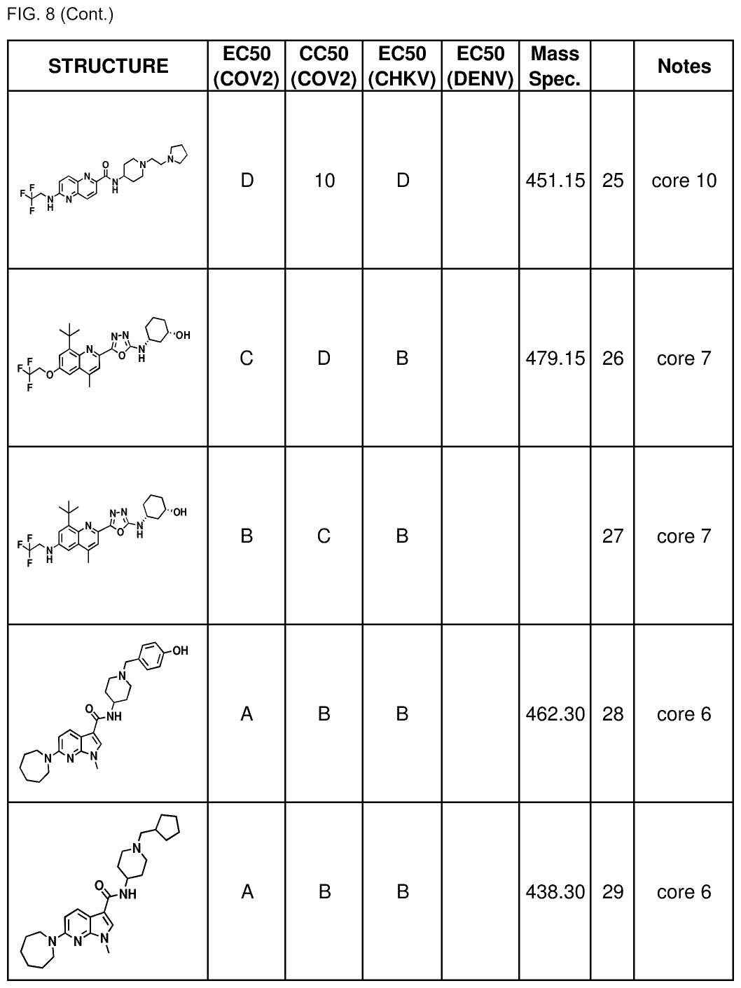

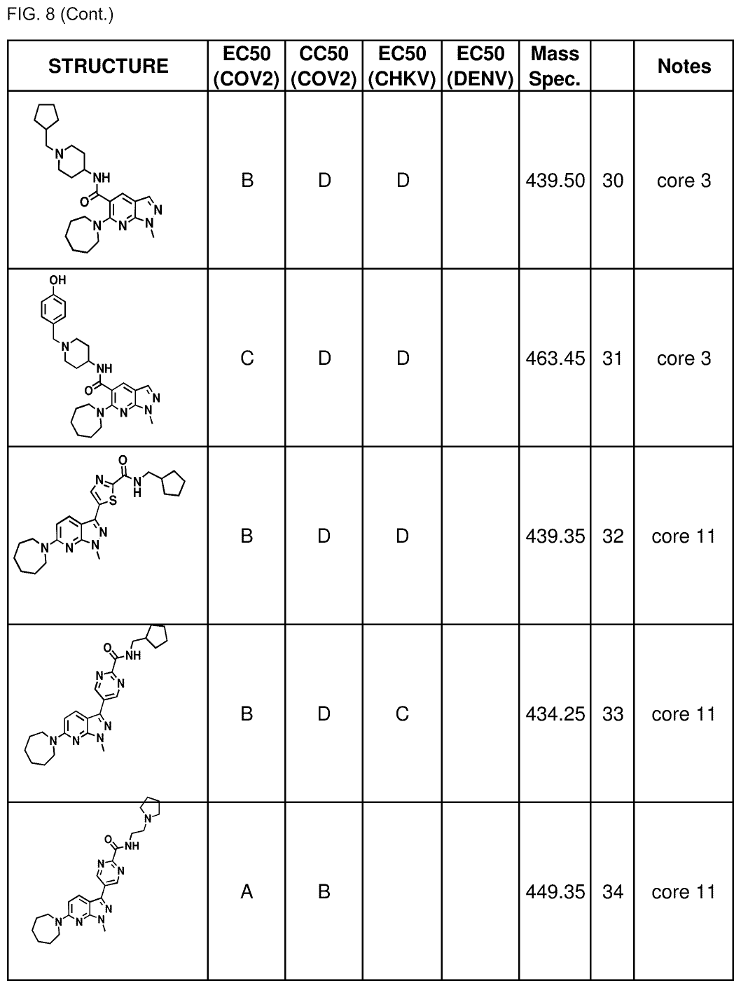

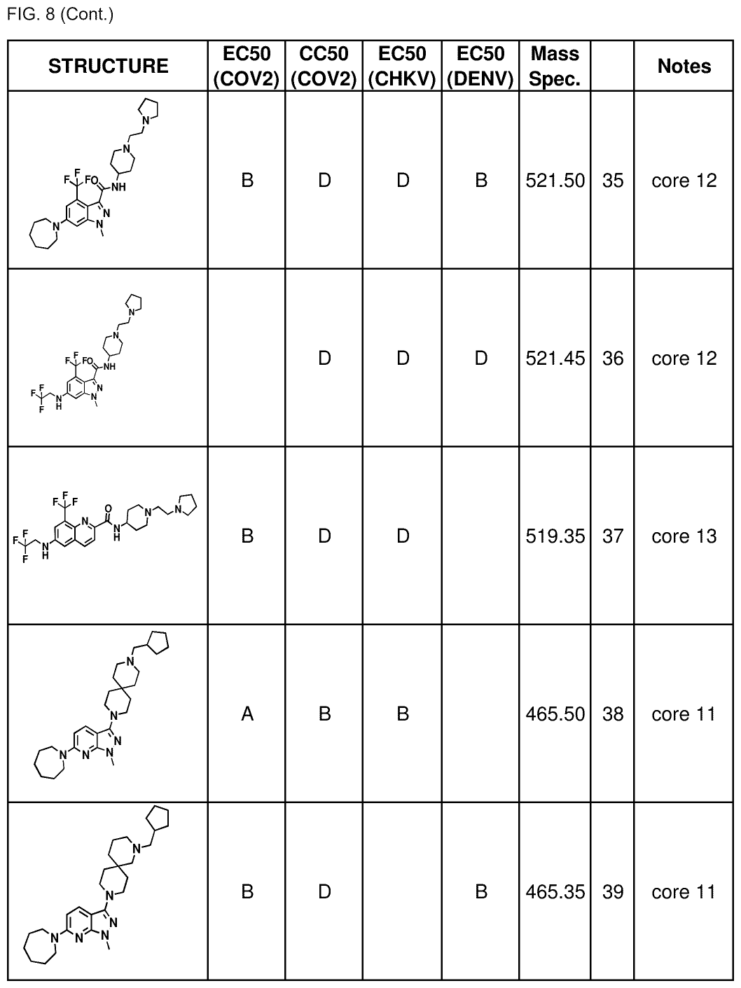

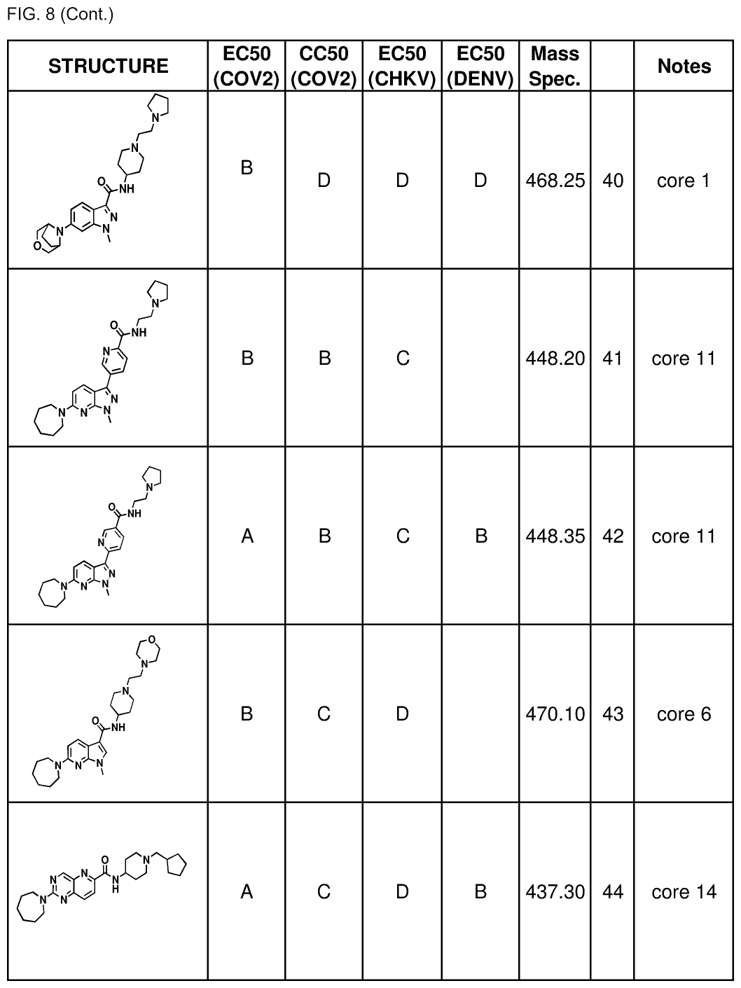

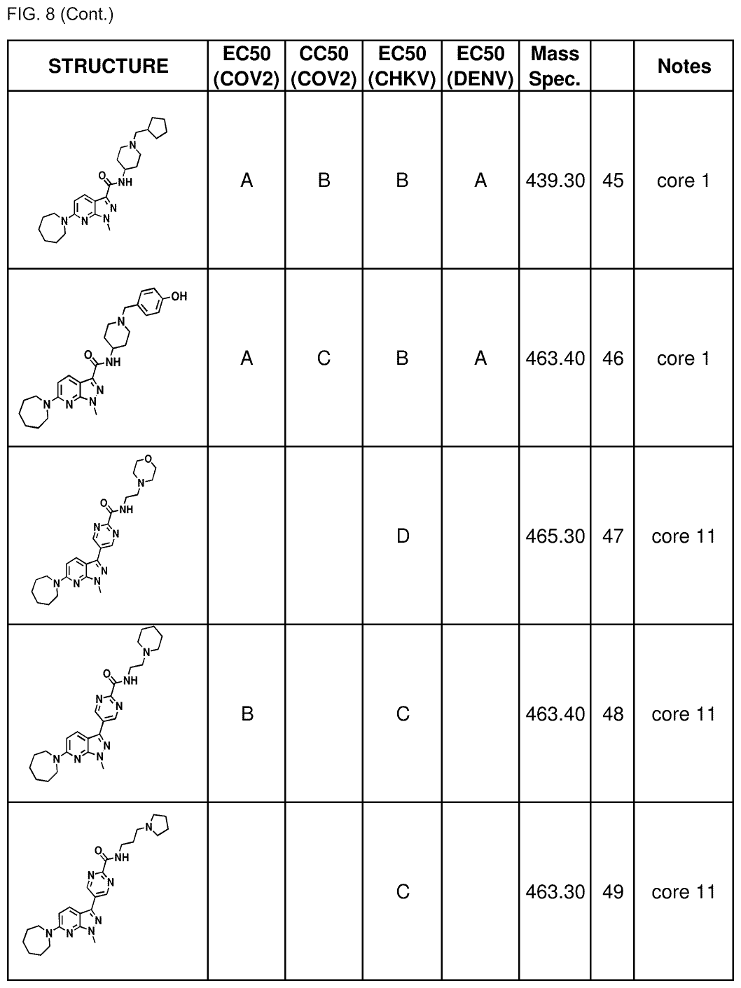

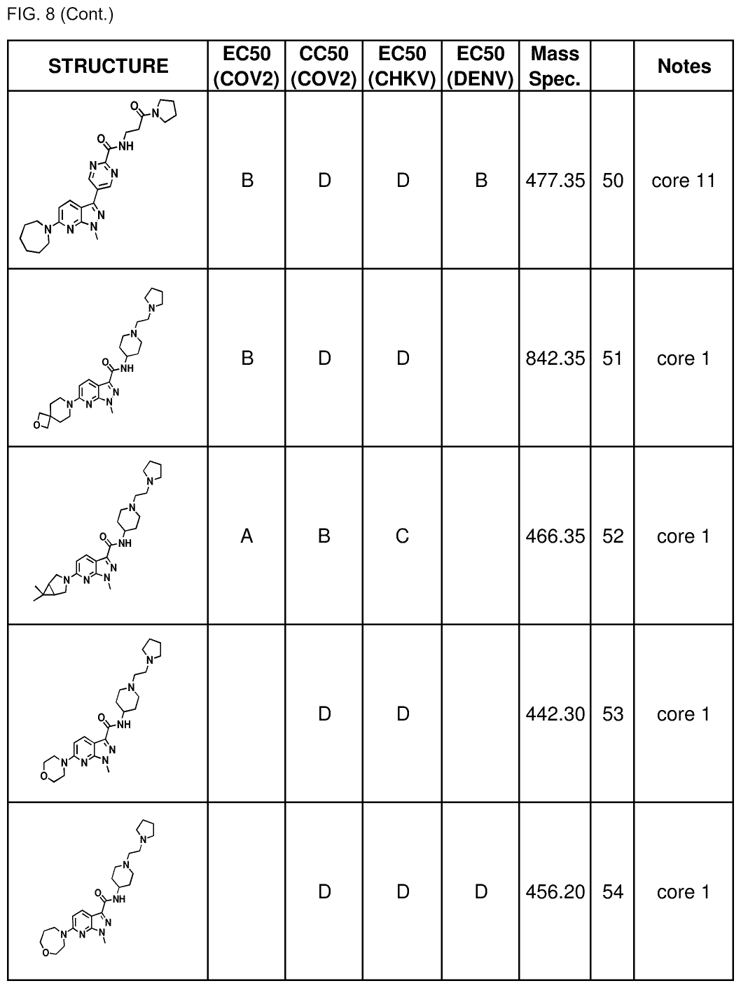

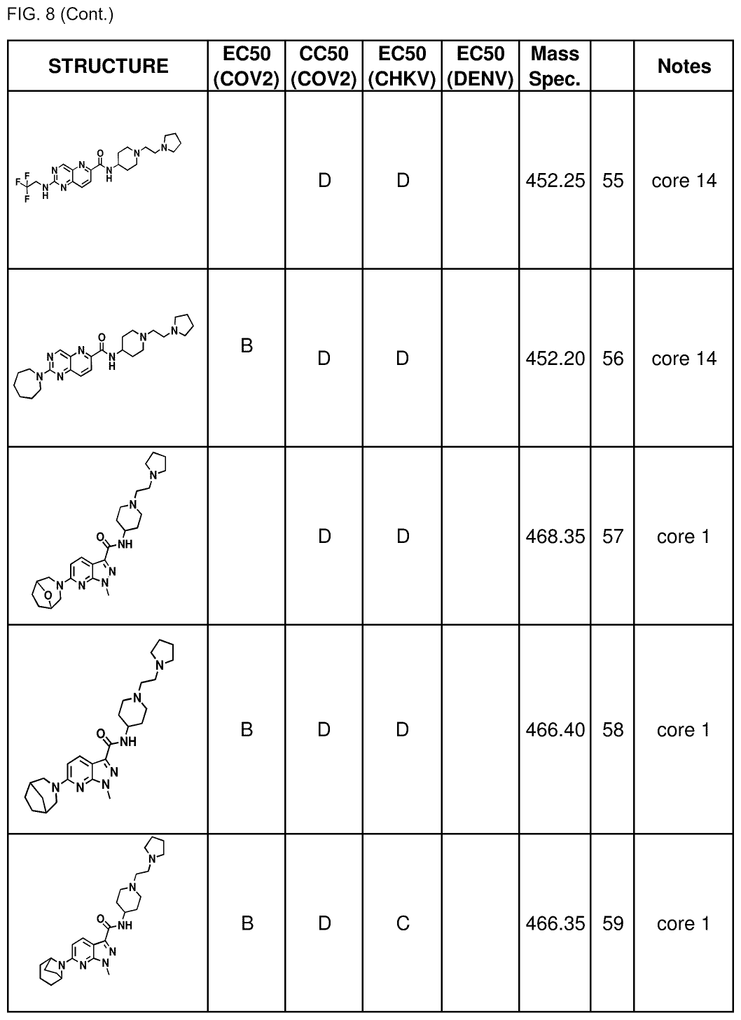

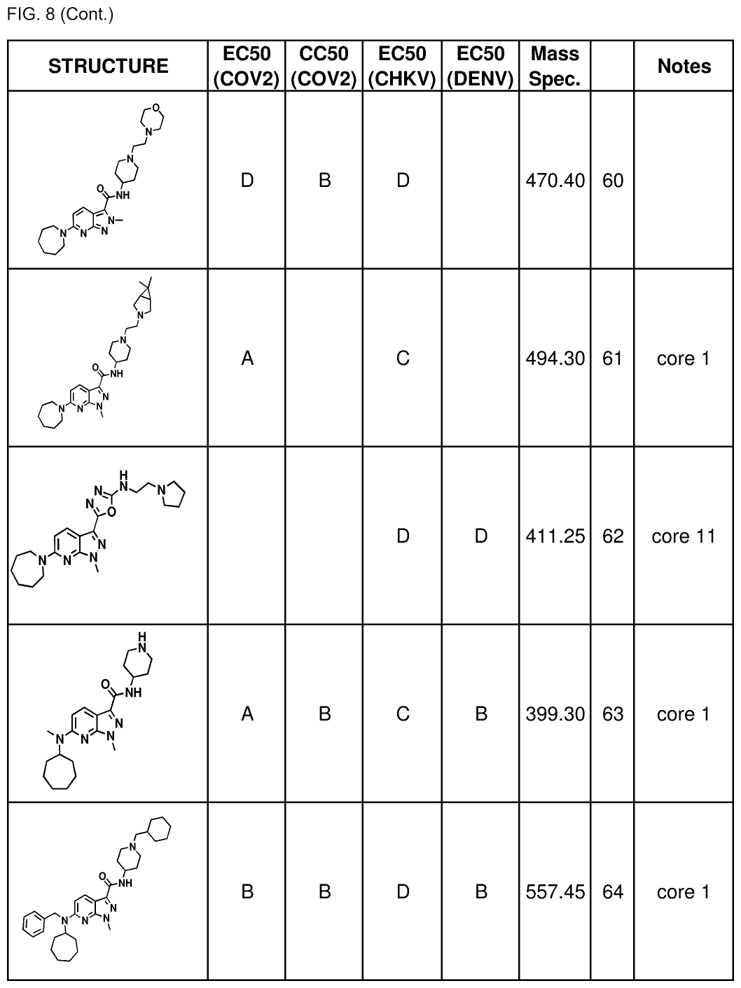

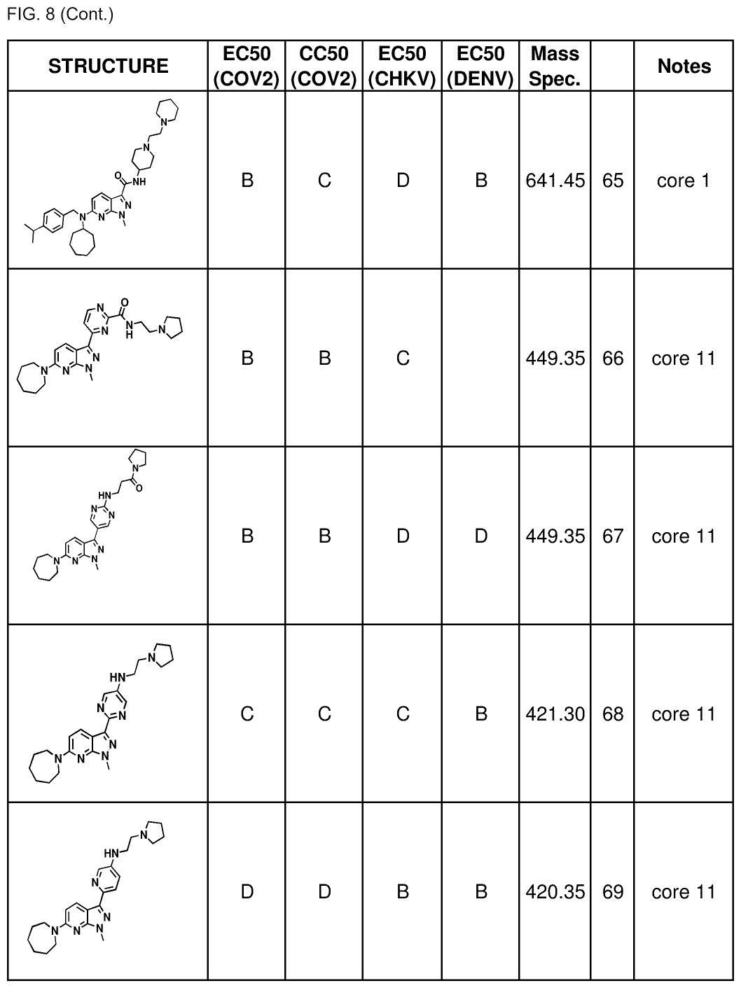

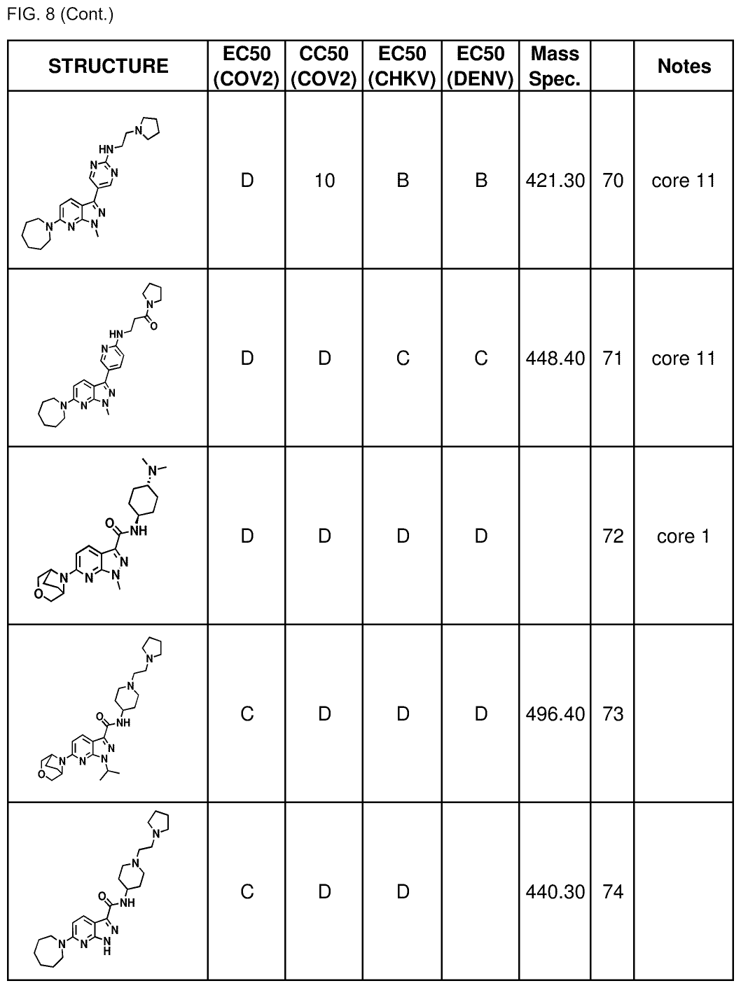

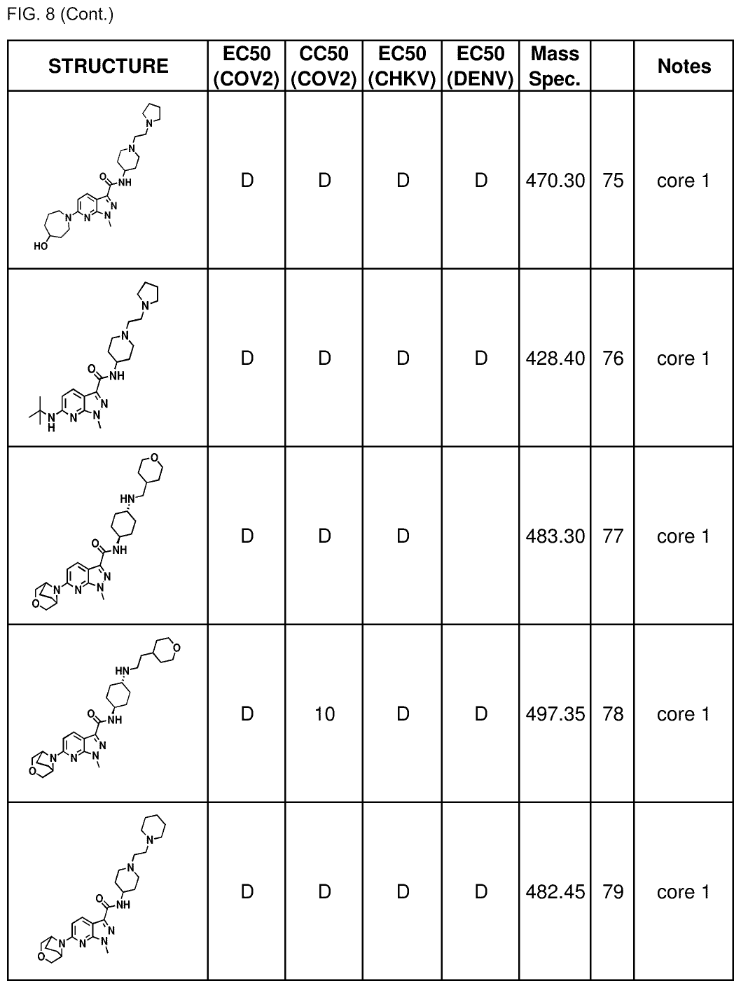

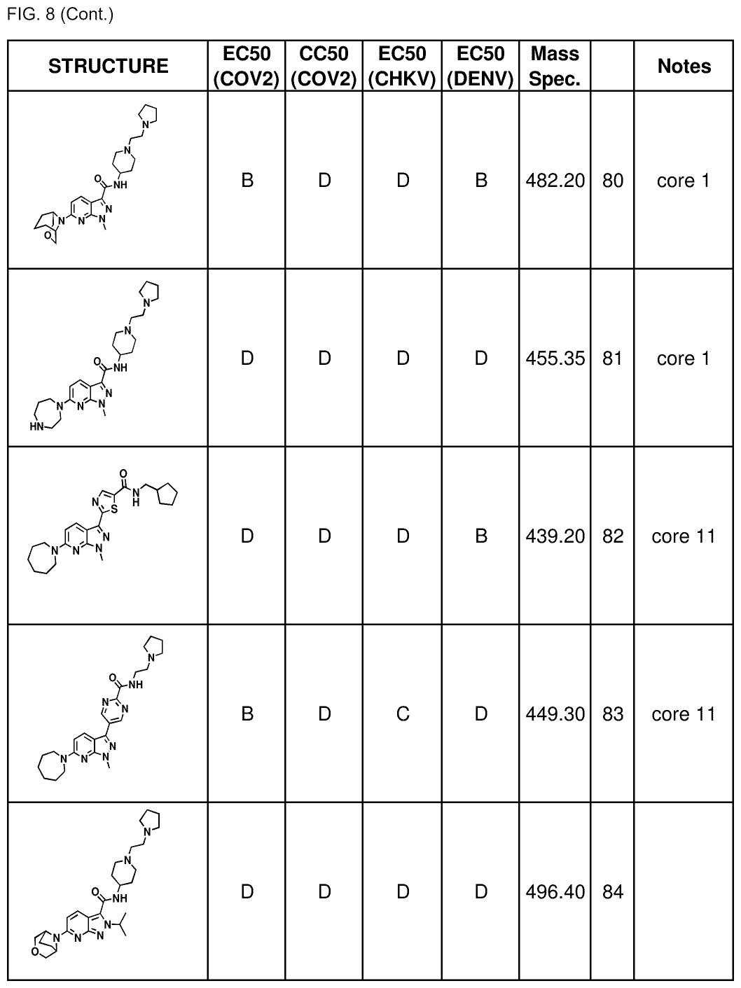

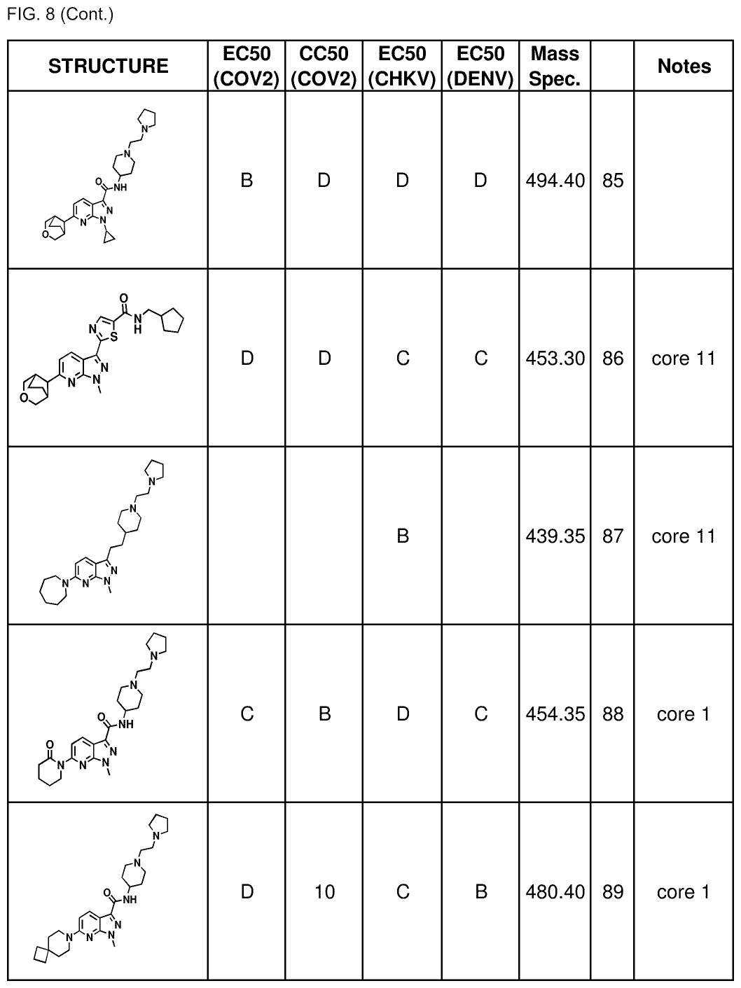

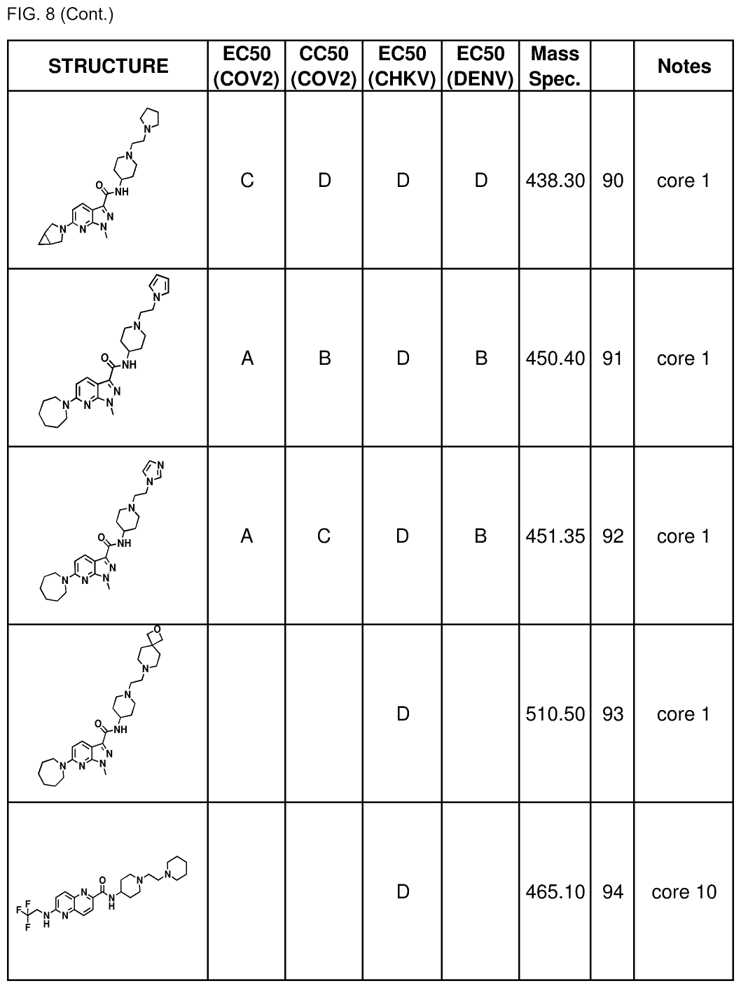

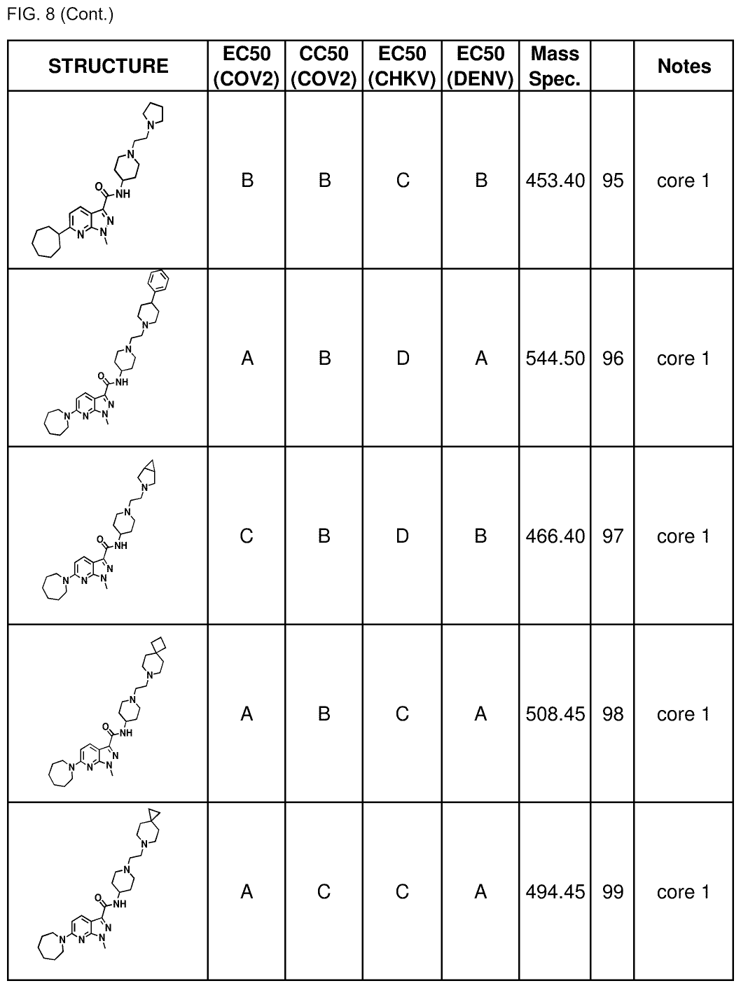

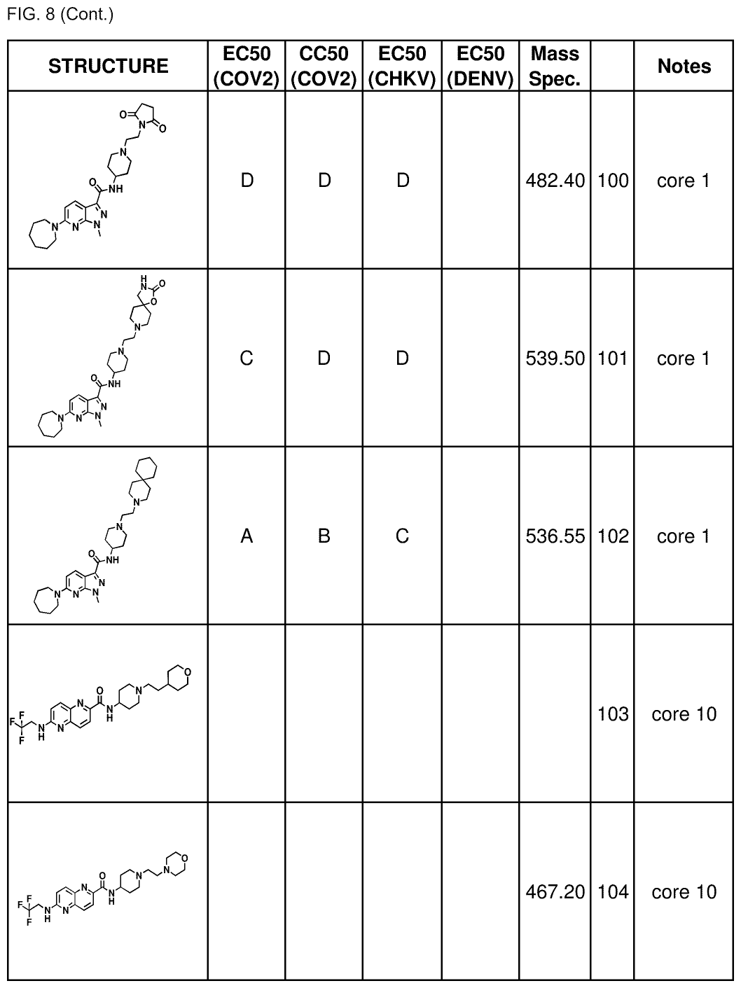

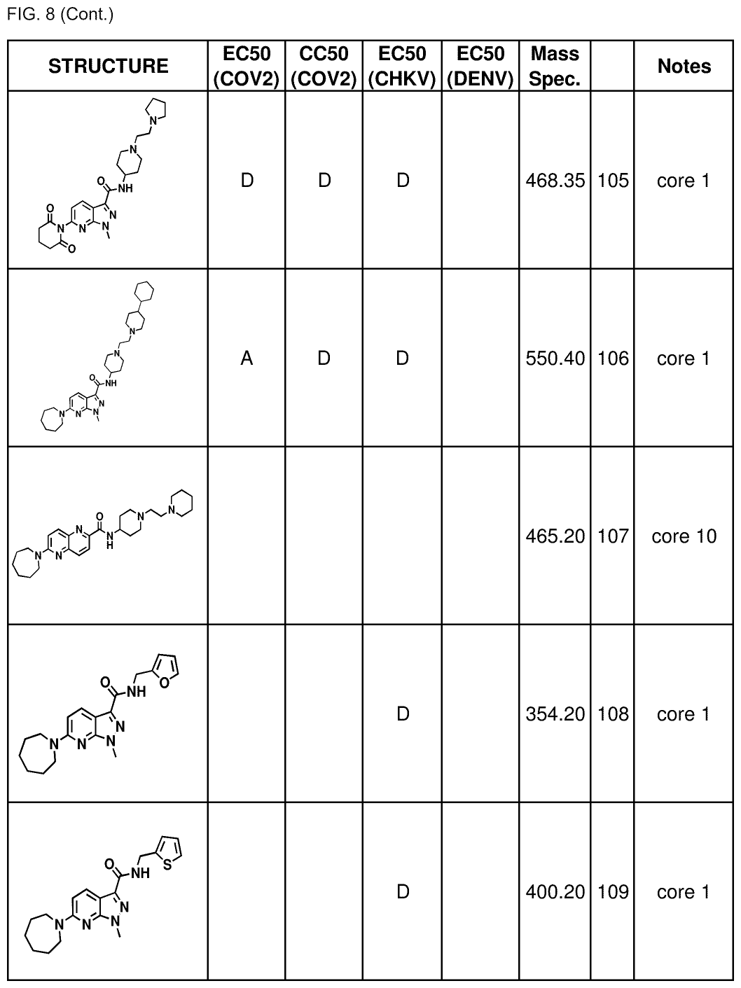

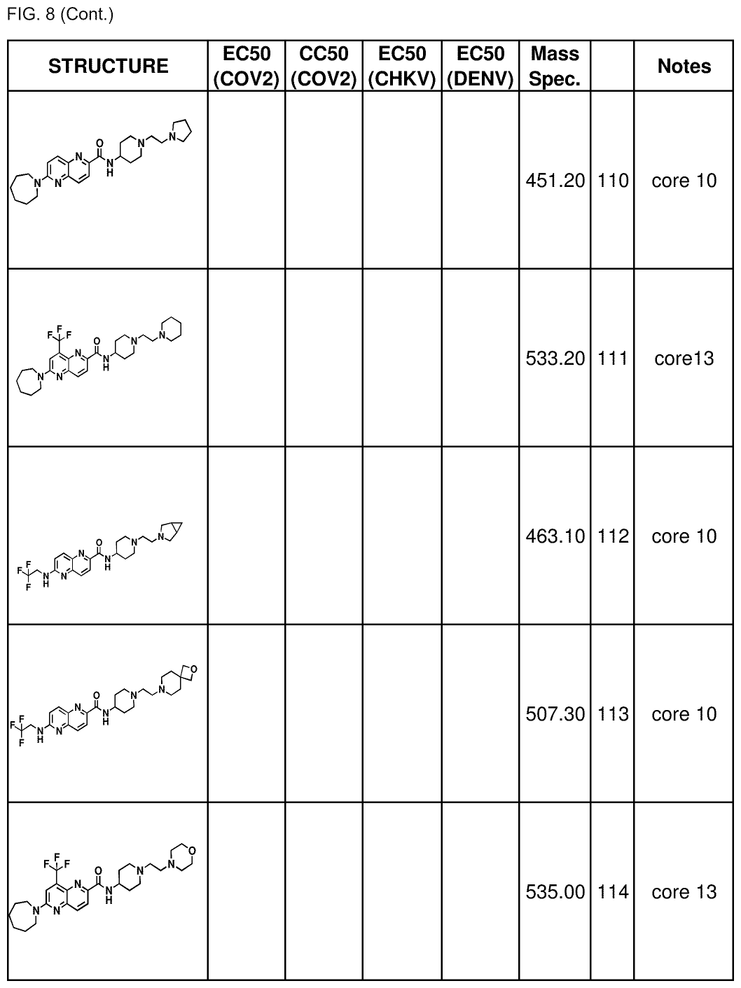

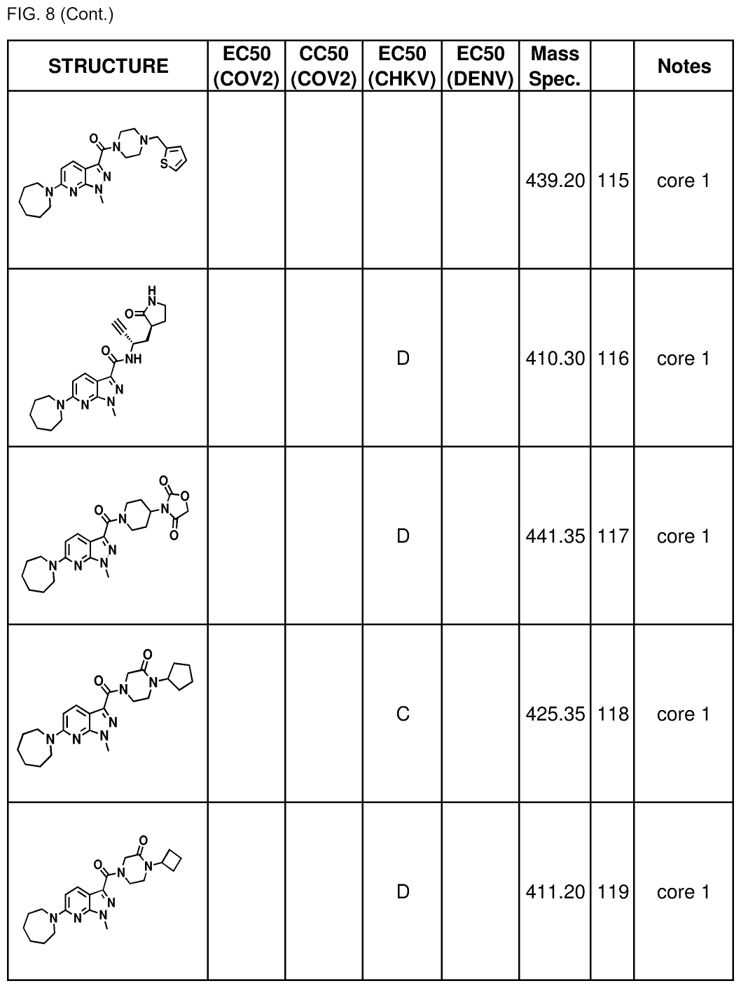

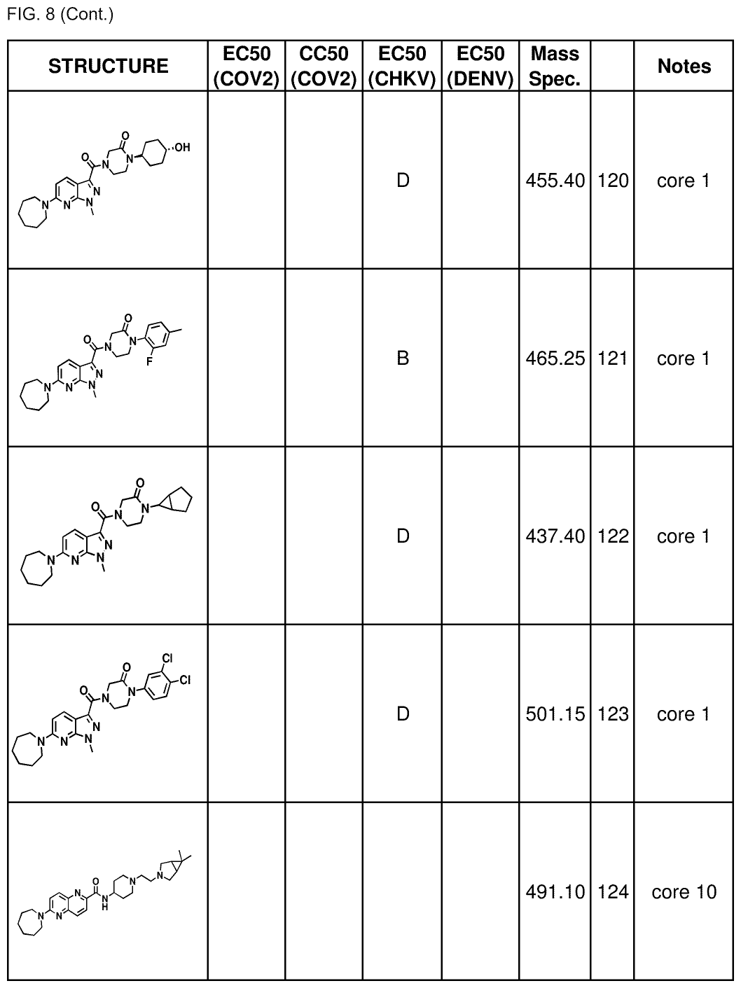

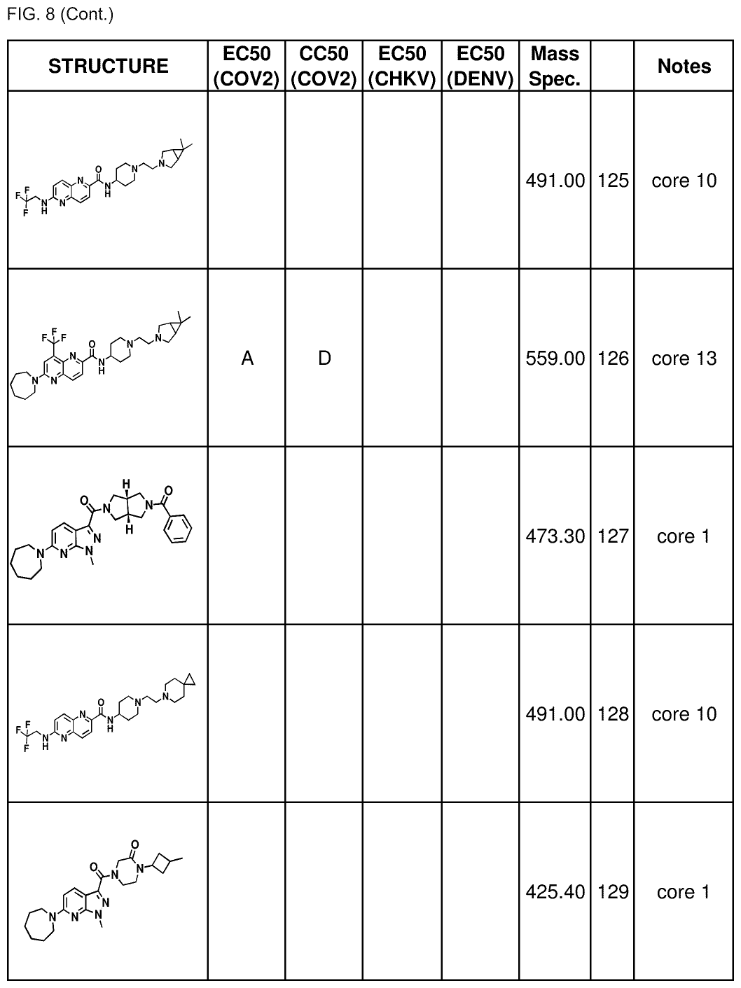

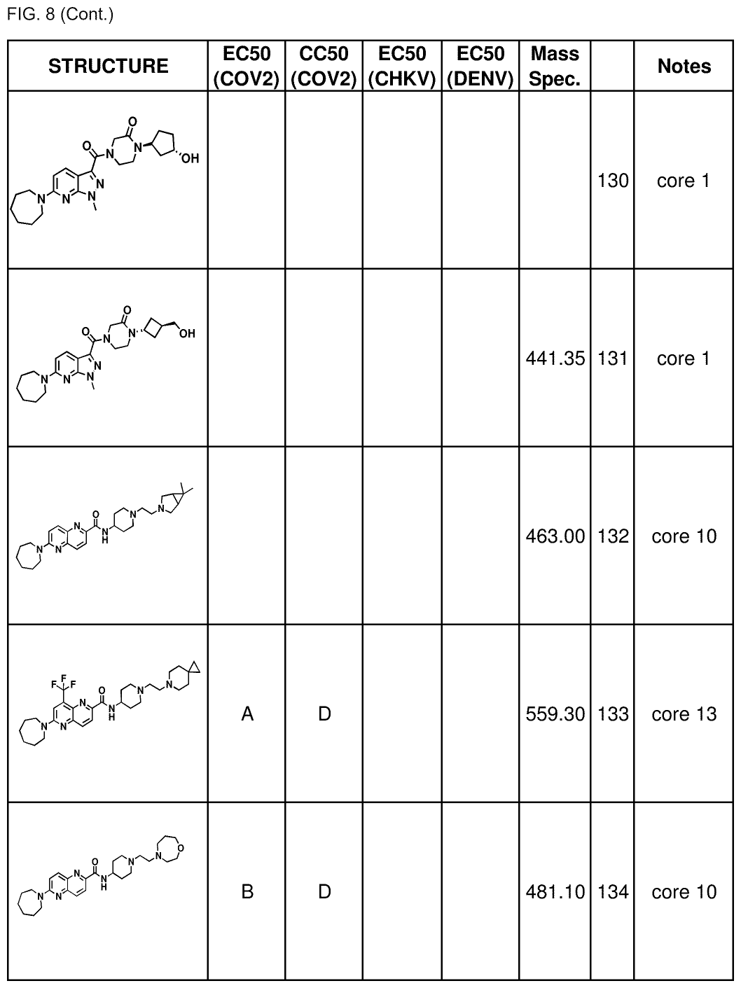

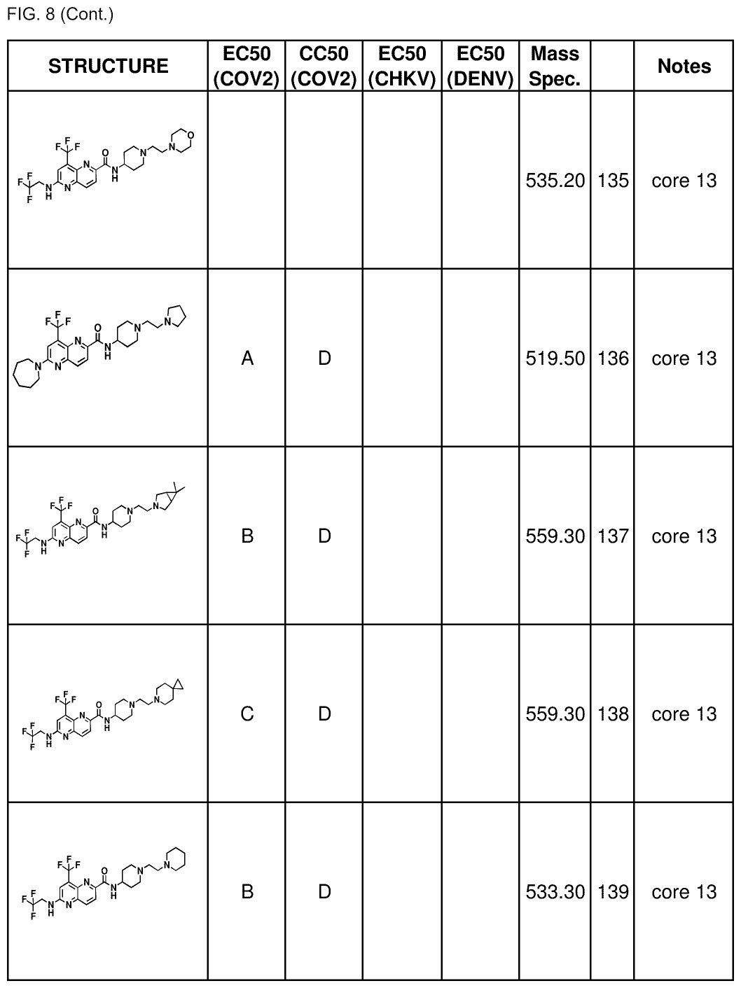

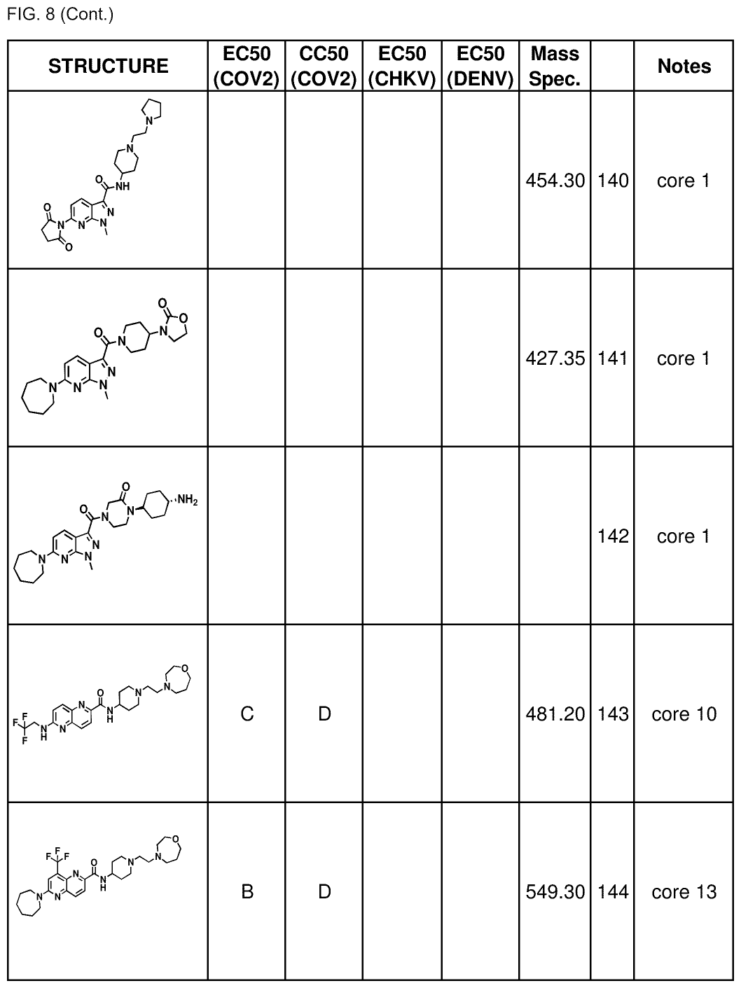

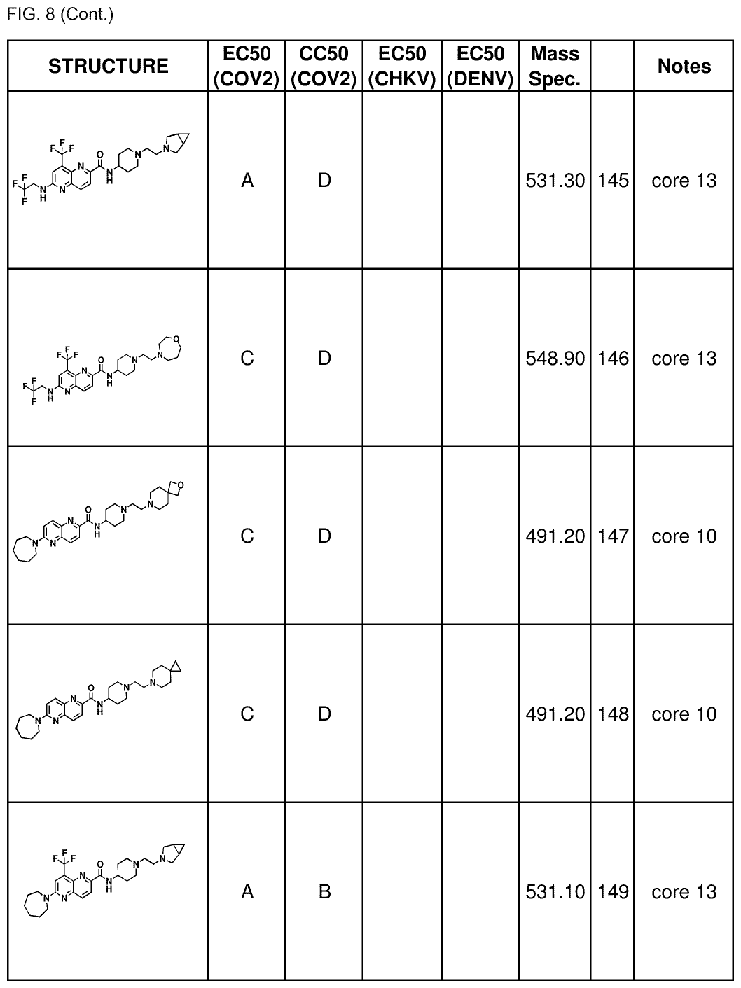

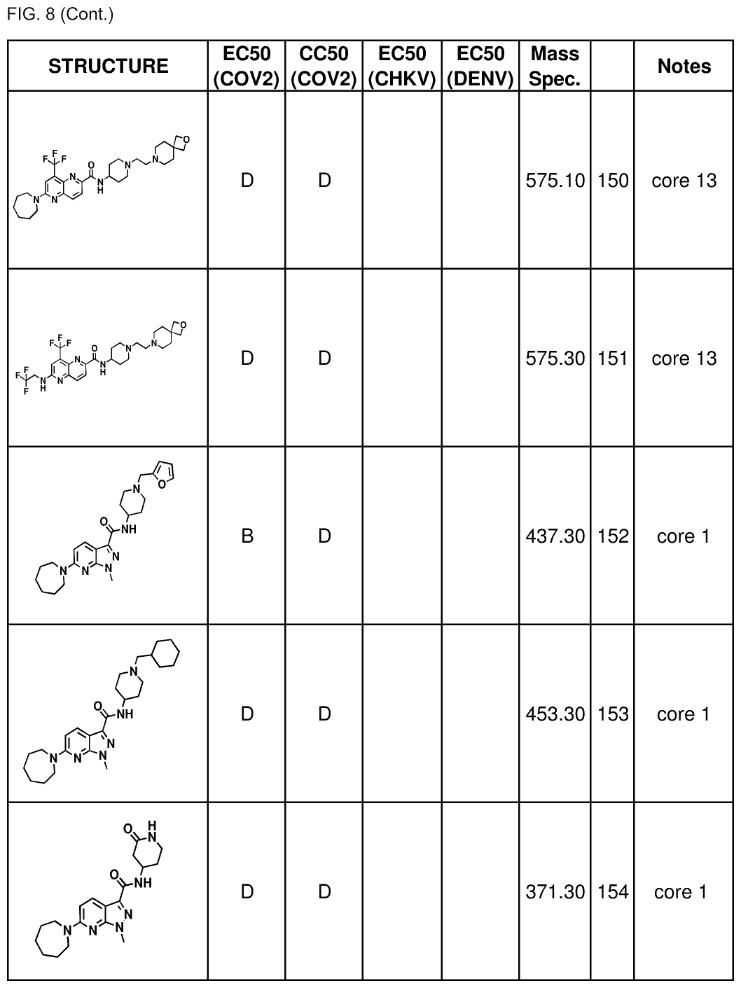

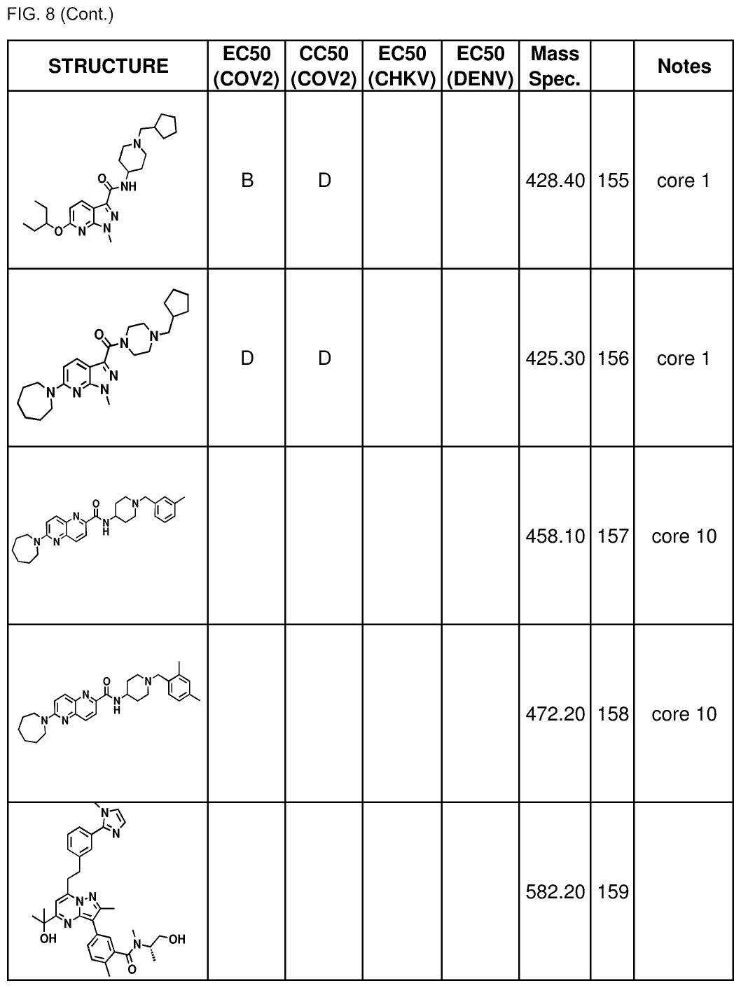

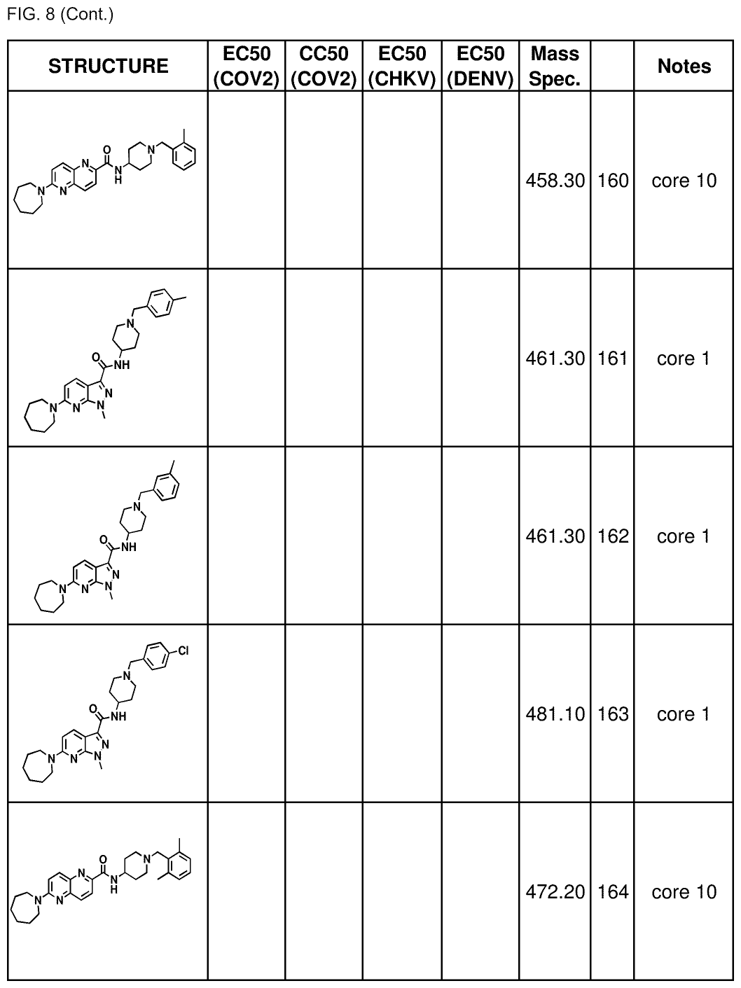

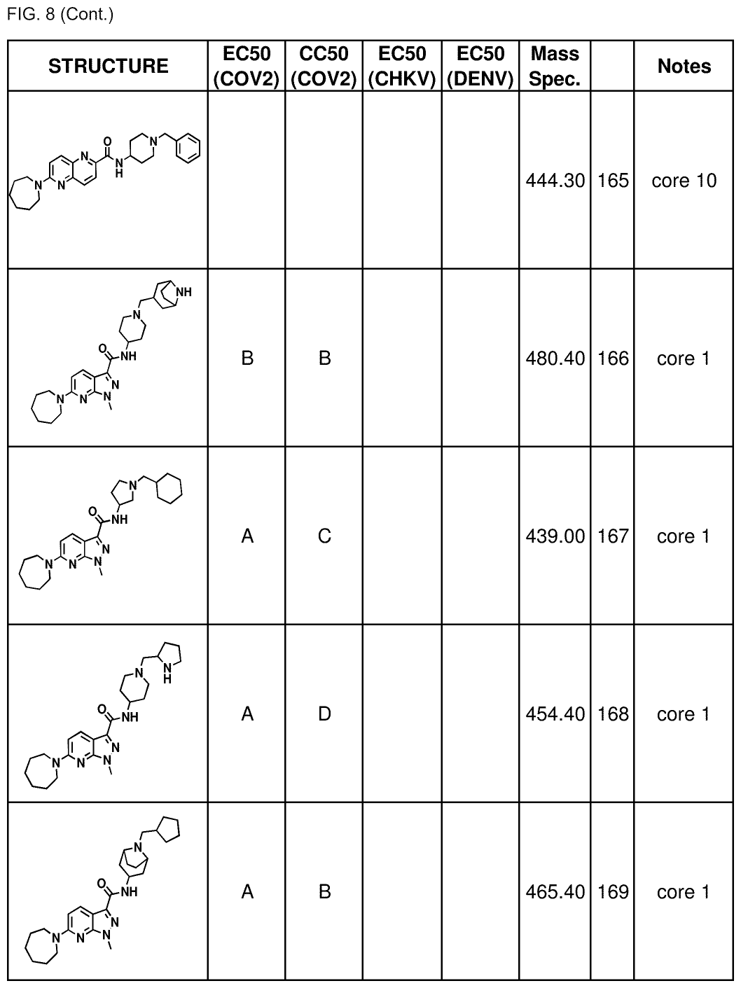

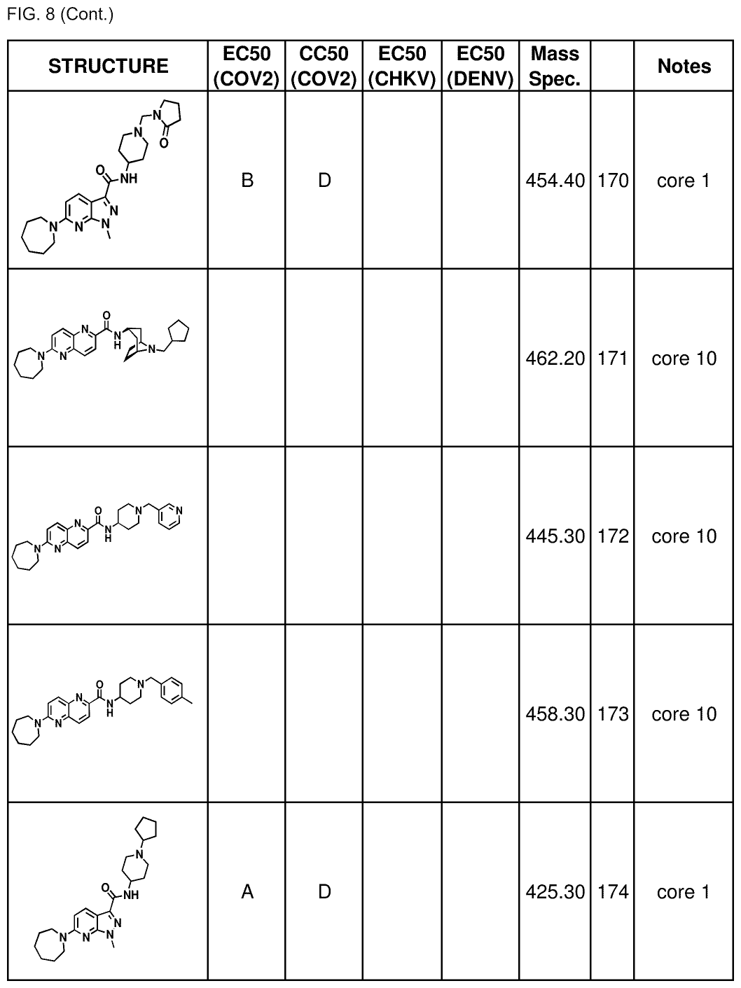

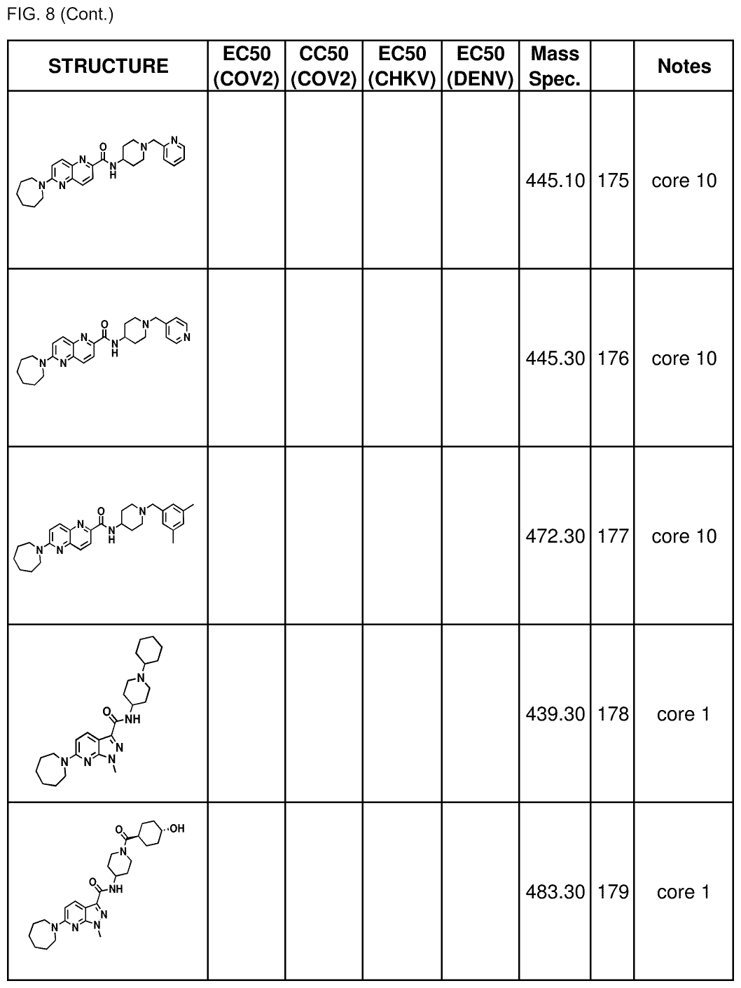

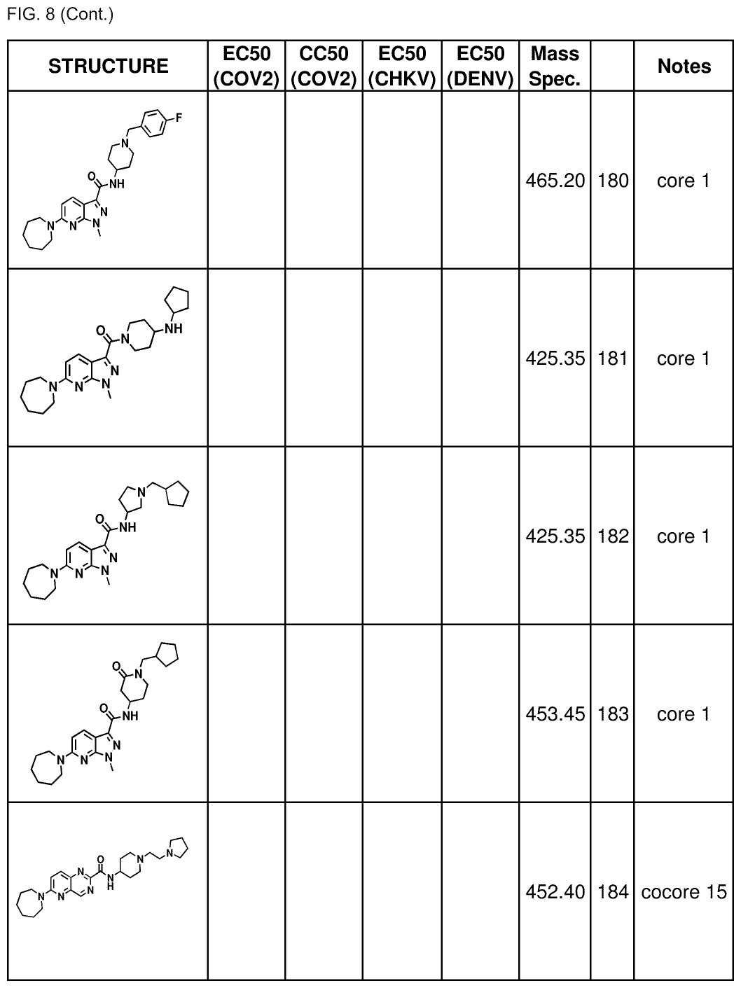

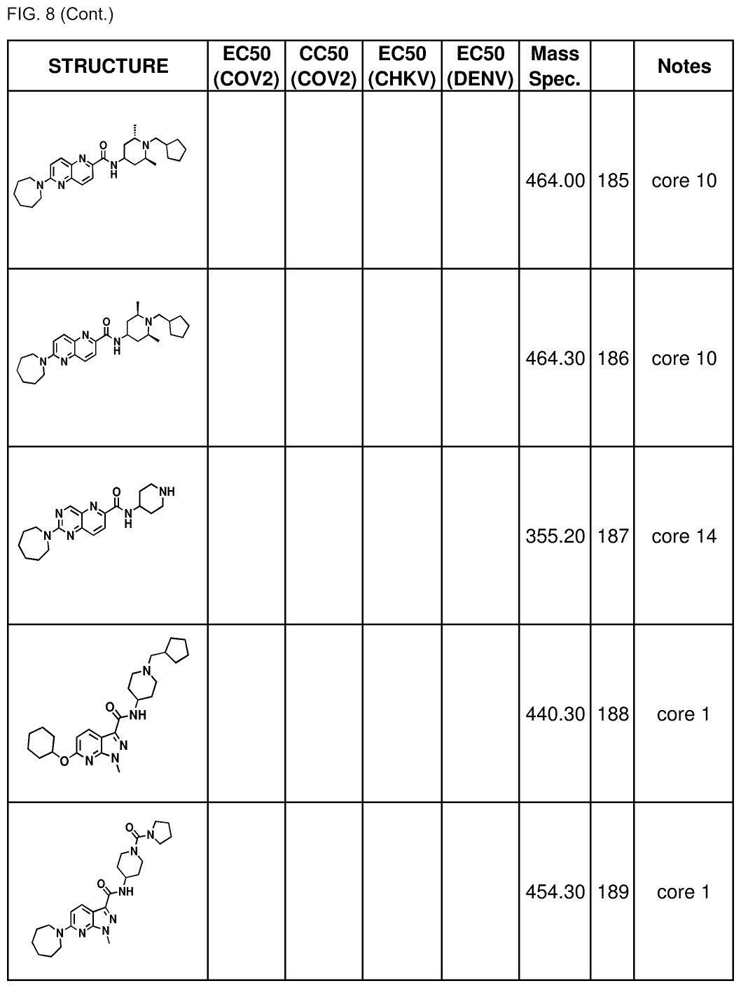

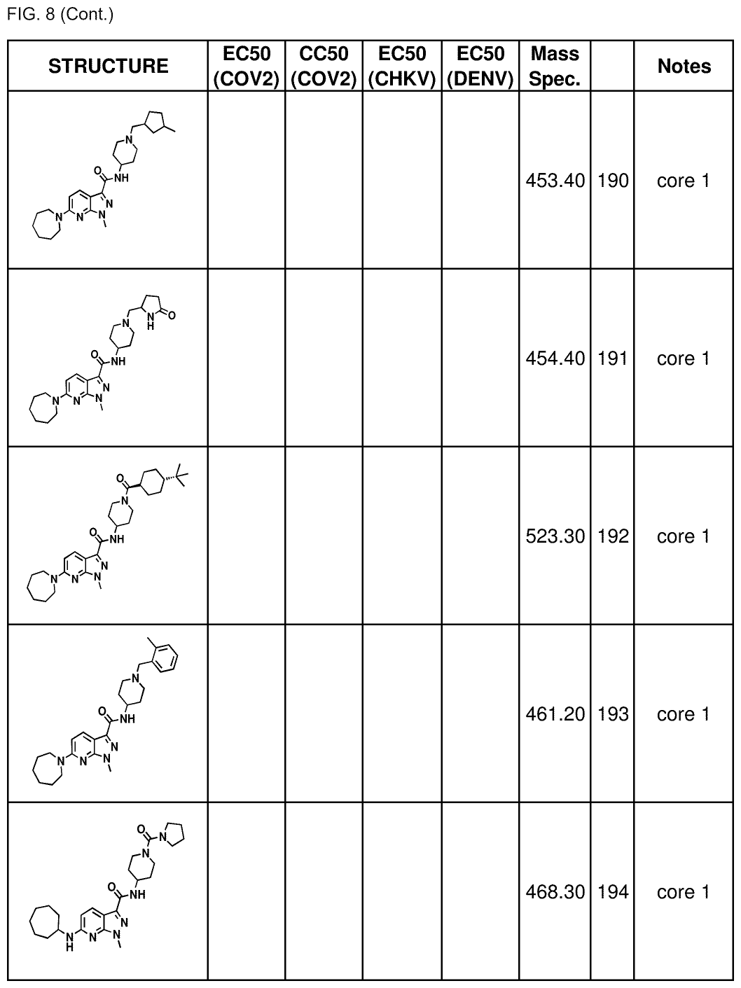

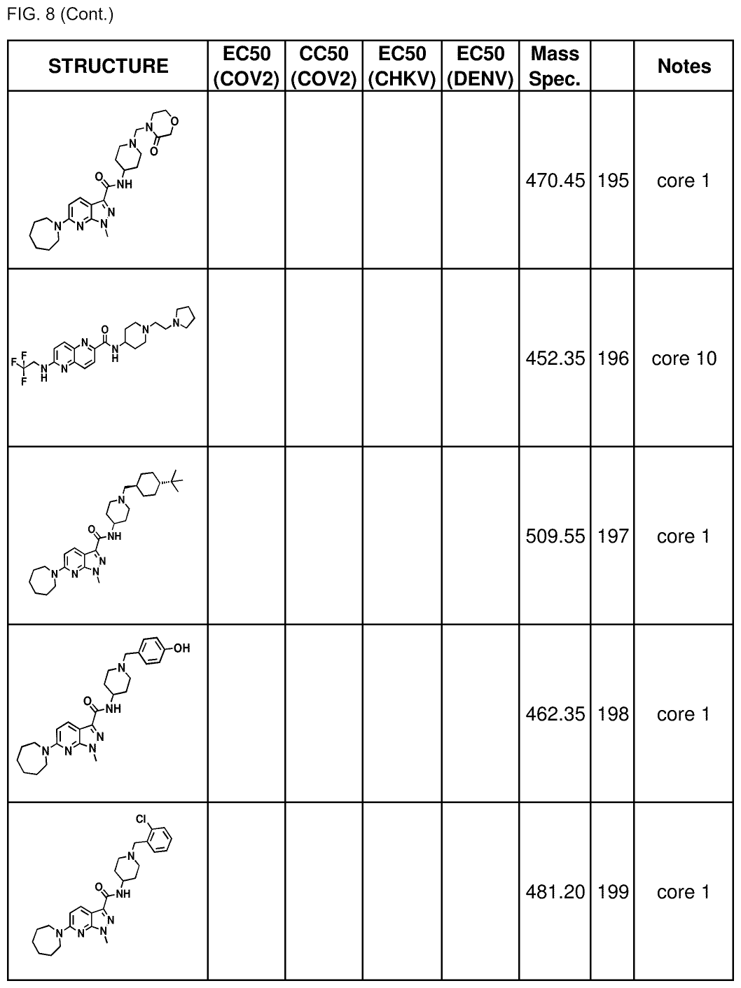

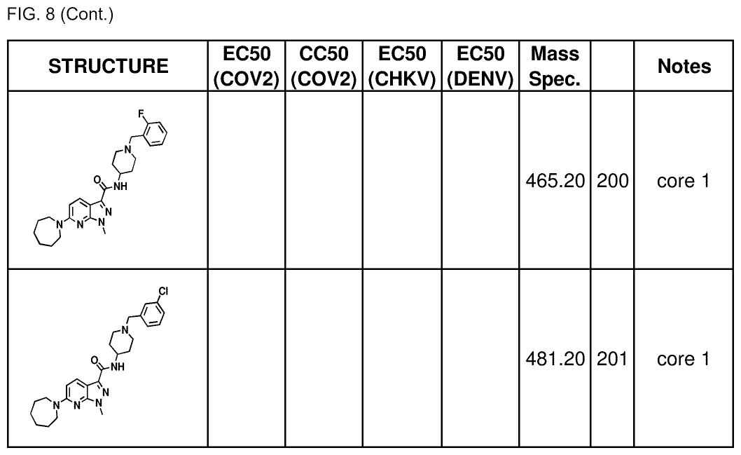

(see, e.g., rows 7-10, 21-24, 30, 38-39, 55-56, and 184 of FIG. 8).

In some cases, a subject agent is a compound, or pharmaceutically acceptable salt thereof, selected from the group consisting of:

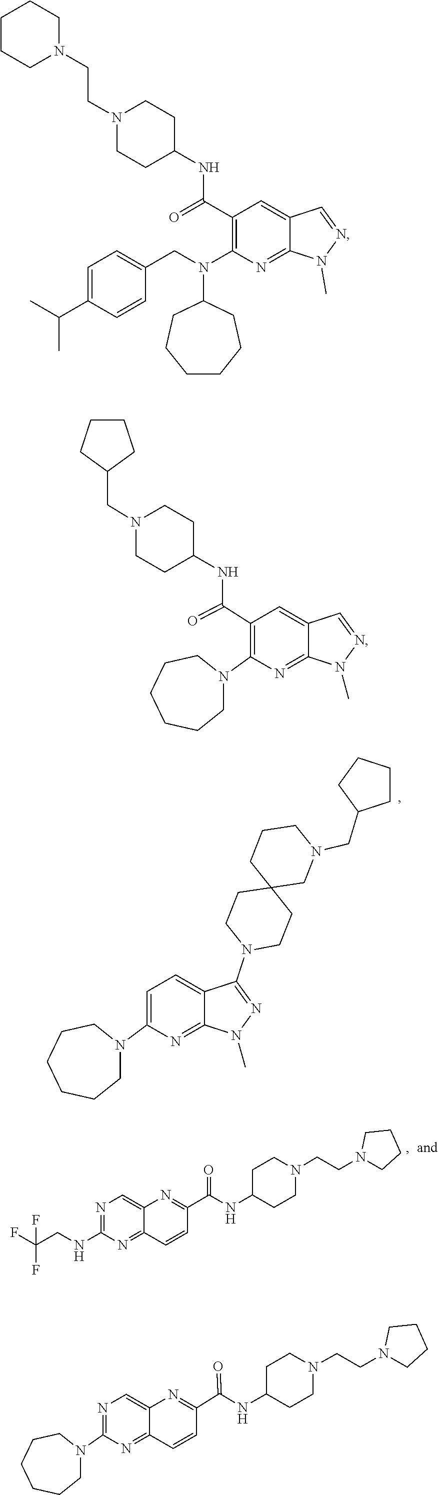 [SMILES: CC(C)c1ccc(CN(c2nc3c(cnn3C)cc2C(=O)NC2CCN(CCN3CCCCC3)CC2)C2CCCCCC2)cc1 ; Cn1ncc2cc(C(=O)NC3CCN(CC4CCCC4)CC3)c(N3CCCCCC3)nc21 ; Cn1nc(N2CCC3(CCCN(CC4CCCC4)C3)CC2)c2ccc(N3CCCCCC3)nc21 ; O=C(NC1CCN(CCN2CCCC2)CC1)c1ccc2nc(NCC(F)(F)F)ncc2n1 ; O=C(NC1CCN(CCN2CCCC2)CC1)c1ccc2nc(N3CCCCCC3)ncc2n1]

(see, e.g., rows 23, 30, 39, and 55-56 of FIG. 8).

In some embodiments, a subject composition includes: (a) a pharmaceutically acceptable carrier, excipient, or diluent; and (b) a compound, or pharmaceutically acceptable salt thereof, disclosed herein. As an illustrative example, in some cases, a subject composition includes: (a) a pharmaceutically acceptable carrier, excipient, or diluent; and (b) a compound, or pharmaceutically acceptable salt thereof, selected from the group consisting of:

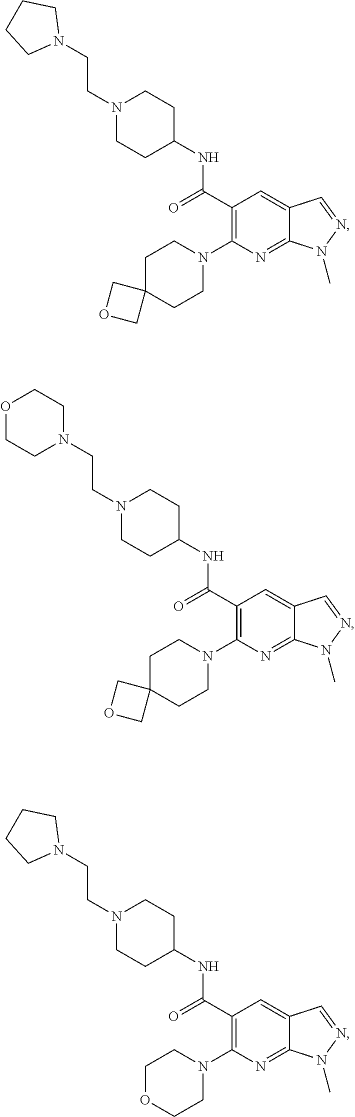 [SMILES: Cn1ncc2cc(C(=O)NC3CCN(CCN4CCCC4)CC3)c(N3CCC4(CC3)COC4)nc21 ; Cn1ncc2cc(C(=O)NC3CCN(CCN4CCOCC4)CC3)c(N3CCC4(CC3)COC4)nc21 ; Cn1ncc2cc(C(=O)NC3CCN(CCN4CCCC4)CC3)c(N3CCOCC3)nc21]

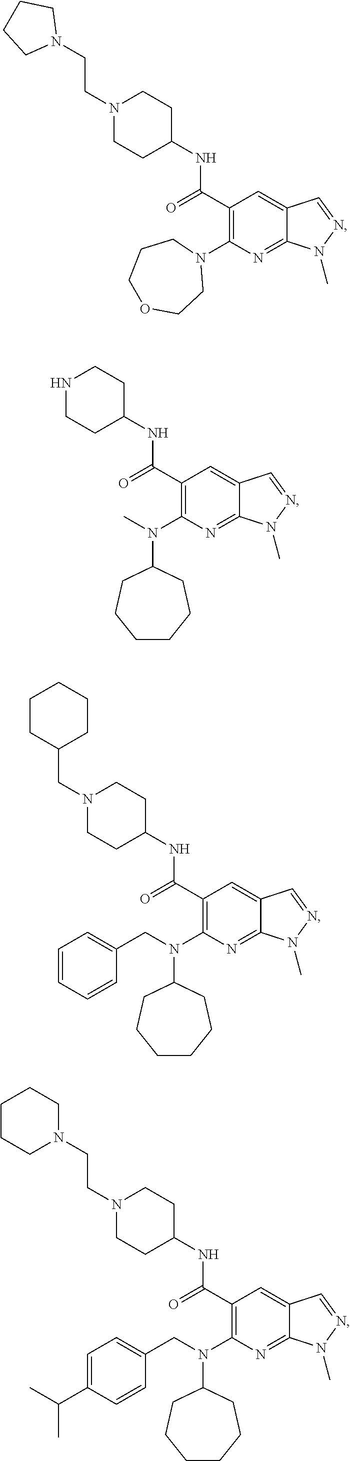 [SMILES: Cn1ncc2cc(C(=O)NC3CCN(CCN4CCCC4)CC3)c(N3CCCOCC3)nc21 ; CN(c1nc2c(cnn2C)cc1C(=O)NC1CCNCC1)C1CCCCCC1 ; Cn1ncc2cc(C(=O)NC3CCN(CC4CCCCC4)CC3)c(N(Cc3ccccc3)C3CCCCCC3)nc21 ; CC(C)c1ccc(CN(c2nc3c(cnn3C)cc2C(=O)NC2CCN(CCN3CCCCC3)CC2)C2CCCCCC2)cc1]

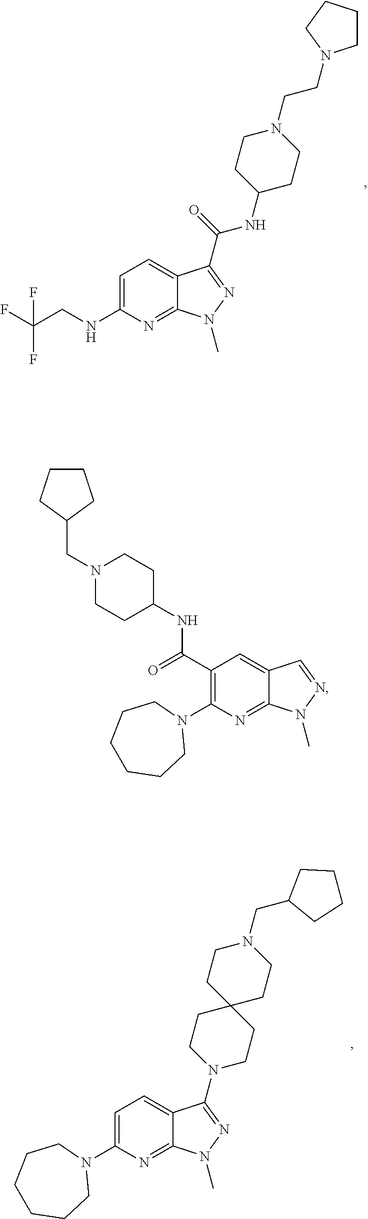 [SMILES: Cn1nc(C(=O)NC2CCN(CCN3CCCC3)CC2)c2ccc(NCC(F)(F)F)nc21 ; Cn1ncc2cc(C(=O)NC3CCN(CC4CCCC4)CC3)c(N3CCCCCC3)nc21 ; Cn1nc(N2CCC3(CCN(CC4CCCC4)CC3)CC2)c2ccc(N3CCCCCC3)nc21]

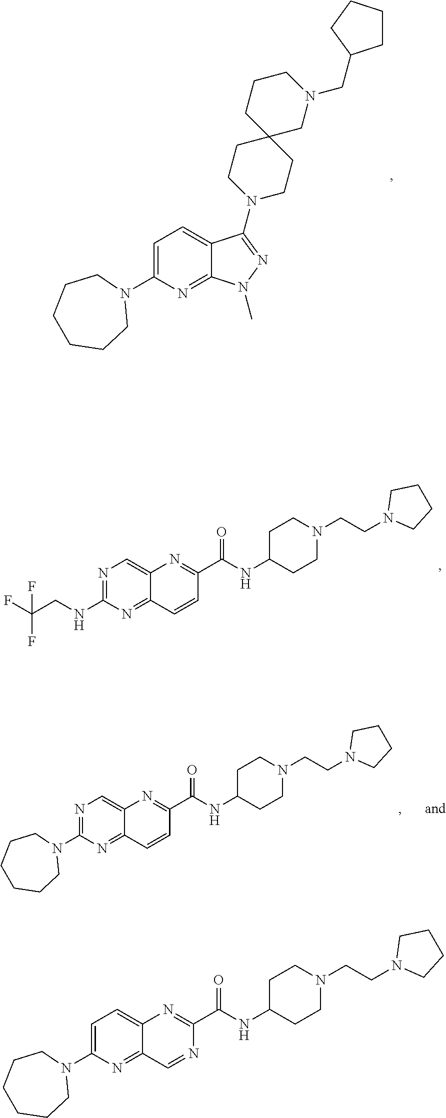 [SMILES: Cn1nc(N2CCC3(CCCN(CC4CCCC4)C3)CC2)c2ccc(N3CCCCCC3)nc21 ; O=C(NC1CCN(CCN2CCCC2)CC1)c1ccc2nc(NCC(F)(F)F)ncc2n1 ; O=C(NC1CCN(CCN2CCCC2)CC1)c1ccc2nc(N3CCCCCC3)ncc2n1 ; O=C(NC1CCN(CCN2CCCC2)CC1)c1ncc2nc(N3CCCCCC3)ccc2n1]

(see, e.g., rows 7-10, 21-24, 30, 38-39, 55-56, and 184 of FIG. 8).

In some such cases, the composition is a medicament for the treatment of fatty liver and/or a viral infection, such as a non-flaviviridae viral infection (e.g., an hepadnaviridae virus infection such as HBV, an alphavirus infection such as ONNV, SFV, VEEV, or CHIKV, a coronaviridae infection such as SARS-CoV-2, a paramyxoviridae infection such as RSV, an orthomyxoviridae infection such as influenza, and the like).

In those embodiments where an active agent is administered to the subject, the active agent(s) may be administered to the subject using any convenient administration protocol capable of resulting in the desired activity. Thus, the agent can be incorporated into a variety of formulations, e.g., pharmaceutically acceptable vehicles, for therapeutic administration. More particularly, the agents of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments (e.g., skin creams), solutions, suppositories, injections, inhalants and aerosols. As such, administration of the agents can be achieved in various ways, including oral, buccal, rectal, parenteral, intraperitoneal, intradermal, transdermal, intracheal, etc., administration.

In pharmaceutical dosage forms, the agents may be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The agents can be utilized in aerosol formulation to be administered via inhalation. The compounds of the present invention can be formulated into pressurized acceptable propellants such as dichlorodifluoromethane, propane, nitrogen and the like.

Furthermore, the agents can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Where the agent is a polypeptide, polynucleotide, analog or mimetic thereof, it may be introduced into tissues or host cells by any number of routes, including viral infection, microinjection, or fusion of vesicles. Jet injection may also be used for intramuscular administration, as described by Furth et al., Anal Biochem. (1992) 205:365-368. The DNA may be coated onto gold microparticles, and delivered intradermally by a particle bombardment device, or “gene gun” as described in the literature (see, for example, Tang et al., Nature (1992) 356:152-154), where gold microprojectiles are coated with the DNA, then bombarded into skin cells. For nucleic acid therapeutic agents, a number of different delivery vehicles find use, including viral and non-viral vector systems, as are known in the art.

Those of skill in the art will readily appreciate that dose levels can vary as a function of the specific compound, the nature of the delivery vehicle, and the like. Preferred dosages for a given compound are readily determinable by those of skill in the art by a variety of means.

An “effective amount” or “effective dose” refers to that amount which is capable of having the desired effect, e.g., ameliorating or delaying progression of the diseased, degenerative or damaged condition, reducing steatosis, reducing virus in an individual, and the like. An effective amount can be determined on an individual basis and will be based, in part, on consideration of the symptoms to be treated and results sought. A “therapeutically effective dose” or “therapeutic dose” or “therapeutically effective amount” or “therapeutic amount” is an amount sufficient to effect desired clinical results (i.e., achieve therapeutic efficacy). A therapeutically effective dose can be administered in one or more administrations. An effective amount (e.g., therapeutically effective amount) can be determined by one of ordinary skill in the art employing such factors and using no more than routine experimentation.

In some embodiments, treatment (to reduce virus in an individual) results in undetectable levels of virus antigen, e.g. HBsAg for HBV, in blood of the infected individual. In some embodiments the level of circulating virus antigen, e.g. HBsAg for HBV, (or virus nucleic acid) is reduced by at least one log relative to the baseline pre-treatment levels; and may be reduced at least 1 log, at least 2 logs, at least 3 logs, or more. In some embodiments the individual achieves, following treatment, seroconversion to a non-positive (for viral antigen) phenotype. In some embodiments the individual is tested for levels of virus (e.g., viral nucleic acid, viral antigen, anti-virus antibodies) following treatment.

In some cases, an effective amount of a subject agent will reduce steatosis (hepatic steatosis). In some cases, progression of steatosis will be blocked and in some cases steatosis will be reduced (i.e., the amount of steatosis observed will be less after administration of the agent than was observed prior to administration). In some cases, steatosis will be reduced by 10% (that is, the amount observed will be 90% of what was present prior to administration) or more (e.g., 20% or more, 35% or more, 50% or more, 70% or more, 85% or more). In some cases, steatosis will be reduced by 30% (that is, the amount observed will be 70% of what was present prior to administration) or more (e.g., 50% or more, 70% or more, 85% or more). In some cases, steatosis will be reduced by 50% or more.

An effective dose (e.g., therapeutically effective dose) can be expected to be in a range of from about 0.001 to about 100 mg/kg body weight per day (for humans), in some cases from about 0.1 to about 50 mg/kg body weight per day, in some cases from about 1 to about 50 mg/kg body weight per day, in some cases about 5 to about 40 mg/kg body weight per day, in some cases about 2 to about 15 mg/kg body weight per day, and in some cases about 25 to about 40 mg/kg bodyweight per day. In some cases, an effective does is in a range of from 5 to 100 mg/kg bodyweight per day (e.g., 5-80, 5-50, 5-40, 5-30, 5-20, 5-10, 8-100, 8-80, 8-50, 8-40, 8-30, 8-20, 10-100, 10-80, 10-50, 10-40, 10-30, or 10-20 mg/kg bodyweight per day). In some cases, an effective does is in a range of from 8 to 100 mg/kg bodyweight per day. In some cases, an effective does is about 8 mg/kg bodyweight per day.

Dosage and frequency may vary depending on the half-life of the agent. It will be understood by one of skill in the art that such guidelines will be adjusted for the molecular weight of the active agent. The dosage may also be varied for localized administration, e.g. intranasal, inhalation, etc., or for systemic administration, e.g. i.m., i.p., i.v., s.c., and the like.

The treatment course may be less than about 12 weeks, less than about 8 weeks, less than about 4 weeks, and may be, for example, from 1-12 weeks, from 2-12 weeks, from 4-12 weeks, from 4-8 weeks, etc. Administration may be once a week, twice a week, every other day, daily, twice a day, every two weeks, etc., and in some embodiments is once a week. In some embodiments, more than one course of treatment is administered. In some cases, the course of treatment is in a range of from 1-24 weeks (e.g., from 1-8 weeks, 1-4 weeks, about 1 week, about 2 weeks, or about 3 weeks). In some cases, the course of treatment is in a range of from 1 day-8 weeks (e.g., from 1 day-6 weeks, 1 day-4 weeks, 1 day-2 weeks, 1 day-7 days, 3 days-8 weeks, 3 days-6 weeks, 3 days-4 weeks, 3 days-2 weeks, or 3 days-7 days). In some cases, the compound is administered for 2 or more days (e.g., 3 or more, 4 or more, 5 or more, 6 or more, 7 or more days, 14 or more days, or 1 month or more). In some cases, the compound is administered for 3 or more days.

A subject agent (e.g., A27, an A27 derivative, etc.) can be administered to an individual by any route of administration appreciated in the art, including but not limited to oral administration, administration by injection (specific embodiments of which include intravenous, subcutaneous, intraperitoneal or intramuscular injection), administration by inhalation, intranasal, or topical administration, either alone or in combination with other agents designed to assist in the treatment of the individual.

The route of administration should be determined based on a number of considerations appreciated by the skilled artisan including, but not limited to, the desired physiochemical characteristics of the treatment. Treatment may be provided for example, 2-8° C. or higher, while also making the formulation useful for parenteral injection. As appropriate, preservatives, stabilizers, buffers, antioxidants and/or other additives may be included. The formulations may contain a divalent cation (including but not limited to MgCl2, CaCl2, and MnCl2) and/or a non-ionic surfactant (including but not limited to Polysorbate-80 (TWEEN 80™), Polysorbate-60 (TWEEN 60™), Polysorbate-40 (TWEEN 40™), and Polysorbate-20 (TWEEN 20™) polyoxyethylene alkyl ethers, including but not limited to BRIJ 58™, BRIJ 35™, as well as others such as TRITONX-100™, TRITONX-114™, NP40™, Span 85 and the PLURONIC® series of non-ionic surfactants (e.g., PLURONIC® 121). Any combination of such components form specific embodiments of the present disclosure.

“Pharmaceutically acceptable salts” and “Pharmaceutically acceptable esters” means salts and esters that are pharmaceutically acceptable and have the desired pharmacological properties. Such salts include salts that can be formed where acidic protons present in the compounds are capable of reacting with inorganic or organic bases. Suitable inorganic salts include those formed with the alkali metals, e.g. sodium and potassium, magnesium, calcium, and aluminum. Suitable organic salts include those formed with organic bases such as the amine bases, e.g., ethanolamine, diethanolamine, triethanolamine, tromethamine, N-methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). Pharmaceutically acceptable esters include esters formed from carboxy, sulfonyloxy, and phosphonoxy groups present in the compounds, e.g., C₁-6 alkyl esters. When there are two acidic groups present, a pharmaceutically acceptable salt or ester can be a mono-acid-mono-salt or ester or a di-salt or ester; and similarly where there are more than two acidic groups present, some or all of such groups can be salified or esterified. Compounds named in this invention can be present in unsalified or unesterified form, or in salified and/or esterified form, and the naming of such compounds is intended to include both the original (unsalified and unesterified) compound and its pharmaceutically acceptable salts and esters. Also, certain compounds named in this invention may be present in more than one stereoisomeric form, and the naming of such compounds is intended to include all single stereoisomers and all mixtures (whether racemic or otherwise) of such stereoisomers.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

The terms “co-administration”, “co-administer”, and “in combination with” include the administration of two or more therapeutic agents (e.g., A27 or any of the compounds described herein and an anti-viral agent, a target cell specific antibody, an antibiotic, and the like) either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

Administration of subject agent (e.g., A27 or any of the compounds described herein) may be combined with co-administration of any number of agents-such as antiviral agents. Such agents may include, for example, the entry inhibitor myrcludex-b, anti-NTBC antibody, an HBV nucleoside analog (e.g. TDF, TAF, ETC), and the like. Administration of a subject agent (e.g., A27 or any of the compounds described herein) may be combined with an anti-viral agent, e.g. HBsAg release inhibitor (nucleic acid polymers), HBV core inhibitors, siRNAs targeting HBV (or HDV), immunodulator (TLR agonists, etc), RT or polymerase inhibitor, therapeutic vaccines, and the like. Other antivirals may include an interferon, including interferon alfa-2b, Peginterferon alfa-2a, interferon-lambda, entecavir, lamivudine, adefovir, telbivudine, tenofovir, sofosbuvir, ledipasvir, ombitasvir, paritaprevir, ritonavir, dasabuvir, grazoprevir, elbasvir, asunaprevir, declatasvir or beclabuvir.

A subject agent (e.g., A27 or any of the compounds described herein) need not be, but is optionally formulated with one or more agents that potentiate activity, or that otherwise increase the therapeutic effect. These are generally used in the same dosages and with administration routes as used herein or from 1 to 99% of the heretofore employed.

A subject agent (e.g., A27 or any of the compounds described herein) may be co-administered with other active agents, such as antibiotics, cytokines, anti-viral agents, etc. Classes of antibiotics include penicillins, e.g. penicillin G, penicillin V, methicillin, oxacillin, carbenicillin, nafcillin, ampicillin, etc.; penicillins in combination with β-lactamase inhibitors, cephalosporins, e.g. cefaclor, cefazolin, cefuroxime, moxalactam, etc.; carbapenems; monobactams; aminoglycosides; tetracyclines; macrolides; lincomycins; polymyxins; sulfonamides; quinolones; cloramphenical; metronidazole; spectinomycin; trimethoprim; vancomycin; etc. Cytokines may also be included, e.g. interferon γ, tumor necrosis factor α, interleukin 12, etc. Antiviral agents, e.g. acyclovir, gancyclovir, etc., may also be used in treatment.

### EXEMPLARY NON-LIMITING ASPECTS OF THE DISCLOSURE

Aspects, including embodiments, of the present subject matter described above may be beneficial alone or in combination, with one or more other aspects or embodiments. Without limiting the foregoing description, certain non-limiting aspects of the disclosure are provided below (see SET A and SET B). As will be apparent to those of ordinary skill in the art upon reading this disclosure, each of the individually numbered aspects may be used or combined with any of the preceding or following individually numbered aspects. This is intended to provide support for all such combinations of aspects and is not limited to combinations of aspects explicitly provided below. It will be apparent to one of ordinary skill in the art that various changes and modifications can be made without departing from the spirit or scope of the invention.

### Set A

1. The method of using the KxL ligand A27 to treat fatty liver.
2. The method of using the KxL ligand A27 to treat fatty liver and/or viral infections.
3. The method of using the KxL ligand A27 to treat HBV infections.
4. The method of using the KxL ligand A27 to treat SARS-CoV-2 infections.
5. The method of using the KxL ligand A27 to treat VEEV infections.
6. The method of 1, where A27 is administered at 8 mg/kg to 100 mg/kg.
7. The method of 2, where A27 is administered at 8 mg/kg to 100 mg/kg.
8. The method of 3, where A27 is administered at 8 mg/kg to 100 mg/kg.
9. The method of 4, where A27 is administered at 8 mg/kg to 100 mg/kg.
10. The method of 5, where A27 is administered at 8 mg/kg to 100 mg/kg.
11. The method of 1-5, where A27 is administered for 3 days or more.
12. The method of 1-5, where A27 is administered for 7 days or more.
13. The method of 1-5, where A27 is administered for 14 days or more.
14. The method of 1-5, where A27 is administered for 1 month or more.
15. The method of 2-5, where A27 is administered in combination with another antiviral agent.
16. The method of using a KxL ligand A27 derivative to treat fatty liver.
17. The method of using a KxL ligand A27 derivative to treat fatty liver and/or viral infections.
18. The method of using a KxL ligand A27 derivative to treat HBV infections.
19. The method of using a KxL ligand A27 derivative to treat SARS-CoV-2 infections.
20. The method of using a KxL ligand A27 derivative to treat VEEV infections. 21.
22. The method of 12-16, where A27 derivative(s) is administered in combination with another antiviral agent.
23. The method of 11 and 18, where an antiviral agent is selected from a direct acting antiviral (e.g., protease inhibitors, polymerase inhibitors), a host-targeting antiviral (e.g., interferons), or other agents that modulate the course of a viral illness.

### Set B

1. A method of treatment, comprising: administering to an individual who has a fatty liver and/or a non-flaviviridae viral infection, a therapeutically effective amount of a compound selected from the group consisting of:
   (a) a compound of Formula I:

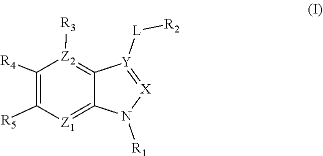

or pharmaceutically acceptable salt thereof, wherein:
   X is C or N;
   Y is C or N;
   Z₁ is C or N;
   Z₂ is C or N;
   R₁ is hydrogen; C₁-C₆ alkyl; C₁-C₆ alkyl substituted with a substituted or unsubstituted C₃-C₈ cycloalkyl, 5-8 membered heterocyclyl, or a 6 membered aryl group; C₂-C₆ alkenyl;
   substituted or unsubstituted C₃-C₈ cycloalkyl, —CO—(C₃-C₈ cycloalkyl), —CO—(C₁-C₆ alkyl), —CO-aryl, —CO-heteroaryl, —CO-heterocyclyl, —SO₂—(C₁-C₆ alkyl), or —SO₂—(C₃-C₈ cycloalkyl) group; or R₁ and R₂ together form a 12-25 membered heterocycle, or R₁ and R₅ together form a 12-25 membered heterocycle;
   L is a bond, —CONH—, —NH—CO—, substituted or unsubstituted C₁-C₅ alkylene, substituted or unsubstituted C₂-C₅ heteroalkylene, a substituted or unsubstituted 5 membered heteroaryl group, or a substituted or unsubstituted 5-7 membered heterocyclyl, C₅-C₇ cycloalkyl, 5-6 membered heteroaryl, or a 6 membered aryl group; or a combination thereof;
   R₂ is —NH₂, —NHR′, —NR′R′, —NHCOR′, —NR′COR′, —NHSO₂R′, —NR′SO₂R′, —NHSO₂NH₂, —NHSO₂NHR′, —NHC(O)NH₂, —NHC(O)NHR′, —N(R′)SO₂NH₂, —N(R)SO₂NHR′, —N(R′)C(O)NH₂, and —N(R′)C(O)NHR′, or a substituted or unsubstituted 5-7 membered heterocyclyl, C₅-C₇ cycloalkyl, 5-6 membered heteroaryl, or a 6 membered aryl group;
   R₃, R₄, and R₅ are independently hydrogen, halo, —CN, —OH, —OR′, —NH₂, —NHR′, —NR′R′, —NHCOR′, —NR′COR′, —NHSO₂R′, —NR′SO₂R′, —NHSO₂NH₂, —NHSO₂NHR′, —NHC(O)NH₂, —NHC(O)NHR′, —N(R′)SO₂NH₂, —N(R′)SO₂NHR′, —N(R′)C(O)NH₂, and N(R)C(O)NHR′, —SO₂R′, —SO₂NH₂, SO₂NHR′, SO₂NR′R′, —CONH₂, —CONHR′, —CONR′R′, —CO₂H, —CO₂R′, or a substituted or unsubstituted C₁-C₆ alkyl, C₃-C₈ cycloalkyl, aryl, heteroaryl, or heterocyclyl group; and
   R′ is a substituted or unsubstituted C₁-C₆ alkyl, C₂-C₆ alkenyl, C₂-C₆ alkynyl, C₃-C₈ cycloalkyl, aryl, heteroaryl, or heterocyclyl group, or two R′ groups together with the nitrogen atom to which they are bonded form a heterocyclic ring;
(b) a compound of Formula II:

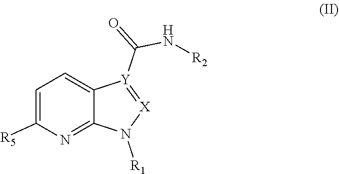

or pharmaceutically acceptable salt thereof, wherein:
   X is C or N;
   Y is C or N;
   R₁ is hydrogen, branched or linear C₁-C₅ alkyl, C₂-C₁₅ alkenyl, unsubstituted or substituted cycloalkyl, —CO-(cycloalkyl), —SO₂-(cycloalkyl) group, or —(CH₂)ₙ—R₁₁, or R₅ and R₁ together form a 12-18 membered heterocycle; n is 1 or 2;
   R₂ is substituted or unsubstituted piperidinyl, 4-pyridyl, pyrrolidinyl, piperazinyl, benzyl, substituted phenyl, or pirazolyl group;
   R₅ is R₅₁R₅₂N— or R₅₄O—;
   R₅₁ is H or substituted or unsubstituted C₁-C₃ alkyl; R₅₂ is C₆-C₈ cycloalkyl, substituted or unsubstituted linear C₁-C₃ alkyl, or branched C₄-C₅ alkyl or R₅₁ and R₅₂ together with the nitrogen atom to which they are bonded form a 6, 7, 8, or 9-membered heterocyclyl ring containing up to 3 heteroatoms optionally substituted, other than the azaindazole moiety to which it is already attached, by a substituted or unsubstituted benzyl acyl, or sulfonyl group; R₅₄ is H, substituted or unsubstituted benzyl group, branched C₃-C₈ alkyl, unsubstituted C₅-C₈ cycloalkyl, or C₅-C₈ cycloalkyl substituted with one or more linear or branched C₁-C₄ alkyl groups; R₁₁ is C₅-C₈ cycloalkyl or substituted or unsubstituted aryl, or substituted or unsubstituted heteroaryl;
(c) a compound of Formula III:

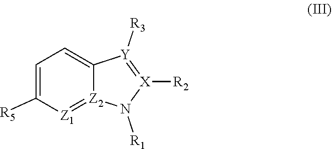

or pharmaceutically acceptable salt thereof, wherein:
   X is C or N;
   Y is C or N;
   Z₁ is C or N;
   Z₂ is C or N;
   R₁ is hydrogen, branched or linear C₁-C₅ alkyl, C₂-C₁₅ alkenyl, unsubstituted or substituted cycloalkyl, —CO-(cycloalkyl), —SO₂-(cycloalkyl) group, or —(CH₂)ₙ—R₁₁, or R₅ and R₁ together form a 12-18 membered heterocycle; n is 1 or 2;
   R₂ is substituted or unsubstituted phenyl, piperidinyl, 4-pyridyl, pyrrolidinyl, piperazinyl, benzyl, substituted phenyl, or pirazolyl group; R₅ is R₅₁R₅₂N— or R₅₄O—;
   R₅₁ is H or substituted or unsubstituted C₁-C₃ alkyl; R₅₂ is C₆-C₈ cycloalkyl, substituted or unsubstituted linear C₁-C₃ alkyl, or branched C₄-C₅ alkyl or R₅₁ and R₅₂ together with the nitrogen atom to which they are bonded form a 6, 7, 8, or 9-membered heterocyclyl ring containing up to 3 heteroatoms optionally substituted, other than the azaindazole moiety to which it is already attached, by a substituted or unsubstituted benzyl acyl, or sulfonyl group; R₅₄ is H, substituted or unsubstituted benzyl group, branched C₃-C₈ alkyl, unsubstituted C₅-C₈ cycloalkyl, or C₅-C₈ cycloalkyl substituted with one or more linear or branched C₁-C₄ alkyl groups; R₁₁ is C₅-C₈ cycloalkyl or substituted or unsubstituted aryl, or substituted or unsubstituted heteroaryl;
   R₃ and R₅ are independently hydrogen, halo, —CN, —OH, —OR′, —NH₂, —NHR′, —NR′R′, —NHCOR′, —NR′COR′, —NHSO₂R′, —NR′SO₂R′, —NHSO₂NH₂, —NHSO₂NHR′, —NHC(O)NH₂, —NHC(O)NHR′, —N(R′)SO₂NH₂, —N(R′)SO₂NHR′, —N(R′)C(O)NH₂, and N(R)C(O)NHR′, —SO₂R′, —SO₂NH₂, SO₂NHR′, SO₂NR′R′, —CONH₂, —CONHR′, —CONR′R′, —CO₂H, —CO₂R′, or a substituted or unsubstituted C₁-C₆ alkyl, C₃-C₈ cycloalkyl, aryl, heteroaryl, or heterocyclyl group;
(d) a compound of Formula IV:

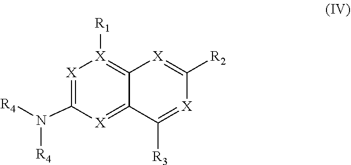

or pharmaceutically acceptable salt thereof, wherein:
   each X is independently selected from C or N;
   R¹ is absent, hydrogen, (C₁-C₈) alkyl, NR⁹R¹⁰, halo, amino, —C≡N, (C₂-C₈) alkenyl, (C₂-C₈) alkynyl, (C C₈) haloalkyl, (C₂-C₈) haloalkenyl, (C₂-C₈) haloalkynyl, (C₁-C₈) alkoxy, (C₁-C₈) haloalkoxy, (C₁-C₈) alkyl (C₁-C₆) alkoxy, 4-7 membered heterocyclyl, 5-6 membered heteroaryl, CH(O), C(O)OR⁸, SF₅, —OH, —SH, (C₁-C₆) hydroxyalkyl, (C₁-C₄) alkylsulfonyl, aminosulfonyl, amino (C₁-C₄) alkylsulfonyl or aryl;
   R² is C(O)NR¹¹R¹², C(O)R¹³, 5-membered heterocycle, 5-membered substituted heterocycle, 5-membered heteroaryl, 5-membered substituted heteroaryl, 6-membered heterocycle, 6-membered substituted heterocycle, 6-membered heteroaryl, 6-membered substituted heteroaryl, 5-membered cycloalkyl, or 5-membered substituted cycloalkyl, 6-membered cycloalkyl, or 6-membered substituted cycloalkyl;
   R³ is H, (C₁-C₈) alkyl, (C₁-C₈) alkenyl, (C₁-C₈) alkynyl, (C₁-C₈) alkoxy, hydroxyl, halo, amino, amido, amino (C₁-C₈) alkylamido, heterocyclyl, sulfonyl, aminosulfonyl, amino (C₁-C₈) alkysulfonyl, cyano, or (C₁-C₃) haloalkyl;
   each R⁴ is independently selected from (C₁-C₈) alkyl, either unsubstituted or substituted with halo, (C₁-C₈) alkoxy, (C₁-C₈) haloaikoxy, S(O)—R⁶, S(O)₂, S(O)₂—R⁶, S(O)₂, C(O)R⁶, C(O)OR⁷ or (C₁-C₈) cycloalkyl; or both R⁴ together form a (C₁-C₈) cycloalkyl, a (C₁-C₈) substituted cycloalkyl, a (C₁-C₈) heterocycloalkyl, a (C₁-C₈) substituted heterocycloalkyl, a (C₁-C₈) aryl, a (C₁-C₈) substituted aryl, a (C₁-C₈) heteroaryl or a (C₁-C₈) substituted heteroaryl;
   R⁶ is H, alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, halo, (C₁-C₄) haloalkyl and cyano;
   R⁷ is H, alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, halo, (C₁-C₄) haloalkyl and cyano;
   R⁸ is H, alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, halo, (C₁-C₄) haloalkyl and cyano;
   R⁹ is H, (C₁-C₈) alkyl, said (C₁-C₈) alkyl being unsubstituted or substituted with one or more halo;
   R¹⁰ is H, (C₁-C₈) alkyl, said (C₁-C₈) alkyl being unsubstituted or substituted with one or more halo, or R⁹ and R¹⁰, together with the nitrogen atom to which they are attached, form a 4 or 5 membered nitrogen containing heterocycle, said 4 or 5 membered nitrogen containing heterocycle being unsubstituted or substituted with one or more halo; R is H, (C₁-C₄) alkyl, (C₁-C₄) haloalkyl, 5-6 membered heterocycle or 5-6 membered heteroaryl;
   R¹¹ is selected from H, (C₁-C₄) alkyl, (C₁-C₄) haloalkyl, a 5-membered heterocycle, 5-membered substituted heterocycle, 5-membered heteroaryl, 5-membered substituted heteroaryl, 6-membered heterocycle, 6-membered substituted heterocycle, 6-membered heteroaryl, 6-membered substituted heteroaryl, 5-membered cycloalkyl, or 5-membered substituted cycloalkyl, 6-membered cycloalkyl, or a 6-membered substituted cycloalkyl;
   R¹² is selected from H, (C₁-C₄) alkyl, (C₁-C₄) haloalkyl, a 5-membered heterocycle, 5-membered substituted heterocycle, 5-membered heteroaryl, 5-membered substituted heteroaryl, 6-membered heterocycle, 6-membered substituted heterocycle, 6-membered heteroaryl, 6-membered substituted heteroaryl, 5-membered cycloalkyl, or 5-membered substituted cycloalkyl, 6-membered cycloalkyl, or a 6-membered substituted cycloalkyl;
   R¹¹ and R¹², together with the nitrogen atom to which they are attached, form a 5-8 membered nitrogen containing heterocycle, said 5-8 membered nitrogen containing heterocycle being unsubstituted or substituted with OH, halo, ═O, or (C₁-C₈) alkyl;
   R¹³ is OH, O—(C₁-C₄) alkyl;
   R¹⁵ is H, (C₁-C₆) alkyl, (C₁-C) haloalkyl, 4-7 membered heterocycle, 5-6 membered heteroaryl, 3-7 membered cycloalkyl, and wherein each of said 4-7 membered heterocycle, 5-6 membered heteroaryl, 3-7 membered cycloalkyl is unsubstituted or substituted at a substitutable position with one or more ═O, OH, (C₁-C₈) alkyl, (C₁-C₈) haloalkyl, (C₁-C₈) alkoxy, amino, or, (C₁-C₈) aminoalkyl; and
   R¹⁶ is H, (C₁-C₈) alkyl, or (C₁-C₈) haloalkyl;
(e) a compound of Formula V:

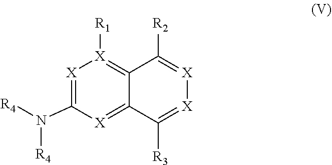

or pharmaceutically acceptable salt thereof, wherein:
   each X is independently selected from C or N;
   R¹ is absent, hydrogen, (C₁-C₈) alkyl, NR⁹R¹⁰, halo, amino, —C≡N, (C₂-C₈) alkenyl, (C₂-C₈) alkynyl, (C C₈) haloalkyl, (C₂-C₈) haloalkenyl, (C₂-C₈) haloalkynyl, (C₁-C₈) alkoxy, (C₁-C₈) haloalkoxy, (C₁-C₈) alkyl (C₁-C₆) alkoxy, 4-7 membered heterocyclyl, 5-6 membered heteroaryl, CH(O), C(O)OR⁸, SF₅, —OH, —SH, (C₁-C₆) hydroxyalkyl, (C₁-C₄) alkylsulfonyl, aminosulfonyl, amino (C₁-C₄) alkylsulfonyl or aryl;
   R² is C(O)NR¹¹R¹², C(O)R¹³, 5-membered heterocycle, 5-membered substituted heterocycle, 5-membered heteroaryl, 5-membered substituted heteroaryl, 6-membered heterocycle, 6-membered substituted heterocycle, 6-membered heteroaryl, 6-membered substituted heteroaryl, 5-membered cycloalkyl, or 5-membered substituted cycloalkyl, 6-membered cycloalkyl, or 6-membered substituted cycloalkyl;
   R³ is H, (C₁-C₈) alkyl, (C₁-C₈) alkenyl, (C₁-C₈) alkynyl, (C₁-C₈) alkoxy, hydroxyl, halo, amino, amido, amino (C₁-C₈) alkylamido, heterocyclyl, sulfonyl, aminosulfonyl, amino (C₁-C₈) alkysulfonyl, cyano, or (C₁-C₃) haloalkyl;
   each R⁴ is independently selected from (C₁-C₈) alkyl, either unsubstituted or substituted with halo, (C₁-C₈) alkoxy, (C₁-C₈) haloaikoxy, S(O)—R⁶, S(O)₂, S(O)₂—R⁶, S(O)₂, C(O)R⁶, C(O)OR⁷ or (C₁-C₈) cycloalkyl; or both R⁴ together form a (C₁-C₈) cycloalkyl, a (C₁-C₈) substituted cycloalkyl, a (C₁-C₈) heterocycloalkyl, a (C₁-C₈) substituted heterocycloalkyl, a (C₁-C₈) aryl, a (C₁-C₈) substituted aryl, a (C₁-C₈) heteroaryl or a (C₁-C₈) substituted heteroaryl;
   R⁶ is H, alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, halo, (C₁-C₄) haloalkyl and cyano;
   R⁷ is H, alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, halo, (C₁-C₄) haloalkyl and cyano;
   R⁸ is H, alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, halo, (C₁-C₄) haloalkyl and cyano;
   R⁹ is H, (C₁-C₈) alkyl, said (C₁-C₈) alkyl being unsubstituted or substituted with one or more halo;
   R¹⁰ is H, (C₁-C₈) alkyl, said (C₁-C₈) alkyl being unsubstituted or substituted with one or more halo, or R⁹ and R¹⁰, together with the nitrogen atom to which they are attached, form a 4 or 5 membered nitrogen containing heterocycle, said 4 or 5 membered nitrogen containing heterocycle being unsubstituted or substituted with one or more halo; R is H, (C₁-C₄) alkyl, (C₁-C₄) haloalkyl, 5-6 membered heterocycle or 5-6 membered heteroaryl;
   R¹¹ is selected from H, (C₁-C₄) alkyl, (C₁-C₄) haloalkyl, a 5-membered heterocycle, 5-membered substituted heterocycle, 5-membered heteroaryl, 5-membered substituted heteroaryl, 6-membered heterocycle, 6-membered substituted heterocycle, 6-membered heteroaryl, 6-membered substituted heteroaryl, 5-membered cycloalkyl, or 5-membered substituted cycloalkyl, 6-membered cycloalkyl, or a 6-membered substituted cycloalkyl;
   R¹² is selected from H, (C₁-C₄) alkyl, (C₁-C₄) haloalkyl, a 5-membered heterocycle, 5-membered substituted heterocycle, 5-membered heteroaryl, 5-membered substituted heteroaryl, 6-membered heterocycle, 6-membered substituted heterocycle, 6-membered heteroaryl, 6-membered substituted heteroaryl, 5-membered cycloalkyl, or 5-membered substituted cycloalkyl, 6-membered cycloalkyl, or a 6-membered substituted cycloalkyl;
   R¹¹ and R¹², together with the nitrogen atom to which they are attached, form a 5-8 membered nitrogen containing heterocycle, said 5-8 membered nitrogen containing heterocycle being unsubstituted or substituted with OH, halo, —O, or (C₁-C₈) alkyl;
   R¹³ is OH, O—(C₁-C₄) alkyl;
   R¹⁵ is H, (C₁-C₆) alkyl, (C₁-C) haloalkyl, 4-7 membered heterocycle, 5-6 membered heteroaryl, 3-7 membered cycloalkyl, and wherein each of said 4-7 membered heterocycle, 5-6 membered heteroaryl, 3-7 membered cycloalkyl is unsubstituted or substituted at a substitutable position with one or more ═O, OH, (C₁-C₈) alkyl, (C₁-C₈) haloalkyl, (C₁-C₈) alkoxy, amino, or, (C₁-C₈) aminoalkyl; and
   R¹⁶ is H, (C₁-C₈) alkyl, or (C₁-C₈) haloalkyl; and
(f) a compound of Formula VI:

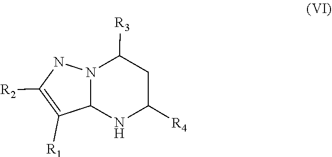

or pharmaceutically acceptable salt thereof, wherein:
   R₁ is hydrogen; halo, haloalkyl C₁-C₆ alkyl; C₁-C₆ alkyl substituted with a substituted or unsubstituted C₃-C₈ cycloalkyl, 5-8 membered heterocyclyl, or a 6 membered aryl group; C₂-C₆ alkenyl;
   substituted or unsubstituted C₃-C₈ cycloalkyl, —CO—(C₃-C₈ cycloalkyl), —CO—(C₁-C₆ alkyl), —CO-aryl, —CO-heteroaryl, —CO-heterocyclyl, —SO₂—(C₁-C₆ alkyl), or —SO₂—(C₃-C₈ cycloalkyl) group;
   R² is C(O)NR¹¹R¹², C(O)R¹³, 5-membered heterocycle, 5-membered substituted heterocycle, 5-membered heteroaryl, 5-membered substituted heteroaryl, 6-membered heterocycle, 6-membered substituted heterocycle, 6-membered heteroaryl, 6-membered substituted heteroaryl, 5-membered cycloalkyl, or 5-membered substituted cycloalkyl, 6-membered cycloalkyl, or 6-membered substituted cycloalkyl;
   R³ is H, (C₁-C₈) alkyl, (C₁-C₈) alkenyl, (C₁-C₈) alkynyl, (C₁-C₈) alkoxy, hydroxyl, halo, amino, amido, amino (C₁-C₈) alkylamido, heterocyclyl, sulfonyl, aminosulfonyl, amino (C₁-C₈) alkysulfonyl, cyano, or (C₁-C₃) haloalkyl;
   R¹¹ is selected from H, (C₁-C₄) alkyl, (C₁-C₄) haloalkyl, a 5-membered heterocycle, 5-membered substituted heterocycle, 5-membered heteroaryl, 5-membered substituted heteroaryl, 6-membered heterocycle, 6-membered substituted heterocycle, 6-membered heteroaryl, 6-membered substituted heteroaryl, 5-membered cycloalkyl, or 5-membered substituted cycloalkyl, 6-membered cycloalkyl, or a 6-membered substituted cycloalkyl or a alkyl-substituted with one or more of a 5-membered heterocycle, 5-membered substituted heterocycle, 5-membered heteroaryl, 5-membered substituted heteroaryl, 6-membered heterocycle, 6-membered substituted heterocycle, 6-membered heteroaryl, 6-membered substituted heteroaryl, 5-membered cycloalkyl, or 5-membered substituted cycloalkyl, 6-membered cycloalkyl, or a 6-membered substituted cycloalkyl
   R¹² is selected from H, (C₁-C₄) alkyl, (C₁-C₄) haloalkyl, a 5-membered heterocycle, 5-membered substituted heterocycle, 5-membered heteroaryl, 5-membered substituted heteroaryl, 6-membered heterocycle, 6-membered substituted heterocycle, 6-membered heteroaryl, 6-membered substituted heteroaryl, 5-membered cycloalkyl, or 5-membered substituted cycloalkyl, 6-membered cycloalkyl, or a 6-membered substituted cycloalkyl or a alkyl-substituted with one or more of a 5-membered heterocycle, 5-membered substituted heterocycle, 5-membered heteroaryl, 5-membered substituted heteroaryl, 6-membered heterocycle, 6-membered substituted heterocycle, 6-membered heteroaryl, 6-membered substituted heteroaryl, 5-membered cycloalkyl, or 5-membered substituted cycloalkyl, 6-membered cycloalkyl, or a 6-membered substituted cycloalkyl
   R¹¹ and R¹², together with the nitrogen atom to which they are attached, form a 5-8 membered nitrogen containing heterocycle, said 5-8 membered nitrogen containing heterocycle being unsubstituted or substituted with OH, halo, ═O, or (C₁-C₈) alkyl; and
   R₄ is hydrogen; halo, haloalkyl C₁-C₆ alkyl; C₁-C₆ alkyl substituted with a substituted or unsubstituted C₃-C₈ cycloalkyl, 5-8 membered heterocyclyl, a 5-8 membered aryl, a 5-8 membered heteroaryl, C₂-C₆ alkenyl; substituted or unsubstituted C₃-C₈ cycloalkyl, —CO—(C₃-C₈ cycloalkyl), —CO—(C₁-C₆ alkyl), —CO-aryl, —CO-heteroaryl, —CO-heterocyclyl, —SO₂—(C₁-C₆ alkyl), or —SO₂—(C₃-C₈ cycloalkyl) group.
   In some embodiments, R₃ is selected from hydrogen, (C₁-C₈) alkyl, halo or haloalkyl. In some instances, R₃ is hydrogen. In some instances, R₃ is a (C₁-C₈) alkyl, such as methyl, ethyl, propyl, i-propyl, butyl, t-butyl, i-butyl or pentyl. In certain instances, R₃ is methyl. In certain instances, R₃ is t-butyl. In some instances, R₃ is halo, such as fluoro, chloro or bromo. In certain instances, R₃ is fluoro. In some instances, R₃ is a haloalkyl, such as CF₃, CCl₃ or CBr₃. In certain instances, R₃ is CF₃.
2. The method of 1, wherein the individual has a non-flaviviridae viral infection selected from the group consisting of: an hepadnaviridae virus infection, an alphavirus infection, a coronaviridae infection, a paramyxoviridae infection, and an orthomyxoviridae infection.
3. The method of 1, wherein the individual has a hepatitis B virus (HBV), Venezuelan equine encephalitis virus (VEEV), O′nyong nyong virus (ONNV), SARS-CoV-2, Semliki Forest virus (SFV), or chikungunya virus (CHIKV) viral infection.
4. The method of 1, wherein the individual has a hepatitis B virus (HBV), Venezuelan equine encephalitis virus (VEEV), SARS-CoV-2, or chikungunya virus (CHIKV) viral infection.
5. The method of any one of 1-4, wherein the compound is co-administered with a second agent.
6. The method of any one of 2-4, wherein the compound is co-administered with a second antiviral agent.
7. The method of any one of 1-6, wherein the compound is administered at a concentration in a range of from at 8 to 100 mg/kg.
8. The method of any one of 1-7, wherein the compound is administered for 3 days or more.
9. The method of any one of 1-8, wherein the compound is A27:

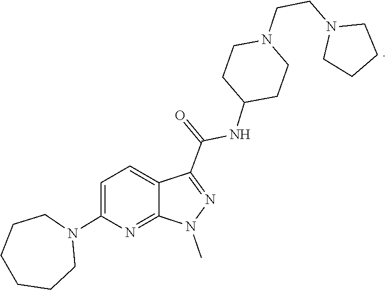 [SMILES: Cn1nc(C(=O)NC2CCN(CCN3CCCC3)CC2)c2ccc(N3CCCCCC3)nc21]

10. A compound, or pharmaceutically acceptable salt thereof, selected from the group consisting of:

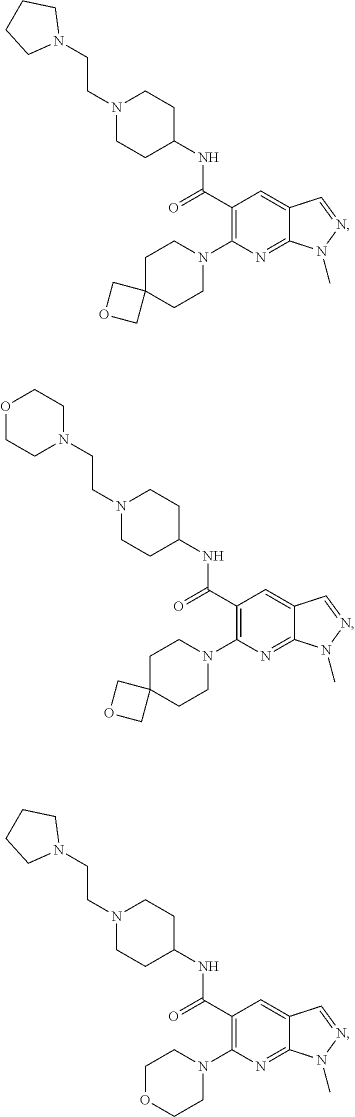 [SMILES: Cn1ncc2cc(C(=O)NC3CCN(CCN4CCCC4)CC3)c(N3CCC4(CC3)COC4)nc21 ; Cn1ncc2cc(C(=O)NC3CCN(CCN4CCOCC4)CC3)c(N3CCC4(CC3)COC4)nc21 ; Cn1ncc2cc(C(=O)NC3CCN(CCN4CCCC4)CC3)c(N3CCOCC3)nc21]

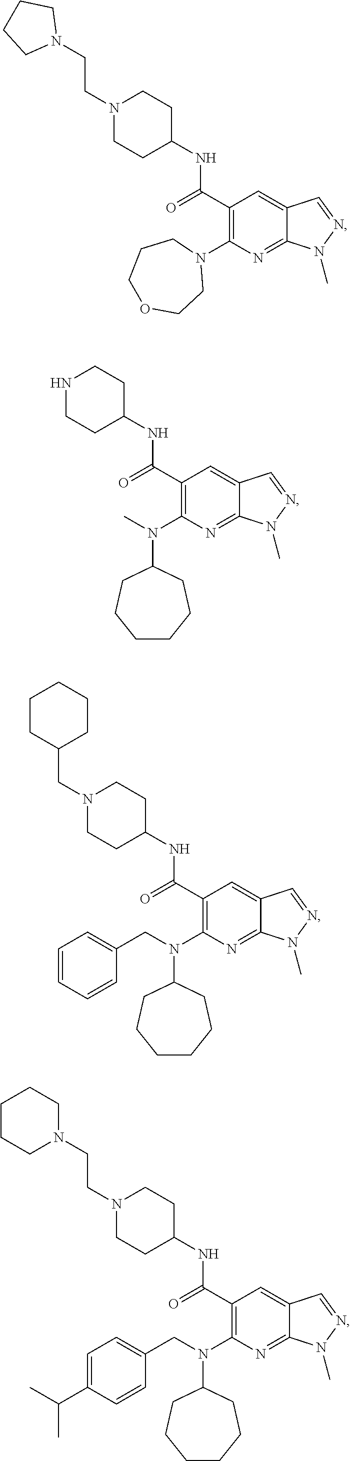 [SMILES: Cn1ncc2cc(C(=O)NC3CCN(CCN4CCCC4)CC3)c(N3CCCOCC3)nc21 ; CN(c1nc2c(cnn2C)cc1C(=O)NC1CCNCC1)C1CCCCCC1 ; Cn1ncc2cc(C(=O)NC3CCN(CC4CCCCC4)CC3)c(N(Cc3ccccc3)C3CCCCCC3)nc21 ; CC(C)c1ccc(CN(c2nc3c(cnn3C)cc2C(=O)NC2CCN(CCN3CCCCC3)CC2)C2CCCCCC2)cc1]

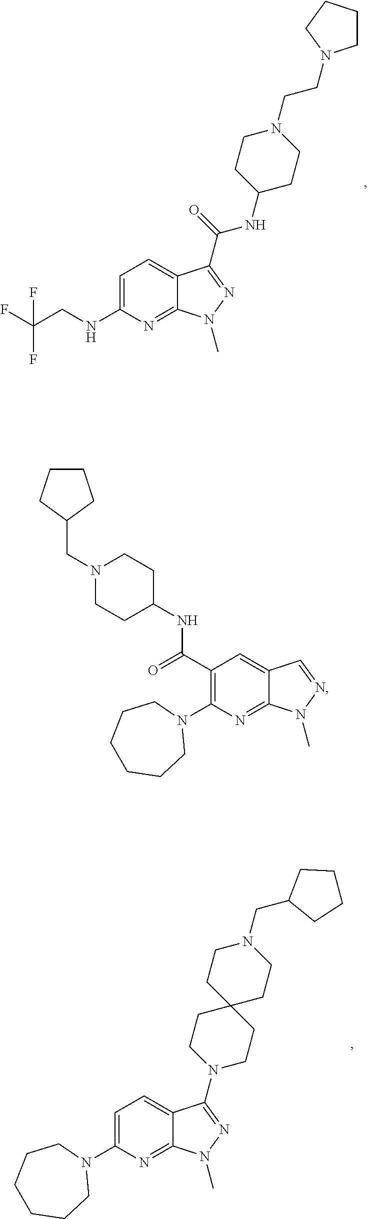 [SMILES: Cn1nc(C(=O)NC2CCN(CCN3CCCC3)CC2)c2ccc(NCC(F)(F)F)nc21 ; Cn1ncc2cc(C(=O)NC3CCN(CC4CCCC4)CC3)c(N3CCCCCC3)nc21 ; Cn1nc(N2CCC3(CCN(CC4CCCC4)CC3)CC2)c2ccc(N3CCCCCC3)nc21]

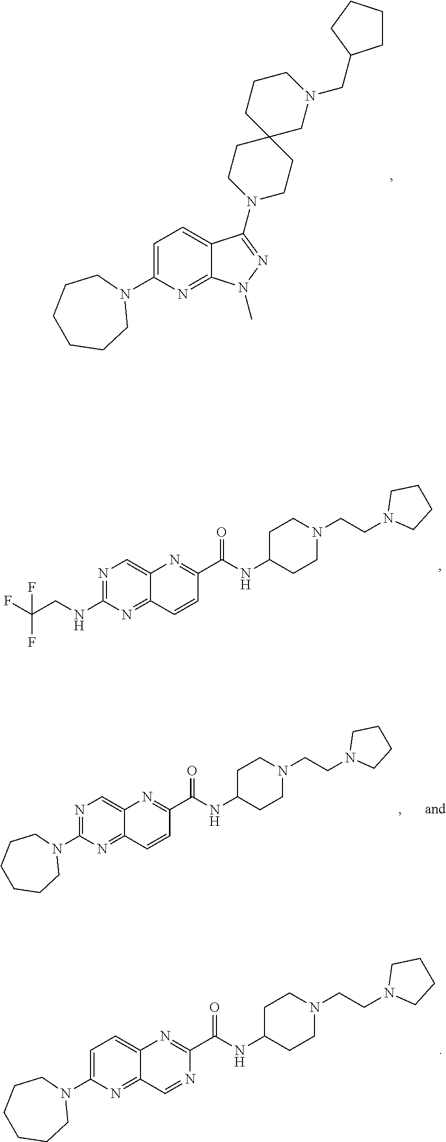 [SMILES: Cn1nc(N2CCC3(CCCN(CC4CCCC4)C3)CC2)c2ccc(N3CCCCCC3)nc21 ; O=C(NC1CCN(CCN2CCCC2)CC1)c1ccc2nc(NCC(F)(F)F)ncc2n1 ; O=C(NC1CCN(CCN2CCCC2)CC1)c1ccc2nc(N3CCCCCC3)ncc2n1 ; O=C(NC1CCN(CCN2CCCC2)CC1)c1ncc2nc(N3CCCCCC3)ccc2n1]

11. The compound, or pharmaceutically acceptable salt thereof, of claim 10, selected from the group consisting of:

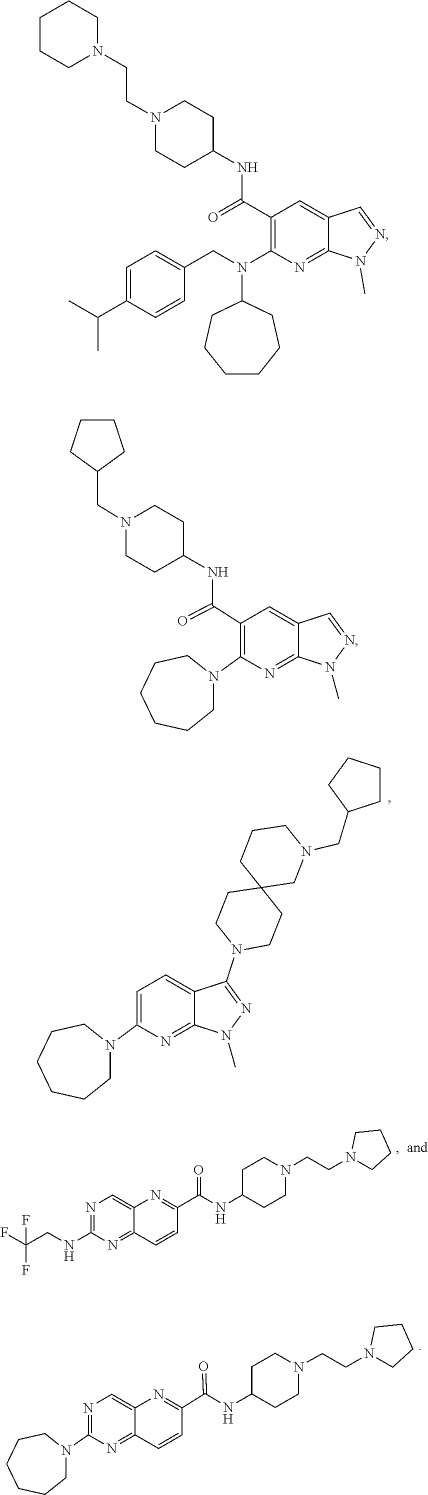 [SMILES: CC(C)c1ccc(CN(c2nc3c(cnn3C)cc2C(=O)NC2CCN(CCN3CCCCC3)CC2)C2CCCCCC2)cc1 ; Cn1ncc2cc(C(=O)NC3CCN(CC4CCCC4)CC3)c(N3CCCCCC3)nc21 ; Cn1nc(N2CCC3(CCCN(CC4CCCC4)C3)CC2)c2ccc(N3CCCCCC3)nc21 ; O=C(NC1CCN(CCN2CCCC2)CC1)c1ccc2nc(NCC(F)(F)F)ncc2n1 ; O=C(NC1CCN(CCN2CCCC2)CC1)c1ccc2nc(N3CCCCCC3)ncc2n1]

12. A composition comprising:
   (a) the compound, or pharmaceutically acceptable salt thereof, of 10 or 11; and
   (b) a pharmaceutically acceptable carrier, excipient, or diluent.
13. The composition of 12, wherein the composition is a medicament for the treatment of fatty liver and/or a viral infection.

The following example(s) is/are offered by way of illustration and not by way of limitation.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, cells, and kits for methods referred to in, or related to, this disclosure are available from commercial vendors such as BioRad, Agilent Technologies, Thermo Fisher Scientific, Sigma-Aldrich, New England Biolabs (NEB), Takara Bio USA, Inc., and the like, as well as repositories such as e.g., Addgene, Inc., American Type Culture Collection (ATCC), and the like.

### Example 1

We discovered that a known small molecule, A27, can bind to apolipoprotein B (apoB), a lipoprotein implicated in cholesterol and lipid droplet formation. Since apoB has been implicated in formation of steatosis, we sought to determine if A27 can modulate the degree of steatosis in cells. Indeed, we discovered that A27 can inhibit formation of steatosis in cultured liver cells. We also discovered that A27 can reduce development of hepatic steatosis in C57/BL6 mice fed a high fat diet.

We further discovered that apoB contains an amphipathic helical domain with a putative binding motif for A27 we term the KxL motif.

Further research showed that this KxL motif is also present in the amphipathic helical domain of hepatitis B surface antigen (HBsAg), the nonstructural protein (NSP) 1 of VEEV, and NSP4 of SARS-CoV-2, and we discovered that A27 had antiviral activity against several of these viruses.

Accordingly, methods are provided herein for treating fatty liver disease and/or viral infections, the method comprising administering an effective amount of A27 or pharmaceutically acceptable salt, hydrate or solvate thereof, or a derivative thereof, to a subject with fatty liver disease and/or viral infection.

We discovered a known small molecule, A27, can bind to apolipoprotein B (apoB), a lipoprotein implicated in cholesterol and lipid droplet formation. The structure of A27 is:

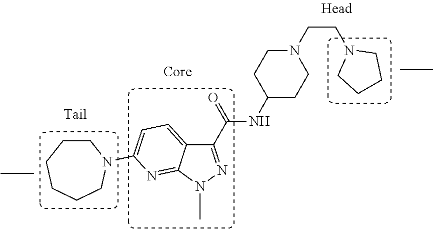

Since ApoB has been implicated in formation of steatosis, we sought to determine if A27 can modulate the degree of steatosis in cells. Indeed, we discovered that A27 can inhibit formation of steatosis in cultured liver cells, e.g. HepG2, with a half effective concentration (EC₅₀) of ˜ 0.5 μM (FIG. 2).

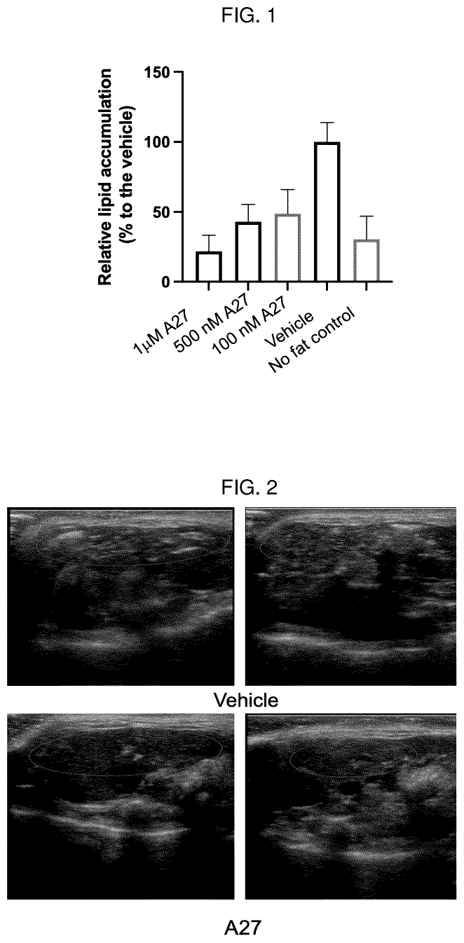

Briefly, fatty medium was prepared by 1:1 mixture of oleic acid and palmitic acid (Sigma Aldrich) with 10% FFA-free bovine serum albumin (BSA) solution (Sigma Aldrich) in PBS. Essentially, 100 mM of oleic acid and palmitic acid was prepared in 99% methanol. Palmitic acid was prepared by shaking at 500 rpm 50° C. for 20 mins. 10 mM of oleic acid and palmitic acid was incubated with 10% BSA in PBS at 50° C. for 30 mins. A final concentration of 100 uM FFA mixture was made in the final cell culture medium. Cells were incubated in fatty medium for 4 days to induce fatty liver. Cells were then treated with A27 at various concentrations for 3 days in the presence of the fatty medium. To determine the degree of steatosis, cells were stained with 1 mg/ml Nile Red solution (Sigma Aldrich) in cell culture medium for 15 mins at 37° C. and florescence signal was measured (see FIG. 1). A dose-dependent decrease in lipid accumulation was observed.

Moreover, A27 reduces hepatic steatosis when administered in mice as measured by liver ultrasound.

To determine the effects of A27 against hepatic steatosis in vivo, cohorts of C57/BL6 mice were first pre-treated for 1 day with A27 at 100 mg/kg/day (equivalent to ˜8 mg/kg/day for a human dose based on body surface area allometric conversion) or vehicle control (40% 2-hydroxypropyl-β-cyclodextrin in water), then placed on high fat diet (60% kcal fat content) for 4 weeks and continued to be treated with A27 or vehicle control during that time. At the end of 4 weeks, liver ultrasound was performed to assess the degree of hepatic steatosis. As shown in FIG. 2, mice on HFD treated with vehicle showed more hepatic steatosis compared to mice treated with A27. All mice tolerated A27 well.

We further discovered that apoB contains an amphipathic helical domain with a putative binding motif for A27 we term the KxL motif.

Further research showed that this KxL motif is also present in the amphipathic helical domain of hepatitis B surface antigen (HBsAg), the nonstructural protein (NSP) 1 of VEEV, and NSP4 of SARS-CoV-2 (FIG. 3).

We then discovered that A27 had antiviral activity against HBV, showing reduction in HBV surface antigen in cells infected with HBV with an EC₅₀ of ˜1 μM (FIG. 4).

We then discovered that A27 also inhibits SARS-CoV-2 in cell culture. Using a luciferase reporter containing infectious clone of SARS-CoV-2 in TMPRSS2-ACE2-Huh7.5 cells, A27 inhibits SARS-CoV-2 genome replication with an EC₅₀ of 803 nM, without any evidence of toxicity.

To determine the in vivo antiviral activity of A27, cohorts of 6-8-weeks old BALB/c mice were intranasally inoculated with mouse-adapted SARS-CoV-2 (105 TCID50 MA10 SARS-CoV-2 (US_WA-1/2020)), followed by treatment with oral daily doses of A27 ranging from 100 mg/kg/day to 200 mg/kg/day twice daily (400 mg/kg/day total dose) for 5 days (equivalent to ˜8 mg/kg/day to ˜32 mg/kg/day for a human dose based on body surface area allometric conversion). This led to a 1-3 logs reduction of viral titers compared to vehicle (40% 2-hydroxypropyl-β-cyclodextrin in water) treated mice (Table 1).

TABLE 1
Viral titers in lungs of Balb/c mice infected with
SARS-CoV-2 treated with STF-3577 compared to vehicles

| | |
|---|---|
| Dose | Viral Load Drop Relative to Vehicle |

| | | |
|---|---|---|
| 100 mg/kg/day | ~1 | log |
| 200 mg/kg twice daily | ~1-3 | log |

We further explored the potential of A27 in combination with other antivirals and found that A27 was synergistic with protease inhibitors against SARS-CoV-2 (e.g. PF-07321332) and additive with a polymerase inhibitor nucleoside analog (e.g EIDD2801). Thus, A27 can be combined with nirmatrelvir or paxlovid.

We then discovered that A27 also inhibits Venezuelan equine encephalitis virus (VEEV) in cell culture. Using a luciferase reporter containing the TC83 strain of VEEV in U87-MG cells, we discovered A27 inhibits VEEV genome replication with an EC₅₀ of 7.9 μM, without any evidence of toxicity.

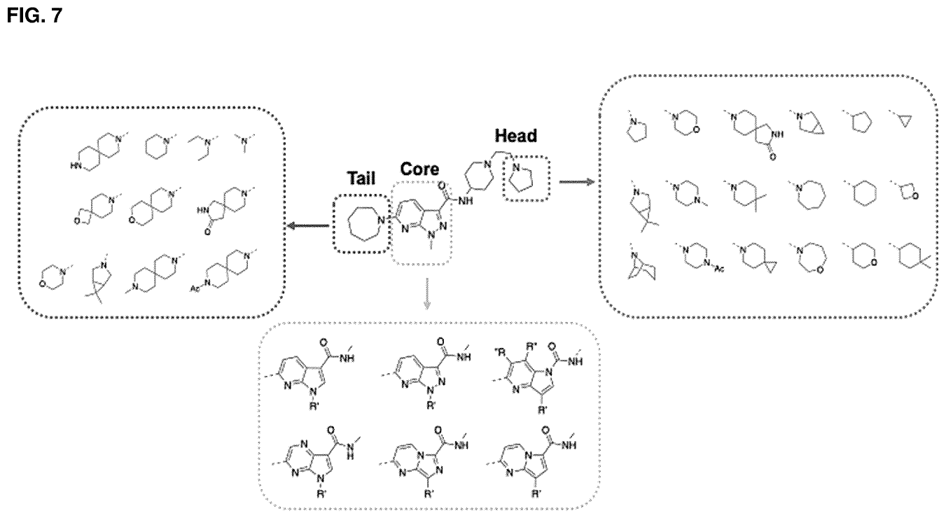

We then began to explore potential derivatives of A27 to further optimize its pharmacokinetics and pharmacodynamic performances. Representative A27 derivatives are compounds of Formulae I-III (illustrated above) and a proposed scheme to synthesize more A27 derivatives is shown in FIG. 7.

### Example 2

New compounds were generated (Example 2) and tested (Example 3).

### Generating New Compounds

### Core Preparations

### 7-Pyrazolopyridine Core (Core 1)

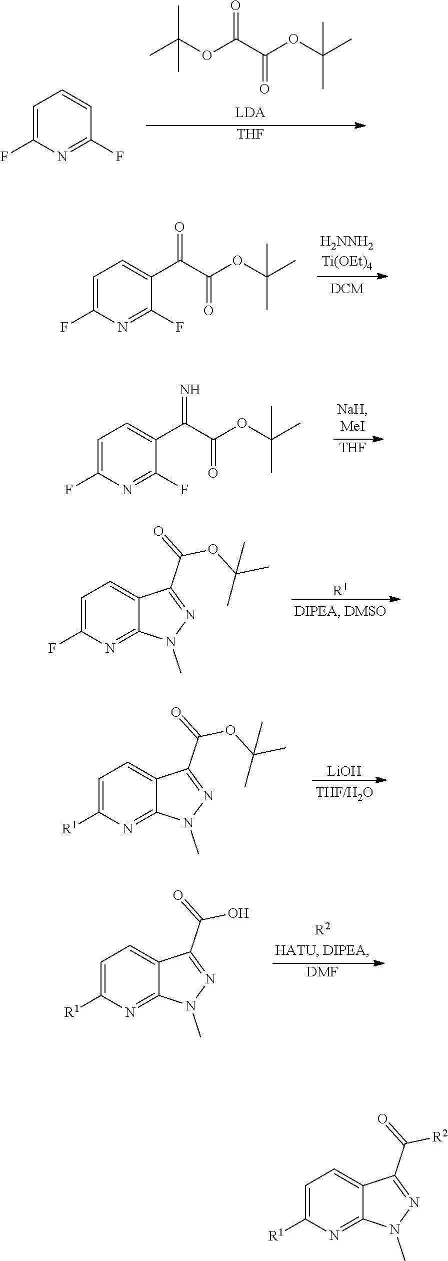

Compounds bearing the pyrazolopyridine core were synthesized according to the general reaction scheme described above using known synthetic procedures from commercially available difluoropyridine and tert-butyloxylate. The keto ester was converted to the hydrazone before cyclization and methylation. The appropriate R¹ amine substituent was installed using standard SNAr methodology. The tert-butyl ester was hydrolyzed under basic lithium hydroxide conditions before the appropriate amide were formed using standard reductive amination conditions from amines that were either commercially available or prepared using standard alkylation or reductive amination conditions.

### tert-butyl 2-(2,6-difluoro-3-pyridyl)-2-oxo-acetate

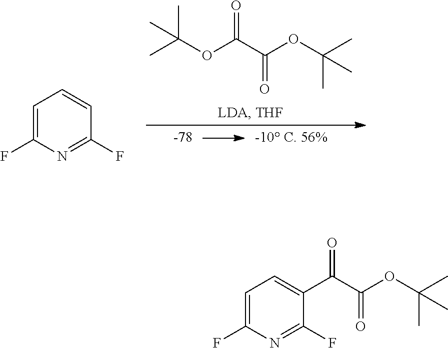 [SMILES: CC(C)(C)OC(=O)C(=O)OC(C)(C)C ; Fc1cccc(F)n1 ; CC(C)(C)OC(=O)C(=O)c1ccc(F)nc1F]

n-Butyllithium solution in hexanes (9 mL, 95.59 mmol) was added dropwise over 5 min to a clear solution of N-isopropylpropan-2-amine (13.4 mL, 95.59 mmol) in anhydrous THF (90 mL) at −78° C. (cooled with an acetone dry ice bath) under a dry atmosphere of nitrogen gas. The dry ice and acetone bath was then replaced with an ice water bath and the reaction mixture was allowed to stir at 0° C. for 30 min. The ice water bath was then again replaced with an acetone dry ice bath. The resulting yellow solution was then added dropwise over 30 min, via cannular transfer, to a solution of 2,6-difluoropyridine (7.89 mL, 86.9 mmol) in anhydrous THF (90 mL) at −78° C. (cooled with an acetone dry ice bath) under a dry atmosphere of nitrogen gas. The reaction mixture was then allowed to stir for 1 hour at −78° C. Finally, a dry solution of ditert-butyl oxalate (22.32 g, 110.36 mmol) and anhydrous THF (80 mL+10 mL rinse) was added slowly over 5 min via cannular transfer to the flask (solution was added running it down the side of the flask to avoid a large exothermic release). The resulting brown mixture was then allowed to stir for 1 hour at −78° C. before replacing the acetone and dry ice bath with an ice brine bath. The reaction mixture was then allowed to stir for an additional 30 min at −10° C. Aqueous saturated ammonium chloride (90 mL) was then added at −10° C. The cooling bath was then removed, and the layers were separated. The aqueous layer was then extracted with ethyl acetate (3×30 mL). The combined organic layers were then washed with brine (50 mL), dried over sodium sulfate, and concentrated down under reduced pressure to produce a brown liquid. The residue was then purified via silica gel chromatography using hexanes and ethyl acetate (gradient 0-10%) to provide tert-butyl 2-(2,6-difluoro-3-pyridyl)-2-oxo-acetate (12.4 g, 48.5 mmol, 56% yield) as a yellow liquid. ¹H NMR (500 MHZ, CDCl₃) δ 8.48 (q, J=8.3 Hz, 1H), 7.01 (dd, J=8.3, 2.7 Hz, 1H), 1.59 (s, 9H). LR/MS (ESI) [M−H] calc: 242.2 [M−H] found: 240.0.

### tert-butyl (Z)-2-(2,6-difluoropyridin-3-yl)-2-hydrazineylideneacetate

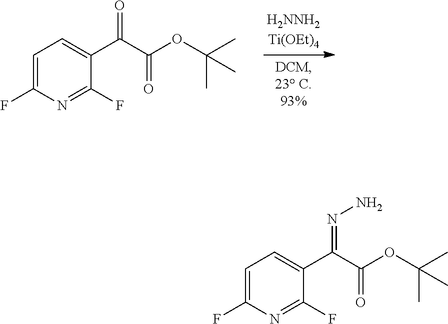 [SMILES: CC(C)(C)OC(=O)C(=O)c1ccc(F)nc1F ; CC(C)(C)OC(=O)/C(=N\N)c1ccc(F)nc1F]

Titanium ethoxide (15.48 mL, 73.85 mmol) and hydrazine monohydrate (3.7 mL, 73.85 mmol) were added in a single portion to a solution of tert-butyl 2-(2,6-difluoro-3-pyridyl)-2-oxo-acetate (9 g, 36.92 mmol) in DCM (40 mL) at 23° C. The reaction mixture was then allowed to stir for 1 hour. The resulting yellow mixture was then diluted in DCM (30 mL) and water (45 mL) and stirred for 30 min. Aqueous saturated ammonium chloride (30 mL) was then added. The resulting mixture was then passed through a pad of celite and rinsed with DCM (50 mL). The layers were then separated, and the aqueous layer was extracted with ethyl acetate (3×20 mL). The combined organic layers were then dried over sodium sulfate and concentrated down under reduced pressure to provide tert-butyl (Z)-2-(2,6-difluoropyridin-3-yl)-2-hydrazineylideneacetate (7.5 g, 34.29 mmol, 93% yield) as a yellow solid. ¹H NMR (500 MHZ, CDCl₃) δ 7.87 (q, J=8.1 Hz, 1H), 6.97 (ddd, J=8.1, 2.8, 0.9 Hz, 1H), 1.51 (s, 9H).

### tert-butyl 6-fluoro-1-methyl-1H-pyrazolo[3,4-b]pyridine-3-carboxylate

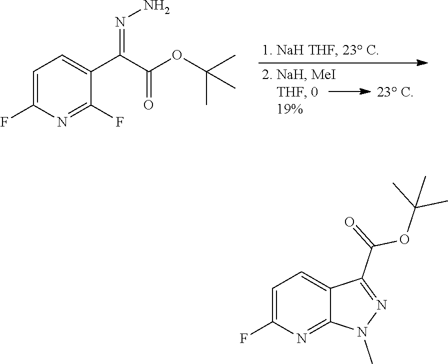 [SMILES: CC(C)(C)OC(=O)/C(=N\N)c1ccc(F)nc1F ; Cn1nc(C(=O)OC(C)(C)C)c2ccc(F)nc21]

A solution of tert-butyl (2E)-2-(2,6-difluoro-3-pyridyl)-2-hydrazinylidene-acetate (8.8 g, 34.29 mmol) in THF (75 mL) was treated with 60% sodium hydride (1.8 g, 73.73 mmol) at 23° C. The resulting mixture was allowed to stir for 20 min at 23° C. The flask was then cooled to 0° C. in an ice water bath. The reaction mixture was then treated with a second portion of 60% sodium hydride (1.8 g, 73.73 mmol). Mel (42.7 mL, 685.83 mmol) was then added dropwise over 5 min. The reaction mixture was then allowed to stir for an additional 1 hour as the cooling bath slowly warmed to ambient temperatures. Aqueous saturated ammonium chloride was then added to the reaction mixture and the layers were separated. The aqueous layer was then extracted with ethyl acetate (3×15 mL). The combined organic layers were then washed with brine (15 mL), concentrated down under reduced pressure and purified via silica gel chromatography using 5-40% ethyl acetate and hexanes to provide tert-butyl 6-fluoro-1-methyl-1H-pyrazolo[3,4-b]pyridine-3-carboxylate (1.9 g, 6.66 mmol, 19%) as a yellow solid. ¹H NMR (500 MHZ, CDCl₃) δ 8.50 (dd, J=8.6, 7.5 Hz, 1H), 6.92 (dd, J=8.5, 1.0 Hz, 1H), 4.15 (s, 3H), 1.69 (s, 9H). LR/MS (ESI) [M+H−tBu−F] calc: 176.0 [M+H−tBu−F] found: 177.9.

### tert-butyl 6-(azepan-1-yl)-1-methyl-1H-pyrazolo[3,4-b]pyridine-3-carboxylate

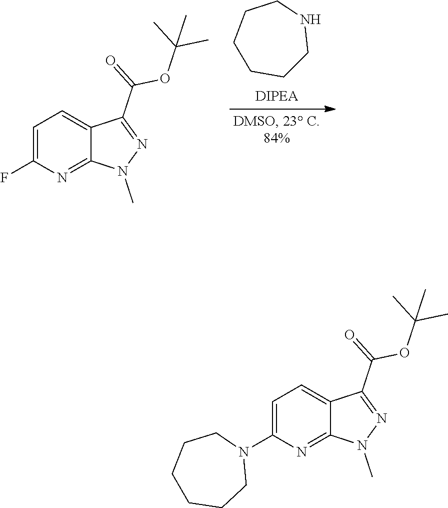

DIPEA (2.63 mL, 15.15 mmol) was added to a mixture of tert-butyl 6-fluoro-1-methyl-1H-pyrazolo[3,4-b]pyridine-3-carboxylate (1.4 g 5.05 mmol) and azepane (2.85 mL, 25.25 mmol) in DMSO (50 mL) at 23° C. The flask was then heated to 150° C. and allowed to stir for 2 hours. The flask was then removed from heat and saturated aqueous ammonium chloride (50 mL) was then added to the reaction mixture. The layers were then separated, and the aqueous layer was extracted using Ethyl acetate (3×25 mL). The combined organic layers were then washed with brine (3×25 mL), dried over sodium sulfate, and concentrated down under reduced pressure. The crude was then purified via Silica gel chromatography using 20-70% ethyl acetate and hexanes to provide tert-butyl 6-(azepan-1-yl)-1-methyl-1H-pyrazolo[3,4-b]pyridine-3-carboxylate (1.5 g, 4.24 mmol, 84% yield) as an orange oil. ¹H NMR (500 MHZ, CDCl₃) δ 8.05 (d, J=9.0 Hz, 1H), 6.58 (d, J=9.0 Hz, 1H), 4.01 (s, 3H), 3.73 (t, J=6.0 Hz, 4H), 1.88-1.78 (m, 4H), 1.66 (s, 9H), 1.55 (dt, J=6.0, 2.7 Hz, 4H). LR/MS (ESI) [M+H] calc: 331.4 [M+H] found: 331.2.

### 6-(azepan-1-yl)-1-methyl-1H-pyrazolo[3,4-b]pyridine-3-carboxylic acid

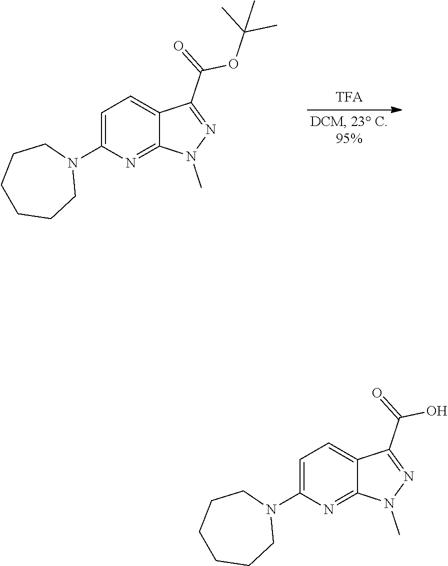 [SMILES: Cn1nc(C(=O)OC(C)(C)C)c2ccc(N3CCCCCC3)nc21 ; Cn1nc(C(=O)O)c2ccc(N3CCCCCC3)nc21]

A solution of tert-butyl 6-(azepan-1-yl)-1-methyl-pyrazolo[3,4-b]pyridine-3-carboxylate (1.47 g, 4.46 mmol) in DCM (15 mL) was treated with TFA (10.23 mL, 133.7 mmol) at 23° C. and allowed to stir for 4 hours. The reaction mixture was then concentrated under reduced pressure. The resulting orange solid was then redissolved in DCM (5 mL) and Toluene (3 mL) and concentrated a second time under reduced pressure to give 6-(azepan-1-yl)-1-methyl-1H-pyrazolo[3,4-b]pyridine-3-carboxylic acid (1 g, 3.63 mmol, 95% yield) as an orange solid. 1H NMR (500 MHZ, CDCl₃) δ 8.22 (d, J=9.3 Hz, 1H), 6.75 (d, J=9.4 Hz, 1H), 4.15 (s, 3H), 3.80 (t, J=5.9 Hz, 4H), 1.89 (p, J=5.3 Hz, 4H), 1.62 (p, J=2.7 Hz, 4H). LR/MS (ESI) [M+H] calc: 275.3 [M+H] found: 275.0.

### tert-butyl 4-(6-(azepan-1-yl)-1-methyl-1H-pyrazolo[3,4-b]pyridine-3-carboxamido) piperidine-1-carboxylate

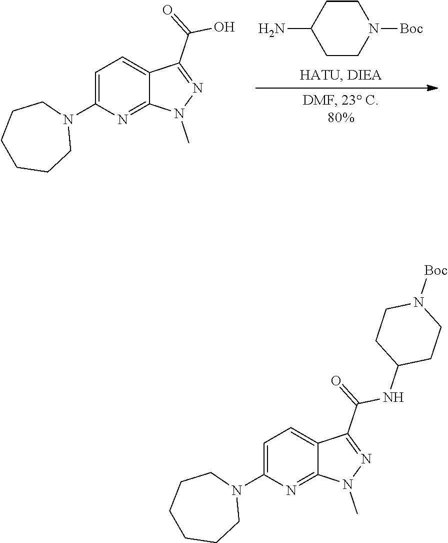 [SMILES: Cn1nc(C(=O)O)c2ccc(N3CCCCCC3)nc21 ; CC(C)(C)OC(=O)N1CCC(N)CC1 ; Cn1nc(C(=O)NC2CCN(C(=O)OC(C)(C)C)CC2)c2ccc(N3CCCCCC3)nc21]

A solution of 6-(azepan-1-yl)-1-methyl-pyrazolo[3,4-b]pyridine-3-carboxylic acid (1.23 g, 4.47 mmol) and tert-butyl 4-aminopiperidine-1-carboxylate (1 g, 4.92 mmol) in DMF (18 mL) was treated with DIPEA (2.72 mL, 15.64 mmol) at 23° C. HATU (2.5 g, 6.7 mmol) was then added and the reaction was stirred for 3 hours. The reaction was then diluted with ethyl acetate (50 mL), washed with brine (30 mL), dried over sodium sulfate, and concentrated down under reduced pressure. The resulting orange solid was then purified via silica gel chromatography using 20-60% hexanes and ethyl acetate to give tert-butyl 4-(6-(azepan-1-yl)-1-methyl-1H-pyrazolo[3,4-b]pyridine-3-carboxamido) piperidine-1-carboxylate (1.7 g, 3.57 mmol, 80% yield) as an orange solid. ¹H NMR (500 MHZ, CDCl₃) δ 8.22 (d, J=9.1 Hz, 1H), 6.58 (d, J=9.0 Hz, 1H), 4.18-3.97 (m, 3H), 3.95 (s, 3H), 3.73 (t, J=6.1 Hz, 4H), 2.02 (dt, J=12.9, 4.2 Hz, 2H), 1.83 (dq, J=8.9, 4.9 Hz, 4H), 1.57 (qt, J=7.9, 4.2 Hz, 4H), 1.47 (s, 9H). LR/MS (ESI) [M+H] calc: 457.6 [M+H] found: 457.3.

### 6-(azepan-1-yl)-1-methyl-N-(piperidin-4-yl)-1H-pyrazolo[3,4-b]pyridine-3-carboxamide

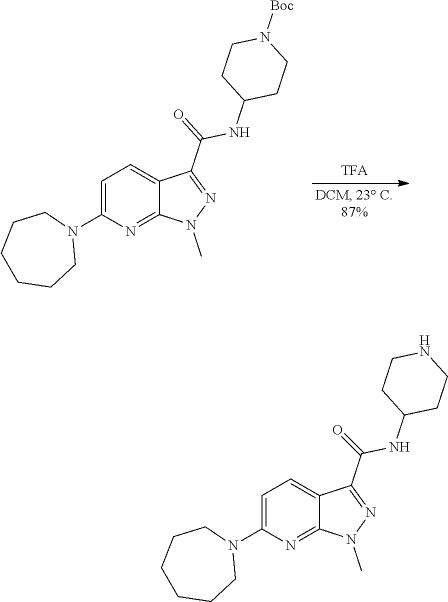 [SMILES: Cn1nc(C(=O)NC2CCN(C(=O)OC(C)(C)C)CC2)c2ccc(N3CCCCCC3)nc21 ; "DCM, 23° C.87%" ; Cn1nc(C(=O)NC2CCNCC2)c2ccc(N3CCCCCC3)nc21 ; "TFA"]

TFA (24.91 mL, 325.51 mmol) was added to a solution of tert-butyl 4-[6-(azepan-1-yl)-1-methyl-pyrazolo[3,4-b]pyridine-3-carbonyl]amino]piperidine-1-carboxylate (3 g, 6.51 mmol) in DCM (26 mL) at ambient temperatures (23° C.) and allowed to stir for 2 hours. The mixture was then concentrated down under reduced pressure. The resulting orange solid was then redissolved in DCM and toluene and reconcentrated down under reduced pressure. The crude was then further purified via silica gel chromatography using 0-10% methanol and DCM to provide 6-(azepan-1-yl)-1-methyl-N-(piperidin-4-yl)-1H-pyrazolo[3,4-b]pyridine-3-carboxamide (2.1 g, 5.65 mmol, 87% yield) as a pale yellow solid. ¹H NMR (500 MHZ, MeOD) δ 8.11 (d, J=9.1 Hz, 1H), 6.70 (d, J=9.1 Hz, 1H), 4.19 (tt, J=10.7, 4.1 Hz, 1H), 3.95 (s, 3H), 3.76 (t, J=6.1 Hz, 4H), 3.52-3.44 (m, 2H), 3.17 (td, J=12.8, 3.1 Hz, 2H), 2.25-2.16 (m, 2H), 1.92-1.87 (m, 2H), 1.84 (hept, J=3.9 Hz, 4H), 1.56 (p, J=2.7 Hz, 4H). LR/MS (ESI) [M+H] calc: 357.5 [M+H] found: 357.1.

### General Reductive Amination for NSP4 Benzyl Derivative Compounds

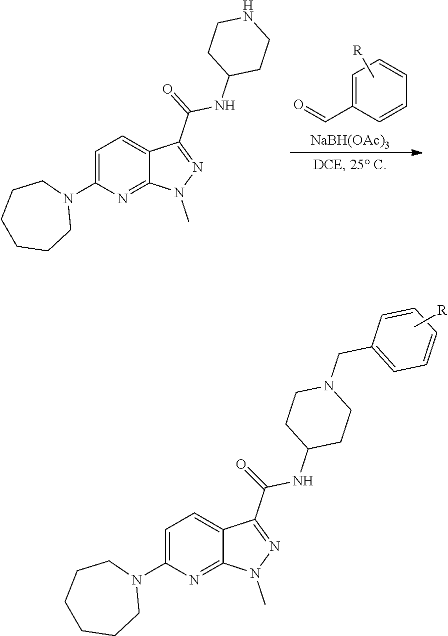

Sodium Triacetoborohydride (1.4 eq) was added to a solution of 6-(azepan-1-yl)-1-methyl-N-(4-piperidyl) pyrazolo[3,4-b]pyridine-3-carboxamide (1 eq) and benzaldehyde derivative (1.5 eq) in anhydrous DCE (0.25 M) under an atmosphere of nitrogen. The reaction mixture was then allowed to stir for 3 hours. Aqueous saturated sodium bicarb was then added to the reaction mixture and extracted with ethyl acetate. The combined organic layers were then dried over sodium sulfate and concentrated down under reduced pressure. The crude was then dissolved in a small amount of methanol using heat to help dissolve. The flask was then placed in an ice bath. Solid product began to crystallize out of solution. Product was then filtered and rinsed with more methanol.

### 6-(azepan-1-yl)-1-methyl-N-(1-(4-methylbenzyl) piperidin-4-yl)-1H-pyrazolo[3,4-b]pyridine-3-carboxamide

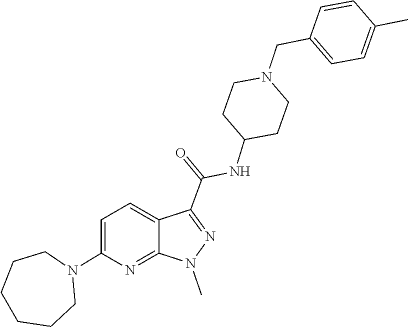 [SMILES: Cc1ccc(CN2CCC(NC(=O)c3nn(C)c4nc(N5CCCCCC5)ccc34)CC2)cc1]

¹H NMR (500 MHZ, MeOD) δ 8.11 (d, J=9.1 Hz, 1H), 7.40 (d, J=8.2 Hz, 2H), 7.33 (d, J=7.9 Hz, 2H), 6.72 (d, J=9.1 Hz, 1H), 4.29 (s, 2H), 4.17 (dt, J=17.8, 7.4 Hz, 1H), 3.95 (s, 3H), 3.78 (t, J=6.1 Hz, 4H), 3.52 (dt, J=11.5, 4.8 Hz, 2H), 3.25-3.07 (m, 2H), 2.40 (s, 3H), 2.32-2.08 (m, 2H), 2.00-1.77 (m, 6H), 1.57 (p, J=2.7 Hz, 4H). LR/MS (ESI) [M+H] calc: 461.6 [M+H] found: 461.2.

### 6-(azepan-1-yl)-N-(1-(4-chlorobenzyl) piperidin-4-yl)-1-methyl-1H-pyrazolo[3,4-b]pyridine-3-carboxamide

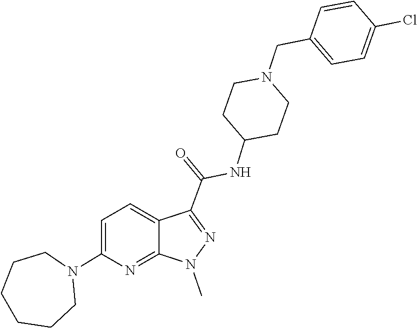 [SMILES: Cn1nc(C(=O)NC2CCN(Cc3ccc(Cl)cc3)CC2)c2ccc(N3CCCCCC3)nc21]

¹H NMR (500 MHZ, MeOD) δ 8.12 (d, J=9.1 Hz, 1H), 7.52 (dd, J=7.5, 1.8 Hz, 1H), 7.39 (dd, J=7.8, 1.5 Hz, 1H), 7.30 (td, J=7.5, 1.5 Hz, 1H), 7.26 (td, J=7.6, 1.9 Hz, 1H), 6.71 (d, J=9.1 Hz, 1H), 3.95 (s, 3H), 3.98-3.89 (m, 1H), 3.78 (t, J=6.1 Hz, 4H), 3.69 (s, 2H), 2.97 (d, J=11.9 Hz, 2H), 2.31 (t, J=11.5 Hz, 2H), 1.96 (d, J=12.8 Hz, 2H), 1.85 (s, 4H), 1.72 (qd, J=11.8, 3.8 Hz, 2H), 1.57 (p, J=2.7 Hz, 4H). LR/MS (ESI) [M+H] calc: 482.0 [M+H] found: 481.1.

### 6-(azepan-1-yl)-N-(1-(4-fluorobenzyl) piperidin-4-yl)-1-methyl-1H-pyrazolo[3,4-b]pyridine-3-carboxamide

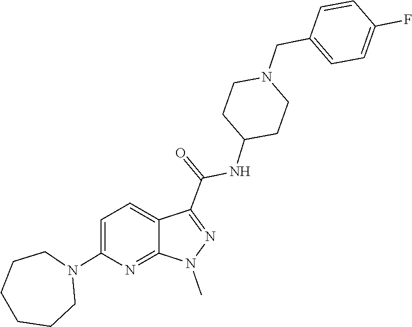 [SMILES: Cn1nc(C(=O)NC2CCN(Cc3ccc(F)cc3)CC2)c2ccc(N3CCCCCC3)nc21]

¹H NMR (500 MHZ, DMSO) δ 8.07 (d, J=9.1 Hz, 1H), 8.01 (d, J=8.3 Hz, 1H), 7.37-7.29 (m, 2H), 7.18-7.10 (m, 2H), 6.73 (d, J=9.1 Hz, 1H), 3.89 (s, 3H), 3.78 (tdd, J=11.4, 8.3, 4.3 Hz, 1H), 3.72 (t, J=6.1 Hz, 4H), 3.44 (s, 2H), 2.79 (d, J=11.1 Hz, 2H), 2.01 (td, J=11.8, 2.6 Hz, 2H), 1.77 (d, J=5.8 Hz, 4H), 1.76-1.69 (m, 2H), 1.63 (qd, J=11.9, 3.8 Hz, 2H), 1.48 (p, J=2.7 Hz, 4H). LR/MS (ESI) [M+H] calc: 465.6 [M+H] found: 465.3.

### Synthesis of 6-(azepan-1-yl)-1-methyl-N-(piperidin-4-yl)-1H-pyrazolo[3,4-b]pyridine-3-carboxamide

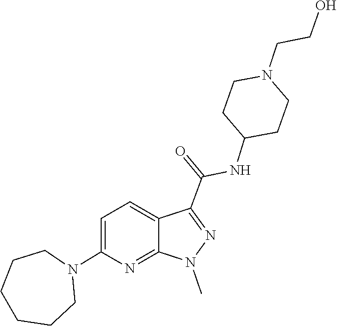 [SMILES: Cn1nc(C(=O)NC2CCN(CCO)CC2)c2ccc(N3CCCCCC3)nc21]

To a solution of 6-(azepan-1-yl)-1-methyl-N-(piperidin-4-yl)-1H-pyrazolo[3,4-b]pyridine-3-carboxamide (0.5 mmol) in DMF (5 mL) was added 2-bromoethanol (1.0 g, 5 mmol), Cs₂CO₃ and NaI. After stirring at 70° C. for 4 h, the reaction solution was diluted with EtOAc and washed with water and brine. The organic layer was dried (MgSO₄) and the solvent was evaporated to dryness under reduced pressure to provide crude 6-(azepan-1-yl)-N-(1-(2-hydroxyethyl) piperidin-4-yl)-1-methyl-1H-pyrazolo[3,4-b]pyridine-3-carboxamide.

### Synthesis of 6-(azepan-1-yl)-1-methyl-N-(1-(2-(pyrrolidin-1-yl)ethyl) piperidin-4-yl)-1H-pyrazolo[3,4-b]pyridine-3-carboxamide

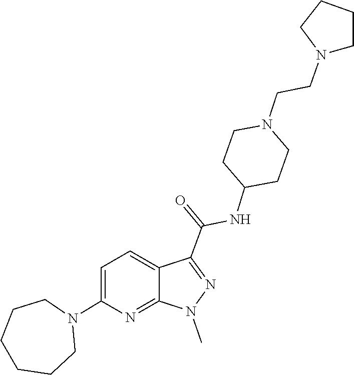 [SMILES: Cn1nc(C(=O)NC2CCN(CCN3CCCC3)CC2)c2ccc(N3CCCCCC3)nc21]

To a solution of 6-(azepan-1-yl)-N-(1-(2-hydroxyethyl) piperidin-4-yl)-1-methyl-1H-pyrazolo[3,4-b]pyridine-3-carboxamide (0.2 mmol) in CH₂Cl (5 mL) was added MsCl (4 mmol) and TEA (1.5 mmol). After stirring at rt for 30 min, the solvent was evaporated to dryness under reduced pressure. The crude mesylate was then dissolved in DMF (2 mL), added pyrrolidine (4 mmol), Cs₂CO₃ (1.0 mmol) and NaI (0.5 mmol), and stirred for 16 h at 80° C. to provide the title product which was purified by reverse-phase prep-HPLC.

¹H NMR (CDCl₃ and CD₃OD, δ ppm): 8.18 (d, 1H), 6.58 (d, 1H), 4.02 (s, 3H), 3.91-3.48 (m, 12H), 3.11-2.95 (m, 4H), 2.80-1.95 (m, 9H), 1.82-1.72 (m, 4H) and 1.49-1.47 (m, 4H).

### 7-Pyrazolopyridine Core (Core 11)

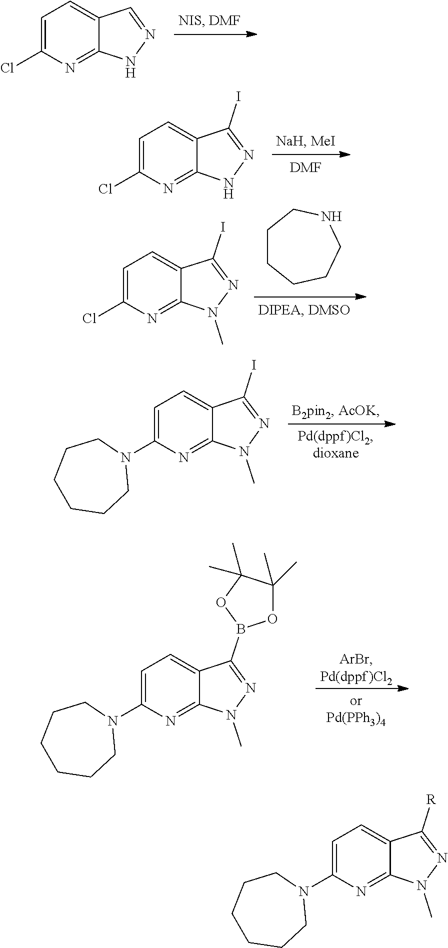

Compounds that did not possess an amide linker were synthesized from commercially available 6-chloro-1H-pyrazolo[3,4-b]pyridine. The core was halogenated using N-iodosuccinamide before methylation with iodomethane. The azepane moiety was installed using standard SNAr chemistry before the core was converted to the boronic pinacol ester following well established literature procedures. The boronic acid was reacted with the appropriate aryl halide using standard Suzuki-Miyaura coupling conditions to furnish the desired targets.

### 7-Pyrazolopyridine Core (Core 3)

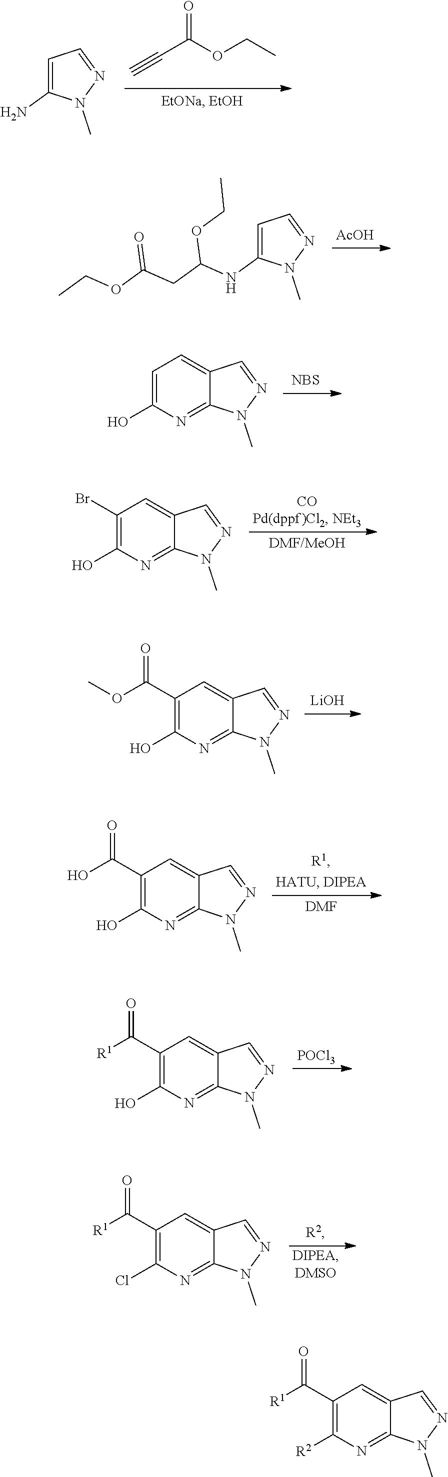

Compounds bearing the pyrazolopyridine core with the 5,6 substitution pattern were synthesized following the general reaction procedure provided. 1-Methyl-1H-pyrazolo[3,4-b]pyridine-6-ol was synthesized from commercially available 1-methyl-1H-pyrazol-5-amine using known condensation chemistry. Treatment of this core using N-bromosuccinamide resulted in bromination at the 5-position. The bromine was converted to the methyl ester using well established palladium-mediated carbonylation chemistry. Basic hydrolysis of the ester was performed before a standard reductive amination was performed with the appropriate R² amine. In order to install the desired R² substituent, the core was treated with phosporyl chloride and the appropriate amine inserted using standard SNAr conditions.

### Azaindole Core (Core 6)

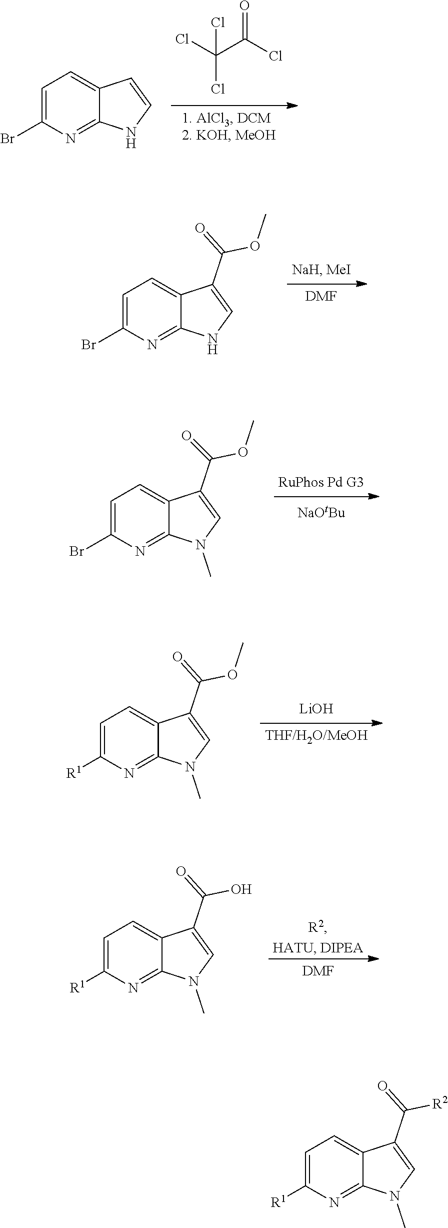

Compounds containing the 7-azaindole core were synthesized from commercially available 6-bromo-7-azaindole. The methyl ester was installed using standard Friedel-Crafts Acylation conditions before the indole was methylated with sodium hydride and methyl iodide. The appropriate R¹ amine was installed using Buchwald-Hartwig chemistry catalyzed by RuPhos Pd G3. Basic hydrolysis of the methyl ester followed by a reductive amination with the appropriate amine (R²) afforded the desired targets.

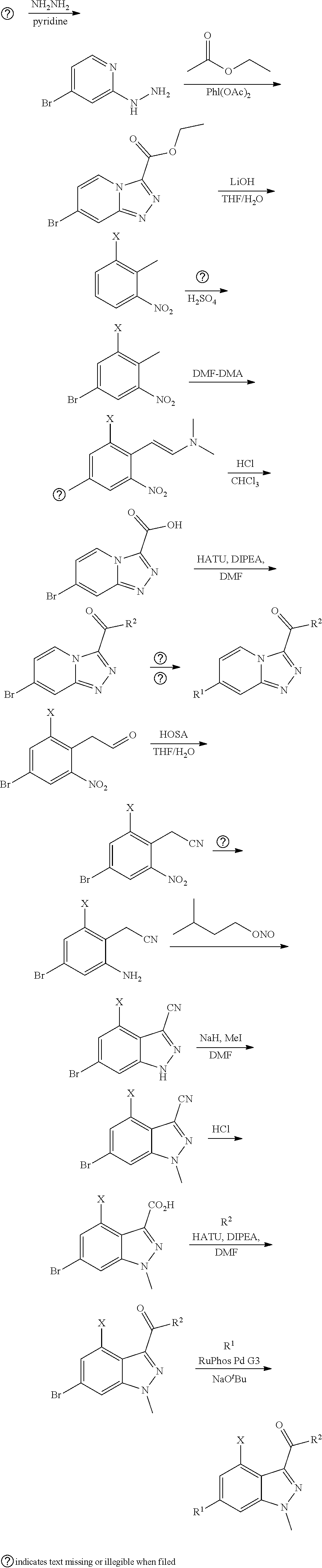

### Indazole Core (Cores 5 and 12)

Compounds containing indazole cores were synthesized via the general procedure described above using known literature procedures. Functionalized indazoles (such as where X═CF₃) could be obtained from the appropriate 2-nitrotoluene. After methylation of the core with iodomethane, the nitrile was hydrolyzed under acidic conditions to furnish the carboxylic acid. The appropriate R² amide was inserted via a standard amide coupling reaction conditions using HATU in DMF. The desired R¹ substituent was installed using standard Buchwald-Hartwig conditions catalyzed by RuPhos Pd G3 to afford the desired compounds.

### Triazolopyridine Core (Core 4)

The triazolopyridine core was synthesized using known literature conditions. Hydrolysis of the ethyl ester with lithium hydroxide afforded the carboxylic acid that could then be coupled with the appropriate R² amine to afford the desired amide. The desired R¹ substituent was installed using an amine under standard Buchwald-Hartwig amination conditions catalyzed with Pd₂(dba)₃ and BINAP.

### Pyrazolopyridine (Core 9)

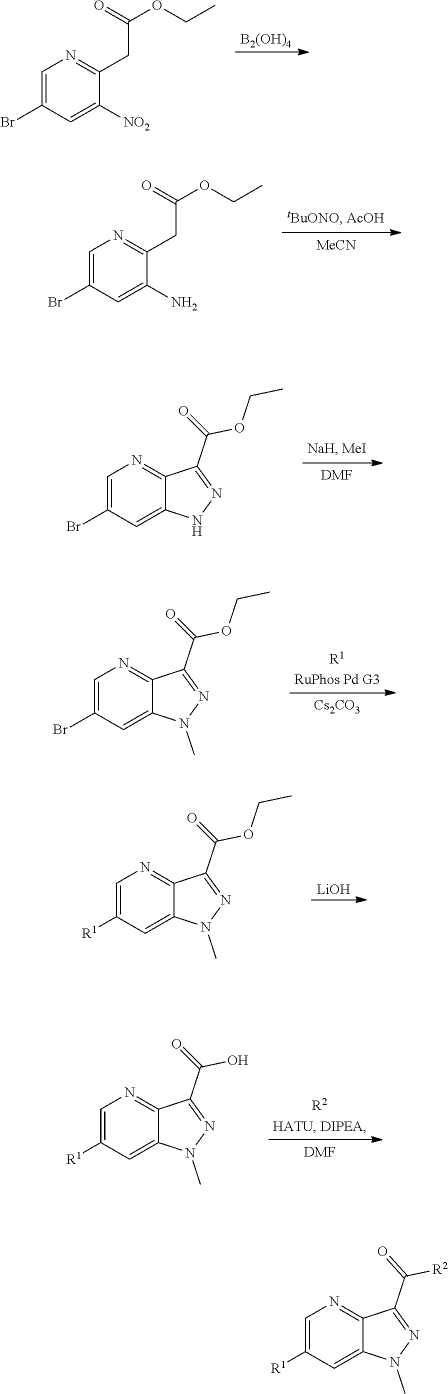

Compounds containing the 5-pyrazolopyridine core were synthesized following literature procedures from ethyl 2-(5-bromo-3-nitropyridine-2-yl) acetate. The appropriate R¹ substituent was installed using standard Buchwald-Hartwig amination conditions using an appropriate amine and catalyzed by RuPhos Pd G3. Lithium hydroxide was used to hydrolyze the ethyl ester to the carboxylic acid before the desired R² amide formed from an amide coupling facilitated by HATU.

### Napthyridine (Core 10)

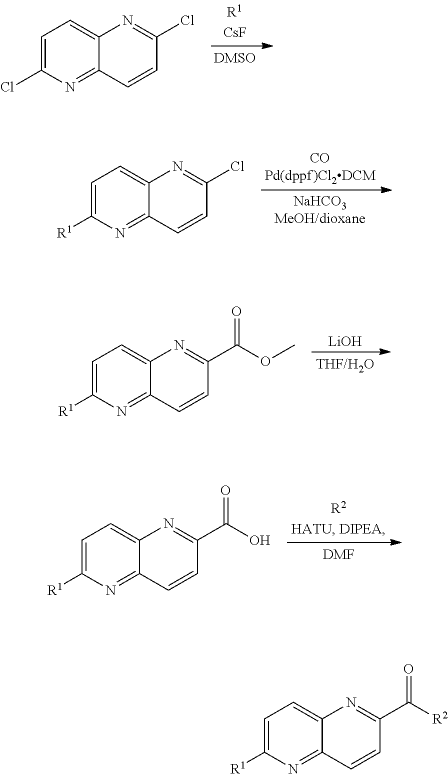

Commercially available 2,6-dichloronapthyridine was obtained to synthesize compounds containing a napthyridine core. The desired R¹ amine substituent was inserted using standard SNAr conditions before the methyl ester was installed using a standard palladium catalyzed carbonylation reaction. The methyl ester was hydrolyzed to the carboxylic acid before an amide coupling with the appropriate R² amine afforded the desired product.

### Pyridopyrimide Core (Core 14)

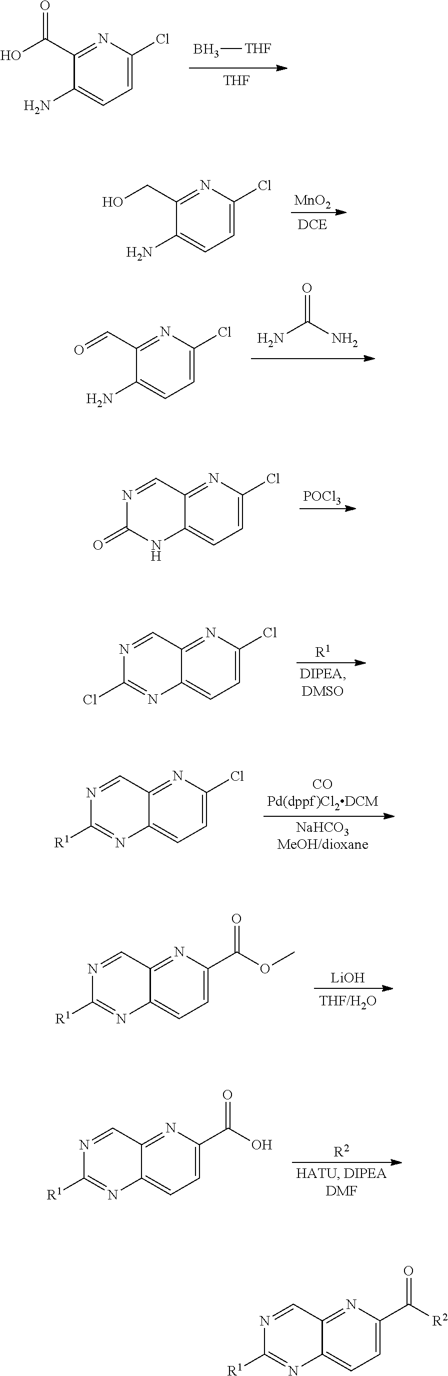

2,6-dichloropyridopyrimidine was synthesized following well established literature procedures. R¹ was inserted using standard SNAr conditions with the appropriate amine. The methyl ester was installed using standard palladium catalyzed carbonylation chemistry using carbon monoxide. The ester was hydrolyzed to the carboxylic acid before an amide coupling was performed using the desired R² amine to afford the desired amide.

### Pyridopyrimidine (Core 15)

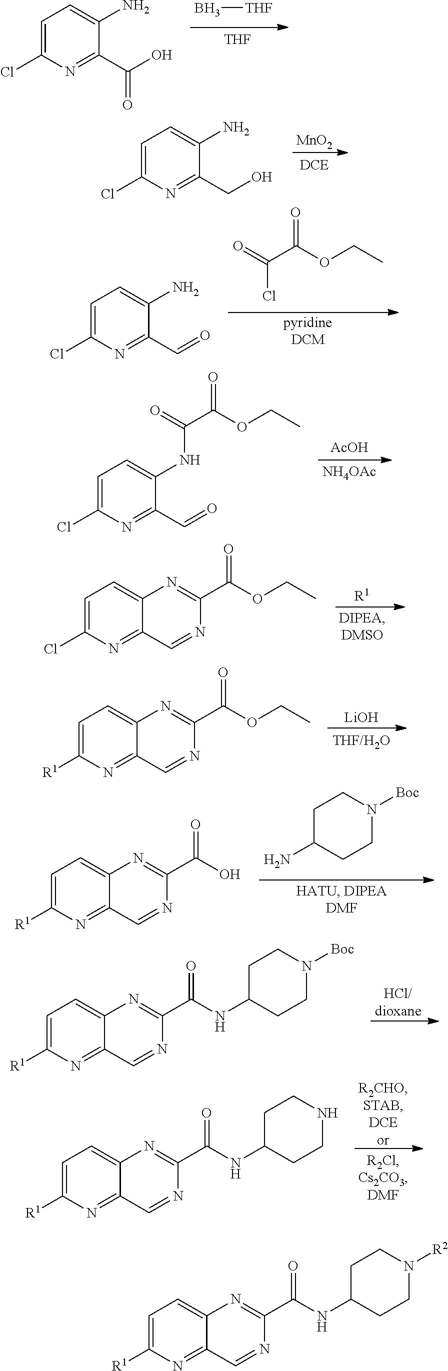

Ethyl 6-chloropyrido[3,2-d]pyrimidine-2-carboxylate was synthesized according to previously published literature. The appropriate R¹ substituent was installed by an SNAr reaction with the appropriate amine. Hydrolysis of the methyl ester afforded a carboxylic acid that was then coupled to tert-butyl 4-aminopiperidine-1-carboxylate using HATU. The Boc group was removed using 4M HCl in dioxane. The resulting HCl salt was then subjected to either a reductive amination with the appropriate R² aldehyde or an alkylation reaction with the appropriate R² halide or triflate.

### Quinoline Core (Core 7)

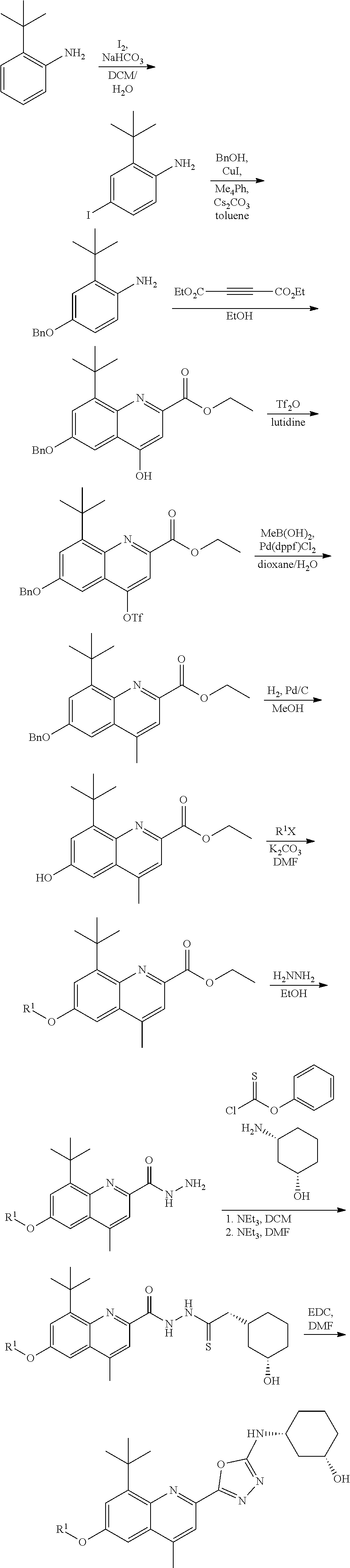

Substituted hydroxyquinoline core was synthesized using previously reported literature procedures. The hydroxy group could either be converted to the triflate and displaced for compounds containing an amine at R¹ or could be alkylated using standard alkylation conditions for compounds possessing an ether linkage at R¹. The ester could then be reacted with hydrazine before condensation with o-phenyl carbonochloridothioate and aminocylcohexanol and subsequent EDC mediated cyclization to afford the oxadiazole linked derivatives.

### General Procedure for R¹ SNAr Alkylations

All R¹ SNAr alkylations were performed as described in the representative example below using a variety of commercially available amines.

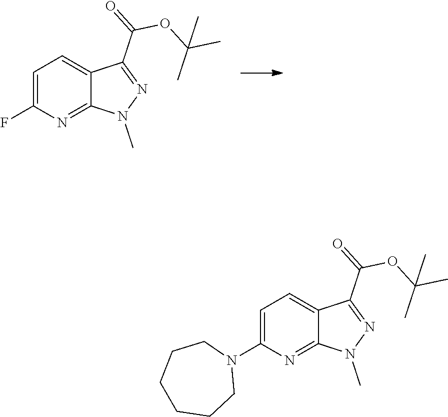

DIPEA (3 eq) was added to a mixture of tert-butyl 6-fluoro-1-methyl-1H-pyrazolo[3,4-b]pyridine-3-carboxylate (1 eq) and azepane (5 eq) in DMSO (0.01 M) at 23° C. The flask was then heated to 150° C. and for 2 h. The flask was then removed from heat and saturated aqueous ammonium chloride was then added to the reaction mixture. The layers were then separated, and the aqueous layer was extracted using ethyl acetate (×3). The combined organic layers were then washed with brine (×3), dried over anhydrous sodium sulfate, and concentrated down under reduced pressure. The crude was then purified via Silica gel chromatography using 20-70% ethyl acetate and hexanes to provide tert-butyl 6-(azepan-1-yl)-1-methyl-1H-pyrazolo[3,4-b]pyridine-3-carboxylate (84% yield) as an orange oil.

### General Procedure for R² Reductive Aminations

All reductive aminations were performed as described in the representative example below using a variety of commercially available amines.

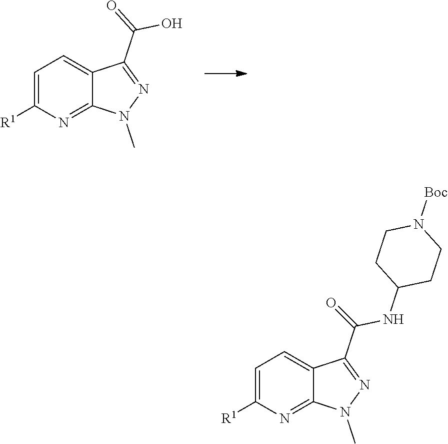

A solution of 6-(azepan-1-yl)-1-methyl-pyrazolo[3,4-b]pyridine-3-carboxylic acid (1 eq) and tert-butyl 4-aminopiperidine-1-carboxylate (1.1 eq) in DMF (0.2M) was treated with DIPEA (3.5 eq) and HATU (1.5 eq) added in a single portion at 23° C. The solution was then allowed to stir overnight. The reaction mixture was then diluted with ethyl acetate (100 mL) and washed with brine (3×30 mL) in a separation funnel. The collected organic layer was dried over anhydrous sodium sulfate and concentrated down under reduced pressure. The resulting brown crude was then purified via silica gel chromatography using 10-50% ethyl acetate and hexanes to give tert-butyl 4-[[6-(azepan-1-yl)-1-methyl-pyrazolo[3,4-b]pyridine-3-carbonyl]amino]piperidine-1-carboxylate (79% yield) as a yellow solid.

Non commercially available amines were prepared using standard literature procedures for alkylations or reductive aminations. Representative examples reactions are provided below.

### Representative Example for Alkylation Reactions

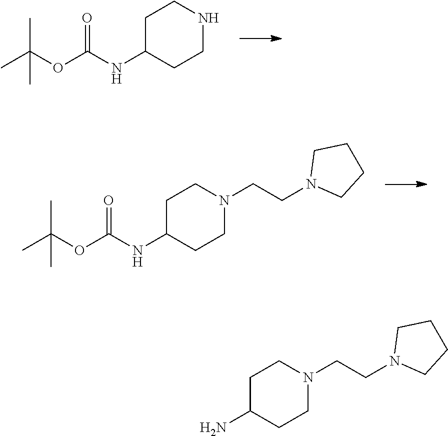

Step 1: tert-butyl N-(4-piperidyl) carbamate (1 eq), 1-(2-chloroethyl)pyrrolidine; hydrochloride (1.1 eq) and K₂CO₃ (3 eq) were suspended in DMF (0.5M) at 23° C. The reaction was warmed to 60° C. for 18 h. The reaction was cooled to RT, diluted with ethyl acetate and washed with 50% brine (×3). The organic layer was dried over anhydrous sodium sulfate and evaporated to afford a crude residue that was purified on a Biotage Selekt eluting a gradient of 0-20% (0.5M NH₃) MeOH in DCM on a 50 g silica column. The desired product (monitored by TLC) was found to elute around 20% and appropriate fractions were combined and concentrated to afford the desired tert-butyl N-[1-(2-pyrrolidin-1-ylethyl)-4-piperidyl]carbamate (47% yield) as an orange solid.

Step 2: To a solution of tert-butyl N-[1-(2-pyrrolidin-1-ylethyl)-4-piperidyl]carbamate (1 eq) in 1,4-Dioxane (0.34 M) was added 4M HCl in Dioxane (4 eq). The mixture was stirred at 23° C. for 3 h. The reaction was concentrated to obtain 1-(2-pyrrolidin-1- ylethyl) piperidin-4-amine (90% yield) as solid yellow hydrochloride salt.

### Representative Example for Reductive Aminations

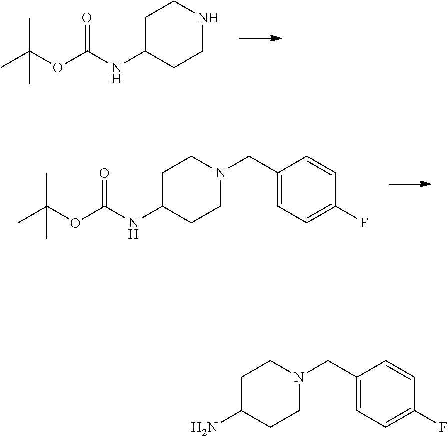

tert-butyl N-(4-piperidyl) carbamate (1 eq) was suspended in DCM (0.4 M) and 4-fluorobenzaldehyde (1 eq) added followed by 3 drops of acetic acid. The reaction was left to mature for 1 h at 23° C. before sodium triacetoxyborohydride (2 eq) was added. The reaction was left to stir at this temperature for 1 h before being quenched by the addition of water. The reaction mixture was washed with additional portions of DCM and saturated sodium bicarbonate. The organic portion was dried over anhydrous sodium sulfate and purified on a Biotage Selekt using a gradient of 0-10% MeOH in DCM on a 50 g silica column. Appropriate fractions were combined and concentrated to afford the desired tert-butyl N-[1-[(4-fluorophenyl)methyl]-4-piperidyl]carbamate (95% yield) as a white solid.

### Example 3

### Testing the New Compounds

The results of the below are depicted in FIG. 8. The concentration where viral replication decreases by 50% is the EC₅₀. The concentration where cellular viability decreases by 50% is the CC50. Activity Key: A=<1 μM; B=1-5 μM; C=5-10 μM; D=>10 μM (Blank=not yet determined).

### SARS-CoV-2

To assess antiviral activity against SARS-CoV-2, Huh7.5-ACE2-TMPRSS2 cells were infected with SARS-CoV-2-Nluc at a MOI of 0.05 for 1 hour and then washed once with PBS prior to addition of compounds. The cells were incubated for 48 hours and the luciferase activity as a surrogate measure for viral replication was measured using the Nano-Glo luciferase assay and the signal was normalized to no drug treated cells. Cellular viability was determined by the presto blue assay.

### Dengue Virus (DENV)

HuH-7 cells were cultivated in DMEM (Corning) with 10% FBS and were seeded at 10.000 cells/well density a day prior to infection in 96-well clear bottom white plates (Greiner/Corning). Cells were infected at MOI 0.01 with 50 ul of DENV2-Renilla Luciferase reporter virus (C38-Rluc2A-C-prM-E-NS region) at 37° C., 5% CO₂ for 1 h in DMEM without FBS. After 1 h, 50 μl of the compounds were added directly to the wells (100 ul final volume per well) in triplicates at final concentrations of 10 μM, 2.5 μM, 625 nM, 156 nM, 39 nM and 9.76 nM (4-fold dilutions) and then incubated for 48 h 37° C., 5% CO2. 0.4% DMSO was used as negative control. After 48 h, the media were taken out, cells were washed once with 200 ul PBS, and then 30 ul of 1× Lysis Buffer (Promega, cat n: E2820) were added to each well. Cells were incubated in a rock shaker (300 RPM) for 30 minutes to ensure complete lysis. Following that, 50 ul of Renilla substrate (Promega, cat n: E2820) were added per well using a injector needle and the luminescence readout was performed at 1 s integration time in Spark luminescence module (Tecan).

Viability assay was performed in parallel as described above, but using 96-well clear bottom black plates (Greiner/Corning). 48 h after treatment, the is replaced with 100 ul/well PrestoBlue viability reagent (Thermo Fisher Scientific) according to manufacturer's instructions. Cells were incubated for 1 h at 37° C., 5% CO₂ and the readout was done at 560/590 nm (excitation/emission) using the fluorescence module of Spark plate reader (Tecan).

### Chikungunya Virus (CHIKV)

CKV181-nLuc and MAYV-nLuc reporter viruses were generated as described in (Ramjag, A. et al. A high-throughput screening assay to identify inhibitory antibodies targeting alphavirus release. Virol. J. 19, 170 (2022). Nano luciferase (nLuc) reporter gene was engineered to the 5′ end of E2 in CHIKV vaccine stain 181/clone 25 (CKV181-nLuc) or MAYV infectious clone. The reporter viruses were reverse genetically generated and titered. Human U2OS cells in 96-well-plates were infected with CKV181-nLuc or MAYV-nLuc at an MOI of 0.5 and treated with serial dilutions of individual compounds at 1 hpi. At 24 hpi, 25 μl of the culture supernatant per well will be transferred to fresh luciferase assay plates and mixed with substrate for nLuc. In parallel cells will be lysed and intracellular ATP will be quantitated with CellTiter-Glo® luminescent cell viability assay. Control cells will be treated with DMSO and their viability and virus production will be set to 100%.

### Example 4

### Orthomyxoviridae (e.g., Influenza)

The tissue-culture-adapted PR8 virus (ATCC-VR-1469) were purchased from ATCC. Confluent MDCK cells in 12-well plates were infected with approximately 70 p.f.u. of virus and incubated for 1 h at 37° C. Cells were then washed with PBS and a 50:50 mix of 1.2% agarose: 2× virus growth DMEM containing oseltamivir or tested compounds at final concentrations of 100 nM or 1 μM. Plates were harvested 72 h later, stained with Crystal Violet and plaques were counted. Compared to oseltamivir treated wells, treatment with STF-00203577-001, STF-00204266-001, STF-00204657-001, or STF-00204460-001 resulted in smaller-sized plaques. Thus, the compounds disclosed herein, including those tested in this example, can be used to treat individuals with an orthomyxoviridae infection (e.g., influenza).

### Paramyxoviridae (e.g., RSV)

RSV-luc5 virus was purchased from https://www.viratree.com/product/rsv-luc5/. HeLa cells were plated in 96-well BLACK clear bottom plates at 5,000 cells/well overnight. Cells were infected RSV-luc5 for 1 hour. Cells were then treated with compounds at various concentrations for 3 days. Luciferase activity was measured using Bright-Glo Luciferase as a read-out for viral replication. Treatment with STF-00203577-001, STF-00204266-001, STF-00204657-001, or STF-00204460-001 significantly inhibited RSV replication with an EC₅₀ of 1.1, 1.88, 2.52, and 1.11 μM, respectively. Thus, the compounds disclosed herein, including those tested in this example, can be used to treat individuals with a paramyxoviridae infection (e.g., RSV).

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112 (f) or 35 U.S.C. § 112 (6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112 (6) is not invoked.

1. A method of treatment, comprising: administering to an individual who has a fatty liver and/or a non-flaviviridae viral infection, a therapeutically effective amount of a compound selected from the group consisting of:
(a) a compound of Formula I: 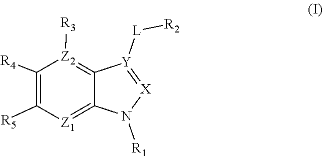
or pharmaceutically acceptable salt thereof, wherein:
X is C or N;
Y is C or N;
Z₁ is C or N;
Z₂ is C or N;
R₁ is hydrogen; C₁-C₆ alkyl; C₁-C₆ alkyl substituted with a substituted or unsubstituted C₃-C₈ cycloalkyl, 5-8 membered heterocyclyl, or a 6 membered aryl group; C₂-C₆ alkenyl; substituted or unsubstituted C₃-C₈ cycloalkyl, —CO—(C₃-C₈ cycloalkyl), —CO—(C₁-C₆ alkyl), —CO-aryl, —CO-heteroaryl, —CO-heterocyclyl, —SO₂—(C₁-C₆ alkyl), or —SO₂—(C₃-C₈ cycloalkyl) group; or R₁ and R₂ together form a 12-25 membered heterocycle, or R₁ and R₅ together form a 12-25 membered heterocycle;
L is a bond, —CONH—, —NH—CO—, substituted or unsubstituted C₁-C₅ alkylene, substituted or unsubstituted C₂-C₅ heteroalkylene, a substituted or unsubstituted 5 membered heteroaryl group, or a substituted or unsubstituted 5-7 membered heterocyclyl, C₅-C₇ cycloalkyl, 5-6 membered heteroaryl, or a 6 membered aryl group; or a combination thereof;
R₂ is —NH₂, —NHR′, —NR′R′, —NHCOR′, —NR′COR′, —NHSO₂R′, —NR′SO₂R′, —NHSO₂NH₂, —NHSO₂NHR′, —NHC(O)NH₂, —NHC(O)NHR′, —N(R′)SO₂NH₂, —N(R)SO₂NHR′, —N(R′)C(O)NH₂, and —N(R′)C(O)NHR′, or a substituted or unsubstituted 5-7 membered heterocyclyl, C₅-C₇ cycloalkyl, 5-6 membered heteroaryl, or a 6 membered aryl group;
R₃, R₄, and R₅ are independently hydrogen, halo, —CN, —OH, —OR′, —NH₂, —NHR′, —NR′R′, —NHCOR′, —NR′COR′, —NHSO₂R′, —NR′SO₂R′, —NHSO₂NH₂, —NHSO₂NHR′, —NHC(O)NH₂, —NHC(O)NHR′, —N(R′)SO₂NH₂, —N(R′)SO₂NHR′, —N(R′)C(O)NH₂, and N(R)C(O)NHR′, —SO₂R′, —SO₂NH₂, SO₂NHR′, SO₂NR′R′, —CONH₂, —CONHR′, —CONR′R′, —CO₂H, —CO₂R′, or a substituted or unsubstituted C₁-C₆ alkyl, C₃-C₈ cycloalkyl, aryl, heteroaryl, or heterocyclyl group; and
R′ is a substituted or unsubstituted C₁-C₆ alkyl, C₂-C₆ alkenyl, C₂-C₆ alkynyl, C₃-C₈ cycloalkyl, aryl, heteroaryl, or heterocyclyl group, or two R′ groups together with the nitrogen atom to which they are bonded form a heterocyclic ring;
(b) a compound of Formula II: 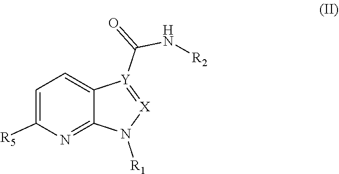
or pharmaceutically acceptable salt thereof, wherein:
X is C or N;
Y is C or N;
R₁ is hydrogen, branched or linear C₁-C₅ alkyl, C₂-C₁₅ alkenyl, unsubstituted or substituted cycloalkyl, —CO-(cycloalkyl), —SO₂-(cycloalkyl) group, or —(CH₂)ₙ—R₁₁, or R₅ and R₁ together form a 12-18 membered heterocycle; n is 1 or 2;
R₂ is substituted or unsubstituted piperidinyl, 4-pyridyl, pyrrolidinyl, piperazinyl, benzyl, substituted phenyl, or pirazolyl group;
R₅ is R₅₁R₅₂N— or R₅₄O—;
R₅₁ is H or substituted or unsubstituted C₁-C₃ alkyl; R₅₂ is C₆-C₈ cycloalkyl, substituted or unsubstituted linear C₁-C₃ alkyl, or branched C₄-C₅ alkyl or R₅₁ and R₅₂ together with the nitrogen atom to which they are bonded form a 6, 7, 8, or 9-membered heterocyclyl ring containing up to 3 heteroatoms optionally substituted, other than the azaindazole moiety to which it is already attached, by a substituted or unsubstituted benzyl acyl, or sulfonyl group; R₅₄ is H, substituted or unsubstituted benzyl group, branched C₃-C₈ alkyl, unsubstituted C₅-C₈ cycloalkyl, or C₅-C₈ cycloalkyl substituted with one or more linear or branched C₁-C₄ alkyl groups; R₁₁ is C₅-C₈ cycloalkyl or substituted or unsubstituted aryl, or substituted or unsubstituted heteroaryl;
(c) a compound of Formula III: 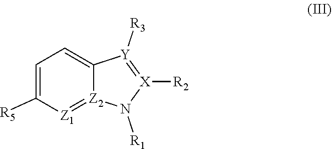
or pharmaceutically acceptable salt thereof, wherein:
X is C or N;
Y is C or N;
Z₁ is C or N;
Z₂ is C or N;
R₁ is hydrogen, branched or linear C₁-C₅ alkyl, C₂-C₁₅ alkenyl, unsubstituted or substituted cycloalkyl, —CO-(cycloalkyl), —SO₂-(cycloalkyl) group, or —(CH₂)ₙ—R₁₁, or R₅ and R₁ together form a 12-18 membered heterocycle; n is 1 or 2;
R₂ is substituted or unsubstituted phenyl, piperidinyl, 4-pyridyl, pyrrolidinyl, piperazinyl, benzyl, substituted phenyl, or pirazolyl group; R₅ is R₅₁R₅₂N— or R₅₄O—;
R₅₁ is H or substituted or unsubstituted C₁-C₃ alkyl; R₅₂ is C₆-C₈ cycloalkyl, substituted or unsubstituted linear C₁-C₃ alkyl, or branched C₄-C₅ alkyl or R₅₁ and R₅₂ together with the nitrogen atom to which they are bonded form a 6, 7, 8, or 9-membered heterocyclyl ring containing up to 3 heteroatoms optionally substituted, other than the azaindazole moiety to which it is already attached, by a substituted or unsubstituted benzyl acyl, or sulfonyl group; R₅₄ is H, substituted or unsubstituted benzyl group, branched C₃-C₈ alkyl, unsubstituted C₅-C₈ cycloalkyl, or C₅-C₈ cycloalkyl substituted with one or more linear or branched C₁-C₄ alkyl groups; R₁ is C₅-C₈ cycloalkyl or substituted or unsubstituted aryl, or substituted or unsubstituted heteroaryl;
R₃ and R₅ are independently hydrogen, halo, —CN, —OH, —OR′, —NH₂, —NHR′, —NR′R′, —NHCOR′, —NR′COR′, —NHSO₂R′, —NR′SO₂R′, —NHSO₂NH₂, —NHSO₂NHR′, —NHC(O)NH₂, —NHC(O)NHR′, —N(R′)SO₂NH₂, —N(R′)SO₂NHR′, —N(R′)C(O)NH₂, and N(R)C(O)NHR′, —SO₂R′, —SO₂NH₂, SO₂NHR′, SO₂NR′R′, —CONH₂, —CONHR′, —CONR′R′, —CO₂H, —CO₂R′, or a substituted or unsubstituted C₁-C₆ alkyl, C₃-C₈ cycloalkyl, aryl, heteroaryl, or heterocyclyl group;
(d) a compound of Formula IV: 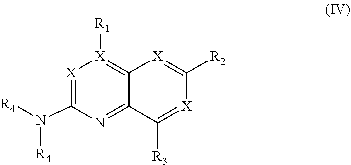
or pharmaceutically acceptable salt thereof, wherein:
each X is independently selected from C or N;
R¹ is absent, hydrogen, (C₁-C₈) alkyl, NR⁹R¹⁰, halo, amino, —C≡N, (C₂-C₈) alkenyl, (C₂-C₈) alkynyl, (C C₈) haloalkyl, (C₂-C₈) haloalkenyl, (C₂-C₈) haloalkynyl, (C₁-C₈) alkoxy, (C₁-C₈) haloalkoxy, (C₁-C₈) alkyl (C₁-C₆) alkoxy, 4-7 membered heterocyclyl, 5-6 membered heteroaryl, CH(O), C(O)OR⁸, SF₅, —OH, —SH, (C₁-C₆) hydroxyalkyl, (C₁-C₄) alkylsulfonyl, aminosulfonyl, amino (C₁-C₄) alkylsulfonyl or aryl;
R² is C(O)NR¹¹R¹², C(O)R¹³, 5-membered heterocycle, 5-membered substituted heterocycle, 5-membered heteroaryl, 5-membered substituted heteroaryl, 6-membered heterocycle, 6-membered substituted heterocycle, 6-membered heteroaryl, 6-membered substituted heteroaryl, 5-membered cycloalkyl, or 5-membered substituted cycloalkyl, 6-membered cycloalkyl, or 6-membered substituted cycloalkyl;
R³ is H, (C₁-C₈) alkyl, (C₁-C₈) alkenyl, (C₁-C₈) alkynyl, (C₁-C₈) alkoxy, hydroxyl, halo, amino, amido, amino (C₁-C₈) alkylamido, heterocyclyl, sulfonyl, aminosulfonyl, amino (C₁-C₈) alkysulfonyl, cyano, or (C₁-C₃) haloalkyl;
each R⁴ is independently selected from (C₁-C₈) alkyl, either unsubstituted or substituted with halo, (C₁-C₈) alkoxy, (C₁-C₈) haloaikoxy, S(O)—R⁶, S(O)₂, S(O)₂—R⁶, S(O)₂, C(O)R⁶, C(O)OR⁷ or (C₁-C₈) cycloalkyl; or both R⁴ together form a (C₁-C₈) cycloalkyl, a (C₁-C₈) substituted cycloalkyl, a (C₁-C₈) heterocycloalkyl, a (C₁-C₈) substituted heterocycloalkyl, a (C₁-C₈) aryl, a (C₁-C₈) substituted aryl, a (C₁-C₈) heteroaryl or a (C₁-C₈) substituted heteroaryl;
R⁶ is H, alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, halo, (C₁-C₄) haloalkyl and cyano;
R⁷ is H, alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, halo, (C₁-C₄) haloalkyl and cyano;
R⁸ is H, alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, halo, (C₁-C₄) haloalkyl and cyano;
R⁹ is H, (C₁-C₈) alkyl, said (C₁-C₈) alkyl being unsubstituted or substituted with one or more halo;
R¹⁰ is H, (C₁-C₈) alkyl, said (C₁-C₈) alkyl being unsubstituted or substituted with one or more halo, or R⁹ and R¹⁰, together with the nitrogen atom to which they are attached, form a 4 or 5 membered nitrogen containing heterocycle, said 4 or 5 membered nitrogen containing heterocycle being unsubstituted or substituted with one or more halo; R is H, (C₁-C₄) alkyl, (C₁-C₄) haloalkyl, 5-6 membered heterocycle or 5-6 membered heteroaryl;
R¹¹ is selected from H, (C₁-C₄) alkyl, (C₁-C₄) haloalkyl, a 5-membered heterocycle, 5-membered substituted heterocycle, 5-membered heteroaryl, 5-membered substituted heteroaryl, 6-membered heterocycle, 6-membered substituted heterocycle, 6-membered heteroaryl, 6-membered substituted heteroaryl, 5-membered cycloalkyl, or 5-membered substituted cycloalkyl, 6-membered cycloalkyl, or a 6-membered substituted cycloalkyl;
R¹² is selected from H, (C₁-C₄) alkyl, (C₁-C₄) haloalkyl, a 5-membered heterocycle, 5-membered substituted heterocycle, 5-membered heteroaryl, 5-membered substituted heteroaryl, 6-membered heterocycle, 6-membered substituted heterocycle, 6-membered heteroaryl, 6-membered substituted heteroaryl, 5-membered cycloalkyl, or 5-membered substituted cycloalkyl, 6-membered cycloalkyl, or a 6-membered substituted cycloalkyl;
R¹¹ and R¹², together with the nitrogen atom to which they are attached, form a 5-8 membered nitrogen containing heterocycle, said 5-8 membered nitrogen containing heterocycle being unsubstituted or substituted with OH, halo, ═O, or (C₁-C₈) alkyl;
R¹³ is OH, O—(C₁-C₄) alkyl;
R¹⁵ is H, (C₁-C₆) alkyl, (C₁-C) haloalkyl, 4-7 membered heterocycle, 5-6 membered heteroaryl, 3-7 membered cycloalkyl, and wherein each of said 4-7 membered heterocycle, 5-6 membered heteroaryl, 3-7 membered cycloalkyl is unsubstituted or substituted at a substitutable position with one or more ═O, OH, (C₁-C₈) alkyl, (C₁-C₈) haloalkyl, (C₁-C₈) alkoxy, amino, or, (C₁-C₈) aminoalkyl; and
R¹⁶ is H, (C₁-C₈) alkyl, or (C₁-C₈) haloalkyl;
(e) a compound of Formula V: 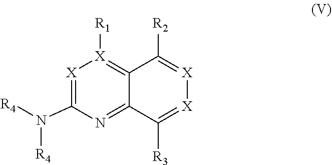
or pharmaceutically acceptable salt thereof, wherein:
each X is independently selected from C or N;
R¹ is absent, hydrogen, (C₁-C₈) alkyl, NR⁹R¹⁰, halo, amino, —C≡N, (C₂-C₈) alkenyl, (C₂-C₈) alkynyl, (C C₈) haloalkyl, (C₂-C₈) haloalkenyl, (C₂-C₈) haloalkynyl, (C₁-C₈) alkoxy, (C₁-C₈) haloalkoxy, (C₁-C₈) alkyl (C₁-C₆) alkoxy, 4-7 membered heterocyclyl, 5-6 membered heteroaryl, CH(O), C(O)OR⁸, SF₅, —OH, —SH, (C₁-C₆) hydroxyalkyl, (C₁-C₄) alkylsulfonyl, aminosulfonyl, amino (C₁-C₄) alkylsulfonyl or aryl;
R² is C(O)NR¹¹R¹², C(O)R¹³, 5-membered heterocycle, 5-membered substituted heterocycle, 5-membered heteroaryl, 5-membered substituted heteroaryl, 6-membered heterocycle, 6-membered substituted heterocycle, 6-membered heteroaryl, 6-membered substituted heteroaryl, 5-membered cycloalkyl, or 5-membered substituted cycloalkyl, 6-membered cycloalkyl, or 6-membered substituted cycloalkyl;
R³ is H, (C₁-C₈) alkyl, (C₁-C₈) alkenyl, (C₁-C₈) alkynyl, (C₁-C₈) alkoxy, hydroxyl, halo, amino, amido, amino (C₁-C₈) alkylamido, heterocyclyl, sulfonyl, aminosulfonyl, amino (C₁-C₈) alkysulfonyl, cyano, or (C₁-C₃) haloalkyl;
each R⁴ is independently selected from (C₁-C₈) alkyl, either unsubstituted or substituted with halo, (C₁-C₈) alkoxy, (C₁-C₈) haloaikoxy, S(O)—R⁶, S(O)₂, S(O)₂—R⁶, S(O)₂, C(O)R⁶, C(O)OR⁷ or (C₁-C₈) cycloalkyl; or both R⁴ together form a (C₁-C₈) cycloalkyl, a (C₁-C₈) substituted cycloalkyl, a (C₁-C₈) heterocycloalkyl, a (C₁-C₈) substituted heterocycloalkyl, a (C₁-C₈) aryl, a (C₁-C₈) substituted aryl, a (C₁-C₈) heteroaryl or a (C₁-C₈) substituted heteroaryl;
R⁶ is H, alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, halo, (C₁-C₄) haloalkyl and cyano;
R⁷ is H, alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, halo, (C₁-C₄) haloalkyl and cyano;
R⁸ is H, alkyl, substituted alkyl, alkenyl, substituted alkenyl, alkynyl, substituted alkynyl, aryl, substituted aryl, acyl, substituted acyl, carboxyl, alkoxycarbonyl, substituted alkoxycarbonyl, aminoacyl, substituted aminoacyl, amino, substituted amino, acylamino, substituted acylamino, halo, (C₁-C₄) haloalkyl and cyano;
R⁹ is H, (C₁-C₈) alkyl, said (C₁-C₈) alkyl being unsubstituted or substituted with one or more halo;
R¹⁰ is H, (C₁-C₈) alkyl, said (C₁-C₈) alkyl being unsubstituted or substituted with one or more halo, or R⁹ and R¹⁰, together with the nitrogen atom to which they are attached, form a 4 or 5 membered nitrogen containing heterocycle, said 4 or 5 membered nitrogen containing heterocycle being unsubstituted or substituted with one or more halo; R is H, (C₁-C₄) alkyl, (C₁-C₄) haloalkyl, 5-6 membered heterocycle or 5-6 membered heteroaryl;
R¹¹ is selected from H, (C₁-C₄) alkyl, (C₁-C₄) haloalkyl, a 5-membered heterocycle, 5-membered substituted heterocycle, 5-membered heteroaryl, 5-membered substituted heteroaryl, 6-membered heterocycle, 6-membered substituted heterocycle, 6-membered heteroaryl, 6-membered substituted heteroaryl, 5-membered cycloalkyl, or 5-membered substituted cycloalkyl, 6-membered cycloalkyl, or a 6-membered substituted cycloalkyl;
R¹² is selected from H, (C₁-C₄) alkyl, (C₁-C₄) haloalkyl, a 5-membered heterocycle, 5-membered substituted heterocycle, 5-membered heteroaryl, 5-membered substituted heteroaryl, 6-membered heterocycle, 6-membered substituted heterocycle, 6-membered heteroaryl, 6-membered substituted heteroaryl, 5-membered cycloalkyl, or 5-membered substituted cycloalkyl, 6-membered cycloalkyl, or a 6-membered substituted cycloalkyl;
R¹¹ and R¹², together with the nitrogen atom to which they are attached, form a 5-8 membered nitrogen containing heterocycle, said 5-8 membered nitrogen containing heterocycle being unsubstituted or substituted with OH, halo, ═O, or (C₁-C₈) alkyl;
R¹³ is OH, O—(C₁-C₄) alkyl;
R¹⁵ is H, (C₁-C₆) alkyl, (C₁-C) haloalkyl, 4-7 membered heterocycle, 5-6 membered heteroaryl, 3-7 membered cycloalkyl, and wherein each of said 4-7 membered heterocycle, 5-6 membered heteroaryl, 3-7 membered cycloalkyl is unsubstituted or substituted at a substitutable position with one or more ═O, OH, (C₁-C₈) alkyl, (C₁-C₈) haloalkyl, (C₁-C₈) alkoxy, amino, or, (C₁-C₈) aminoalkyl; and
R¹⁶ is H, (C₁-C₈) alkyl, or (C₁-C₈) haloalkyl; and
(f) a compound of Formula VI: 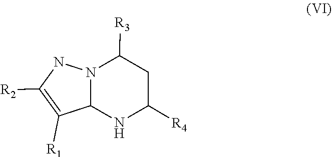
or pharmaceutically acceptable salt thereof, wherein:
R₁ is hydrogen; halo, haloalkyl C₁-C₆ alkyl; C₁-C₆ alkyl substituted with a substituted or unsubstituted C₃-C₈ cycloalkyl, 5-8 membered heterocyclyl, or a 6 membered aryl group; C₂-C₆ alkenyl; substituted or unsubstituted C₃-C₈ cycloalkyl, —CO—(C₃-C₈ cycloalkyl), —CO—(C₁-C₆ alkyl), —CO-aryl, —CO-heteroaryl, —CO-heterocyclyl, —SO₂ (C₁-C₆ alkyl), or —SO₂—(C₃-C₈ cycloalkyl) group;
R² is C(O)NR¹¹R¹², C(O)R¹³, 5-membered heterocycle, 5-membered substituted heterocycle, 5-membered heteroaryl, 5-membered substituted heteroaryl, 6-membered heterocycle, 6-membered substituted heterocycle, 6-membered heteroaryl, 6-membered substituted heteroaryl, 5-membered cycloalkyl, or 5-membered substituted cycloalkyl, 6-membered cycloalkyl, or 6-membered substituted cycloalkyl;
R³ is H, (C₁-C₈) alkyl, (C₁-C₈) alkenyl, (C₁-C₈) alkynyl, (C₁-C₈) alkoxy, hydroxyl, halo, amino, amido, amino (C₁-C₈) alkylamido, heterocyclyl, sulfonyl, aminosulfonyl, amino (C₁-C₈) alkysulfonyl, cyano, or (C₁-C₃) haloalkyl;
R¹¹ is selected from H, (C₁-C₄) alkyl, (C₁-C₄) haloalkyl, a 5-membered heterocycle, 5-membered substituted heterocycle, 5-membered heteroaryl, 5-membered substituted heteroaryl, 6-membered heterocycle, 6-membered substituted heterocycle, 6-membered heteroaryl, 6-membered substituted heteroaryl, 5-membered cycloalkyl, or 5-membered substituted cycloalkyl, 6-membered cycloalkyl, or a 6-membered substituted cycloalkyl or a alkyl-substituted with one or more of a 5-membered heterocycle, 5-membered substituted heterocycle, 5-membered heteroaryl, 5-membered substituted heteroaryl, 6-membered heterocycle, 6-membered substituted heterocycle, 6-membered heteroaryl, 6-membered substituted heteroaryl, 5-membered cycloalkyl, or 5-membered substituted cycloalkyl, 6-membered cycloalkyl, or a 6-membered substituted cycloalkyl R¹² is selected from H, (C₁-C₄) alkyl, (C₁-C₄) haloalkyl, a 5-membered heterocycle, 5-membered substituted heterocycle, 5-membered heteroaryl, 5-membered substituted heteroaryl, 6-membered heterocycle, 6-membered substituted heterocycle, 6-membered heteroaryl, 6-membered substituted heteroaryl, 5-membered cycloalkyl, or 5-membered substituted cycloalkyl, 6-membered cycloalkyl, or a 6-membered substituted cycloalkyl or a alkyl-substituted with one or more of a 5-membered heterocycle, 5-membered substituted heterocycle, 5-membered heteroaryl, 5-membered substituted heteroaryl, 6-membered heterocycle, 6-membered substituted heterocycle, 6-membered heteroaryl, 6-membered substituted heteroaryl, 5-membered cycloalkyl, or 5-membered substituted cycloalkyl, 6-membered cycloalkyl, or a 6-membered substituted cycloalkyl
R¹¹ and R¹², together with the nitrogen atom to which they are attached, form a 5-8 membered nitrogen containing heterocycle, said 5-8 membered nitrogen containing heterocycle being unsubstituted or substituted with OH, halo, ═O, or (C₁-C₈) alkyl; and
R₄ is hydrogen; halo, haloalkyl C₁-C₆ alkyl; C₁-C₆ alkyl substituted with a substituted or unsubstituted C₃-C₈ cycloalkyl, 5-8 membered heterocyclyl, a 5-8 membered aryl, a 5-8 membered heteroaryl, C₂-C₆ alkenyl; substituted or unsubstituted C₃-C₈ cycloalkyl, —CO—(C₃-C₈ cycloalkyl), —CO—(C₁-C₆ alkyl), —CO-aryl, —CO-heteroaryl, —CO-heterocyclyl, —SO₂—(C₁-C₆ alkyl), or —SO₂—(C₃-C₈ cycloalkyl) group.
In some embodiments, R₃ is selected from hydrogen, (C₁-C₈) alkyl, halo or haloalkyl. In some instances, R₃ is hydrogen. In some instances, R₃ is a (C₁-C₈) alkyl, such as methyl, ethyl, propyl, i-propyl, butyl, t-butyl, i-butyl or pentyl. In certain instances, R₃ is methyl. In certain instances, R₃ is t-butyl. In some instances, R₃ is halo, such as fluoro, chloro or bromo. In certain instances, R₃ is fluoro. In some instances, R₃ is a haloalkyl, such as CF₃, CCl₃ or CBr₃. In certain instances, R₃ is CF₃.

2. The method of claim 1, wherein the individual has a non-flaviviridae viral infection selected from the group consisting of: an hepadnaviridae virus infection, an alphavirus infection, a coronaviridae infection, a paramyxoviridae infection, and an orthomyxoviridae infection.

3. The method of claim 1, wherein the individual has a hepatitis B virus (HBV), Venezuelan equine encephalitis virus (VEEV), O′nyong nyong virus (ONNV), SARS-CoV-2, Semliki Forest virus (SFV), or chikungunya virus (CHIKV) viral infection.

4. The method of claim 1, wherein the individual has a hepatitis B virus (HBV), Venezuelan equine encephalitis virus (VEEV), SARS-CoV-2, or chikungunya virus (CHIKV) viral infection.

5. The method of claim 1, wherein the compound is co-administered with a second agent.

6. The method of claim 2, wherein the compound is co-administered with a second antiviral agent.

7. The method of claim 1, wherein the compound is administered at a concentration in a range of from at 8 to 100 mg/kg.

8. The method of claim 1, wherein the compound is administered for 3 days or more.

9. The method of claim 1, wherein the compound is A27:

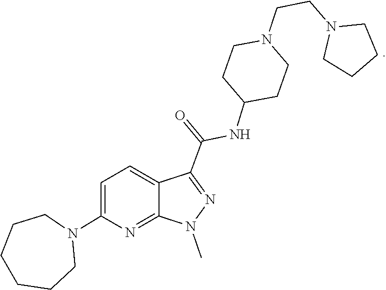 [SMILES: Cn1nc(C(=O)NC2CCN(CCN3CCCC3)CC2)c2ccc(N3CCCCCC3)nc21]

10. A compound, or pharmaceutically acceptable salt thereof, selected from the group consisting of:

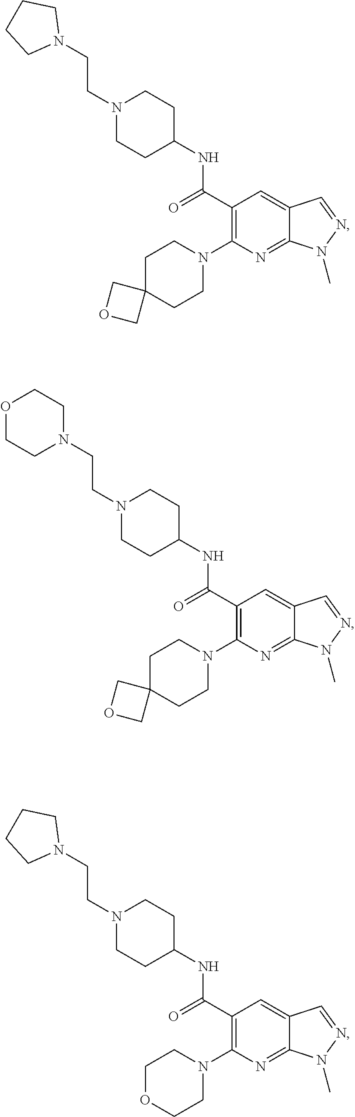 [SMILES: Cn1ncc2cc(C(=O)NC3CCN(CCN4CCCC4)CC3)c(N3CCC4(CC3)COC4)nc21 ; Cn1ncc2cc(C(=O)NC3CCN(CCN4CCOCC4)CC3)c(N3CCC4(CC3)COC4)nc21 ; Cn1ncc2cc(C(=O)NC3CCN(CCN4CCCC4)CC3)c(N3CCOCC3)nc21]

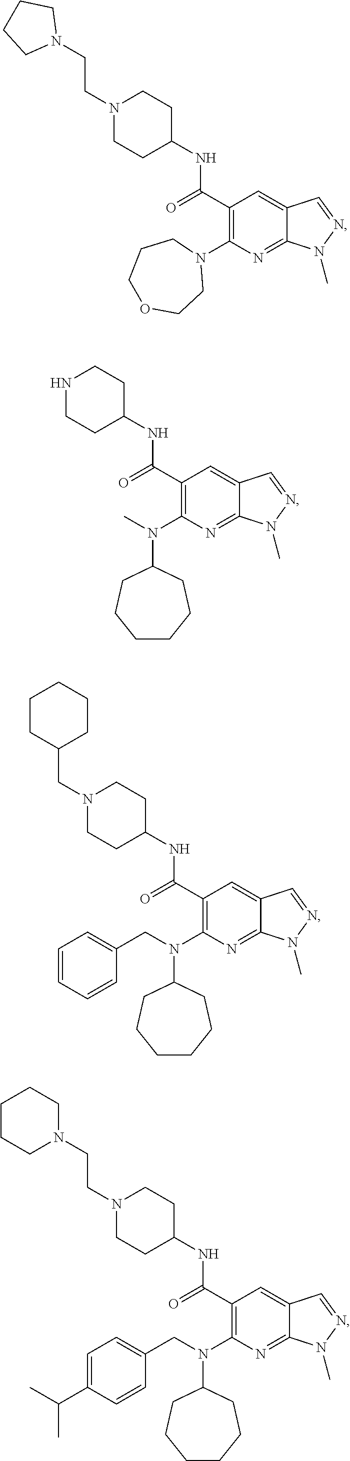 [SMILES: Cn1ncc2cc(C(=O)NC3CCN(CCN4CCCC4)CC3)c(N3CCCOCC3)nc21 ; CN(c1nc2c(cnn2C)cc1C(=O)NC1CCNCC1)C1CCCCCC1 ; Cn1ncc2cc(C(=O)NC3CCN(CC4CCCCC4)CC3)c(N(Cc3ccccc3)C3CCCCCC3)nc21 ; CC(C)c1ccc(CN(c2nc3c(cnn3C)cc2C(=O)NC2CCN(CCN3CCCCC3)CC2)C2CCCCCC2)cc1]

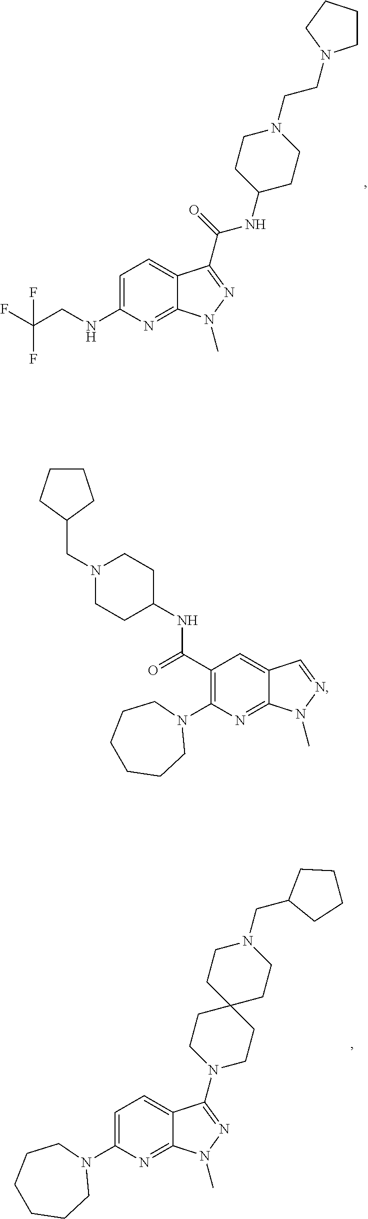 [SMILES: Cn1nc(C(=O)NC2CCN(CCN3CCCC3)CC2)c2ccc(NCC(F)(F)F)nc21 ; Cn1ncc2cc(C(=O)NC3CCN(CC4CCCC4)CC3)c(N3CCCCCC3)nc21 ; Cn1nc(N2CCC3(CCN(CC4CCCC4)CC3)CC2)c2ccc(N3CCCCCC3)nc21]

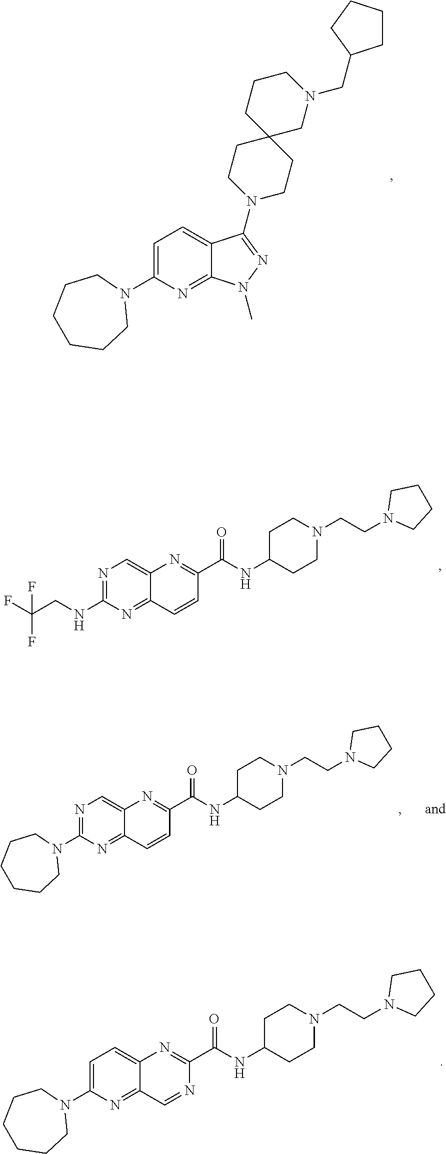 [SMILES: Cn1nc(N2CCC3(CCCN(CC4CCCC4)C3)CC2)c2ccc(N3CCCCCC3)nc21 ; O=C(NC1CCN(CCN2CCCC2)CC1)c1ccc2nc(NCC(F)(F)F)ncc2n1 ; O=C(NC1CCN(CCN2CCCC2)CC1)c1ccc2nc(N3CCCCCC3)ncc2n1 ; O=C(NC1CCN(CCN2CCCC2)CC1)c1ncc2nc(N3CCCCCC3)ccc2n1]

11. The compound, or pharmaceutically acceptable salt thereof, of claim 10, selected from the group consisting of:

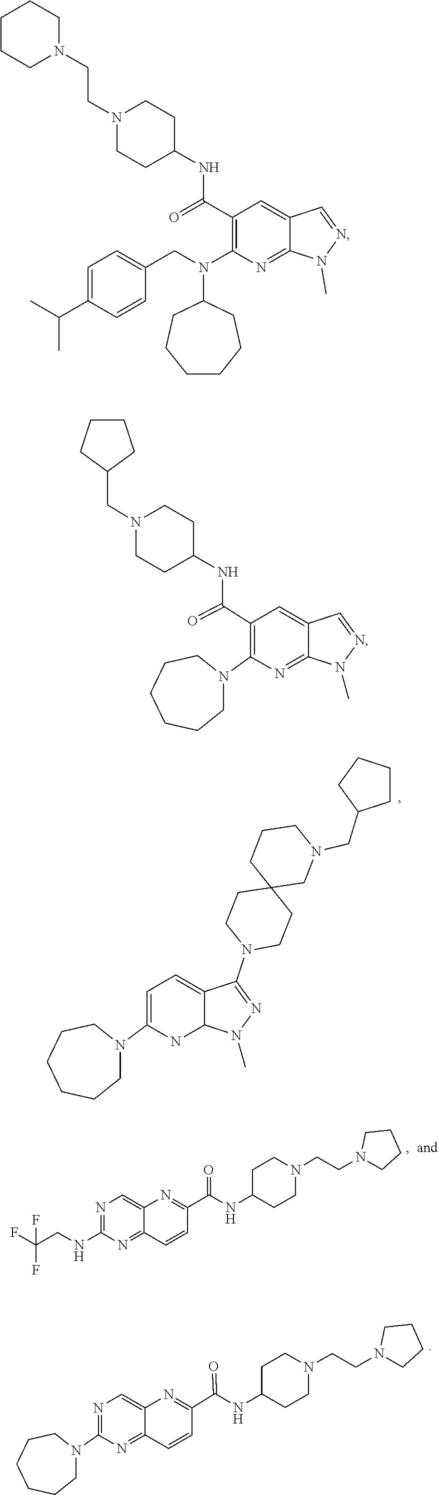 [SMILES: CC(C)c1ccc(CN(c2nc3c(cnn3C)cc2C(=O)NC2CCN(CCN3CCCCC3)CC2)C2CCCCCC2)cc1 ; Cn1ncc2cc(C(=O)NC3CCN(CC4CCCC4)CC3)c(N3CCCCCC3)nc21 ; CN1N=C(N2CCC3(CCCN(CC4CCCC4)C3)CC2)C2=CC=C(N3CCCCCC3)NC21 ; O=C(NC1CCN(CCN2CCCC2)CC1)c1ccc2nc(NCC(F)(F)F)ncc2n1 ; O=C(NC1CCN(CCN2CCCC2)CC1)c1ccc2nc(N3CCCCCC3)ncc2n1]

12. A composition comprising:
(a) the compound, or pharmaceutically acceptable salt thereof, of claim 10; and
(b) a pharmaceutically acceptable carrier, excipient, or diluent.

13. The composition of claim 12, wherein the composition is a medicament for the treatment of fatty liver and/or a viral infection.
